(19) United States
(12) Patent Application Publication
(10) Pub. No.: US 2010/0035945 A1
(43) Pub. Date: Feb. 11, 2010

(54) SMALL MOLECULE INHIBITORS OF BACTERIAL DAM DNA METHYLTRANSFERASES

(76) Inventors: Xiaodong Cheng, Decatur, GA (US); John R. Horton, Stone Mountain, GA (US); Zhe Yang, Atlanta, GA (US); Daniel Kalman, Atlanta, GA (US); Xing Zhang, Decatur, GA (US); Stanley Hattman, Rochester, NY (US); Albert Jeltsch, Ritterhude (DE)

Correspondence Address: GREENLEE WINNER AND SULLIVAN P C, 4875 PEARL EAST CIRCLE, SUITE 200, BOULDER, CO, 80301, US

(21) Appl. No.: 11/720,971

(22) PCT Filed: Dec. 6, 2005
(86) PCT No.: PCT/US05/44277

§ 371 Date: Aug. 7, 2009

Related U.S. Application Data
(60) Provisional application No. 60/634,066, filed on Dec. 6, 2004.

(51) Int. Cl. A61K 31/427; G06G 7/60; C12N 9/10; A61P 31/04

(52) U.S. Cl. 514/370; 703/11; 435/193

(57) ABSTRACT

Disclosed are compounds, crystal structures, data representations, methods of using, and methods of identifying compounds in relation to DNA methylation and inhibition of methylation. In embodiments, DNA methylation is by DNA-adenine methyltransferases (Dam). In an embodiment, compounds are used to treat a host suspected of infection by a pathogenic organism. In an embodiment, virulence of a pathogenic bacterium is modified by treatment with an agent capable of inhibiting a bacterial Dam enzyme. In an embodiment, compounds and methods are disclosed regarding Dam inhibitors.

### STATEMENT REGARDING FEDERALLY SPONSORED RESEARCH OR DEVELOPMENT

This invention was made with government support under GM49245 awarded by the National Institutes of Health. The government has certain rights in the invention.

### BACKGROUND OF THE INVENTION

Pathogenic bacteria cause a variety of disease in humans, which manifest in a range of symptoms from mild to severe, and can lead to death. Worldwide infectious diseases are a leading cause of death. Pathogenic bacteria are of particular concern given the development of increased multi-drug resistance and horizontal transfer of resistance genes. This development of bacterial resistance to antibiotics is an ongoing and increasing problem. There is a continued need for new classes of antibiotics and, in particular, antibiotics that are less likely to lose efficacy due to resistance development by bacteria. The present invention provides a new class of antibiotics that interfere with DNA methylation by inhibiting DNA adenine methylase (“Dam”). Because Dam is required for virulence in a variety of bacteria, inhibiting Dam reduces virulence. Inhibitors of Dam are particularly beneficial as antibiotics because they do not affect mammalian cell DNA-MTases and, accordingly, have minimal toxicity for the host organism. In addition, because only bacterial virulence is reduced, the opportunity for bacteria to develop resistance to Dam inhibitors is also reduced.

DNA methylation is a process whereby methyl groups are added to DNA and provides a mechanism to control gene expression. Accordingly, DNA methylation plays an important role in a large number and variety of biological processes. DNA from most prokaryotes and eukaryotes contains the methylated bases 4-methylcyosine (N4mC), 5 methylcytosine (5mC) and 6-methyladenine (N6mA). Modifications by methylation are introduced after DNA replication by DNA methyltransferases (“MTases”). DNA MTases catalyze methyl group transfer from donor S-adenosyl-L-methionine (“AdoMet”) to produce S-adenosyl-L-homocysteine (AdoHcy) and methylated DNA (FIG. 1). Generally, MTases recognize a specific sequence and utilize a “base flipping” mechanism (Klimasauskas et al., 1994) to rotate the target base within that sequence out of the DNA helix and into the MTases active-site pocket.

While most prokaryote DNA MTases are components of restriction-modification systems and function as part of a phage defense mechanism, some MTases are not associated with cognate restriction enzymes; e.g. the E. coli DNA adenine MTase (Dam), which methylates an exocyclic amino nitrogen (N6) of the Adenosine in GATC sequence (FIG. 1) (Hattman et al., 1978; Lacs and Greenberg, 1977). Dam Mtase gene orthologs are widespread among enteric bacteria and their bacteriophages (see review by Hattman & Malygin, 2004).

Dam methylation is important in prokaryotic DNA replication. For example, there is a cluster of GATC sites near the origin of replication of E. coli and Salmonella typhimurium, all of which are conserved between the two species. It is the hemimethylated GATC sites, produced immediately following DNA replication, that regulate the timing and targeting of a number of cellular functions (Messer & Noyer-Weidner, 1988). For example, SeqA specifically binds these hemimethylated GATC sites, causes delay of their full methylation (Guarne et al. 2002; Kang et al. 1999; Lu et al. 1994) and, in part, controls DNA replication.

DNA-adenine methylation at specific GATC sites plays a central role in bacterial gene expression, DNA replication, mismatch repair, and is essential for bacterial virulence for many Gram-negative bacteria. Dam methylation regulates the expression of certain genes in E. coli (Oshima et al. 2002; Lobner-Olesen et al. 2003), and the expression and secretion of Yop virulence proteins under non-permissive conditions in Yersinia pseudotuberculosis (Julio et al. 2002). The expression of pyelonephritis-associated pili (Pap) in uropathogenic E. coli is epigenetically controlled by the binding of the global regulator Lrp to a hemimethylated GATC site (Hernday et al. 2003). In addition, Dam methylation is important in the E. coli mismatch repair system formed by MutSI and MutH (Modrich, 1989; Yang, 2000). In contrast, DNA-adenine methylation has not been observed in humans or other higher eukaryotes.

The mechanism of DNA methylation and base flipping by the EcoDam enzyme has been extensively studied. EcoDam methylates DNA in a processive reaction, in which EcoDam transfers up to 55 methyl groups without dissociation from the DNA molecule (Urig et al., 2002). In such a mode of action, EcoDam exchanges AdoHcy for AdoMet while staying bound to the DNA duplex leading to a processive methylation of the DNA, a mechanism that also holds for other solitary MTases (i.e., no cognate restriction enzyme) (Berdis et al. 1998; Renbaum and Razin, 1992). In contrast, MTases belonging to a restriction-modification system often exhibit a distributive mechanism (as processive methylation of DNA interferes with the biological function of restriction-modification systems) (Jeltsch, 2002). The high processivity is essential to rapidly restore full methylation after replication.

DNA adenine methylation plays an essential role in bacterial virulence (Heithoff et al. 1999; Garcia-Del Portillo et al. 1999). The present invention, therefore, inhibits virulence by inhibiting Dam methylation. The involvement of Dam as a virulence factor was first described for Salmonella enterica serovar Typhimurium, where the dam mutant was out-competed by wildtype in establishing fatal infections in mice and where mice previously infected with the dam mutant were less susceptible to superinfection by the wildtype (Low et al. 2001). Salmonella is one of the most common enteric (intestinal) infections in the U.S. In some states (e.g. Georgia, Maryland) it is the most common, and overall it is the second most common, foodborne illness (usually slightly less frequent than a Campylobacter infection). According to the CDC, approximately 500 to 1,000 persons, or 31% of all food-related deaths are caused by Salmonella infections in the U.S. every year. Salmonella is a type of bacteria that causes typhoid fever and many other infections of intestinal origin. Typhoid fever, rare in the U.S., is caused by a particular strain designated Salmonella typhi. But illness due to other Salmonella strains, called “salmonellosis,” is common in the U.S. Today, the number of known strains (technically termed “serotypes” or “serovars”) of this bacterium total over 2,300 (from CDC web site). It was first shown in Salmonella typhimurium that Dam methylation regulates a bacterium's use of its armament of molecular tools to dodge the immune defenses of mammals. A dam⁻ mutant was avirulent to mice at 10,000 times the LD₅₀ of dam⁺ bacteria, although the mutant bacteria appeared to grow normally. Moreover, infecting mice with dam⁻ mutant cells offered protection against further infection by wild type dam⁻. Accordingly, Dam is an appealing target for drug design (Heithoff et al. 1999; Low et al. 2001).

Yersinia Dam: Yersinia pestis is a species of bacteria that causes plague, an infection that leads to death quickly and that has caused several major epidemics in Europe and Asia over the last 2,000 years. One of the best known was called the Black Death because it turned the skin black. This plague epidemic in the 14th century killed more than one-third of the population of Europe within a few years. In some cities, up to 75 percent of the population died within days, with fever and ulcerated swellings on their skin. The last urban plague epidemic in the United States occurred in Los Angeles in 1925. Since then, an average of 13 cases of plague have been diagnosed each year, primarily in the Southwest, with about 80 percent occurring in the desert areas of New Mexico, Arizona or Colorado and about 9 percent in California. Worldwide, up to 3,000 cases of plague are reported to the World Health Organization each year. Plague is considered one of the most dangerous agents of biological warfare and could be utilized by terrorists in pneumonic form (identified as potential bioterrorism agents by the CDC).

E. coli Dam: Even though, E. coli is a major facultative inhabitant of the large intestine, it is one of the most frequent causes of some of the many common bacterial infections, including cholecystitis, bacteremia, cholangitis, urinary tract infection, and traveler's diarrhea, and other clinical infections such as neonatal meningitis and pneumonia. There are hundreds of strains of this bacterium. One strain, Escherichia coli O157:H7, is an emerging cause of foodborne illness. It produces large quantities of one or more related, potent toxins that cause severe damage to the lining of the intestine. These toxins (verotoxin (VT), shiga-like toxin) are closely related or identical to the toxin produced by Shigella dysenteriae. Escherichia coli O157:H7 infection often leads to bloody diarrhea, and occasionally to kidney failure.

Klebsiella Dam: Although the role of Dam methylation in growth and virulence of Klebsiella has not been established in the art, we examine it because Klebsiella pneumoniae infections are common in hospitals where they cause pneumonia (characterized by emission of bloody sputum) and urinary tract infections in catheterized patients. Klebsiella infections tend to occur in people with a weakened immune system. In fact, K. pneumoniae is second only to E. coli as a urinary tract pathogen. Klebsiella infections are encountered far more often now than in the past especially in neonatal intensive care units. This is probably due to the bacterium's antibiotic resistance properties. Klebsiella species may contain resistance plasmids (R-plasmids) which confer resistance to such antibiotics as ampicillin, carbenicillin, and penicillin. Often, two or more powerful antibiotics are used to help eliminate a Klebsiella infection. To make matters worse, the R-plasmids can be transferred to other enteric bacteria not necessarily of the same species. Accordingly, there is a need for a new class of compounds to inhibit Klebsiella Dam, and thereby effectively treat these opportunistic hospital infections.

In addition, inactivation of Dam MTase attenuates Haemophilus influenzae virulence (Watson et al. 2004). Dam is associated with virulence factors for a growing list of bacterial pathogens including Neisseria meningitides, Yersinia pseudotuberculosis, Vibrio cholerae, Pasteurella multocida, Haemophilus influenzae and Yersinia enterecolitica. (see Low et al. 2001 and Table 1). Although Dam methylation is not essential for viability in many organisms, dam is an essential gene in Vibrio cholerae and Yersinia pseudotuberculosis, under tested growth conditions (Julio et al. 2001). Overproduction of Dam in Yersinia pseudotuberculosis attenuates virulence, secretion of several outer proteins (Yops) and heightened immunity (Julio et al. 2002), although the effect may be indirect through the inhibition of SeqA binding to hemimethylated GATC sites (Lobner-Olesen et al. 2005). A similar rationale may apply to dam plasmid attenuation of virulence in Pasteurella multocida which causes bovine respiratory disease (Chen et al. 2003). Among the Dam molecules examined to date, the Shigella flexnerii dam mutant shows the least effect on virulence (Honma et al. 2004).

Dam inhibitors are useful in reducing and/or preventing virulence associated with a number of pathogenic bacteria. For example, enteropathogenic E. coli (EPEC) is a significant public health concern, especially in developing countries, where it contaminates water supply and causes infant diarrhea (Gill and Hamer, 2001; Goosney et al. 2000; Knutton et al. 19989; Levine and Edelman, 1984), resulting in two million infant deaths per year. EPEC is closely related to enterohemorrhagic E. coli O157:H7 (EHEC), which causes diarrhea and hemorrhagic colitis that can lead to hemolytic uremic syndrome (Riley et al. 1983) and death. In Western nations EHEC is endemic in cattle (Mead et al. 1999), and has been a major source of contamination of ground beef (USDA, 2002). EHEC kills about 60 people per year and infects about 74,000 people in the United States alone (Mead et al. 1999). Currently, antibiotics are contraindicated for EHEC infections because they cause lysis and release of Shiga toxin, which causes renal failure and death. Development of drugs which inhibit expression of virulence factors offers a means to treat EHEC infections.

The present invention provides a method for rational design of, and screening to identify, specific inhibitors of Dam to reduce virulence of pathogenic bacteria. These specific inhibitors can be used to treat humans, as well as other higher eukaryotes that do not have detectable DNA-adenine methylation (Jeltsch, 2002). Accordingly, specific GATC methylation inhibitors can have broad anti-microbial action without affecting host function. There are a number of advantages for targeting factors that influence virulence over, for example, essential enzymes, and include: (1) selection of pathogenic over non-pathogenic bacteria without being toxic to non-pathogenic bacteria; (2) lack of immediate toxicity reduces the risk of rapid development of drug resistance; and (3) continued initial propagation of the pathogen allows the host to mount a stable immune response. Dam deletion mutants of Salmonella can be used as a live attenuated vaccine conferring cross-protective immunity (Dueger et al. 2001, 2003; Heithoff et al. 1999). However, dam mutants would have deficient mismatch DNA repair and consequently an increased rate of spontaneous mutation, which would not be a desirable trait for a live vaccine strain. Compounds having the capacity to affect virulence without affecting growth are less likely to elicit resistance compared to conventional antibiotics. Antibiotic resistance is one of the single greatest public health challenges facing humanity and developing compounds to affect virulence in a range of pathogens can significantly and positively impact treatment of infectious diseases. Because Dam inhibitors can affect the viability of many human bacterial pathogens, they may have widespread applicability in an era of bioterrorism concern. Inhibition of Dam by small molecule inhibitors provides a basis for identifying and developing a new class of antibiotics with broad anti-microbial action. We have determined the three-dimensional structures of two Dam MTases in complexes with DNA: the bacteriophage T4 Dam MTase and the E. coli Dam MTase. These high-resolution structures are used to identify, as well as rationally design, specific Dam MTase inhibitors. These inhibitors are useful in treating a host infected with pathogenic bacteria.

### BRIEF SUMMARY OF THE INVENTION

The present invention is for compounds and method of treating pathogenic organisms in a host. In particular, the invention provides a method to identify compounds capable of modifying activity of a DNA methyltransferase, including modifying activities of AdoMet-dependent MTases from pathogenic bacteria. AdoMet-dependent MTases and related proteins include: HhaI DNA MTase, HhaI MTase-DNA complex, PvuII endonuclease-DNA complex, PvuII DNA MTase, protein arginine MTases PRMT3 and PRMT1, small molecule histamine MTase and its complex with inhibitor, Dnmt3b PWWP domain, MBD4 glycosylase domain, histone H3 Lys9 MTase DIM-5 and its complex with substrate H3 peptide, phage T4 Dam and its complexes with DNA specifically and nonspecifically, protein glutamine-N5 MTase HemK, a nucleosomal dependent histone H3 lysine 79 MTase Dot1p, and HinP1I endonuclease, E. coli Dam and Dam from other pathogenic bacteria. In an embodiment Dam or Dim-5 enzyme activity is modified. In an embodiment Dam enzyme activity is modified. The Dam enzyme activity can be increased or it can be decreased. In a preferred embodiment the Dam enzyme activity is inhibited.

The identification method can be conducted by providing a three-dimensional structure of a Dam enzyme or a Dam enzyme complex. The Dam enzyme can be the entire protein. Alternatively, the Dam enzyme can be a portion of the entire protein, wherein the portion contains one or more of an AdoMet binding pocket, channel into and out of the pocket, a hinge region between the catalytic and DNA binding domains, a DNA binding surface, a unique surface pocket, or any other region that can affect Dam enzyme activity. The structure can be from a Dam enzyme complexed with one or more of the methyl donor (e.g. AdoMet) and DNA. A modifier candidate structure is provided and an interaction energy value calculated from a simulated docking interaction with the candidate structure and the Dam enzyme. A candidate structure is identified as capable of modifying Dam enzyme activity by assessing the interaction energy value. The assessment can be done relative to a reference or “cut-off” value.

In an embodiment the Dam enzyme structure is that obtained from a bacteriophage or a bacterium. In an embodiment the Dam enzyme structure is that obtained from E. coli. The structure can be from any source, so long as the structure has sufficient resolution so that a meaningful interaction energy value can be obtained from the simulated docking interaction. Preferred structures are obtained from X-ray crystallography, including those crystal structures deposited with the Protein Data Bank and summarized in Table 8.

The docking interaction preferably occurs at a docking site. Docking sites for Dam include an active site where the methyl donor donates a methyl group to the DNA base and/or a pocket formed between the catalytic and DNA binding domains and/or the methyl donor binding sites. Docking sites include AdoMet binding pocket, a channel into and out of the pocket, a hinge region between the catalytic and DNA binding domains, a DNA binding surface, a unique surface pocket, and other sites that can specifically affect DAM enzyme activity.

The methods of the present invention include computer-assisted drug design wherein, based on the enzyme's 3-dimensional structure, an inhibitor candidate structure is generated by calculating an interaction energy value between the generated structure and the enzyme structure. The enzyme can be a Dam enzyme, and the Dam enzyme structure can be obtained from any organism, including from a pathogenic bacteria. The Dam enzyme structure can be obtained from an E. coli Dam.

Compounds identified by any of these methods can be further assessed as capable of modifying Dam enzyme activity using known in vitro and/or in vivo assays, including by biochemical assays (e.g. non-cell based), cell-based, and whole-animal studies.

DNA methylation in a bacterium can be inhibited by providing the compound identified by the present invention and contacting the bacterium with the compound in an amount sufficient to inhibit DNA methylation in the bacterium. In an embodiment the bacterium contains a methylase, and preferably a Dam methylase and/or a cell-cycle regulated DNA adenine methylase. The bacterium can contain a methylase; in a particular embodiment, the methylase is capable of adenine methylation at GATC or GANTC sites.

Compounds have been identified by the screening methods and verified as inhibiting DNA methylation by biochemical and whole-cell assays. These compounds can be used to inhibit DNA methylation by Dam in an organism by contacting the organism with the compound. In an embodiment the compound is Dam-iZ1, wherein Dam-iZ1 has structural formula:

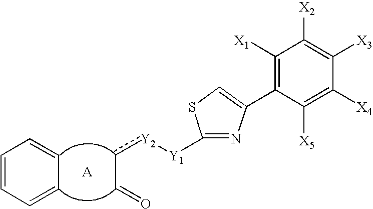

Wherein A is a non-aromatic 5 or 6 member ring and wherein one or more of the ring members of A can be C, N, O or S, and A can be optionally substituted. Examples of preferred structures for A (with the * indicating bond location to Y₂) are:

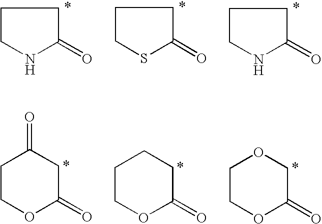

Each of X₁-X₅ is independently selected from the group consisting of H, halide, OH, OCH₃, alkyl and alkylhalide. Y₁ is NH or CH₂. Y₂ is N or CH. The dashed double bond to Y₂ indicated the bend can be single or double. In a specific embodiment Y₂ binds to A at the site indicated.

Compound Dam-iZ1 can be NCI 659390:

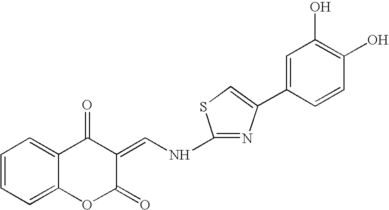

Compound Dam-iZ1 can be NCI 658343:

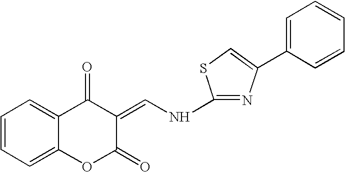

Compound Dam-iZ1 can be NCI 657589:

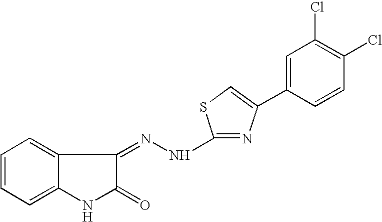

The term “aryl” refers to a group containing an unsaturated aromatic carbocyclic group of from 6 to 22 carbon atoms having a single ring (e.g., phenyl), one or more rings (e.g., biphenyl) or multiple condensed (fused) rings, wherein at least one ring is aromatic (e.g., naphthyl, dihydrophenanthrenyl, fluorenyl, or anthryl). Aryls include phenyl, naphthyl and the like. Aryl groups may contain portions that are alkyl, alkenyl or akynyl in addition to the unsaturated aromatic ring(s). The term “alkaryl” refers to the aryl groups containing alkyl portions, i.e., -alkylene-aryl and -substituted alkylene-aryl). Such alkaryl groups are exemplified by benzyl, phenethyl and the like.

Alkyl, alkenyl, alkynyl and aryl groups are optionally substituted as described herein and may contain 1-8 non-hydrogen substituents dependent upon the number of carbon atoms in the group and the degree of unsaturation of the group.

The term “heteroaryl” refers to an aromatic group of from 2 to 22 carbon atoms having 1 to 4 heteroatoms selected from oxygen, nitrogen and sulfur within at least one ring (if there is more than one ring). Heteroaryl groups may be optionally substituted.

As to any of the above groups which contain one or more substituents, it is understood, that such groups do not contain any substitution or substitution patterns which are sterically impractical and/or synthetically non-feasible. The compounds of this invention include all novel stereochemical isomers arising from the substitution of disclosed compounds.

Any of Compound Dam-iZ1, NCI-DTP Diversity Set compound numbers 659390, 658343, 657589, and any compound identified by the methods of the present invention can be used to inhibit DNA methylation by Dam in an organism by contacting the organism with any one or more of these compounds. In an embodiment, the organism is a bacterium, including an E. coli bacterium. In an embodiment the DNA methylation is inhibited by inhibition of a Dam methylase. In an embodiment, the concentration of compound to inhibit Dam is between about 10 μM and 400 μM. In an embodiment the concentration to inhibit Dam is between about 20 μM and 200 μM. In an embodiment the concentration to inhibit Dam is about 20 μM.

The invention includes methods of treating a host suspected of infection with a pathogenic bacterium comprising administering to the host a compound identified by any of the methods of the present invention, including a compound selected from the group consisting of Dam-iZ1 and NCI-DTP Diversity Set compound numbers 659390, 658343, and 657589. In an embodiment, the method of treating the host suspected of infection with a pathogenic bacterium reduces a virulence parameter of the bacterium. As used herein, virulence parameter is used broadly to refer to, for example, replication, adherence to host, colonization, motility, gene expression, metabolism, heat shock response, and other measurable parameters that are associated with virulence.

In an embodiment, the invention provides a method of treating a host suspected of infection with a pathogenic bacterium comprising administering to the host a compound capable of modification of pathogenesis by inhibiting a methylase. In an embodiment, the methylase is a Dam methylase. In an embodiment, the modification of pathogenesis involves a modification of virulence. In an embodiment, the modification of virulence is without a substantial effect on bacterial cell division.

In an embodiment, the invention provides a crystal of Escherichia coli Dam. In an embodiment, the invention provides a crystal of a Escherichia coli Dam complex. In an embodiment, the complex comprises E. coli Dam and cognate DNA. In an embodiment, the complex comprises E. coli Dam and noncognate DNA. In an embodiment, the complex further comprises a cofactor or cofactor analog. In an embodiment, the cofactor or cofactor analog is selected from the group consisting of AdoMet, AdoHcy, and sinefungin. In a particular embodiment, the crystal has a set of atomic structure coordinates of FIG. 37. In an embodiment, the invention provides a data representation of one or more crystals as described herein.

### BRIEF DESCRIPTION OF THE DRAWINGS

FIG. 1: DNA adenine methylation by Dam. Dam catalyzes the transfer of a methyl group from AdoMet to the N6 atom in the adenine residue in GATC sequences.

FIG. 2: T4Dam-AdoHcy structure. A. Ribbon representation of the binary structure of T4Dam in complex with AdoHcy (in ball-and-stick model). B. The hairpin loop contains conserved residues involved in DNA (sequence-specific and non-specific) interactions. C. Sequence alignment of the hairpin loop of selected Dam MTase orthologs. Sty: Salmonella typhimurium; Sma: Serratia marcescens; Ype: Yersinia pestis; Vch: Vibrio cholerae.

FIG. 3: T4Dam-AdoHcy-12mer DNA structure. Two orthogonal views of nonspecific binding of the Dam complex to a 12mer DNA. A. Molecule A binds to a single DNA molecule, while molecule B binds at the joint of two DNA molecules. B. is a view down the helical axis of the DNA. C. The hairpin loop of molecule B is near the DNA joint, but does not make any specific contact with the DNA.

FIG. 4: Ternary structure of T4Dam-AdoHcy-13-mer DNA. This structure has been deposited (PDB 1YF3). A. The two DNA molecules, shown at right with the helical axes projecting out of the page, are shifted relative to one another perpendicularly to the DNA axis. B. Schematic summary of the protein-DNA contacts in the nonspecific complex (molecule A) and the ¼-site recognition complex (molecule B). C. F111 of the hairpin loop of the joint binding Dam (molecule B) stacks with the 5′ Thy. D. Specific interactions are observed for R116-Gua, P126-Thy, and M114-Thy.

FIG. 5: Structure of T4Dam-AdoHcy-15-mer DNA. This structure has been deposited (PDB 1YFJ). A. All joints between two DNA duplexes are occupied by Dam molecules, labeled as C or D, while only one specific GATC site is bound by molecule E B. F111 in the hairpin loop of Dam molecule C stacks with two 5′ Thy. C. Specific interactions are mediated by R116, P126, M114, S112, G128, and R130.

FIG. 6: Intercalation of the T4Dam F111. (A) Interactions between molecule E and a canonical GATC site. A dashed light-blue circle labels the flipped-out Ade. The region of intercalation of T4Dam into the DNA is labeled by a dashed dark-blue circle and shown enlarged in the right panel. F111 of molecule E intercalates between the AT base pair and the Thy:S112 “base-amino acid” pair. (B) Chemical structures of AdoMet, AdoHcy, and sinefungin. (C) Active-site conformation in the presence of sinefungin (PDB code 1YFL). An invariant N-terminal residue K11 interacts with the side chains of D171 and Y174 as well as the backbone carbonyl oxygen of G9; the same D171-K11-Y174 interactions were observed in the binary structure of T4 Dam-Ado Hey (Yang et al., 2003). The D171-K11-Y174 interaction is likely to be critical for normal function since a K11S substitution virtually abolishes enzyme activity (V. G. Kossykh, S. L. Schlagman, and S. H., unpublished data). The amino group of K11 is also close to the ring N1 atom of the target Ade. The mutant of the corresponding Lys in M.EcoRV (K16R) showed an altered specificity toward the target base (Roth and Jeltsch, 2001).

FIG. 7: Interactions with a Noncanonical Site. (A) F111 intercalation by molecule E into the central AT stacking of the DNA molecule depicted in orange effectively causes a one-base-pair lengthening. The expansion results in two disordered nucleotides (shaded) of the neighboring duplex (magenta). (B) Interactions between molecule D and a noncanonical site. The 5′-overhanging Thy of the magenta DNA is pushed out and apparently becomes disordered, resulting in the Cyt of the next base pair stacking with F111 of Dam molecule D. (C) Detailed interactions of R130 and the external G:C base pair and S112-Cyt.

FIG. 8: Biochemical Analysis of EcoDam Variants. (A) Schematic summary of protein-DNA base contacts in the specific complex and sequence alignment of the β hairpin loop of T4Dam (G110-T131), recognition sequence GATC), EcoDam (G118-K139, recognition sequence GATC), and EcoRV (C122-P143, recognition sequence GATATC). The flipped target base is labeled as a shaded X. Point mutations made in the EcoDam are indicated (note the differences in numbering of residues). It should be noted that the normal in vivo substrate for T4Dam is phage DNA containing glucosylated 5-hydroxymethyl-Cyt (hmCyt) in place of Cyt. Phage hmCyt-containing DNAs (with or without the presence of glucosylation) are not methylated by EcoDam (Hattman, 1970). As seen in the structures presented here, neither of the Cyt bases in the palindromic GATC site (Cyt1 or Cyt4) makes contact with T4Dam. In the specific complex, the shortest distance between the protein and these bases is 6.3 Å from Cyt4 to V178 and 7.7 Å from Cyt1 to K129. In this regard, EcoDam has insertions in both places, viz. six additional residues adjacent to V178 and two additional residues adjacent to K129 (see FIG. 1 of Yang et al., 2003). These additional residues in EcoDam may sterically clash with the hydroxymethyl group on either hmCyt base (or both) and prevent the enzyme from methylating the DNA. Single-turnover DNA methylation rates (B) and DNA binding affinities (C) of wild-type EcoDam and its variants. EcoDam variants were cloned, expressed in E. coli, and purified to homogeneity.

FIG. 9: Specificity Profiles of EcoDam. (A-E) Single-turnover methylation rates of wild-type and variants are given for the cognate GATC (light-blue bars) as well as all nine near-cognate substrates. On the horizontal axis, the three positions of the GATC site that are mutated are given (G= GATC, T= GATC, C= GATC). On the right axis the new base introduced at each position is specified (for an example, see FIG. 12). The methylation rates of the respective pairs of enzyme and substrate are given on the vertical axis. (A) Wild-type, (B) R124A, (C) P134A, (D) P134G, and (E) L122A. (F-H) Specificity factors of EcoDam variants for recognition of the fourth (S4) (F) and third positions (S3) (G) of the GATC sequence and overall specificity factors (H). All values are given as relative changes with respect to the wild-type. The specificity factor of wild-type EcoDam was 540; the value was increased at least 30-fold in the case of the L122A variant. Because no activity at near-cognate sites could be detected with the L122A variant, the specificity factor given here is a lower limit, indicated by the arrow. The specificities of the R124A, P134A, and P134G variants were dramatically reduced. The specificity factors of all other variants did not show large deviations when compared with the wild-type enzyme.

FIG. 10: Snapshots of T4Dam-DNA complex structures illustrated by orientation of the protein hairpin loop relative to the DNA axis: (A) Non-specific complexes with R130 involved in phosphate contact; (B) non-specific complex with R116 involved in phosphate contact; (C) the 1/4-site complex with R116 involved in base-specific contacts, N118 and R130 in phosphate contacts; (D) the 3/4-site complex with R116 and R130 involved in base-specific contacts, N118 in phosphate contact; (E) interaction with a non-canonical site; and (F) a full-site complex.

FIG. 11: Schematic Summary of the Protein-DNA Contacts for the (A) ¾ site complex; (B) in the non-canonical site; and (C) the specific full-site.

FIG. 12: Specificity Profiles of EcoDam Variants. The specificity profiles of WT EcoDam and the Y119A, N120A and S, R137A, Y138A and K139A variants are shown. In the figure the single turnover methylation rates of wild type and variants are given for the cognate GATC (light blue bars) as well as all nine near-cognate substrates. On the horizontal axis the three positions of the GATC site that are mutated are given (G=GATC, T=GATC, C=GATC). On the right axis the new base introduced at each position is specified. The methylation rates of the respective pair of enzyme and substrate are given on the vertical axis

FIG. 13: Structure of EcoDam-AdoHcy-12mer DNA. For clarity, the second DNA molecule is not shown. (B) is a view down the helical axis of the DNA molecule.

FIG. 14: Structure of EcoDam-AdoHcy-12mer DNA. (A) Two DNA duplexes (bold and not bold) are stacked head-to-end, with one GATC site in the middle of each duplex and one in the joint of two duplexes. The nucleotides in extrahelical positions are shaded in blue circles. (B) Molecule A binds to the GATC site in the middle of each DNA duplex, while EcoDam molecule B binds to the joint of two DNA duplexes. (C) EcoDam contains two domains: a seven-stranded catalytic domain that harbors the binding site for AdoHcy (represented by a stick model) and a DNA binding domain consisting of a five-helix bundle and a β-hairpin loop that is conserved in the family of GATC-related MTase orthologs. N-terminal residues 7 to 10, colored in cyan, also interact with the DNA (see E). (D) Comparison of EcoDam and T4Dam. In T4Dam, the six-residue shorter active-site loop is involved in forming a closed-flap cofactor binding site (Yang et al., 2003), while the residues between strands β6 and β7 are disordered (Yang et al., 2003) and become ordered only when they involved in crystal packing contacts (Horton et al., 2005). (E) Summary of the protein-DNA contacts of molecule A (red) and molecule B (grey). Backbone mediated interactions are indicated with main chain amine (N) or carbonyl (O). For simplicity, only single water (w) mediated interactions are shown. Focusing on a single DNA duplex (blue), 20 out of 22 phosphate groups interact with three EcoDam molecules (A, B, and symmetry-related molecule B). Thus, the choice of the length (12 base pairs) and the end sequence of the oligonucleotide used for crystallization optimally maximized the DNA-protein interactions and DNA-mediated protein-protein interactions in the crystal lattice of packing. The only two phosphate groups that are not involved in EcoDam interactions are the 5′phosphates of the two Thy of the central GATC site—which are the missing phosphates in the joint GATC site. The immediate flanking phosphate groups of the orphan Thy have either no interaction (5′ phosphate) or only weak interaction (3′ phosphate) with S198 (with higher thermal B-factor), the first ordered residue after the unstructured loop (residues 188-197). The less constrained conformation allows bond rotations about the DNA backbone at the orphaned site, which moves the Thy to an extrahelical position and disrupts the Thy-N120 interaction.

FIG. 15: EcoDam-DNA base interactions. (A) The target Ade is bound in an alternative nucleotide-binding site, on the outside edge of the active-site pocket formed by the DPPY motif (left panel). The target Ade is superimposed with an omit (base and ribose) electron density map contoured at 3.5σ above the mean (middle panel). Large rotations about three bonds of the DNA backbone drive the insertion of Ade into the active site (right panel). The transferable methyl group, modeled onto the sulfur atom of AdoHcy, would lie out of the plane of the Ade base, consistent with the target nitrogen lone pair deconjugated and positioned for an in line direct methyl group transfer (indicated by an arrow), as seen in the M.TaqI-DNA complex (Goedecke et al., 2001). (B) The hairpin loop of molecule A (red) in the major groove of the blue DNA duplex with a central GATC site. (C) Interaction with the first base pair (G:C) of GATC. Dotted lines indicate hydrogen bonds. (D) The flipped orphan Thy, superimposed with an omit electron density map contoured at 3.5σ above the mean, stacked with the side chain of R137. Local conformational changes of the orphan base have also been observed in the M.HaeIII-DNA (base repairing) (Goedecke et al., 2001; Reinisch et al., 1995) and M.TaqI-DNA structures (base shifting toward the center of helix) (Goedecke et al., 2001; Reinisch et al., 1995). Double base flipping has been previously observed in the structure of the DNA repair enzyme endonuclease IV and its DNA substrate (Hosfield et al., 1999) as well as in the stopped-flow fluorescence studies with the MutY adenine DNA glycosylase using 2AP-containing DNA (Bernards et al., 2002). However, in the structure of MutY DNA lesion complex, the oxoG lesion lies completely in the DNA complex, while the Ade flipped out (Fromme et al., 2004). Taken together, these studies suggest that the oxoG, like the orphan Thy bound with EcoDam, can be in either an intrahelical or extrahelical location. (E) Interaction with the third base pair (T:A) of GATC. A methyl group is modeled onto the exocyclic amino nitrogen N6 atom of the Ade. Double arrows indicate van der Waals contacts. (F) Interaction with the fourth base pair (C:G) of GATC. (G) The orphan Thy-N120 interaction in the joint of two DNA duplexes. The Thy-N120 interaction is similar to other protein side chain-orphaned base interactions of base-flipping enzymes, such as those for Thy-S112 of T4Dam (Horton et al., 2005) and Gua-Q237 of M.HhaI (Klimasauskas et al., 1994). (H) The hairpin loop of molecule B (red) in the joint of two DNA molecules (green and blue). The interactions with the first, third, and fourth bases pairs are identical with that of molecule A (see panel B).

FIG. 16: Recognition of the first base pair by N-terminal K9. (A) Pair-wise sequence alignment of EcoDam and T4Dam in two regions: the β hairpin loop and the N-terminal loop. The residues colored in red were targets for site-directed mutagenesis. (B-C) Specificity profile of EcoDam wide type (B) and the K9A variant (C). The single turnover methylation rates of the wild type and the K9A variant are given for the cognate, hemimethylated GATC substrate (light blue bars) as well as for all nine near-cognate hemimethylated substrates. On the horizontal axis the three positions of the GATC site that are mutated are given (G=GATC, T=GATC, C=GATC, M=N6mA). On the right axis the new base introduced at each position is specified. The methylation rates of the respective pair of enzyme and substrate are given on the vertical axis (note the logarithmic scale). (D) Specificity factor (defined in Experimental Procedures) of EcoDam variants for recognition of the first position of the GATC sequence (S1). The values are given as relative changes with respect to the wild type. Because no activity could be detected at near-cognate sites modified at the third or fourth base pair of GATC with the K9A variant, the S1 factor given here is a lower limit, indicated by the arrow. The specificity factors of wild type EcoDam and the K9A were calculated using the data given in panels B and C; the data for all other variants were taken from (Horton et al., 2005).

FIG. 17: Base flipping by EcoDam and its variants. (A) Fluorescence intensities of several DNA substrates in the presence of EcoDam. The figure displays the fluorescence of 2AP at the position of target Ade (blue curve), the orphan Thy (orange curve), the Gua1 position of the first pair (green curve), and the immediate 5′ position to the GATC (red curve). The pink curve displays free DNA (the hemimethylated G-2AP-TC) as a control and the black curve is for free enzyme. (B) Changes of relative fluorescence of hemimethylated G-2AP-TC during binding of EcoDam and its variants. (C) Stopped-flow studies of base flipping using substrates containing the 2AP probe at the position of the target Ade (blue curve) and the orphan Thy (orange curve). The blue curve shows a biphasic reaction in which a fluorescence increase during the first 100 msec is followed by a decrease in fluorescence after 1 sec. (D) Stopped-flow studies of base flipping using substrates containing the 2AP at the target position (blue curve) and with three near-cognate substrates that carry a one base pair substitution at the first (pink curve), third (green curve) or fourth base pair (red curve) of the recognition site. (E-G) Stopped-flow studies of base flipping with EcoDam variants with various substrates: (E) R124A, (F) P134G, and (G) K9A.

FIG. 18: Discrimination between unmethylated and hemimethylated DNA. Methylation of unmethylated (squares) and hemimethylated (diamonds) oligonucleotide substrates by (A) EcoDam (WT) and (B) L122A variant.

FIG. 19: Structure of a non-canonical complex. (A) EcoDam molecule C preferentially binds at the joint of two DNA duplexes, which mimics an altered recognition site, consistent with structural data for T4Dam (Horton et al., 2005) and biochemical data for other DNA MTases (Cheng and Roberts, 2001; Klimasauskas and Roberts, 1995). Here, the presence of partial recognition site (notably the 5′ G:C base pair) was sufficient for stable complex formation with EcoDam. The blue circle indicates a disordered Ade. (B) Schematic summary of the protein-DNA contacts. Two DNA duplexes (green and blue) are stacked head-to-end, with one T:T mispair in the joint of two duplexes. Backbone mediated interactions are indicated with main chain amine (N) or carbonyl (O). (C-H) Details of DNA interactions with EcoDam molecule C. (I) DNA sequence comparison of the non-canonical site and the Pap GATC flanking sequences. (J) Organization of pap regulatory sequence. Numbers (1-6) indicate six leucine-responsive-regulatory-protein (Lrp) binding sites (Hernday et al., 2003). Among the six Lrp binding sites, sites 2 and 5 contain GATC sequence (top panel). Model of Dam molecules (in red or green circles) which travel along the DNA to methylate their respective target Ade (in red or green shading), and could be trapped at one of the non-cognate sites (boxed in red or green).

FIG. 20: Structural comparison of canonical and non-canonical complexes. (A) Superposition of the canonical complex (molecule A, colored in grey, and DNA, colored in blue) and the non-canonical complex (molecule C), with the fourth base pair and its interaction with R124 being shown. Only the base atoms of G:C pair and the side chain atoms of R124 were used for superposition. (B) The DNA backbone of the canonical complex is colored in blue and the non-canonical complex in magenta. The R124-Gua4 interaction takes place with the right-side DNA. Intercalation by Y119 would move the left-side DNA of the non-canonical complex with one-base-pair lengthening along the helix axis (indicated by the arrow). (C) Comparison of the DNA in the two complexes, using the right-side portion for superposition. The non-canonical duplex on left-side portion is rotated by ˜30° about the helix axis.

FIG. 21: (A) Organization of pap regulatory sequence. Six Lrp binding sites are located between the divergent papBA pilin and papl promoters (adapted from Hernday et al. 2003). (B) Among the six pap sites, sites 2 and 5 contain GATC sequence (boxed). (C) Design of an experiment to study the molecular basis for the lack of processivity in methylation of pap sites.

FIG. 22: Flow-chart summary of structure-based in silico screening.

FIG. 23: Cofactor AdoHcy docking. Superimposition of the experimentally determined AdoHcy conformations in T4Dam and DIM-5. The AdoHcy in T4Dam is in an extended conformation—most frequently observed in widespread class I MTases such as the DNA MTases (Schubert et al. 2003). However, the extended conformation is significantly different from the folded conformation observed in the SET domain of histone Lys MTases (HKMTs) such as DIM-5. Such different conformations of the cofactor may provide a good target to design inhibitors that are selective for class I (T4Dam, DNMTs, and PRMTs) versus class V (SET HKMTs) MTases. The sphere centers generated from the cofactor can reproduce the experimentally determined binding mod of AdoHcy in (B) DIM-5 (left) and T4Dam (right).

FIG. 24: (A) The two domain structure of T4Dam, catalytic domain (dark) and DNA binding domain (light). There is a deep cavity (indicated by sphere centers represented by dots) between the two domains. Preliminary DOCK screening revealed unique small molecules (NSC48693 and NSC159165) that may bind in either the AdoHcy binding pocket (top) or the cavity between the two domains (bottom). (B) The small but important structural difference between T4Dam-AdoHcy (top) and M.DpnII-AdoMet (bottom). (G) Superimposition of top 30 hits for the cofactor-binding site onto the cofactor analog AdoHcy.

FIG. 25: DOCK results of DIM-5. (A) AdoHcy binding site; (B) Small molecule NSC106221 docked into the AdoHcy binding site; (C) Target Lys-containing peptide binding site; (D) Small molecule NSC322921 docked into the Lys-binding channel.

FIG. 26: Summary of the approximately 2000 compounds from the NCI “Diversity Set” used in the initial ISS. Each entry corresponds to one compound with an NSC identifier and a SMILES string containing information of atom connections and bond types.

FIG. 27: Summary of the 82 compounds identified by the ISS and examined in more detail. Each entry corresponds to one compound with an NSC identifier, molecular weight, and chemical structure drawing. DIM-5 inhibitory compounds correspond to entries 1-36; Dam inhibitory candidates correspond to entries 41-80. Histamine Methyltransferase Inhibitors are entries 37-40 and 81-82 (not shown).

FIG. 28: Energy score rankings obtained from the ISS for the top 100 compounds. Each entry has an NSC identifier and energy score. (A) and (B) are scores for the DIM-5 inhibitors and (C) and (D) are scores for the Dam inhibitors.

FIG. 29: List of additional compounds chosen for their structural similarity to lead compound NSC 659390.

FIG. 30: EPEC causes formation of actin-filled membrane protrusions on the surface of host epithelial cells. Under fluorescent microscopy EPEC is labeled green, actin labeled orange, and DNA (both bacterial and nuclear) is blue. Scale bar is 10 microns.

FIG. 31: Microscopy images of (A) uninfected 3T3 cells and (B-C) infected 3T3 cells. Cells in (C) are treated with 20 μM G6 (compound #78—NSC 659390) and stained with FITC phalloidin to label actin (middle column and green in Merge) and DAPI to label 3T3 and bacterial nuclei (left column and blue in Merge). Actin pedestals are visible as bright actin staining (e.g. arrow). No actin pedestals are observed with G6. Scale bar 20 μm.

FIG. 32: Growth curves of EPEC with and without 20 μM G6 compound and with 20 μM B11 (compound #23—an antibiotic). G6 had no effect on bacterial growth compared to the antibiotic.

FIG. 33: Effects of G6 on EPEC virulence, (a-d) are uninfected 3T3 cells, (e-h) are 3T3 cells infected with EPEC, (i-l) are 3T3 cells infected with EPEC also treated with 20 μM G6. Cells are stained with FITC phalloidin for actin, DAPI to label bacteria, and α-Tir pAb-Cy3 to label the bacterial virulence factor Tir (which is secreted into host cells). Tir staining (see arrow in g and observed as red under fluorescent microscopy) is evident at the tips of actin pedestals (arrow in f, and observed as green under fluorescent microscopy). With G6 treatment, no pedestals (j) or Tir staining (k) is observed next to attached bacteria (arrows in (i)). Scale, 10 μm.

FIG. 34: (A) Methylation sensitive digestions of pUC19 DNA isolated from bacterial treated compounds. (B) Design of a more sensitive and high-throughput bacterial-based assay.

FIG. 35: C57BL/6 mice were infected with EPEC or C. rodentium. Bacterial load of colon tissue is determined 7 days post infection (pi) by grinding colon pieces, plating on MacConkey agar, and counting colonies (colony forming units (CFU) per gram of colon tissue. Neither EOEC nor C. rodentium were detectable in uninfected mice. (B) C57BL/6 mice are infected with C. rodentium or EPEC following pretreatment with 20 mg streptomycin. Colon tissue is harvested from mice 7 days pi and analyzed by H & E stain. (Scale bar= 200 μm). (C) Colons from mice infected with EPEC or C. rodentium (day 14) are harvested and analyzed for myeloperoxidase activity, a measure of neutrophil recruitment to the colon. *p<0.05 compared with uninfected colons.

FIG. 36: Sequence data for selected Dam MTase orthologs (from FIG. 1 of Yang et al. Nature Structural Biology 10:849-855) (2003) for bacteriophage T4 (T4Dam), Escherichia coli (EcoDam), restriction-modification MTases EcoRV, and DpnIIA. Invariant and conserved residues are shown as highlighted white characters and bold characters, respectively. The secondary structure of T4Dam is shown above the sequence (cylinders for helices, arrows for strands).

FIG. 37: Three-dimensional coordinate structure of EcoDam. The figure shows the X-ray coordinates of the EcoDam ternary (EcoDam-AdoMet-12mer DNA) complex as described in the Examples and is used for ISS to identify Dam inhibitor candidates.

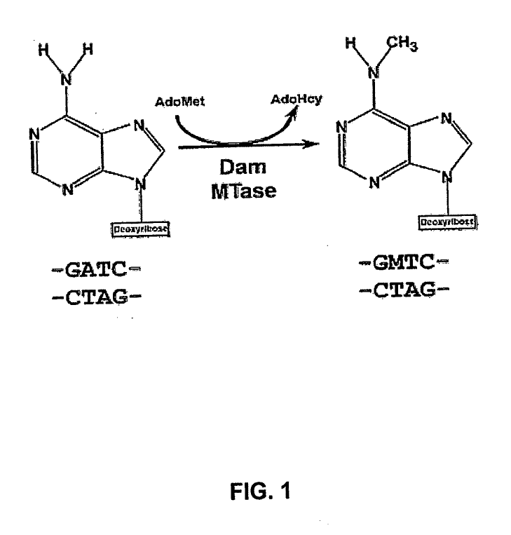

### DETAILED DESCRIPTION OF THE INVENTION

The invention may be further understood by the following non-limiting examples. All references cited herein are hereby incorporated by reference to the extent not inconsistent with the disclosure herewith. Although the description herein contains many specificities, these should not be construed as limiting the scope of the invention but as merely providing illustrations of some of the presently preferred embodiments of the invention. For example, thus the scope of the invention should be determined by the appended claims and their equivalents, rather than by the examples given. In general the terms and phrases used herein have their art-recognized meaning, which can be found by reference to standard texts, journal references and contexts known to those skilled in the art. The following definitions are provided to clarify their specific use in the context of the invention.

List of abbreviations: A/E (attaching and effacing); ATCC (American Type Culture Collection); Dam (DNA-adenine MTase); AdoHcy (S-adenosyl-L-homocysteine); AdoMet (S-adenosyl-L-methionine); EcoDam (E. coli Dam); EHEC (enterohemorrhagic E. coli O157:H7); EPEC (enteropathogenic E. coli); HTA (high throughput assay); ISS (in silico screening); MTases (methyltransferases); NCI (National Cancer Institute); PDB (Protein Data Bank).

General Crystallization and Structure Determination Techniques. Dam enzymology and assay development have been examined. See, for example, Roth & Jeltsh (2000); Urig et al. (2002); Humeny et al. (2003); Liebert et al. (2004); Horton et al. (2004). Utilizing standard procedures for protein purification, crystallization and structure determination, we have solved de novo structures of many AdoMet-dependent MTases and related proteins: HhaI DNA MTase, HhaI MTase-DNA complex, PvuII endonuclease-DNA complex, PvuII DNA MTase, protein arginine MTases PRMT3 and PRMT1, small molecule histamine MTase and its complex with inhibitor, Dnmt3b PWWP domain, MBD4 glycosylase domain, histone H3 Lys9 MTase DIM-5 and its complex with substrate H3 peptide, phage T4 Dam and its complexes with DNA specifically and nonspecifically, protein glutamine-N5 MTase HemK, a nucleosomal dependent histone H3 lysine 79 MTase Dot1p, and HinP1I endonuclease.

Once the enzymes are purified and concentrated to approximately 10 mg/ml, the crystallization conditions are searched using three screens (300 conditions) currently available in the lab. If further screens are necessary, we can use other commercially available screens of thousands of conditions, including different precipitants, buffers, etc. We screen the crystallization in three parallel lines: First, for the apo-enzyme, second, for the binary complex of MTase-AdoMet or AdoHcy complex, the cofactor is added during the last column of purification (usually a gel filtration column) and during the concentration step, and third, for the ternary complex of MTase-AdoHcy-DNA, the protein/DNA ratio as well as the DNA length and sequence is varied. In addition, we use hemimethylated GATC for crystallization (N6-methyl-Ade in one of the strands) because it is the nature substrate present immediately following DNA replication.

We use three approaches to solve the structure of EcoDam and other Dam molecules when X-ray diffraction quality crystals are obtained: (1) Molecular replacement; (2) Multi- or singly-wavelength anomalous diffraction (MAD or SAD) of Seleno-Met; and (3) Multiple isomorphous replacement (MIR) of heavy atom derivatives.

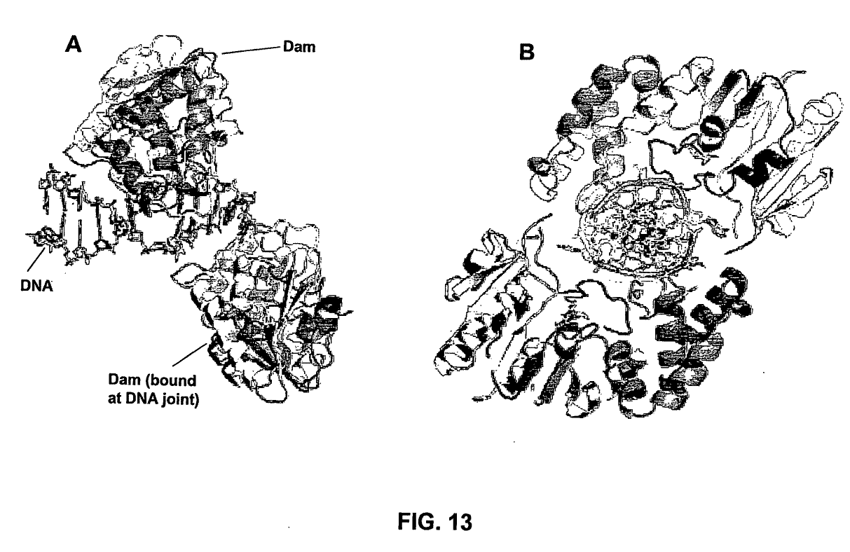

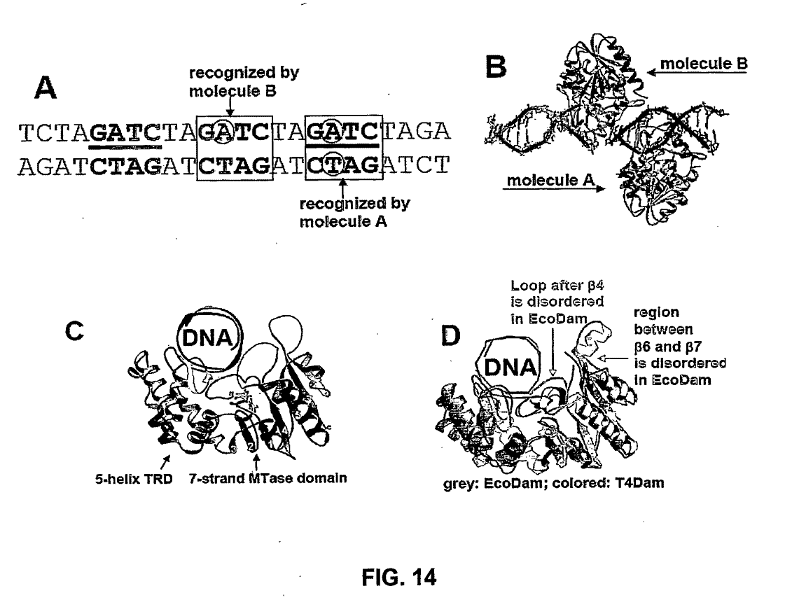

Molecular replacement: For the EcoDam structure determination (see FIG. 13), we use T4Dam coordinates as the starting model for rotational- and translational-function searches. E. coli and T4Dam proteins share 25% sequence identity and 46% homology. We modified T4Dam model by replacing the non-conserved side chains to alanines and deleting several small loop regions. The model is put into three rigid groups (the catalytic domain, the DNA binding domain, and DNA itself) and the molecular replacement searches are successfully completed utilizing program CNS (Brunger et al., 1998).

We use the same approach to solve the Dam structures of Salmonella and H. influenzae when the crystals become available. In addition, the molecular replacement solution can also be used to locate the Se or mercury sites via an anomalous difference Fourier map in the MAD or MIR data (below). A combination of the molecular replacement and experimental phases can greatly improve the quality of the electron density map and make it suitable for interpretation of the structure. We used a similar approach to solve the structure of HemK (Yang et al., 2004).

Multi- or single-wavelength anomalous diffraction (MAD or SAD) of Seleno-Met: EcoDam contains three methionines, and we have replaced the methionines in the protein with Se-Met by overexpressing the protein in the medium that supplies Se-Met. Preliminary X-ray data have been collected at APS SERCAT beamline for two wavelengths near the Se-absorption edge at ˜2.3 Å resolution; however these data were not needed because we solved the structure by molecular replacement (above).

Multiple isomorphous replacement (MIR) of heavy atom derivatives: If needed, isomorphous heavy atom derivatives are obtained by soaking the crystals in a variety of reagents containing heavy atoms. We initially focus on mercurial compounds. The mercury atom reacts with the sulfur atom of cysteine and EcoDam contains five cysteine residues. The first T4Dam structure was solved via mercury derivatives (Yang et al., 2003).

Any one of a variety of Dam molecules from pathogenic bacteria can be crystallized to obtain a high-resolution three-dimensional structure via X-ray crystallography. For example, Dam molecules can be obtained from Salmonella enterica serovar typhimurium, Yersinia pestis, and Klebsiella pneumoniae. The three enzymes (278, 271 and 275 residues, respectively) are similar in size to E. coli Dam (278 residues). Kpn Dam has been expressed in E. coli and purified. In addition, Shigella flexnerii and Salmonella pseudotuberculosis dam genes have been cloned and the proteins have been expressed in E. coli and are catalytically active (data not shown). The purified Dam protein is used to obtain a crystal structure by crystallographic methods known in the art.

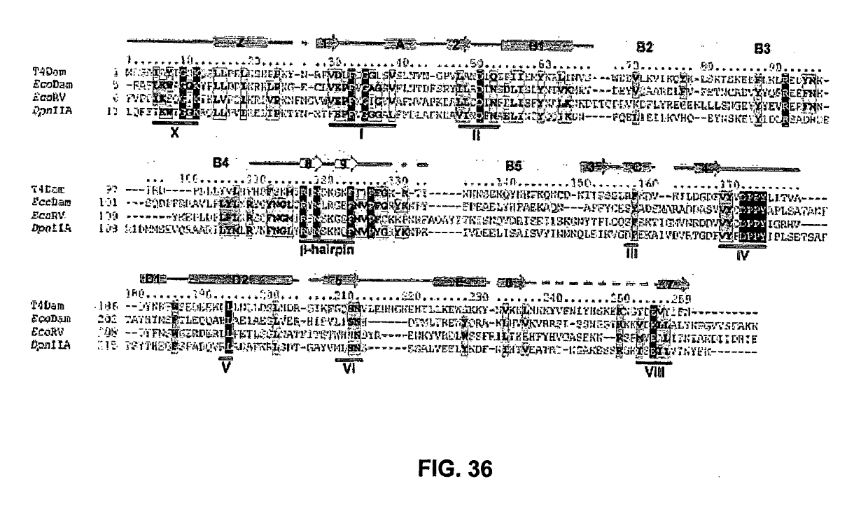

The Kpn genomic DNA was obtained from ATCC (Manassas, Va.); the Dam gene was acquired from the genomic DNA using PCR (Dam sequence are available from publicly accessible databases, e.g. see e.g. FIG. 36 for the amino acid sequence of EcoDam and T4Dam). We express both GST-KpnDam (containing a thrombin site after the GST) and (His)6-tagged KpnDam using pET plasmids. Both expressed in E. coli strain BL21(DE3). For purification, the T4Dam protocol, as described previously (Kossyk et al. 1995, Yang et al. 2003), can be followed. In addition, E. coli Dam is expressed in a pET system. Salmonella and Yersinia Dam expression constructs are available (Dr. Michael Mahan). Other Dam molecules can be similarly obtained by obtaining the corresponding bacterial genome from publicly available sources, including the ATCC, and extracting the Dam gene from the genome by, for example, PCR.

### Example 1

### X-Ray Crystallography of T4DAM and T4DAM-DNA Complexes

T4Dam structure has been solved by X-ray crystallography. See Yang et al. “Structure of the bacteriophage T4 DNA adenine methyltransferase Nature Struct. Biol. 10: 849-55 (2003) and Horton et al. “Transition from nonspecific to specific DNA interactions along the substrate recognition pathway of Dam methyltransferase” Cell 121. 349 61 (2005), both incorporated by reference, and specifically incorporated by reference for crystallographic methods, data and solution structure of T4Dam. The coordinates of the binary and ternary structures of T4Dam are deposited in the Protein Data Bank (see Table 8 for a summary of structures deposited with the PDB and PDB ID Nos).

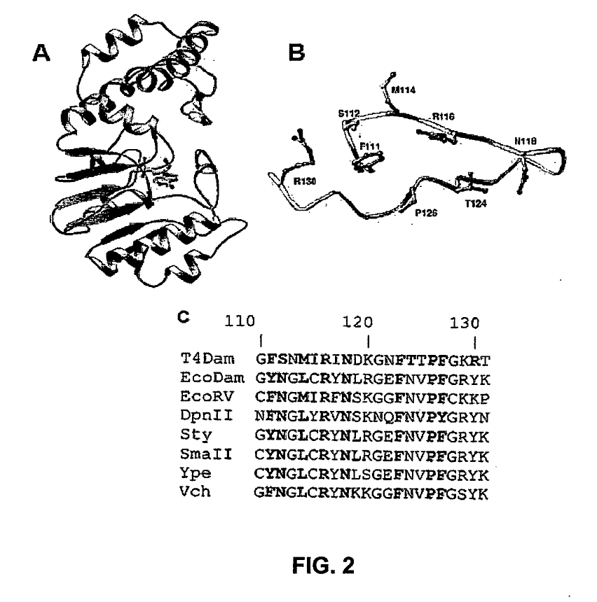

Bacteriophage T4Dam contains two domains: (i) a seven-stranded catalytic domain harboring the binding site for AdoHcy and (ii) a DNA binding domain consisting of a five-helix bundle and a beta-hairpin loop. (FIG. 2A-B) that is conserved in the family of GATC-related MTase orthologs (FIG. 2C).

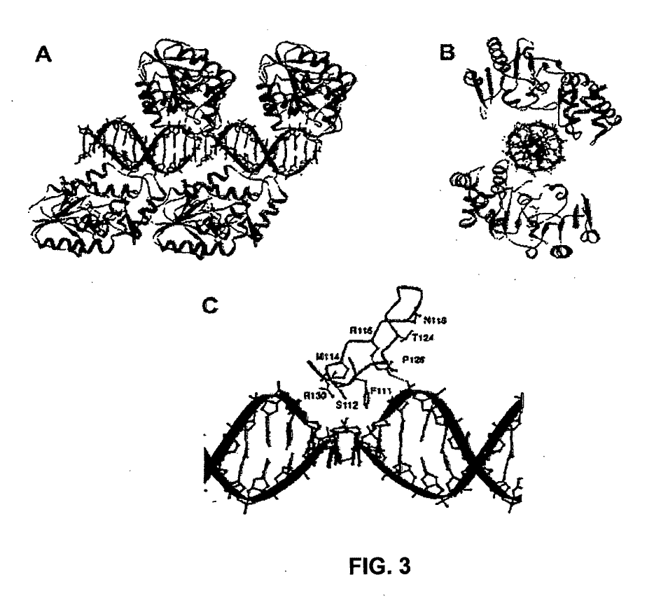

Structure of non-specific T4Dam-DNA-AdoHcy complex: We crystallized a ternary complex of T4Dam with both AdoHcy and a synthetic 12 base pair DNA (ACAGGATCCTGT)—the minimum substrate for T4Dam (Hattman and Malygin, 2004). In the crystal, the DNA duplexes are stacked head-to-end, forming a pseudo-continuous DNA duplex. FIG. 3A. Surprisingly, the sequence-specific T4Dam does not bind at the GATC site. FIG. 3A-C. Rather it binds DNA in a nonspecific “loose” mode that contains two Dam monomers per synthetic duplex. FIG. 3A-B.

An explanation for the non-specific binding between T4Dam and DNA is that T4Dam methylates DNA with multiple GATC sites in a processive manner; i.e., more than one methyl group may be transferred per bound Dam monomer. In the ternary crystal structure, the T4Dam-AdoHcy complex may be on the duplex in a fashion that corresponds to the stage following methyl transfer. That is, it is not in contact with the GATC target site; rather it contacts the phosphodiester backbone and is primed for diffusion and/or exchange of AdoHcy with AdoMet. This ternary structure provides a rare snapshot of an enzyme poised for linear diffusion along the DNA.

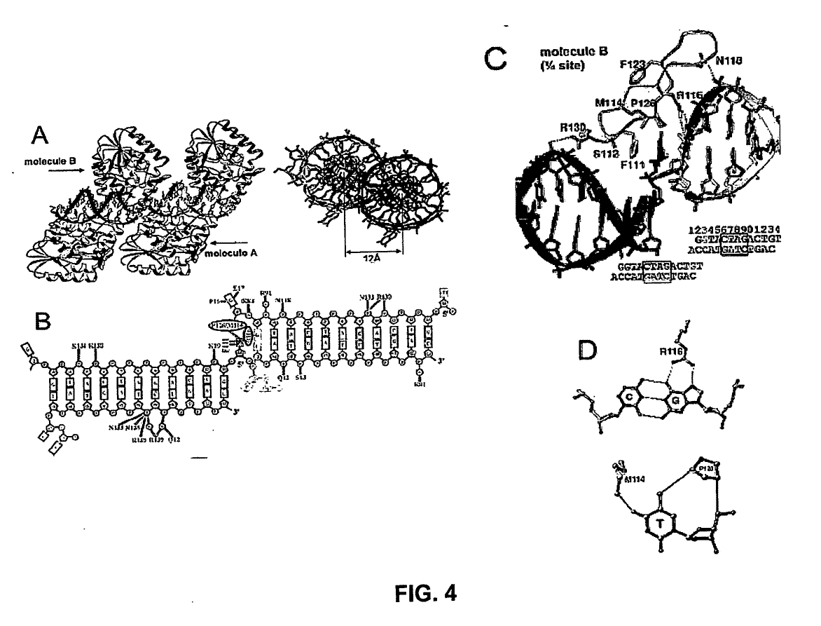

Structure of a semi-specific complex: In addition to the blunt-end GATC-containing 12mer DNA, we also use a 13mer specific DNA with a 5′ overhang Ade in one strand (ACCATGATCTGAC) and a 5′-overhang Thy in the other strand (TGTCAGATCATGG), so that the Ade and Thy are base paired at the joint. As with the non-specific binding, two Dam molecules bind one DNA duplex, except the helical axis of the two DNA molecules are shifted relative to on another by about 12 Å (FIG. 4A). The T4Dam binds the DNA joint making hydrogen bonding interactions via R116 with the Gua at the G:C base pair at position 3 (FIG. 4B-C). Surprisingly, the next G:C pair at position 2 and the overhanging Ade at position 1 are opened up (via DNA melting) (FIG. 4B). The overhanging Thy of the next DNA molecule approaches, becomes extra helical, and stacks with the Cyt of the G:C pair, while the phenyl ring of F111 stacks on the other side (FIG. 4B). The methyl group of the Thy is in van der Waals contact with P126, while the 04 atom is in contact with M114 (FIG. 4C). The residues involved in the interactions (R116, F111, P126, and M114) are highly conserved amino acids in the family of GATC MTases (see FIG. 2C). Without wishing to be bound to a specific theory, it appears the molecule is forcing the sequence at the joint to mimic part of the recognition sequence.

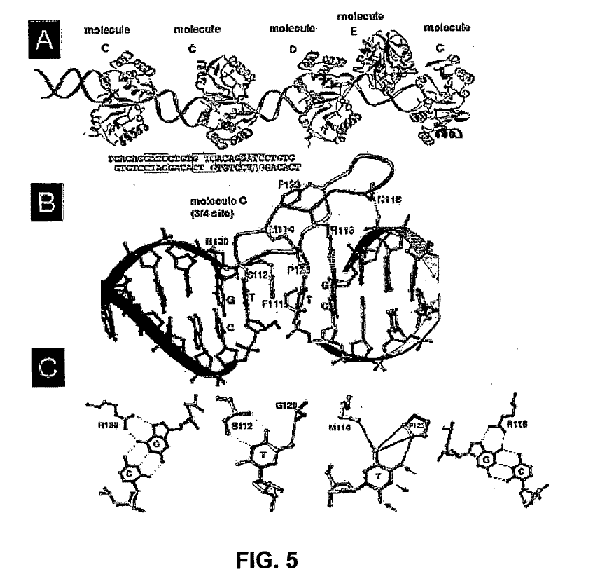

Structure of a ternary complex containing both semi-specific and specific contacts: In addition to the 12mer and 13mer, we constructed a 15mer oligo (TCACAGGATCCTGTG) with the end sequence mimicking part of the recognition sequence. In addition, we also reduced the ratio of protein to DNA. We observed: (1) all of the joints between neighboring DNA molecules are occupied by a Dam molecule (molecules B, C, D, and E in FIG. 5A). The stacking of two DNA molecules is mediated via F111, which stacks with 5′ Thy bases from two neighboring DNA molecules (FIG. 5B). (2) More specific interactions are observed in the joint of the oligonucleotides with R116, P126 and M114 interacting with one half site, and S112 and R130 interacting with the second half site (FIG. 5C). (3) Because the protein to DNA ratio is reduced, only one molecule (molecule F in FIG. 5A) binds to the specific GATC site in the middle of the oligo, making specific interactions with a target Ade flipped out from the duplex DNA (not shown). Tables 2 and 3 summarize the properties of various T4Dam-DNA-AdoHcy crystals.

These observations indicate the Dam enzyme preferentially binds at the joint of two DNA molecules, which mimics damaged DNA or altered recognition sites. This is surprising but consistent with biochemical data, which suggest that binding specificity for DNA MTases is determined by the nucleotides flanking the target nucleotide and DNA MTases bind more tightly to substrates containing mismatches at the target base (Cheng and Roberts, 2001). In other words, DNA MTases do not depend on the flappable target base for their binding specificity. For Dam, having only one-half of the recognition site on one strand appears to be sufficient for stable complex formation provided that the 5′ G:C base pairs are present at both ends of the palindrome (Hattman and Malygin, 2004). This is what we observe for the joint formed by the 15mer duplex DNA.

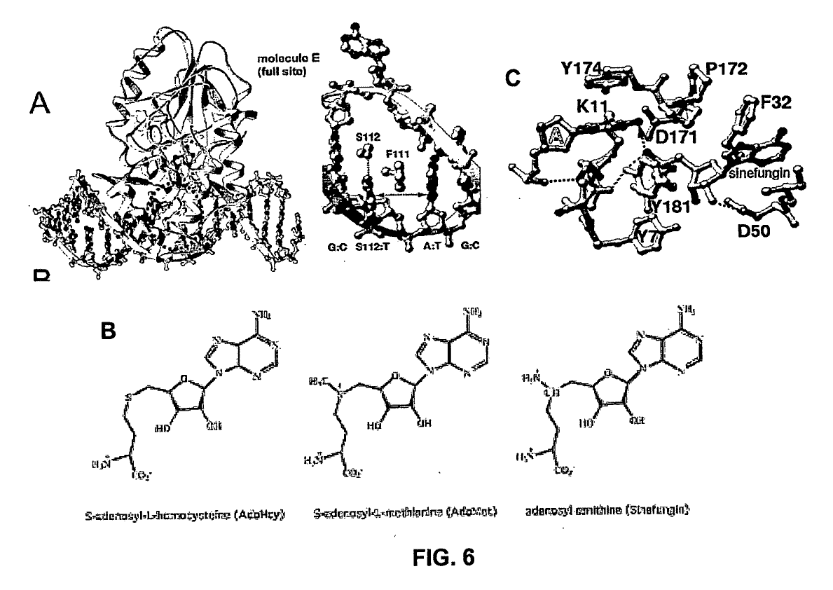

Full-Site Recognition involves a Protein Side Chain Intercalation. Only one T4Dam (molecule E) occupies a GATC site (orange DNA) (FIG. 6A). The β hairpin makes nearly the same specific interactions with DNA bases in the major groove as observed on the 3/4 site. F111 and S122 both insert their side chains into the DNA from the major-groove side (FIG. 6A). Although the target Ade is flipped out of the duplex, its electron density was not very well ordered in the active site (see below for details of active-site interactions). The side chain of S112 occupies the space left by the flipped Ade, forming two hydrogen bonds with the “orphaned” Thy, similar to that observed in the 3/4-site complex. This S112 interaction restores hydrogen bonding to the polar edge of the orphaned Thy and replaces its stacking to the flanking base pairs (FIG. 6A). The Thy-S112 interaction is similar to other protein-side-chain-orphaned base interactions such as those for Gua-Q237 of DNA-cytosine MTase HhaI (Klimasauskas et al., 1994), Thy-Y162 of human 3-methyladenine DNA glycosylase (Lau et al., 1998), and Cyt-N149 of human 8-oxoguanine DNA glycosylase (Bruner et al., 2000).

The phenyl ring of F111 intercalates into the DNA helix and stacks between the adjacent A:T base pair and the Thy:S112 “base-amino acid” pair, resulting in a local doubling in helical rise (FIG. 6A). The intercalation of amino acids between DNA base pairs from the major-groove side has been described for several protein-DNA complexes. In the M.HaeIII-DNA complex, Ile221 lies between the stacked bases and opens a gap in the DNA so that the orphaned Gua pairs with an adjacent Cyt (Reinisch et al., 1995). In the very short patch-repair endonuclease-DNA complex, three aromatic residues intercalate into the DNA next to the TG mismatch (Tsutakawa et al., 1999). In the HincII restriction endonuclease-DNA complex, a Gln side chain intercalates between two base pairs on either side of the recognition site (Horton et al., 2002). In addition, intercalation by the repair enzyme formamido-pyrimidine-DNA glycosylase, in which the F111-M75 residue pair is stacked between the A:T base pair and the base-amino acid pair Cyt:R109, has been observed from the minor-groove side of DNA (Serre et al., 2002).

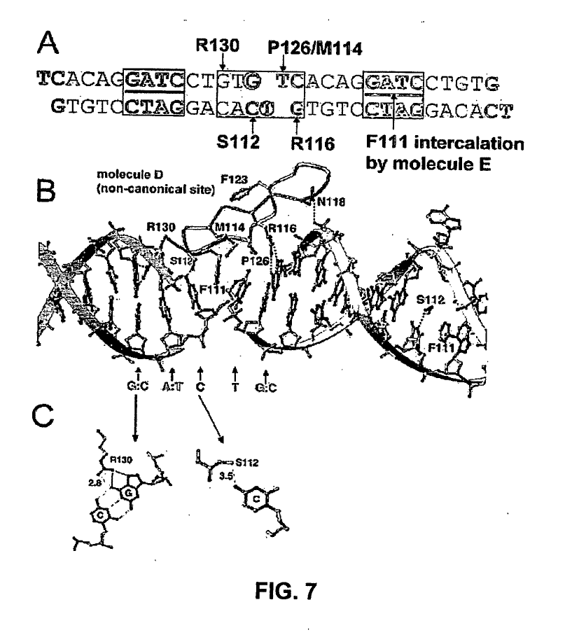

Interaction with a Noncanonical Site. F111 intercalation by molecule E into the central AT stacking effectively causes a one-base-pair lengthening of the DNA molecule depicted in orange (FIGS. 7A and B). The expansion is propagated toward one end of the DNA molecule, resulting in two disordered nucleotides of the neighboring duplex (magenta). The 5′-overhanging Thy of the magenta DNA is pushed out and apparently becomes disordered, resulting in the Cyt of the next base pair stacking with F111 of Dam molecule D. The side chain of S112 approaches the Cyt base with the side chain hydroxyl oxygen and the exocyclic amino nitrogen N4 of the Cyt at a van der Waals distance, partly because of repulsion force between the N4 amino nitrogen (NH2) and the main chain amide nitrogen (NH) (FIG. 7C). The interaction between S112 and Cyt is sufficient to displace the complementary Gua and make it disordered. The side chain of R130 skips the next A:T base pair and interacts with the Gua of the adjacent downstream G:C base pair (FIG. 7C). Since the presence of a Gua downstream of a GATC (or modified TATC) site does not support catalysis (data not shown), this complex exemplifies the interaction of T4Dam with an isolated TC dinucleotide site in the DNA, which does not lead to DNA methylation.

Stabilization of the Flipped Adenine in the Presence of Sinefungin. Thus far we had prepared ternary complexes using the methylation reaction product AdoHcy. The protein-AdoHcy interactions for each protamer are nearly identical to those described in the T4Dam-AdoHcy binary complex (Yang et al., 2003). In the full-site recognition complex between Dam molecule E and the orange DNA (FIG. 6A), the target Ade is flipped out but not fully ordered in the active site. We reasoned that product AdoHcy might signal the enzyme to release from the target site in order to exchange for AdoMet prior to the next methyl transfer. Thus, stable binding of the flipped Ade in the active-site pocket probably requires AdoMet, as has been suggested for EcoDam (Liebert et al., 2004). Therefore, we use the AdoMet analog sinefungin (adenosyl ornithine) to prepare a new ternary complex because it also carries a formal positive charge on the amino group (FIG. 6B).

The new crystal contains two T4Dam molecules (not shown), one bound in the joint of two DNA duplexes, similar to the Dam C molecules in FIG. 5B, and the other bound to the specific GATC site in the middle of one duplex, similar to the Dam E molecule in FIG. 6A. The flipped Ade is surrounded (via hydrogen bonds, π stacking, and hydrophobic interactions) by amino acids belonging to the conserved catalytic D171-P-P-Y174 motif (Malone et al., 1995), Y181, K11, and sinefungin (FIG. 6C). The Ade N6-amino group that becomes methylated forms a pair of hydrogen bonds; one is to the side chain of D171, and the other is to the backbone carbonyl oxygen between the two proline residues P172 and P173. The target amino nitrogen is at a distance of less than 3 Å away from the sinefungin amino group, which is out of the plane of the constrained Ade base. This structural arrangement suggests that the target nitrogen lone pair is deconjugated and positioned for an inline direct methyl-group transfer as suggested for the Taql DNA adenine MTase (Goedecke et al., 2001). The amino group of sinefungin forms a hydrogen bond with the hydroxyl of Y181, which in turn interacts with the main chain carbonyl of T8. The opposite face of the flipped Ade is in a face-to-face tt stacking with the aromatic ring of Y174.

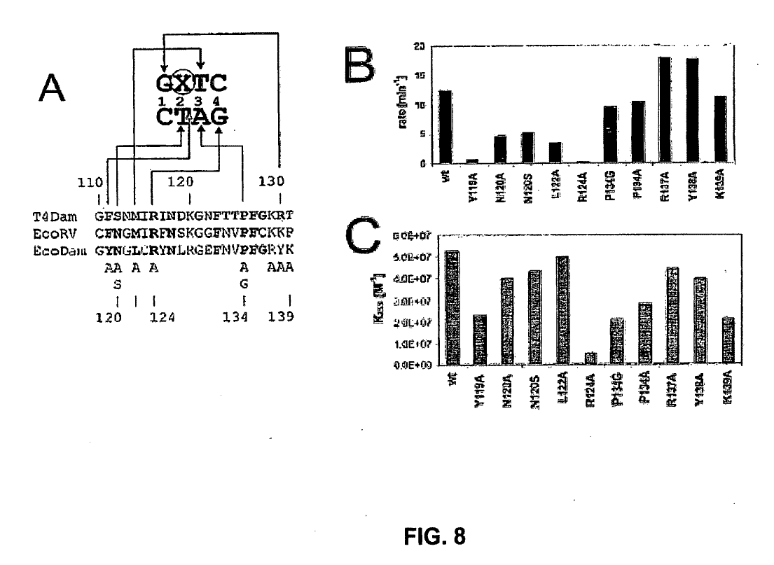

Biochemical Analysis of EcoDam Variants. EcoDam has considerable sequence similarity (25% identity) to T4Dam (Hattman et al., 1985) but has significantly higher sequence conservation with Dam enzymes from pathogenic bacteria. For example, the E. coli and S. typhimurium Dam proteins are 92% identical (differing at only 22 of 278 residues) and have no gaps in their alignment. Because of the biological importance of the Dam family, we investigated whether the T4Dam structures contribute to understanding the function of these orthologs. To this end we studied the effects of substituting Ala for residues in EcoDam (FIG. 8A) that correspond to those involved in T4Dam-specific interaction with its target GATC site. This includes Y119 (F111 in T4Dam), N120 (S112 in T4Dam), L122 (M114 in T4Dam), R124 (R116 in T4Dam), and P134 (P126 in T4Dam). All of these residues are highly conserved among Dam orthologs. In addition, we mutated residues R137, Y138, and K139 since these could assume the function of T4Dam Arg130 (FIG. 8A).

The R124A and Y119A variants were the most strongly affected by the Ala substitution; their catalytic activity was reduced more than 100-fold (FIG. 8B, and see T4Dam R116 and F111 in FIG. 5B). N120A, N120S, and L122A were affected only slightly. DNA binding by the R124A variant was reduced 10-fold (accounting for only one-tenth of the drop in catalytic activity), while binding of Y119A, P134A, P134G, and K139A was reduced 2- to 3-fold (FIG. 8C). The other variants (N120A, N120S, L122A, R137A, Y138A, and K139A) did not display any appreciable difference in DNA binding compared to the wild-type.

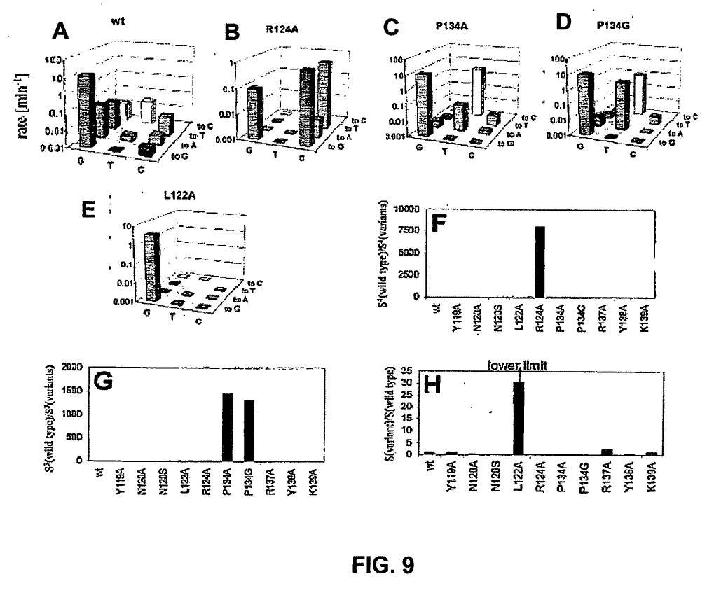

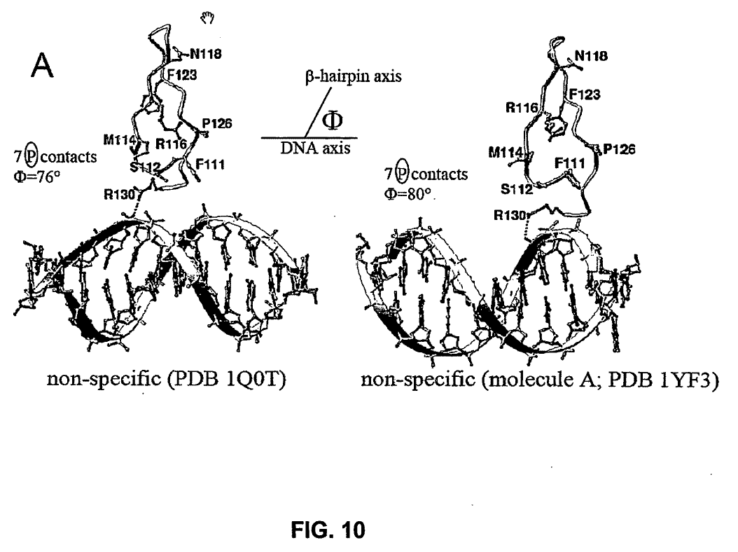

To further investigate the process of DNA recognition, the rate of DNA methylation by the wild-type and variant enzymes was determined using duplexes containing a single hemimethylated target (N6-methyl-Ade in the bottom strand, third base pair in FIG. 8A). This ensured that only one strand of the DNA was subject to methylation (i.e., the Ade of the top strand, second base pair in FIG. 8A). The duplexes contained the canonical GATC site or a variant with a single base substitution at either the first, third, or fourth base pair of the target sequence (see FIG. 8A); these variant sites are designated here as “near-cognate” sites (a total of nine). In this fashion, a specificity profile of the wild-type enzyme and its variants was obtained (FIG. 9). Wild-type EcoDam is a very specific enzyme because near-cognate sites were modified 100- to 1000-fold more slowly than the cognate site (FIG. 9A). The first position of the GATC sequence is recognized less accurately than the third and fourth base, in agreement with earlier findings (Liebert et al., 2004). It is interesting that the contact by R130 of T4Dam to this base is not well conserved among other members of the Dam family (e.g., substituted by Y in EcoDam; see FIG. 8A) and that the R130-Gua1 contact is not yet formed in the 1/4-site complex (compare FIG. 4 and FIG. 5). EcoDam variants altered at residues that might be involved in the recognition of the first base pair (R137A, Y138A, and K139A) did not exhibit any strong changes in methylation activity or specificity (FIG. 12).

In contrast to the first position, the third and fourth bases of GATC are recognized more accurately. At both positions, transitions (Thy3 to Cyt or Cyt4 to Thy) are less deleterious than transversions, indicating that conservative exchanges are more tolerable. The contact between T4Dam R116 and Gua4 (FIG. 5C) is conserved among Dam MTases (e.g., FIG. 8A). We determined the specificity profile of the corresponding EcoDam R124A variant (FIG. 9B). In agreement with the T4Dam structure, GATG and GATT sites were methylated by R124A faster than the canonical GATC site. In contrast, wild-type EcoDam methylation of these two near-cognate sites was three orders of magnitude slower than methylation of GATC. Thus, while R124A has a 100-fold-reduced rate of DNA methylation at GATC sites relative to wild-type EcoDam, it methylated GATG and GATT sites 2- to 3-fold faster than GATC and 30- to 40-fold faster than the wt enzyme modified GATG or GATT. Therefore, R124A has lost the discriminatory requirement for a C:G base pair at the fourth position of GATC. In order to analyze this information more quantitatively, we have defined a specificity factor by integrating the relative methylation activities at all near-cognate sites (Experimental Procedures). A comparison of specificity factors for the recognition of position 4 (S4) reveals that the R124A variant has an 8000-fold-changed relative preference for methylation of near-cognate sites modified at the fourth position (FIG. 9F). No other variant showed such a strong change in S4. Furthermore, the R124A variant retained (or even increased) its specificity for the first and third positions in GATC, so this is a base pair-specific change (FIG. 9B).

We found a similar base pair-specific loss of specificity associated with T4Dam residues P126 and M114, which recognize the T:A base pair at the third position of GATC (see FIG. 8A). Naturally occurring variant phage enzymes (T2Damh and T4Damh), which efficiently modify GACC sites in addition to the canonical GATC site (Brooks and Hattman, 1978), contain a P126S substitution (Miner et al., 1989). In addition, P126G, -A, or -C substitutions behaved in a Damh-like fashion (Miner et al., 1989). In this regard, it is perhaps not surprising that EcoDam P134A and P134G variants had normal catalytic activity at GATC sites (FIG. 8B). However, P134A exhibited a significant increase in methylation rates of GAAC and GACC substrates, with GACC being modified at almost the same rate as canonical GATC (FIG. 9C). This change in preference corresponds to a more than 100-fold loss in sequence discrimination at the third base when compared to wild-type EcoDam. Further shortening of the side chain of P134 to glycine eliminated discrimination between GAAC and GACC, which were methylated at a rate about 10-fold lower than GATC (FIG. 9D). The change in P134A and P134G recognition of the third base pair is illustrated in FIG. 9G, where the specificity factor for recognition of the third base (S3) is compared for all variants. This ratio is shifted 1200- to 1500-fold in comparison to wild-type EcoDam. However, these changes do not result in simple loss of specificity at the third position: GATC sites are still preferred about 10-fold relative to GA(A/C)C, while GAGC sites are methylated at least 1000-fold more slowly.

The activity of the L122A variant of EcoDam (M114 in T4Dam; FIGS. 5B and C) is not appreciably reduced (FIG. 8B). Intriguingly, however, no methylation activity was detectable for any of the near-cognate sites (FIG. 9E). This indicates that the L122A variant has a significantly improved specificity (FIG. 9H). This can be rationalized by assuming that the side chain of L122 is required to stabilize the whole protein-DNA interface. Whereas the L122A change alone does not severely reduce catalytic activity on the normal GATC substrate, a combination of L122A with the change of any of the base pairs in the recognition site may disturb synergistically the protein-DNA interface, and this could explain the complete loss of activity.

In addition to residues making base-specific contacts, we studied the aromatic residue that intercalates into the DNA (Y119 in EcoDam, F111 in T4Dam; FIG. 6A) and the adjacent hydrophilic residue that contacts the orphaned Thy (N120 in EcoDam, S112 in T4Dam; FIG. 6A). As shown in FIG. 8B, Y119A was the second-most-affected variant. This suggests that intercalation of the aromatic ring into the DNA is an important step in enzyme catalysis, possibly involved in initiating or stabilizing base flipping. In contrast, removal of the side chain of N120 (N120A) had only a minor effect on methylation rate, although the structure of the specific T4Dam-DNA complex suggests an important role for this amino acid in base flipping. This finding is consistent with the fact that base flipping is fast and not rate limiting in catalysis for EcoDam and other DNA MTases (Allan et al., 1999, Beck and Jeltsch, 2002 and Liebert et al., 2004).

DNA recognition by proteins is essential for specific expression of genes in any living organism. Although the principle of proteins recognizing DNA sequences by contacts in the major groove has been known for decades (Seeman et al., 1976), there is no general code allowing one to deduce amino acid motifs from their target DNA sequences. Notable exceptions are the C2H2-type zinc fingers, where the DNA recognition process is sufficiently understood to define a DNA recognition code of this family of proteins (Pabo et al., 2001). Consequently, the rational design not only of DNA-interacting enzymes but also of even noncatalytic proteins is still in its infancy.

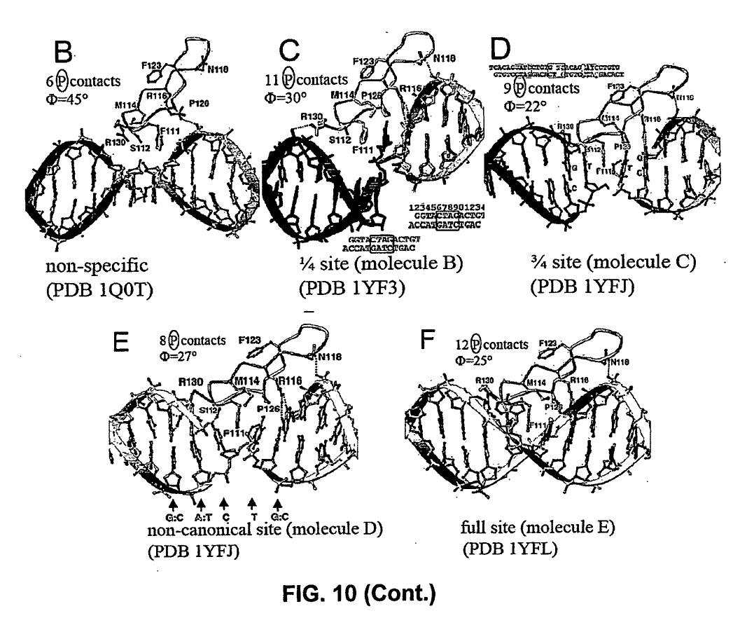

Here we describe six unique T4Dam-DNA interactions along the substrate-recognition pathway (FIG. 10). Surprisingly, both protein and DNA components undergo very little overall conformational change upon binding. The protein structures of the nonspecific, semispecific, and specific complexes can be superimposed within 0.4-0.8 Å of root-mean-square deviation with that of the binary T4Dam-AdoHcy complex (PDB code 1Q0S). The DNA component is primarily in the B form, except for the one-base-pair expansion caused by F111 intercalation and the flipping of the target nucleoside out of the DNA helix and into the enzyme's catalytic pocket in the specific complex. However, three prominent orientations of T4Dam relative to the DNA helical axis were observed. The 6 hairpin loop, whose axis is defined in parallel to the β strands forming the hairpin, sits almost perpendicular (≈80°) to the DNA axis in the nonspecific complexes (FIG. 10A), where R130 forms one phosphate contact. Nonspecific interactions also occur in the DNA minor groove (FIG. 10B), where the protein tilts to ≈45° relative to the DNA and the second Arg of the hairpin loop (R116) forms one of the phosphate interactions. Direct interactions with bases occur only in the DNA major groove (FIG. 10C-F), where the angle between the axes of the β hairpin and DNA is ≈30° in the 1/2-site complex and ≈25° in the 3/4-site and full-site complexes. These results suggest that T4Dam moves along the DNA and rotates up and down as a rigid body relative to the DNA.

Interestingly, the phosphate-interacting residues R95 and N118, which hydrogen bond with the first and second phosphates 5′ to the Gua4 in the specific complex (or in any complex involving R116-Gua4 interaction; FIG. 10C-F), are not involved in any DNA interaction in the nonspecific complexes (FIGS. 10A-B). Two different pairs of residues (Q12 and S13, and R130 and N133) interact with the two phosphates 5′ to each Gua of the GATC palindrome in the nonspecific complex (see FIG. 4B). In contrast, two Arg residues (R130 and R116) can switch roles from a purely electrostatic interaction with the DNA phosphate in the nonspecific complexes (FIGS. 10A-B) to a highly specific binding mode with base pairs of the specific or semispecific complexes (FIGS. 10C-F). A similar switch in interaction with DNA was observed for the residue R22 of E. coli lac repressor (Kalodimos et al., 2004). This switch effectively reorients T4Dam, thereby positioning the enzyme's active-site pocket to accommodate the flipped target base. After catalysis, the enzyme moves away from the target site and rotates back into the perpendicular orientation, exposing the active site to solvent and allowing AdoHcy to exchange for AdoMet. This mechanism ensures that base flipping and methyl transfer specifically occur in a complex with cognate GATC sites and that AdoHcy/AdoMet exchange is possible after each turnover without dissociation from the DNA.

Our data suggest a temporal order for the formation of specific contacts during the one-dimensional sliding of T4Dam along the DNA. The contact of R116 to the fourth base pair of the GATC site is observed in the 1/4- and 3/4-site recognition complexes. Next, the contacts of P126 and M114 to the third base pair are formed. All of these residues are strictly conserved within the Dam MTase family. The contact of R130 to Gua1 that is specific to T4Dam is formed later. This result agrees with a similar conclusion drawn from rapid kinetics experiments with M.EcoRV variants (Beck and Jeltsch, 2002). In this enzyme, substitutions of amino acids conserved in the enzyme family (such as N136A) interfered with specific complex formation at an early state, while substitutions of amino acids characteristic of EcoRV (such as R145A) interfered with complex formation at later stages. This finding might illustrate a general pathway for changes of DNA specificity of proteins and enzymes during molecular evolution. The recent study on human DNA-repair protein O6-alkylguanine-DNA alkyltransferase (AGT) suggested that the recruitment of multiple AGT molecules to the same region of DNA might aid the search for DNA damage through a process of directional bias (Daniels et al., 2004). However, such directional bias was only observed for the repair of single-stranded DNA by AGT but not for double-stranded DNA, and the system cannot be directly compared to the Dam MTases because T4Dam and EcoDam move along double-stranded DNA (FIG. 5A), whereas AGT forms polymers.

We analyzed the biochemical effects of altering the contacts described above in double-mutant cycles (Fersht et al., 1992). This involved shortening the respective amino acid side chains and using DNA substrates with near-cognate sites. We found that it was possible to predictably design MTase variants that no longer recognize one specific base pair within their recognition site. The EcoDam R124A variant displayed a change in specificity because it had a significantly higher catalytic activity toward a near-cognate site. In addition, the EcoDam P134A variant (the analog of the T4Damh MTase) methylated a near-cognate site at almost the same rate as wild-type EcoDam modified the canonical site, indicating a broadened specificity (FIG. 9).

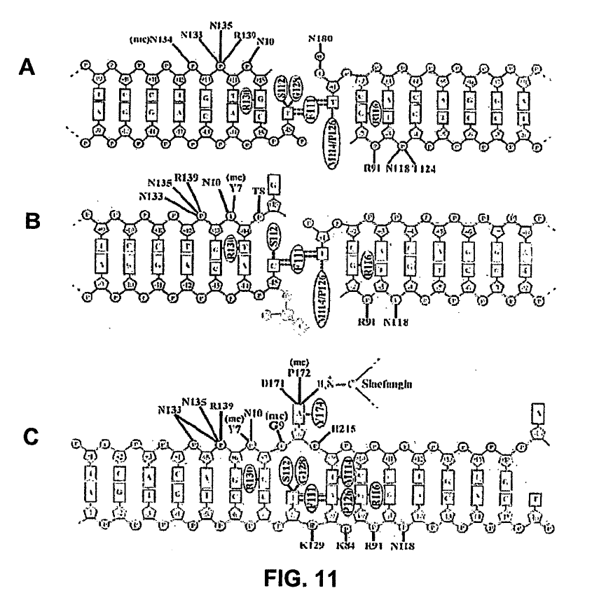

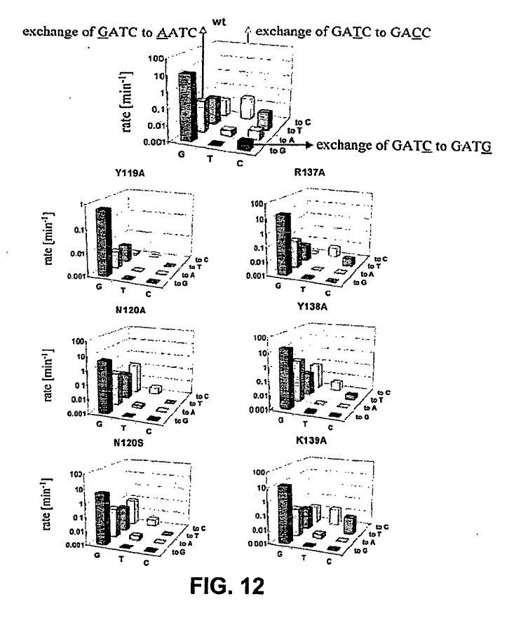

FIG. 11 summarizes the protein-DNA contacts for the (A) 3/4 site; (B) non-canonical site; and (C) specific full site. FIG. 12 graphically summarizes the effects of various EcoDam variants on the rate of methylation.

We have identified two types of protein DNA contacts, discriminatory and antidiscriminatory. A discriminatory contact is one that stabilizes the transition state of enzymatic catalysis and specifically accelerates the reaction with the cognate site. The contact between R116 of T4Dam (R124 of EcoDam) and the Gua4 is an example of a discriminatory contact. Disruption of the contact by removal of the amino acid side chain led to a strongly reduced activity of the enzyme variant. An antidiscriminatory contact, e.g., the contact between P126 of T4Dam (P134 of EcoDam) and the third base pair of the recognition site, is one that does not significantly accelerate the reaction with the cognate site but disfavors activity at near-cognate sites because steric clashes may occur if the wrong DNA sequence is bound. This would strongly interfere with methylation of most noncanonical DNA sequences and lead to an efficient counterselection against methylation of nontarget sites. This is illustrated by the high activity and broadened specificity of EcoDam variants P134A and P134G.

Comparison with restriction-modification MTase M.DpnII: T4Dam and MTase M.Dpnii (Tran et la. 1998) both recognize and methylate the same GATC sequence, and have quite similar structure, but differ substantially in their processivity. It has been suggested that processive enzymes, like T4Dam, tend to more fully enclose their substrates. Breyer and Matthews (2001). However, since the M.DpnII-DNA complex structure is currently not available, a direct structural comparison with T4Dam cannot be made.

The cofactor (e.g. AcoHcy) binding site in all structures of T4Dam examined thus far is in a closed acidic pocket. In contrast, an open conformation was observed in the binary M.DpnII-AdoMet structure, where the AdoMet is largely visible and the pocket is opened up (Tran et al 1998). This difference suggests that the exchange of AdoHcy with AdoMet, the rate limiting step in the overall T4Dam methylation process (Malygin et al. 2000), requires a conformational change in the protein. This conformational change in T4Dam, which includes Trp185, was demonstrated by quenching of intrinsic tryptophan fluorescence that results from T4Dam binding either AdoMet of AdoHcy (Tuzikov et al. 1997)

Transition from Nonspecific to Specific DNA Interactions along the Substrate-Recognition Pathway of Dam Methyltransferase. DNA methyltransferases methylate target bases within specific nucleotide sequences. Three structures are described for bacteriophage T4 DNA-adenine methyltransferase (T4Dam) in ternary complexes with partially and fully specific DNA and a methyl-donor analog. We also report the effects of substitutions in the related Escherichia coli DNA methyltransferase (EcoDam), altering residues corresponding to those involved in specific interaction with the canonical GATC target sequence in T4Dam. We have identified two types of protein-DNA interactions: discriminatory contacts, which stabilize the transition state and accelerate methylation of the cognate site, and antidiscriminatory contacts, which do not significantly affect methylation of the cognate site but disfavor activity at noncognate sites. These structures illustrate the transition in enzyme-DNA interaction from nonspecific to specific interaction, suggesting that there is a temporal order for formation of specific contacts.

Structural snapshots along the substrate recognition pathway. In the study of T4Dam (FIG. 10), we caught many snapshots of DNA interactions along the substrate recognition pathway by using different lengths of oligonucleotides (to allow more than one Dam molecule bind one piece of DNA), different end sequence of the DNA duplex (to represent part of the GATC target sequence), different ratio of protein to DNA (to reduce nonspecific binding), and methyl donor analog sinefungin (to stabilize the flipped target Ade in the active site pocket). The opportunity to acquire these many snapshots of T4Dam has relevance to understand the entire GATC-related MTase orthologs as well as other processive DNA binding enzymes. However, because T4Dam and EcoDam only share 25% sequence identity and most of the non-identical residues are located on the surface, it is necessary to identify many interfaces of EcoDam-DNA interactions, following the leads of T4Dam. Through structural studies of EcoDam, we delineate those protein-DNA interfaces that are unique to EcoDam and target them for in silico screening (ISS). Similarly, specific parts of the AdoMet binding pocket and unique cavities on the EcoDam surface are used for ISS.

Information on various structure of the enzyme in complex with DNA and mechanistic insights into the DNA methylation process impacts the ISS process and thus, the identification of possible leads, because one can design inhibitors that are selective for certain conformational states. Determining the areas of protein surface responsible for non-specific and specific DNA interactions assists in targeting these areas individually in ISS. Inhibitors interfering with specific DNA binding prevent the transition of non-specific to specific DNA interaction, or they can interfere with sliding of the enzyme along the DNA. Inhibitors that block AdoMet exchange can affect processivity.

### Example 2

### X-Ray Crystallography of E. Coli Dam

Adenine methylation in hemimethylated GATC sites produced by DNA replication regulates bacterial cell functions including gene expression, mismatch repair, and virulence in many Gram-negative bacteria. The widespread and conserved enzyme DNA adenine methyltransferase (Dam) in γ-proteobacteria methylates GATC sites by scanning the genome. Structures have been solved for Escherichia coli Dam (EcoDam), interacting with a cognate and a non-cognate site in the presence of cofactor analogs. The non-cognate complex allowed identification of a potential DNA binding element, TA(G/A)AC, immediately flanking GATC sites in many Dam-regulated promoters. Accompanied by biochemical studies, the structures reveal a chronological order of formation of specific enzyme-DNA interactions. Contacts to the non-target strand in the second (3′) half of the GATC site are established early in the recognition pathway, initially to the fourth, and then to the third base pair. Then, intercalation of specific protein side chains into the DNA helix between the second and third base pairs occurs in concert with flipping of the target Ade. Contact to the first Gua in GATC is established later. The flipped target Ade bound to an alternative base-binding site suggests a possible late intermediate in the base-flipping pathway. The orphan Thy can adopt an intrahelical or extrahelical position.

We report two crystal structures of EcoDam, bound to cognate or non-cognate DNA, in the presence of a cofactor analog. The non-cognate DNA complex allowed us to identify a potential DNA binding element. TA(G/A)AC. immediately flanking GATC sites in many Dam-regulated promoters, which suggests a mechanism of regulation of dam methylation in bacterial DNA. Together with parallel biochemical studies, we verified structural predictions and reconciled the effect of site-directed mutations on DNA binding, target-sequence specificity and base flipping. By combining structural and kinetic data we also determined the sequential order for formation of specific contacts between the enzyme and the DNA and base flipping. The flipped target Ade bound to an alternative base-binding site suggests a possible late intermediate in the base-flipping pathway. The ‘orphan’ Thy can adopt an intrahelical or extrahelical position.

His Tag-EcoDam is expressed in HMS174(DE3) cells and purified using Ni²⁺-affinity, UnoS, and S75 Sepharose sizing columns. A 0.5-liter induced culture yields approximately 7 mg purified His Tag-EcoDam. In the last purification step and during concentration, cofactor analog AdoHcy or sinefungin is added is added to the protein at approximately 2:1 molar ratio. Concentrated binary complexes are mixed with oligonucleotide duplex (synthesized by New England Biolabs, Inc) at a protein to DNA ratio of about 2:1 and allowed to stand on ice for at least two hours before crystallization. Final protein concentration for crystallization is about 15 mg/mL. In hanging drop crystallization trials with AdoHcy, the ternary complex crystals appeared under low salt conditions of 100 mM KCl, 10 mM MgSO₄, 5-15% PEG400, and 100 mM buffer (MES or HEPES) pH 6.6-7.4 (the cognate crystal form in Table 4). In crystallization trials with sinefungin, the ternary complex crystals grew under similar low salt conditions, but resulted in different cell dimensions (the non-cognate crystal form in Table 4).

Structural determination of the cognate ternary complex proceeded by molecular replacement with the program REPLACE (Tong and Rossmann, 1997) using protein coordinates of a DpnM monomer structure (PDB 2DPM) (Tran et al., 1998). The DpnM model was modified based on pair-wise sequence alignment of EcoDam with DpnM; differing amino acids in DpnM were changed to those in EcoDam and visually given the best rotamer using the program O (Jones et al., 1991), and some amino acids were deleted in the loop regions. DNA molecules were built manually into densities of difference maps. Refinement proceeded with the program CNS (Brunger et al., 1998). Structure of the non-cognate ternary complex was determined using a protein monomer from the refined cognate complex structure as a search model.

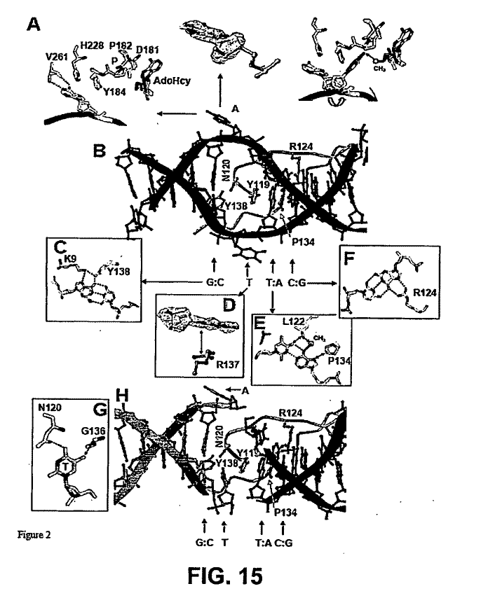

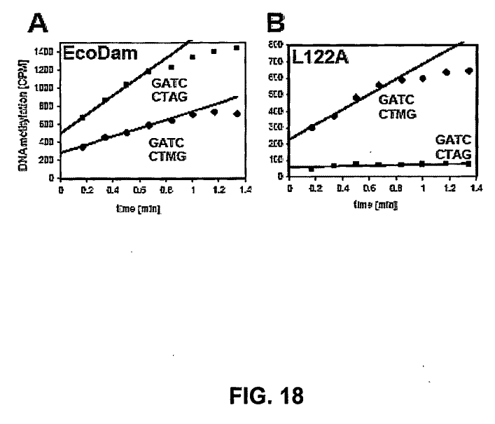

Site directed mutagenesis was performed as described (Jeltsch and Lanio, 2002). EcoDam wild type and its variants were purified as described (Horton et al., 2005). DNA binding was analyzed using surface plasmon resonance in a BiaCore X instrument as described (Horton et al., 2005). Methylation of oligonucleotide substrates (purchased from Thermo Electron, Dreieich, Germany in purified form) was carried out as described (Horton et al., 2005). Methylation experiments were performed in 50 mM Hepes (pH 7.5), 50 mM NaCl, 1 mM EDTA, 0.5 mM DTT, 0.2 μg/μl BSA containing 0.76 μm [methyl-³H]AdoMet (NEN) at 37° C. as described (Roth and Jeltsch, 2000) using single-turnover-conditions with 0.5 μM oligonucleotide substrate and 0.6 μM enzyme for specificity analysis (FIGS. 15B-C) and 0.25 μM enzyme for the study of interaction with hemimethylated DNA (FIG. 18). The sequence of the 20-mer oligonucleotide substrate was a duplex of 5′-GCGACAGTGATCGGCCTGTC-3′ and 5′-GACAGGCCGMTCACTGTC GC-3′ where M is N6-methyl-Ade. In addition nine substrates with near cognate sites, differing in one base pair from GATC at the first, third or fourth position, were used. To compare the recognition of the first position of the target sequence by different variants, a specificity factor was defined as the ratio between the rates of methylation of all near-cognate sites modified at other positions and the rates of methylation of substrates modified at the first position, viz.

S1=(k_{GATG}+k_{GATA}+k_{GATT}+k_{GAGC}+k_{GAAC}+k_{GACC})/(k_{AATC}+k_{TATC}+k_{CATC})

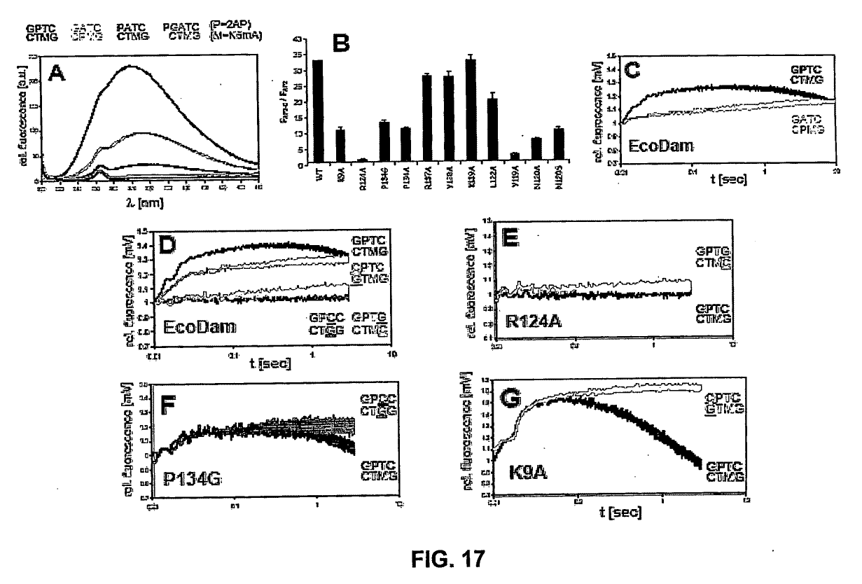

To measure equilibrium base flipping, the fluorescence change of oligonucleotides containing 2AP was detected in the absence and presence of EcoDam using 2 μM of enzyme and 0.5 μM DNA in 50 mM Hepes (pH 7.5), 50 mM NaCl containing 100 μM AdoHcy at ambient temperature (FIG. 17). The 2AP fluorescence was excited at 313 nm in a F2810 spectrofluorimeter (Hitachi). Emission spectra were recorded between 320 and 500 nm. Emission and excitation slits were set to 2.5 nm and the data were analyzed by integration of the fluorescence peak after subtraction of the background signal from the buffer sample alone. The kinetics of base flipping were investigated by stopped-flow experiments performed in an SF-3 stopped flow device (BioLogic, Claix, France) as described (Liebert et al., 2004) using enzyme and DNA at equal concentrations (350 nM) at ambient temperature. The enzyme was pre-incubated in buffer containing 50 mM Hepes (pH 7.5), 50 mM NaCl and 10 μM AdoMet and rapidly mixed with DNA in the same buffer (FIG. 17C). The 2AP fluorescence was excited at 313 nm and emission was observed using a 340 nm cutoff filter. The dead time of the experiments was 3.1 ms.

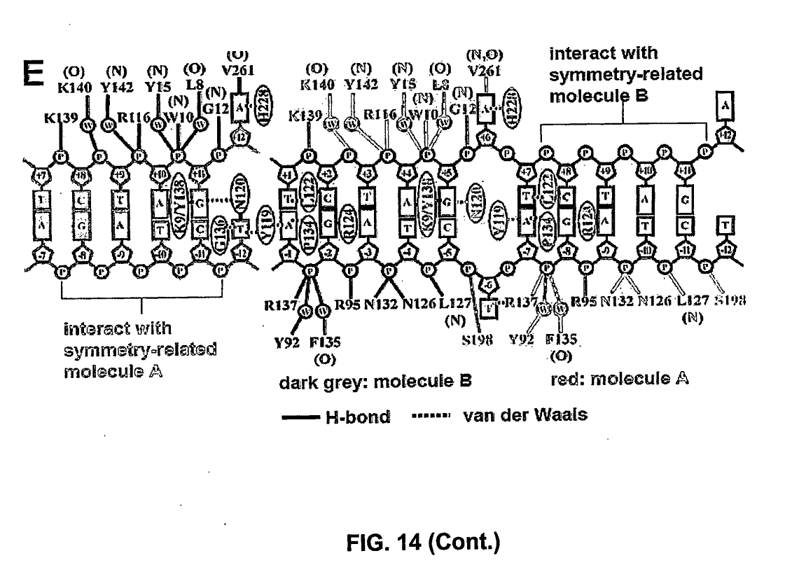

We crystallized a ternary complex containing EcoDam, AdoHcy, and a 12-mer oligodeoxynucleotide duplex containing a single centrally located GATC target site (FIG. 13). The end sequence of the duplex was chosen such that the sequence at the joint of two molecules mimics a GATC target site if the DNA duplexes are stacked head-to-tail (FIG. 14A). Design of the blunt-end oligonucleotide was based on the observation that T4Dam preferentially binds at the joint of two duplexes (Horton et al., 2005). The resulting crystals diffracted X-rays to 1.89 Å resolution (cognate crystal form in Table 5).

It is difficult to produce E. coli Dam crystals, and is particularly difficult without DNA. Utilizing insight obtained from successful crystallization of T4Dam, we designed an oligonucleotide with the following properties: (1) optimized length to maximize the DNA-mediated protein-protein contacts in the crystal packing lattice; and (2) the two end-sequences of the oligonucleotide to mimic a GATC site if the two DNA duplexes are stacked head to tail. Accordingly, we use 12-mer DNA 5′-TCTAGATCTAGA-3′. In addition, protein to DNA ratio (>2:1) is varied so that all joints between neighboring DNA duplexes and the central GATC site are occupied by Dam molecules. With these properties, we successfully crystallized EcoDam in complex with DNA and AdoHcy. The crystals diffracted X-rays to higher resolution. In particular, orthorhombic crystal form (space group P2₁2₁2₁) with unit cells of a=44.8 Å, b=70.2 Å, c=96.5 Å were grown with 5-15% PEG 400, 100 mM KCl, 10 mM MgSO₄, and 100 mM buffer (MES or HEPES) pH 6.6-7.4. A data set diffracted to 1.89 Å resolution is shown in Table 5. The structure was solved by molecular replacement using T4Dam as an initial search model, and the model refined to an R-factor of 0.186 and R-free of 0.215 (FIG. 13).

A different hexagonal crystal form (space group of P3_{(1,2)}21) with the same 12 bp DNA has also been observed (unit cells a=b=159.5 Å, c= 93.7 Å) under the conditions of 1.5 M Li(SO₄)₂, 50 mM MgSO₄, 100 mM buffer (MES or HEPES) pH 6.8-7.2.

Overall structure of EcoDam: Two EcoDam monomers (molecules A and B) and one DNA duplex are contained in the crystallographic asymmetric unit. EcoDam molecule A primarily binds to a single DNA duplex, while EcoDam molecule B binds the joint between the two DNA duplexes (FIG. 14B). EcoDam, like T4Dam (Yang et al., 2003), contain two domains: a seven-stranded catalytic domain harboring the binding site for AdoHcy and a DNA binding domain consisting of a five-helix bundle and a β-hairpin loop (residues 118-139, red in FIGS. 14B and 14C) that is conserved in the family of GATC-related MTase orthologs (Yang et al., 2003). The two protein molecules are highly similar with a root-mean-square deviation of 0.07 Å comparing 241 pairs of Cα atoms. Two regions are disordered in both molecules (FIGS. 14C and 14D): residues 188-197 immediately after the active-site D181-P-P-Y184 motif (after strand β4) and residues 247-259 between strands β6 and β7.

EcoDam-DNA phosphate interactions: The EcoDam molecule spans ten base pairs, four base pairs on 5′ side and five on 3′ side of the flipped-out target Ade (FIG. 14E), whether they are from a single DNA duplex (EcoDam molecule A) or the joint between two 12-mer DNA duplexes (EcoDam molecule B). Five phosphate groups 5′ to the Ade residues in both strands are in contact with a single EcoDam molecule. The phosphate interactions with the non-target strand seem to be more important than those with the target strand. This is suggested by the fact that there are four conserved residues (R95, N126, N132, and R137) among the side chains making direct interactions with the phosphate groups, all of which interact with three consecutive phosphate groups flanking the Gua of the fourth GATC base pair of the non-target strand (FIG. 14E).

EcoDam-DNA base interactions: The methylation target, the Ade of the second base pair in GATC (Ade2), flips out from the DNA helix (FIG. 15A). The specific interactions with the remaining bases of the site occur in the DNA major groove. Like T4Dam, the amino acids residues from the β-hairpin (red in FIG. 15B) make the majority of base specific interactions, but K9 from the N terminal loop also forms a base contact (cyan in FIG. 14C). Two regions—the β-hairpin and the N terminal loop—are connected tightly together through many intra-molecular interactions including hydrogen binding of the main chain amide nitrogen and carbonyl oxygen of K9 with the N115 side chain carbonyl and amide, respectively (not shown). The following sections describe EcoDam recognition of the first, third and fourth base pair, and its interaction with the target base pair, including protein side chain intercalation and DNA base-flipping.

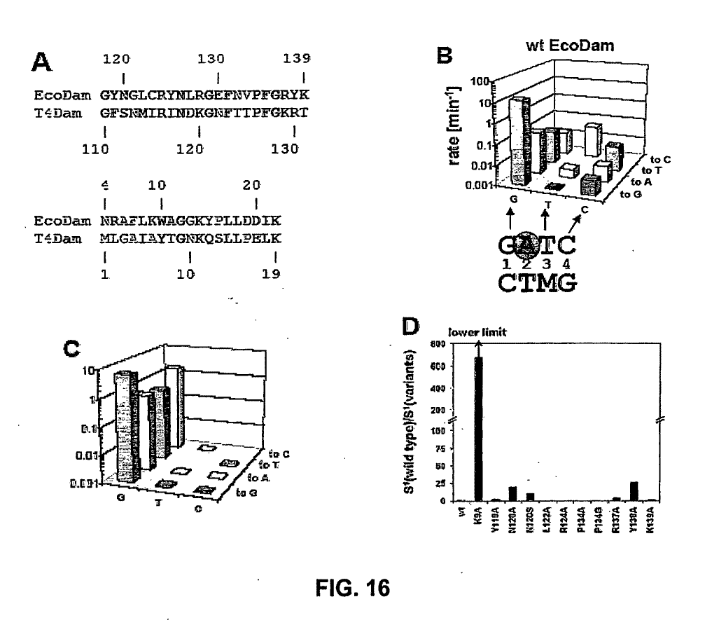

Recognition of the first base pair by N-terminal K9: Their recognition of the first base pair is one of the most interesting deviations between T4Dam and EcoDam. In the T4Dam structure, the first Gua of the GATC site is contacted by R130 with bifurcated hydrogen bonds to the N7 and O6 atoms of Gua1 (Horton et al., 2005). R130 is located at the end of the β-hairpin, but it is not conserved among the Dam-related MTases (FIG. 16A). Previously, we examined the involvement of EcoDam Y138 (which on the basis of the alignment directly corresponds to R130 in T4Dam; FIG. 16A) and the two flanking residues —R137 and K139—for a role in the recognition of the first base pair; however changing these residues to Ala did not affect the DNA recognition of EcoDam strongly (Horton et al., 2005). The EcoDam structure shows that Gua1 interacts, via the N7 and 06 atoms respectively, with two side chains, K9 and Y138 (FIG. 15C). At the position corresponding to EcoDam K9, T4Dam has an Ala (FIG. 16A) whose β-carbon points towards the DNA, but does not contact it. The EcoDam K9A variant shows slightly reduced catalytic activity (˜60% of wild type) (comparing the light blue bars in FIGS. 16B and 16C) and DNA binding (˜70%) (data not shown).

To further investigate DNA recognition by EcoDam, we compared the rates of DNA methylation of the canonical versus variant duplexes, all containing a single hemimethylated target (FIG. 16B) to ensure that only one strand of the DNA was subject to methylation. The variant duplexes contained a single base substitution at either the first, third, or fourth base pair of the target sequence; these variant sites are designated here as “near-cognate” site (a total of nine) (FIG. 16B). The results showed that relative to GATC, near-cognate substrates that carrying a base pair substitution at the first position were methylated by wild type EcoDam at a 100- to 1000-fold reduced rate (FIG. 16B). In contrast the K9A variant showed a loss of specificity at the first base pair; because relative to GATC the rate of methylation of CATC was only four-fold lower, and AATC and TATC methylation was 10-fold reduced (FIG. 16C). In addition, the K9A variant was unable to methylate any of the near-cognate sites, carrying a substitution in the third or fourth base pair, demonstrating an increased discrimination for these positions. This is probably due to the disruption of some additional protein-DNA contacts (by mutation of the DNA sequence) that is required for catalysis.

A specificity factor (S1) for the recognition of Gua1 was calculated for K9A, which is given by the average of the methylation rates of all near cognate substrates carrying an alteration at the first base pair divided by the average methylation rate of all other near cognate substrates. On the basis of S1, in comparison to wild type EcoDam, K9A has an at least 800-fold reduced recognition of the first base pair (FIG. 16D), whereas all other variants displayed only minor effects. These results demonstrate that the EcoDam K9-Gua1 contact (via N7 atom) is important for recognition of the first base pair in GATC. Y138A (loses its interaction with the O6 atom of Gua1, FIG. 15C) and N120A (loses its n-stacking with Gua1, FIG. 15B) also show small changes in specificity factor S1, indicating that S1 correctly identified all three side chains that are in the vicinity of Gua1.

Interaction with the target Ade and base flipping: Incorporation of the nucleotide analog 2-aminopurine (2AP) into synthetic oligodeoxynucleotide duplexes has been used extensively to probe conformational changes, such as base flipping (Allan et al., 1998; Allan and Reich, 1996; Holz et al., 1998; Stivers, 1998), because 2AP fluorescence increases dramatically when it is removed from the stacking environment of double helical DNA (Ward et al., 1969). Fluorescence changes of a hemimethylated G-2AP-TC substrate, which carries 2AP at the position of the target Ade, was correlated with base flipping by EcoDam (Liebert et al., 2004). Base flipping by EcoDam comprises two steps: (i) flipping of the target base out of the DNA helix, and (ii) binding of the flipped base into the active site pocket of the enzyme (formed by the D181-P-P-Y184 motif). Target base flipping leads to a complete loss of the stacking interactions of the Ade with the neighbor bases which causes a strong increase in fluorescence. During binding of the flipped base into the active site pocket it stacks to aromatic residue(s), which leads to a reduction of 2AP fluorescence during this step of trapping (Liebert et al., 2004). Rapid kinetic measurements with a hemimethylated G-2AP-TC substrate demonstrated that, in the presence of AdoMet, base flipping by EcoDam was a biphasic process (FIG. 17C). The initial flipping was very fast, but insertion of the flipped base into the active site pocket was slower. However, in the absence of coenzyme AdoMet or the presence of AdoHcy, the slow phase of fluorescence reduction was not observed. This suggested that binding of the flipped target base into the active site pocket does not occur if no AdoMet is bound to the enzyme (Liebert et al., 2004).

In agreement with these observations, in the current structure formed in the presence of AdoHcy, the flipped target Ade lies against the protein surface (side chains of Y184 and H222) outside the active-site pocket (FIG. 15A, left panel). The imidazole ring of H222 makes a cation-π interaction with the Ade ring. In addition, the ring nitrogen atom N1 and the exocyclic amino nitrogen N6 atom of the Ade form a hydrogen bond with the main chain amide nitrogen and carbonyl oxygen of V261, respectively. Comparison of the DNA conformation with that in the T4Dam complex (Horton et al., 2005) reveals that simple but large rotations (>120°) around only three dihedral bonds would drive insertion of the flipped Ade into the active-site pocket (FIG. 15A, right panel).

The existence of a conformation in which the flipped Ade is not bound to the active site suggests that the base flipping occurs through a series of intermediates (Banerjee et al., 2005; Horton et al., 2004; Liebert et al., 2004). The Ade-binding mode observed here in the EcoDam complex could mimic a late intermediate in the Ade-flipping pathway; viz., one just before insertion of the base into the active-site pocket. Alternatively, the conformation could be viewed as an intermediate formed immediately after release from the active-site pocket. Therefore, we reasoned that product AdoHcy might signal the enzyme to exchange for AdoMet prior to the binding of the flipped Ade into the active site pocket. This coupling could be mediated by the dynamic conformations of the loop adjacent to the active-site (see FIG. 14D); the corresponding loop in T4Dam contacts the bound cofactor with several aromatic residues (Yang et al., 2003). The loop is unstructured in the current EcoDam model, but it might adopt a stable structure after the binding of AdoMet, which could trigger insertion of the flipped Ade into the active-site pocket.

Interaction with the orphan Thy: a double base flipping: The conformation of the orphan Thy (opposite the flipped Ade) represents a major difference between the EcoDam-DNA complexes formed by molecule A versus molecule B. Unexpectedly, in molecule A the orphan Thy in the center is also flipped out of the DNA helix, where it is stabilized by the n-stacking interactions with the guanidino group of R137 (FIG. 15D). In contrast, the orphan Thy in molecule B hydrogen bonds with the amide side chain of N120 (FIG. 15G). There, N120 inserts its side chain into the helical space vacated by the flipped Ade. These results illustrate that the orphan Thy can adopt at least two different conformations. Therefore, we examined whether Thy-flipping occurs in solution. To this end, we used a substrate that has 2AP substituted in place of the Thy to be orphaned. Under equilibrium binding conditions in the presence of AdoHcy, a 6-fold fluorescence increase was observed (FIG. 17A), which indicates a significant loss of Thy stacking interactions. Because in the intrahelical conformation at the DNA joints (bound by molecule B) there is no obvious change in the stacking interaction of the Thy and no changes in the DNA conformation (bending or unwinding), this protein-induced change in fluorescence intensity indicates that Thy-flipping also occurs in solution. Fast kinetics experiments demonstrated that Thy-flipping also takes place in the presence of AdoMet. Since Thy-flipping is slower than target Ade-flipping it suggests that the two events are not coupled (FIG. 4C). Thy-flipping Was also observed with the R137A variant (data not shown), indicating that docking of the flipped Thy to R137 (FIG. 15D) is not required for flipping, and that there might be other alternative extrahelical conformations for the flipped Thy.

Y119 intercalation is necessary for base flipping: The Y119 aromatic ring intercalates into the DNA duplex and stacks between the third base pair of GATC and the Thy:N120 “base-amino acid” pair in the joint (FIG. 15H) or the side chain of N120 in the center (FIG. 15B), resulting in a local doubling in helical rise. The helical expansions in the middle and end of the DNA duplex effectively increase the length of the DNA such that it corresponds to 14 base pairs, matching the crystal a axis with the length of ˜46 Å. Previously, we have shown that substitution of Y119 by Ala led to a strong reduction in catalytic activity (Horton et al., 2005). Fluorescence studies reveal an almost complete loss of detectable base flipping either in the presence of AdoHcy (FIG. 17B) or AdoMet (data not shown)—Y119A was the second most important residue (after R124) in base flipping (FIG. 17B). Therefore, intercalation of Y119 into the DNA is a necessary event for base flipping. The invasion of N120 into the DNA helix is of less importance; its substitution by Ala reduced catalytic activity (Horton et al., 2005) and Ade-flipping only about 2-3 fold (FIG. 17B) (Horton et al., 2005).

Recognition of the third base pair: discrimination of unmethylated and hemimethylated DNA. The third base pair of GATC makes van der Waals contacts with two hydrophobic side chains of L122 and P134 (FIG. 15E). Hemimethylated GATC sites produced during DNA replication are the natural in vivo substrates for the Dam MTase. We modeled a methyl group onto the exocyclic amino nitrogen N6 atom of Ade3 (FIG. 15E): the methyl group sits between the side chains of L122 and P134, but the L122-CH₃ contact distance (˜3.6 Å) is much shorter than that of P134-CH₃ (˜4.9 Å). Thus, we studied the influence of residue L122 on EcoDam interaction with hemimethylated DNA duplexes. As shown in FIG. 18A, the rate of methyl transfer with the unmethylated substrate was roughly twice as fast as with the hemimethylated substrate. This finding is expected because the unmethylated substrate has twice the number of target sites as the hemimethylated one. If the initial EcoDam binding is random with respect to the two strands, then each binding event to the unmethylated substrate is productive and leads to methylation. In contrast, 50% of the binding events with the hemimethylated substrate will be unproductive, because EcoDam will be positioned such that the methylated Ade would be at the target position. However, the L122A variant showed a drastically altered behavior (FIG. 18B); viz., it was almost inactive on unmethylated DNA, while modifying the hemimethylated substrate at a rate similar to wild type EcoDam.

The mechanism of this pronounced change in the catalytic properties of L122A is not clear. Without wishing to be bound by any particular theory, we postulate that the Ala at position 122 interacts with the methyl group of methylated Ade3, to compensate for the loss of the contact between L122 and Thy3 (see FIG. 15E). It is interesting to note that a single point mutation (L122A), which reduced the size of an aliphatic hydrocarbon side chain, was sufficient to convert EcoDam into a bona fide maintenance MTase with pronounced preference for hemimethylated DNA. The mammalian maintenance MTase, Dnmt1, has a high preference for hemimethylated CpG sites over unmethylated CpG sites (Fatemi et al., 2001; Hermann et al., 2004), and it plays a central role in the propagation of CpG methylation patterns in mammals (Grace Goll and Bestor, 2005). The mechanistic basis for this selectivity of Dnmt1 is unknown, but it must be based on the selective activation of the enzyme by the presence of single methyl groups at hemimethylated CpG sites. Our results provide an example of such recognition of a single methyl group.

Recognition of the fourth base pair: the first step of specific DNA interactions: The Gua in the fourth base pair of GATC interacts via its O6 and N7 atoms with the guanidino group of R124 in a bifurcated hydrogen bonding pattern (FIG. 15F). R124-Gua4 interaction is identical to that observed in T4Dam, and we have previously shown that R124 has a critical role in DNA recognition by EcoDam (Horton et al., 2005). The R124A variant had an overall reduction in catalytic activity but methylated two near-cognate substrates (GATT and GATG) faster than the canonical GATC, demonstrating that the interaction of R124 and Gua4 (“discriminatory contact”) is required to activate the enzyme for catalysis (Horton et al., 2005).

As shown in FIG. 17D, wild type EcoDam shows no detectable change in 2AP fluorescence with substrates containing sequence changes at the third or fourth base pair (green or red lines in FIG. 17D), whereas base flipping occurs with substrates containing a base substitution in the first base pair. Conversely, no base-flipping signal was detected with the R124A variant (FIG. 17E), which correlates with its pronounced reduction in catalytic activity.

These findings demonstrate that there is a coupling between DNA recognition and base flipping by EcoDam. The contacts of the β-hairpin loop with second half of the recognition sequence (the third and fourth base pairs) are required to position the enzyme on the target sequence. In particular, we hypothesize that the Gua4 base contact by R124, and its flanking phosphate contacts by conserved residues (see above), positions EcoDam on the DNA duplex such that other residues involved in base flipping (such as Y119) and DNA recognition (such as L122 and P134) can approach the DNA and induce base flipping. This notion is further supported by the next structure.

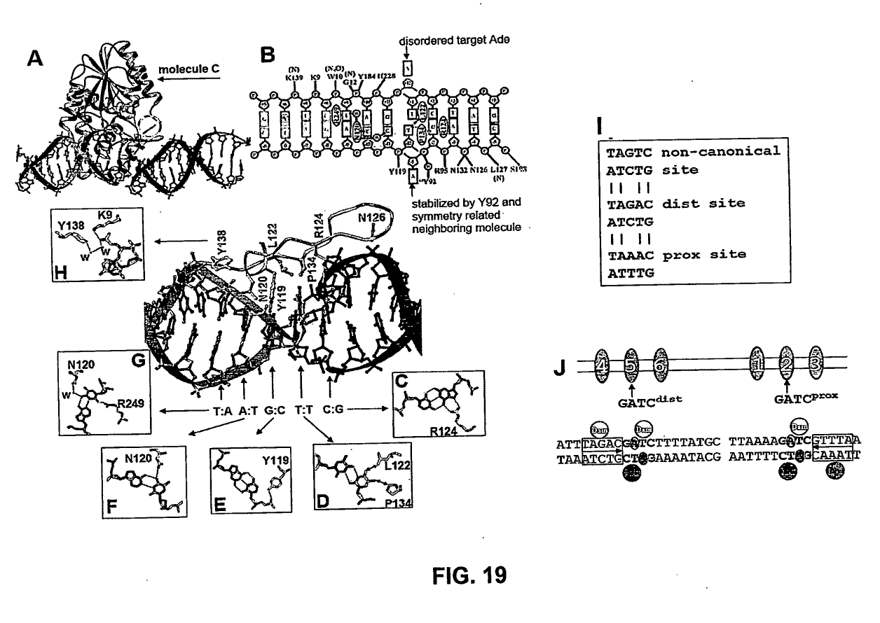

Interaction with a non-canonical site: implication in regulation of pap expression: A second crystal form was produced in the presence of the AdoMet analog sinefungin (adenosyl ornithine) (Table 4) and the same 12-mer blunt-end oligodeoxynucleotide duplex for crystallization. There were at least three unexpected observations. First, although an EcoDam molecule (designated as molecule C, to distinguish it from the A and B molecules shown in FIG. 14) was bound to the joint between neighboring DNA duplexes, no EcoDam molecule was bound to the specific GATC site in the middle of the duplex (FIG. 19A). Second, each DNA duplex formed only 11, instead of 12, base pairs stacked head-to-tail along the crystal a axis with a length of ˜36 Å (average helical rise per base pair of ˜3.3 Å). The shorter length left insufficient space for a second EcoDam molecule to bind in the middle of the DNA duplex. Third, the electron density maps indicate that the two 3′ Ade bases at the ends of each DNA duplex were flipped out (with one being disordered and the other stabilized) and the two 5′ Thy bases formed a T:T mismatch at the joint of the two DNA molecules (FIG. 19B). It is unclear what caused both Ades to become extrahelical.

Five base pairs in the joint are in contact with molecule C (FIG. 19C): three from the green DNA, the T:T mispair, and one from the blue DNA (designated a non-canonical site). The interaction of the 5′ Gua (blue DNA) with R124 (FIG. 19C) and the interactions of its 5′ phosphates are identical with those of molecules A and B. One Thy of the T:T mispair, the one displacing the Ade3, has van der Waals interactions with L122 and P134 (FIG. 19D). Other residues, previously identified as involved in intercalation (Y119), base-amino acid pair (N120), and the first base pair recognition (K9 and Y138), are located in the major groove of the green DNA. It is as if they were positioned for invasion into the DNA, but then switched their roles to phosphate contacts (Y119 and K9), base contacts (N120), or water-mediated DNA interactions (Y138) (FIGS. 19E-H). An additional base contact is formed in the minor groove of the green DNA by R249 (FIG. 19G), which is part of the disordered loop between strands β6 and β7 in molecules A and B. Taken together, these interactions suggest EcoDam is able to bind a non-canonical sequence of 5′-TAGTC-3′ or 5′-GTCTA-3′ (FIG. 191), although we did not intend to design such a site.

The Pap regulon contains two GATC sites (FIG. 19J). In contrast to most GATC site in the E. coli genome, these sites are not always completely methylated after DNA replication but their methylation state determines in part the phase variation of pili formation, which occurs without a DNA sequence change (Hernday et al., 2003). Accordingly, the failure to methylate these sites may be due to the binding of regulatory proteins that block access of EcoDam (Hernday et al., 2003) or to an inherent loss of enzyme activity at these sites due to the particular sequence of the DNA. Interestingly, a recent study showed that methylation of these two sites is nonprocessive in the absence any regulators, which suggested that sequences flanking these GATC sites might prevent the processivity of EcoDam (Mashhoon et al., 2004). This is reminiscent of an earlier observation where the ability of EcoDam to methylate a particular GATC depended on the immediate flanking DNA sequences (Bergerat et al., 1989). After inspecting the Pap GATC flanking sequences (FIG. 19J) we were surprised to find that sequence elements (TAGAC or TAAAC), with high similarity to the non-canonical TAGTC site, are present immediately flanking both GATC sites (FIGS. 191 and J). The two TA(G/A)AC elements are in opposite orientations and they differ at the third base pair, which has no direct base contact in the structure of our non-canonical complex (FIG. 19E). Encouraged by the identification of these potential DNA binding elements), we searched the literature for EcoDam-regulated promoters and found that this element (TANAC) is present in many cases (Table 6). These data raise the possibility that the TANAC elements can trap EcoDam before it binds to or after it leaves the GATC site. As the element overlaps with the papl responsive element (Hernday et al., 2003), the trapped EcoDam could interfere with Lrp-papl binding and contribute to the regulation of pap expression.

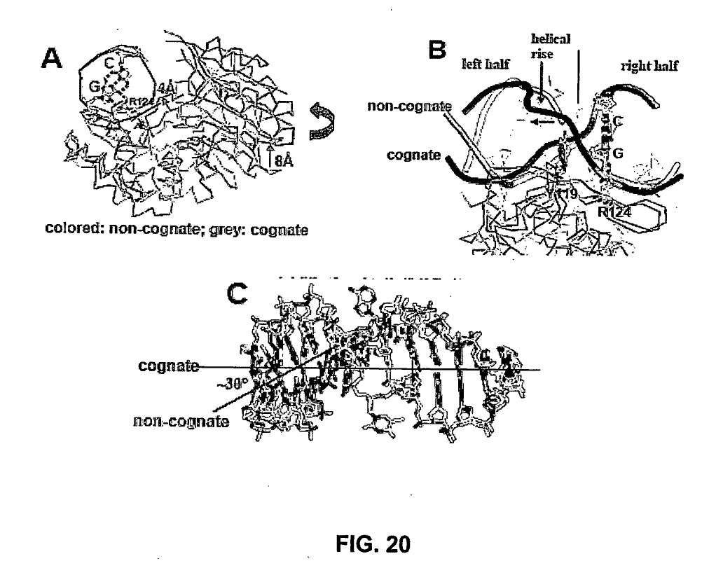

Coupling of base flipping and DNA recognition: A central mystery of DNA methylation concerns the mechanism by which DNA MTases cause flipping of the target base within their recognition sequences. The present structures of the cognate and non-cognate complexes shed some light on this process. Comparison of the DNA conformation in the non-canonical site (bound with molecule C) with that in the canonical site (bound with molecule A or B) reveals the detailed conformational changes that take place in the earlier stages of DNA recognition and the final stage of base flipping. Shown in FIG. 20A is a least-square superimposition of the two EcoDam (molecules A and C), using the R124:Gua4 pair only to determine the superimposition. The protein component displays a rigid hinge movement towards the DNA from the non-cognate complex to the cognate complex: the N-terminal loop in the DNA interface moved approximately 4A and the residues in the outer surface away from the DNA moved approximately 8-9 Å (compare the overall r.m.s. deviation of ˜0.3 Å between the two protein components). The two DNA duplexes show high concordance in the interaction pattern of right half including the fourth base pair (right side of FIG. 20B), with the backbone of non-target strand being held in place through electrostatic interaction with R95, hydrogen-bonding interactions with the side chains of N126, N137, the main chain of L127, and the conserved Gua4-R124 interaction. On the other hand, the helix conformation of the left half (left side of FIG. 20B) is markedly different in the two structures. Inspection of the backbone conformation reveals that shifting the left flank of the non-canonical duplex (by Y119 intercalation) along the helix axis and rotating approximately 30° about the helix axis would result in the conformation of the canonical complex (FIG. 20C). During this process, the protein component does not require any major conformational changes, almost all significantly important side chains (such as Y119 shown in FIG. 20B) line up in the DNA major groove of both complexes. Y119 and K9 switch roles from interactions with the DNA phosphate in the non-canonical complex to a highly specific binding mode in the canonical complex. The intercalation by Y119 (which deeply penetrates the DNA helix) is an essential step to interrupt helical staking on both strands and enforce the one-base-pair lengthening of the DNA molecule, resulting in correct contacts between the first G:C base pair and the side chains of K9 and Y138 and the base flipping of substrate Ade in the second base pair. It is interesting to note that the length of 5-base pair recognition in the non-cognate complex is the same as the 4-base pair plus one intercalation step in the cognate complex.

To study the coupling of base flipping and DNA recognition, we investigated base flipping by EcoDam variants with an altered specificity by P134G and P134A (which show reduced discrimination at the third base pair) and K9A (which has relaxed recognition of the first base pair). In agreement with their high catalytic activities P134G (FIG. 17F) and P134A (data not shown), exhibited only a small reduction in the amplitude of the fluorescence change, but no detectable changes in the kinetics of base flipping. However, both variants induced base flipping of the substrate with altered sequence at the third base pair (green line in FIG. 17F), which did not occur with wild type EcoDam (green line in FIG. 17D). K9A behaved in a similar fashion: base flipping of substrates carrying a base pair substitution at the first position of the target site was more efficient than with wild type EcoDam (compare the cyan lines in FIG. 17G and FIG. 17D). We conclude that the change in specificity of these variants is based on a change in their flipping Ade in near-cognate sites.

On the basis of the structural comparison shown in FIG. 20 and the foregoing discussion, the contacts to the non-target strand of the right-side DNA—R124-Gua4 and its 5′ phosphate interactions—are established early in the recognition pathway, probably before the target base Ade2 is actually flipped. Close approach of the enzyme to the DNA then requires a T:A base pair at the third position. Then, Y119 and N120 can intercalate into the DNA and base flipping takes place. The contact to the first base pair is established later. Thus, the formation of the specific complex of EcoDam resembles the closure of a zipper from the fourth to the first base pair. With the left-side interactions firmly engaged and the flipped Ade positioned on the outside edge of the active site (FIG. 15A, left panel), simple bond rotation about the DNA backbone at the extrahelical nucleotide put the target Ade into the active site. This movement only takes place after AdoMet binding when the unstructured loop immediately following the active-site becomes ordered and enables the formation of closed active site. A flexible conformation of the orphan Thy could have a role in the dynamics of the base flipping process. Only after these events have taken place, and the target Ade has entered the active site, does catalysis of methyl transfer from AdoMet take place (see FIG. 15A, right panel). A binding to a non-cognate element, TA(G/A)AC, which also requires a R124-Gua interaction in the 3′ end, traps EcoDam in the Dam-regulated promoters, affect EcoDam processivity and contribute to the regulation of gene expression.

Compounds identified from the in silico screen (ISS) (see Example 3) can be further studied structurally by co-crystallization with Dam. The structural information obtained from these co-crystals can be used to identify site(s) of structural variability to generate derivatives around the same core chemical structure, via synthesis of a compound library, with more desirable properties.

Crystallization of mutant Dam or pap-associated GATC substrate to address processivity of DNA methylation: EcoDam methylates DNA in a highly processive reaction (Urig et al., 2002). After each methylation event the coenzyme product AdoHcy must be exchanged with AdoMet before next round of reaction. Processive methylation requires that this exchange occur while the enzyme stays bound to the DNA. An inhibitor that prevents the sliding of the enzyme along the DNA and/or blocks AdoHcy/AdoMet exchange can affect processivity, and thereby inhibit methylation.

In T4Dam structure, a channel connects the coenzyme binding site and the solvent (not shown). This channel is important for processive methylation by Dam, as it can allow the exchange of coenzyme without releasing the enzyme from DNA. In the context of this invention, the channel provides an additional docking site unique for Dam, with the potential of finding more specific inhibitors. These inhibitors can either prevent AdoHcy/AdoMet exchange or they can diffuse into the AdoMet binding pocket and sterically interfere with AdoMet binding. These possible modes of action can be distinguished by comparing the effects of the inhibitors on AdoMet binding and processivity of DNA methylation. Ile51 forms one wall of the channel in T4Dam. We have changed the corresponding residue (Ile55 in EcoDam) to Trp and Arg in an attempt to block the channel and interfere with processivity. Initial results show that blocking the channel by the I55W substitution strongly compromises activity. The I55R variant is as active as the wildtype enzyme on short oligonucleotide substrates. The processivity of I55R mutant is examined using the assay described in Urig et al. (2002), and measure the K_{d} of AdoMet binding to both mutants. Co-crystal structures of these mutants with coenzyme indicates whether/how these substitutions affects coenzyme interaction. If this channel indeed affects coenzyme binding/exchange, we can pursue ISS of this site to identify additional Dam inhibitors.

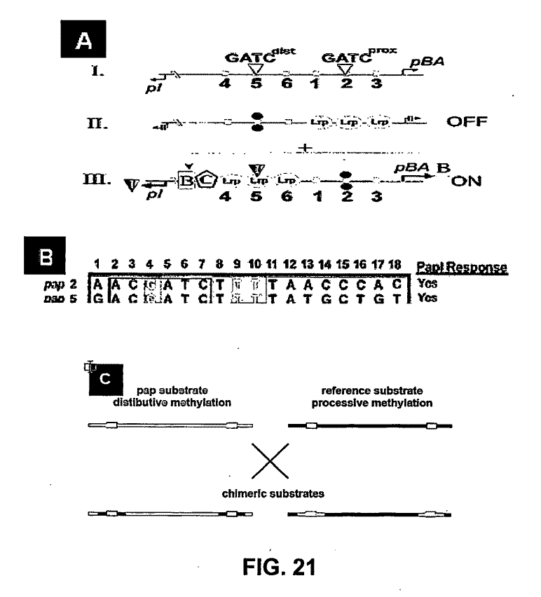

The Pap regulon contains two GATC sites separated by 103 bp (FIG. 21). The methylation state of these two GATC sites in part determines the phase variation of pili formation, which occurs without a DNA sequence change (Hernday et al., 2003). Based on Hernday et al. (2003), the reason EcoDam only methylates one of the sites is due to the regulatory proteins Lrp and Papl binding and blocking Dam access. However, a recent study showed that methylation of these two sites are nonprocessive in the absence any Lrp or Papl, and suggested that sequences flanking these GATC sites might prevent the processivity of EcoDam (Mashhoon et al., 2004). We can reproduce this finding and investigate its mechanism. One possibility is that the conserved sequence flanking both GATC sites forms additional DNA interaction with the enzyme, and interferes with the initial binding or product release; both would affect processivity. Alternatively, the sequences in between the two Pap sites (separated by 103 bp) could prevent efficient sliding of the enzyme and thereby interfere with processive methylation. To discriminate between these two models we can create two chimeric substrates of the Pap substrate and a normal DNA sequence (FIG. 21C). One substrate contains the two Pap GATC sites with 5 flanking base pairs connected by normal DNA sequence, while the other contains two normal Dam sites separated by the pap intermediate sequence. EcoDam should modify at least one of these substrates non-processively. If the flanking sequence contributes to processivity, we can determine the structure of EcoDam with Pap-associated GATC sites with flank sequences. If additional protein-DNA interactions are observed, we can generate targeted mutant proteins and examine their processivity and sequence specificity.

In bacteria usually all MTase target sites are methylated. However, the ability of an MTase to regulate the expression of genes critically depends on the existence of a methylation pattern, which means that certain sites must be protected against the constitutive methylation. It is very likely that some signals in the sequence context of the pap-sites contribute to this protection and the loss of processivity of EcoDam in methylation of the pap site could be one effect. The identification of these signals that prevent processive methylation can help find other bacterial genes that are differentially methylated, and whose expression may be modulated by Dam methylation. This approach can help to understand how dam methylation regulates pathogenicity of bacteria.

### Example 3

### In Silico Screen

Given the recent success in identifying the same inhibitor of the TGF-β receptor kinase by two different routes—one using ISS and one using high-throughput screening (HTS) (Sawyer et al., 2003; Singh et al., 2003), it is evident that the appropriately guided ISS approaches can be as successful as HTS (Liu et al., 2004) (Waszkowycz, 2002). In addition, molecular docking is less labor intensive. For example, it has a 6% hit rate, compared with <0.2% for HTS in the screen for tuberculosis target dihydrodipicolinate reductase against the Merck chemical collection (Paiva et al., 2001).

Two major goals must be considered during ISS. First is the need to identify compounds that effectively inhibit Dam methylation. Second is the need for those compounds to be specific for bacterial Dam molecules versus other MTases. With respect to the first consideration, we target a number of potential binding sites on EcoDam. From the crystallographic structures, we expect to have many snapshots of Dam molecules proceeding from non-binding pockets to target via ISS. Potential sites include the AdoMet binding pocket and channel(s) into and out of the pocket, the hinge region between the catalytic and DNA binding domains, DNA binding surfaces (specific and non-specific), and unique surface pockets. Notably, conformational changes in the presence of AdoMet/AdoHcy or DNA or flipped target Ade may influence the size or shape or particular cavities; these attributes are checked by structural comparison of the different forms of Dam characterized via crystallography. In addition, by targeting the cofactor-bound Dam structure, inhibitors may be identified that are active even in the presence of high levels of AdoMet (since such high levels can exist intracellularly). Final selection of binding sites includes homology considerations, with the goal of obtaining broad-spectrum antibiotics, as well as the quality of sites for binding of compounds. The latter will be determined by performing preliminary docking against the putative sites, with the quality of each site determined based on docking scores and geometries.

Selectivity of the inhibitors for Dam versus other MTases is a very important criterion for a successful antibiotic. A compound selective for Dam should have a high probability of binding to other Dam proteins but a low probability of binding to non-Dam MTases. For the latter, compounds selected from our initial screen (50,000 compounds, see below) are also screened against the following non-Dam MTases: PRMT1—a protein arginine MTase (Zhang and Cheng, 2003), and DIM-5—a histone H3 Lys9 MTase (Zhang et al., 2002) (Zhang et al., 2003) and the binding energies with these proteins are incorporated into the selectivity score described in the next section. Such selective screening is especially important for inhibitors targeting the cofactor binding region, as the potential for a lack of selectivity is the highest in this functionally similar region of the protein. We also screen the compounds against Salmonella Dam, such that compounds that score favorably against both E. coli and Salmonella Dam, but not to other non-Dam MTases, are preferentially selected for biological testing. Because there is currently no 3D structure of Salmonella Dam, we produce a homology model based on E. coli Dam using the program Modeller (Sali and Blundell, 1993). Accuracy of the modeling is aided by the fact that there are only 22 residues different between the E. coli and Salmonella Dam (92% identity), with almost the same number of amino acids (278 vs. 277) and no gaps between them. Once a crystallographic structure for Salmonella Dam is obtained, the structure can be used for docking in a manner equivalent to Dam from T4 and E. coli.

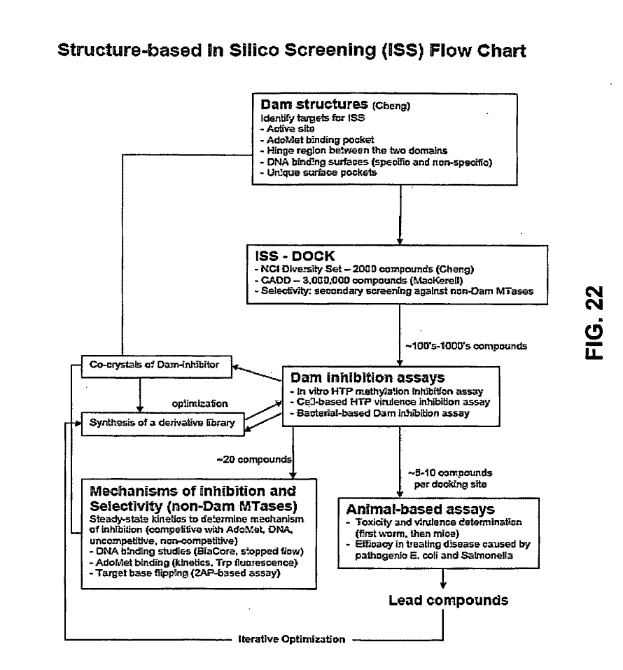

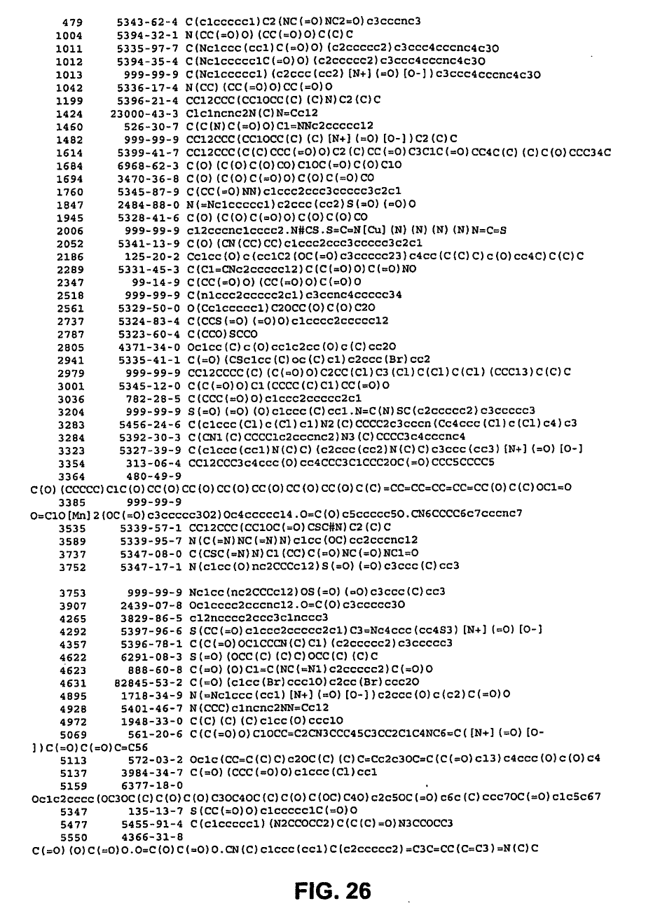

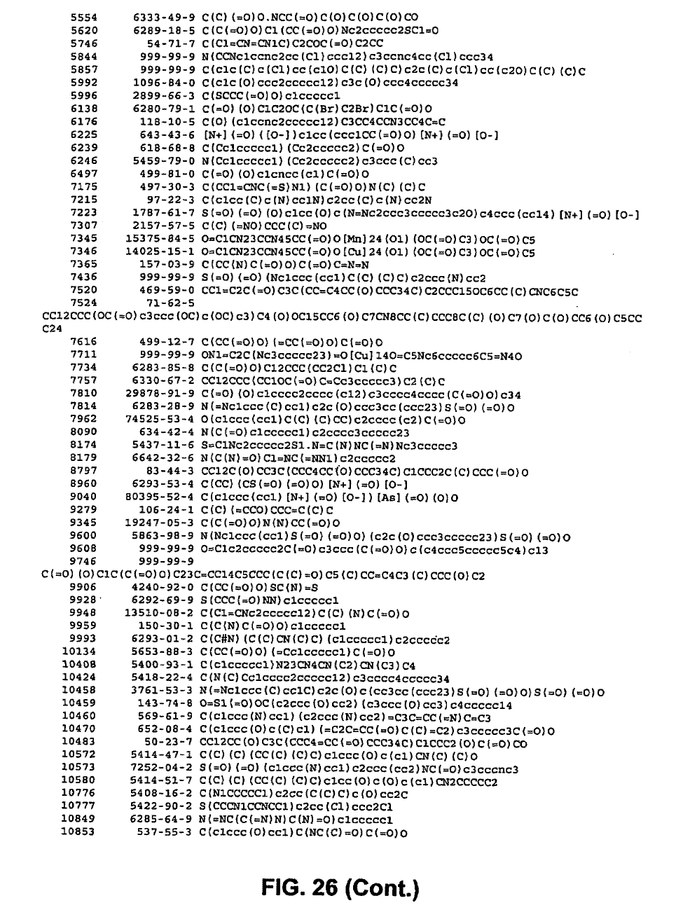

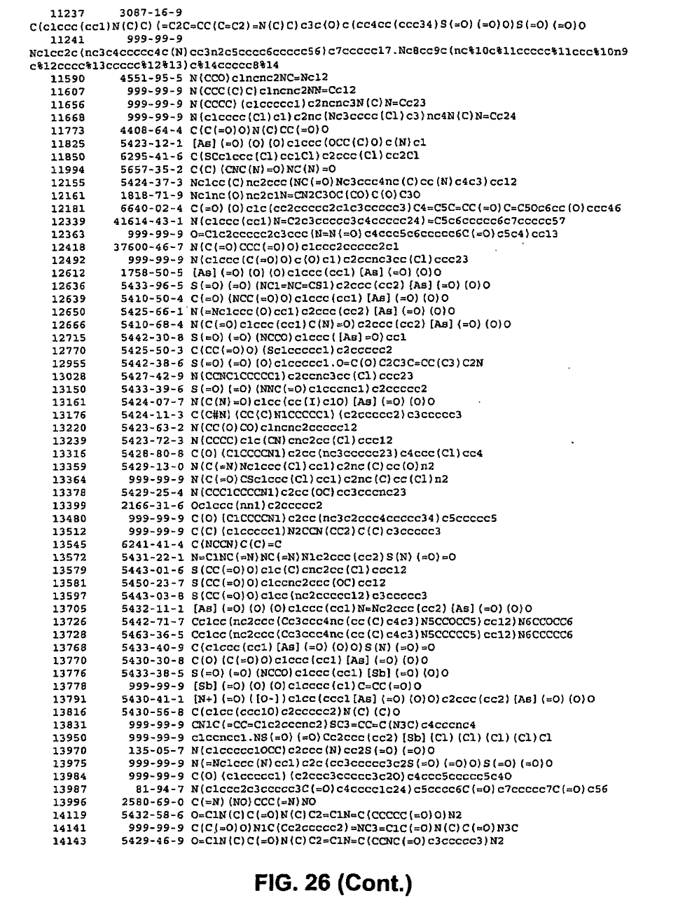

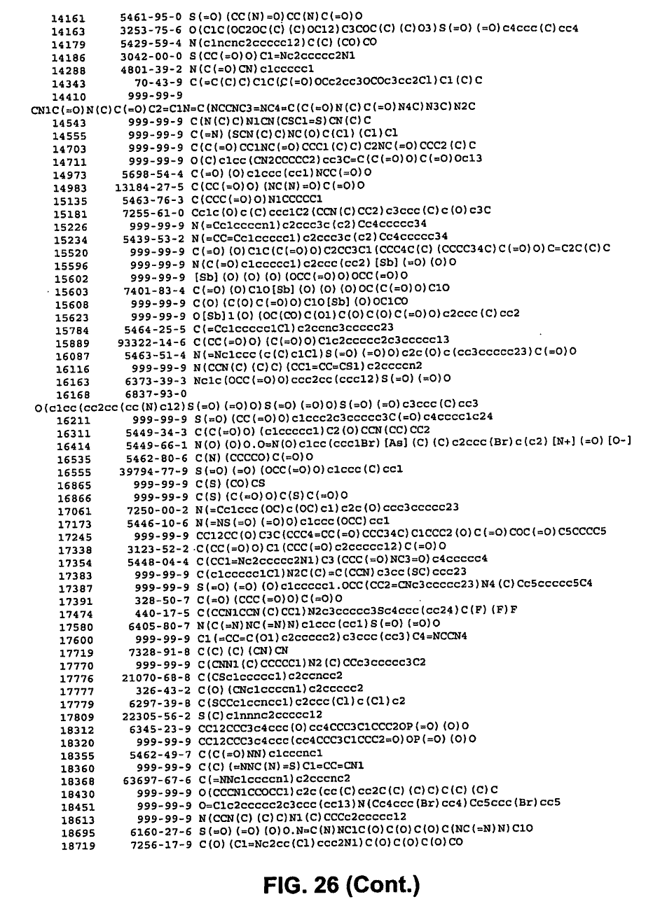

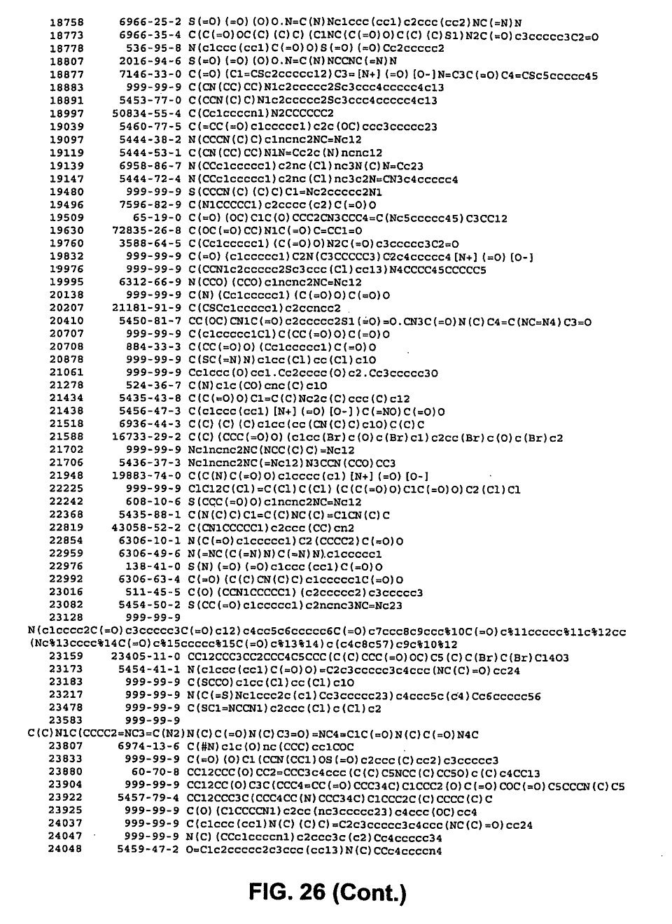

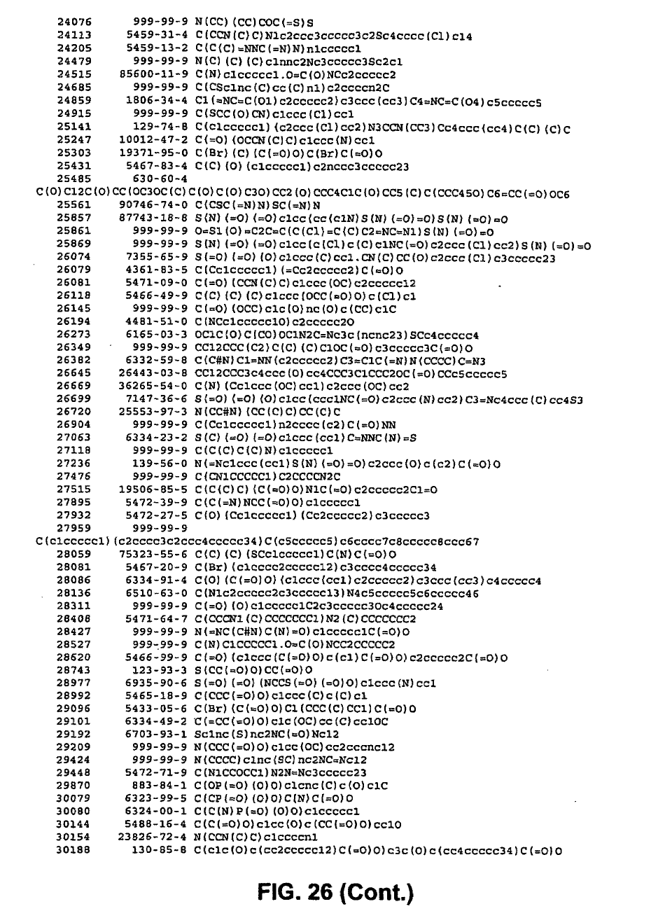

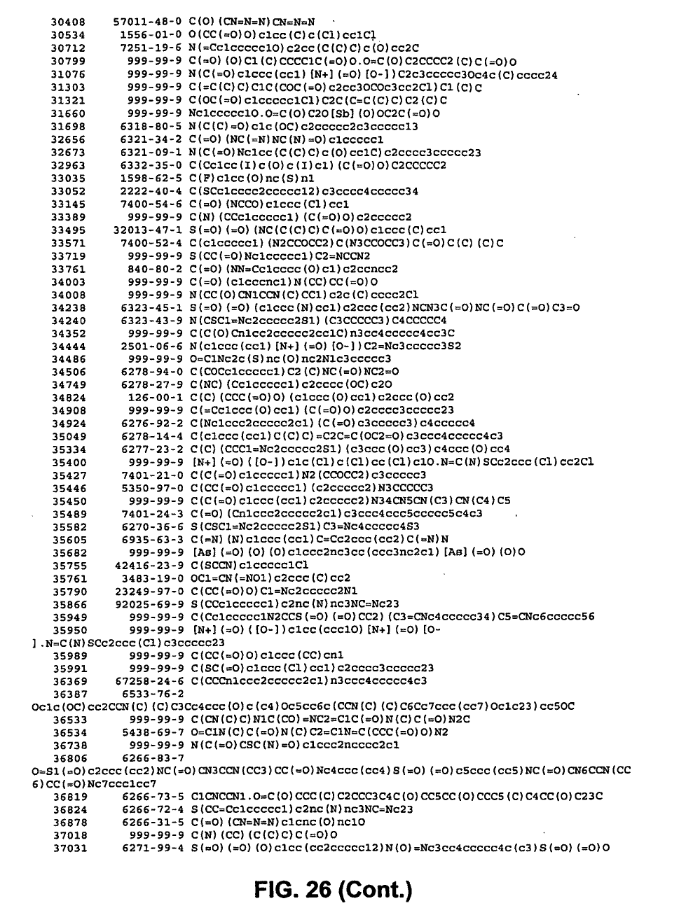

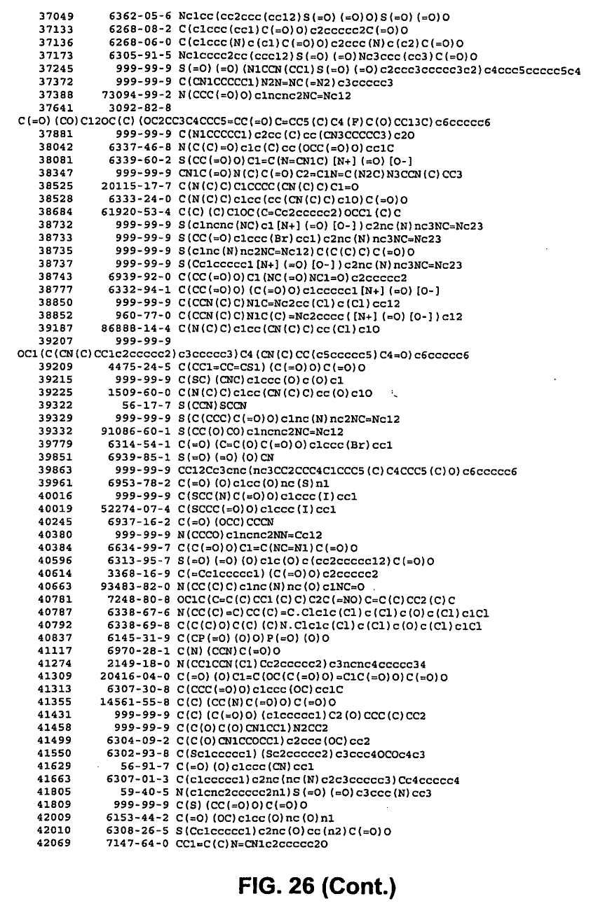

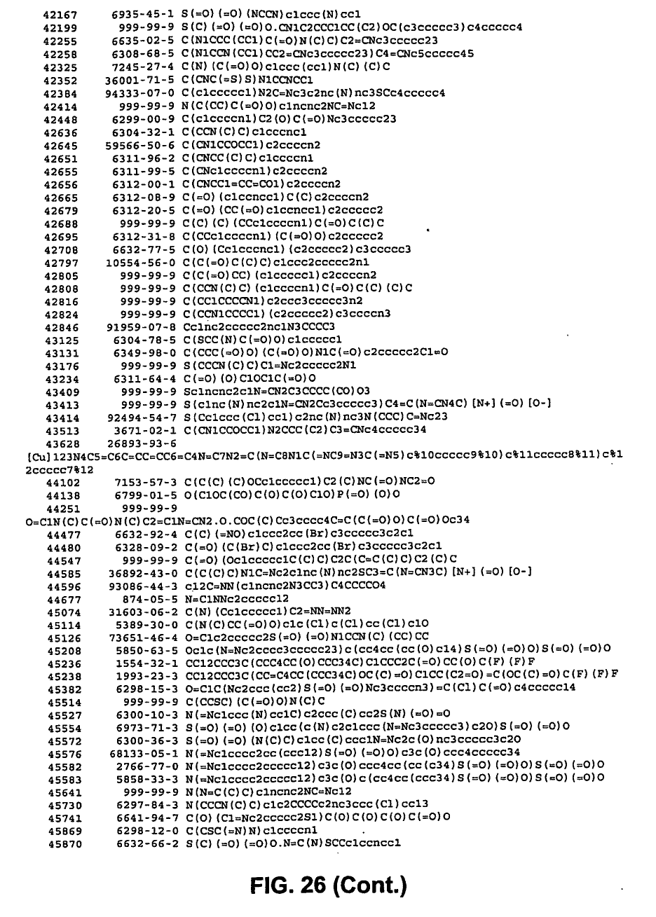

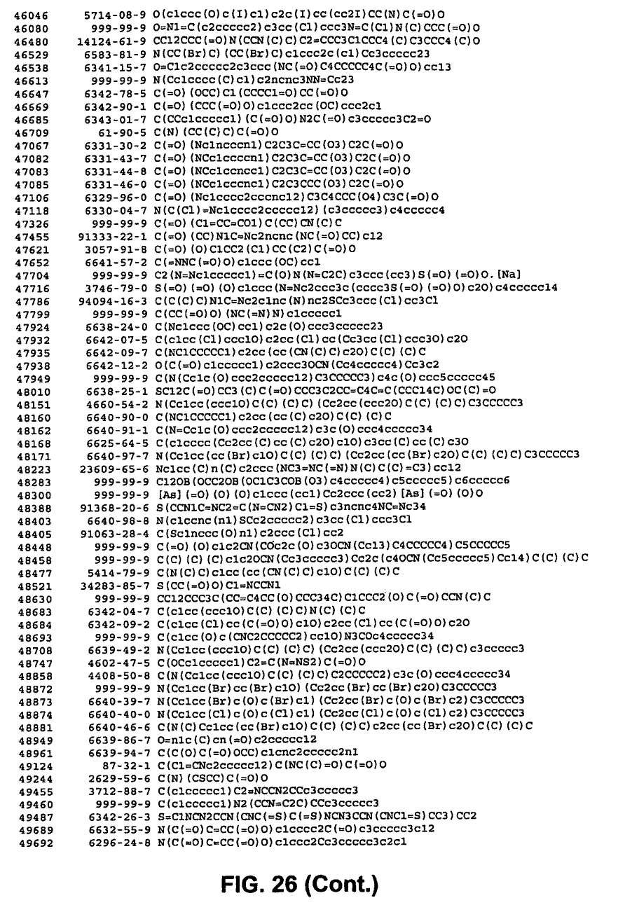

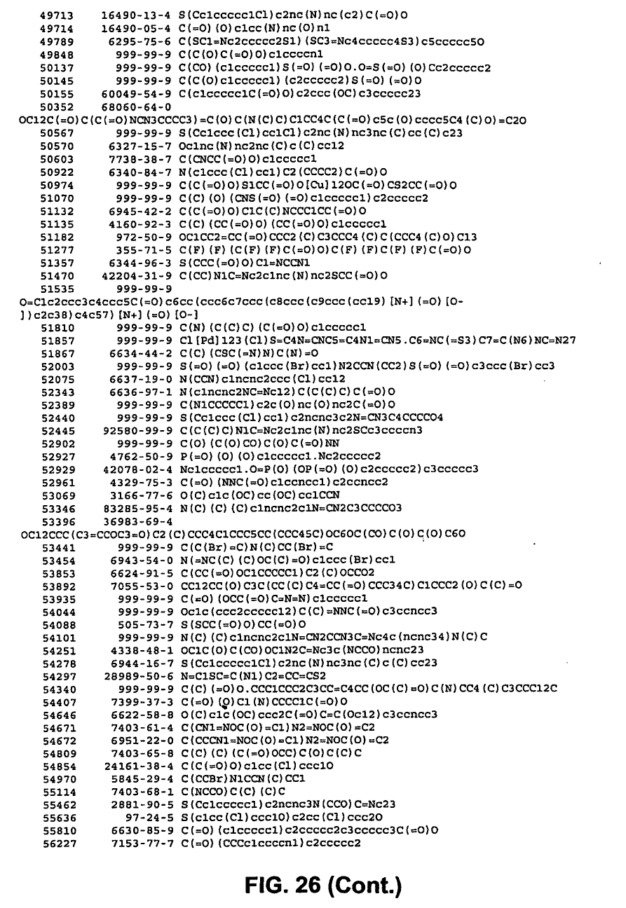

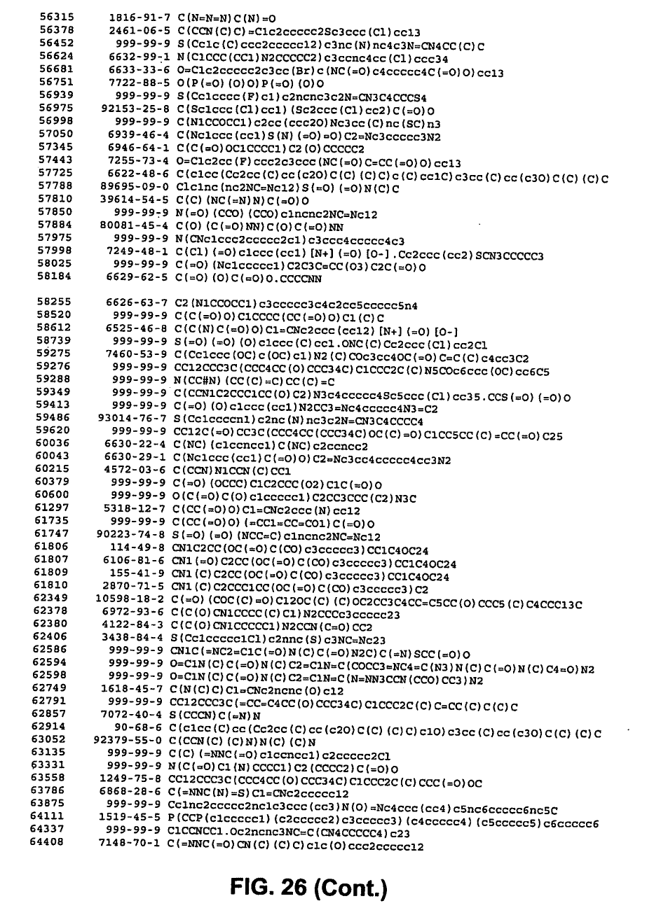

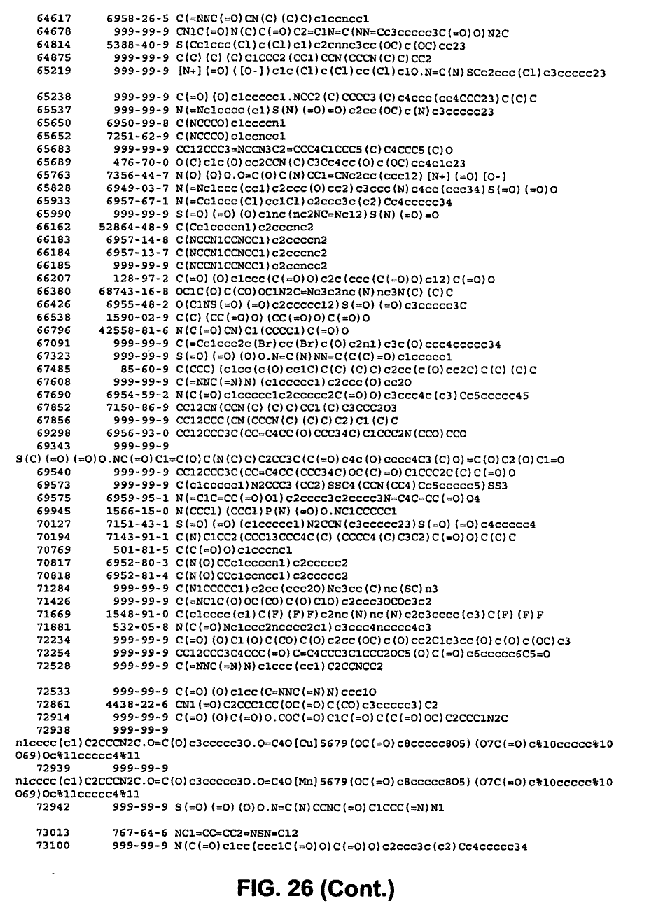

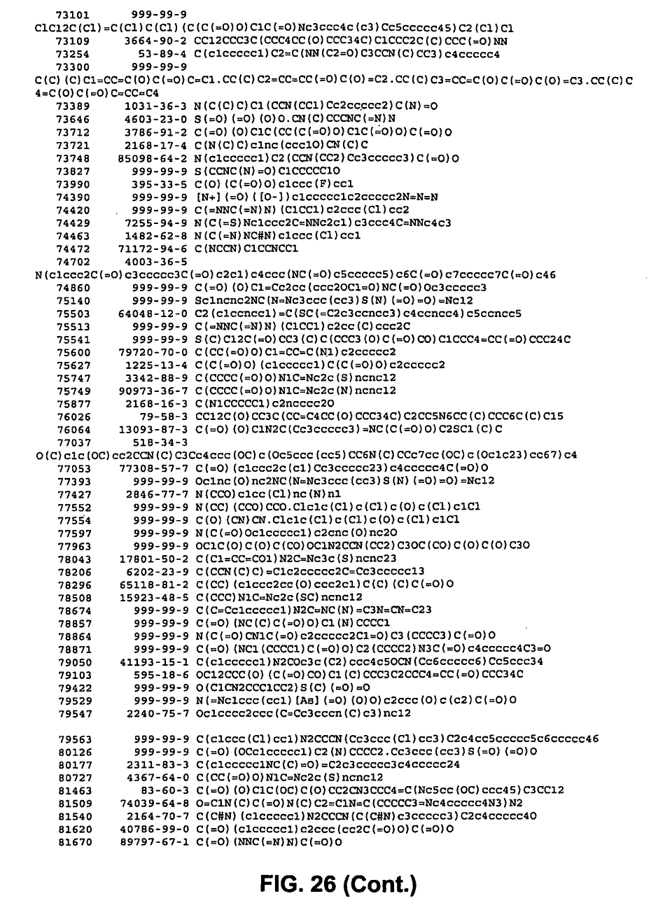

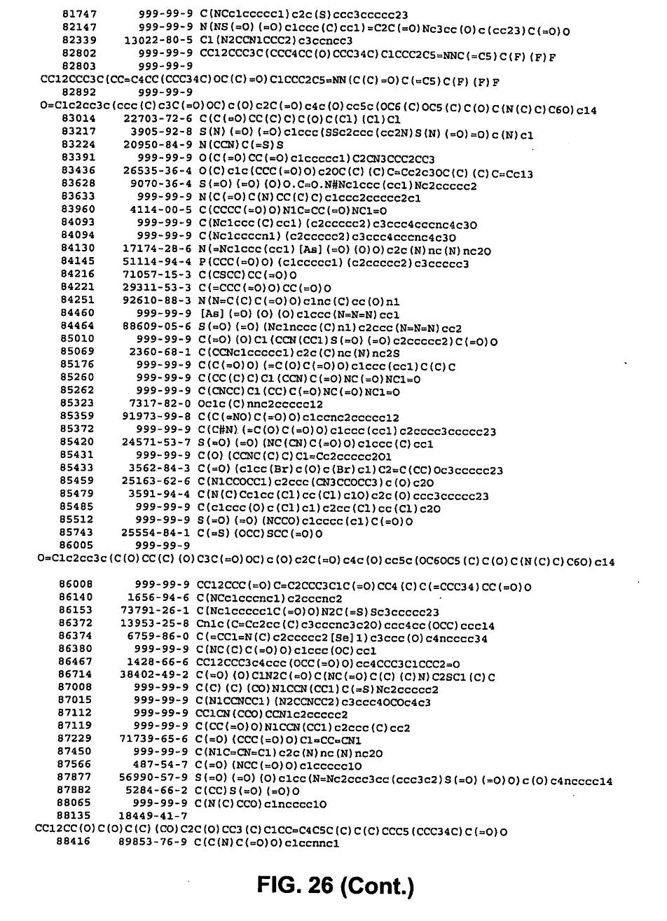

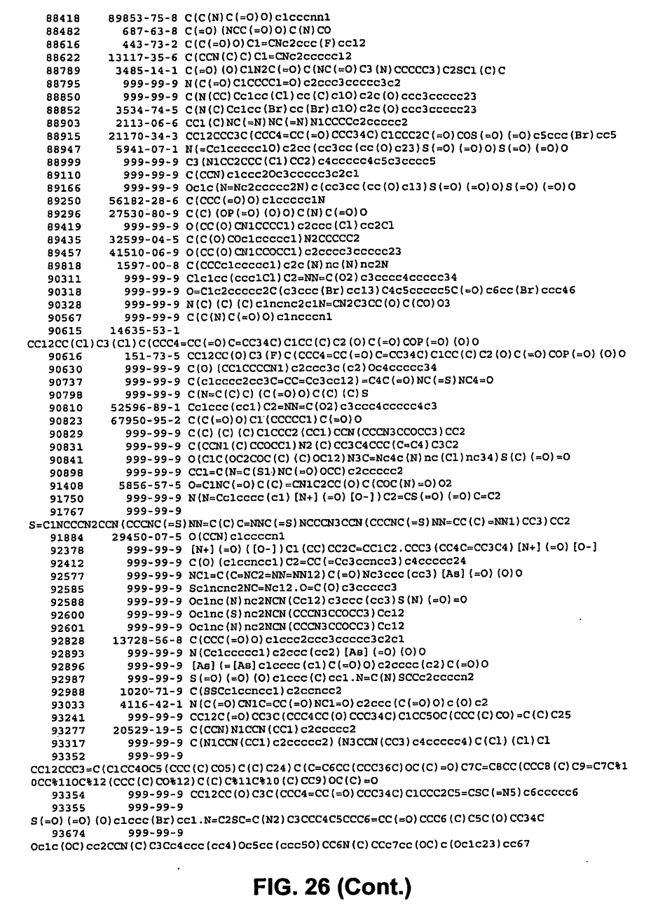

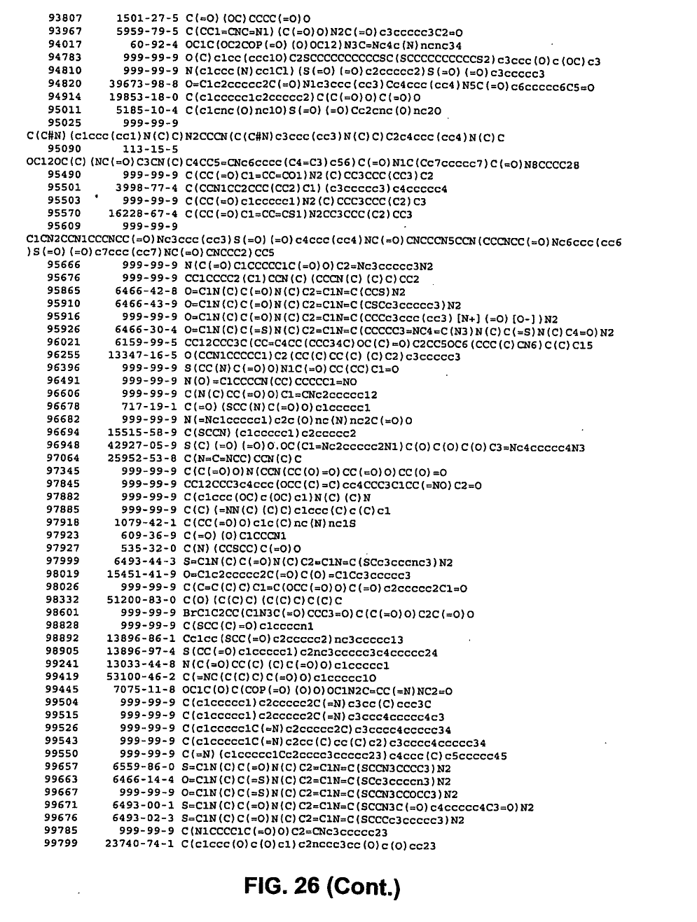

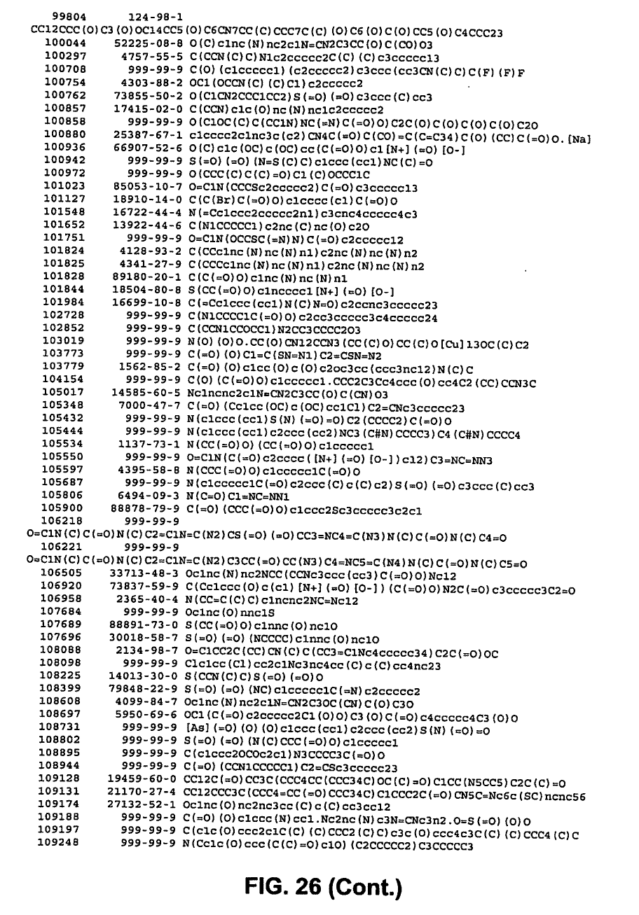

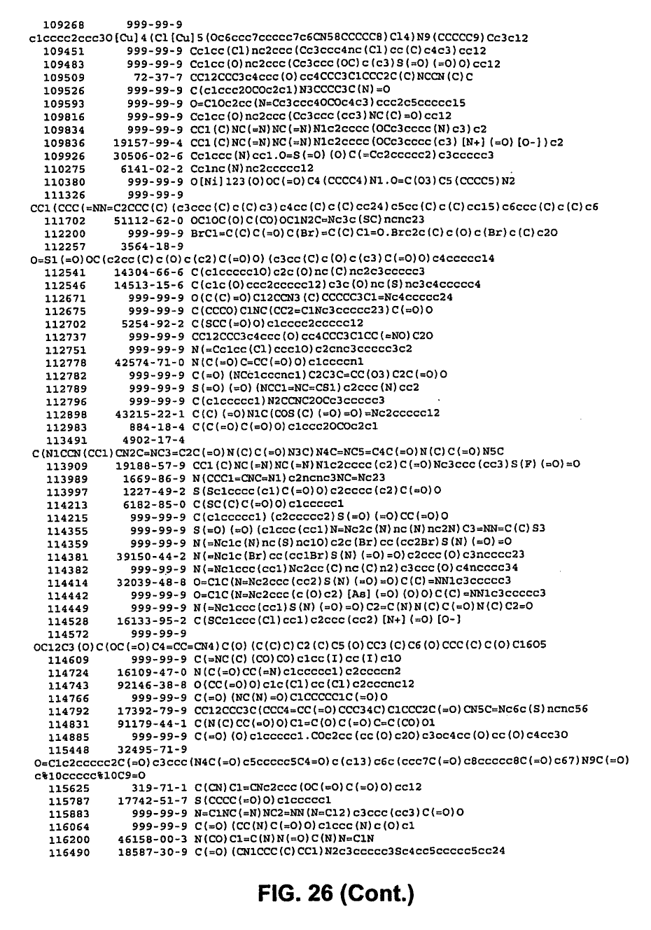

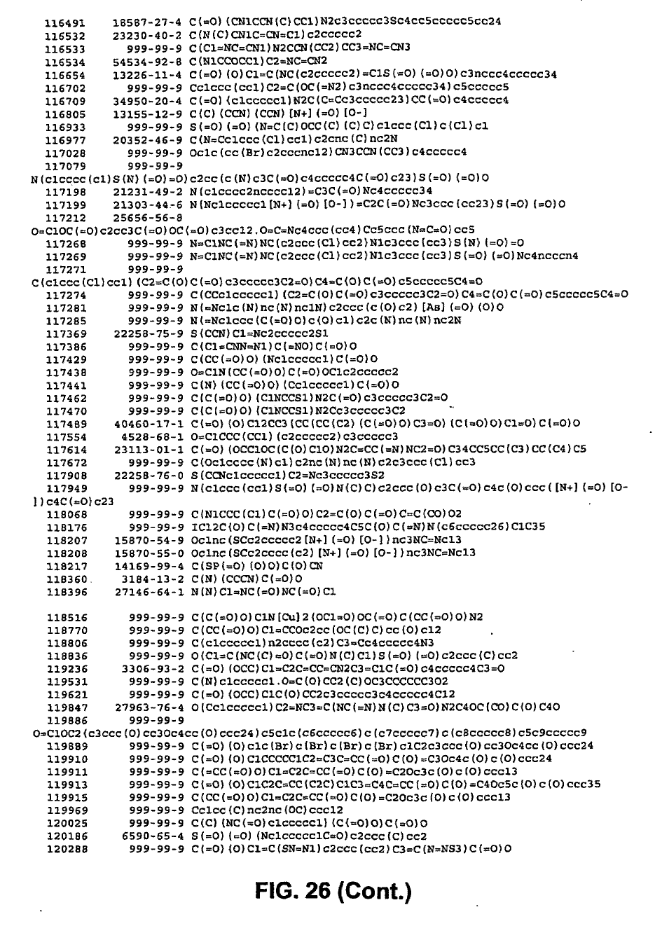

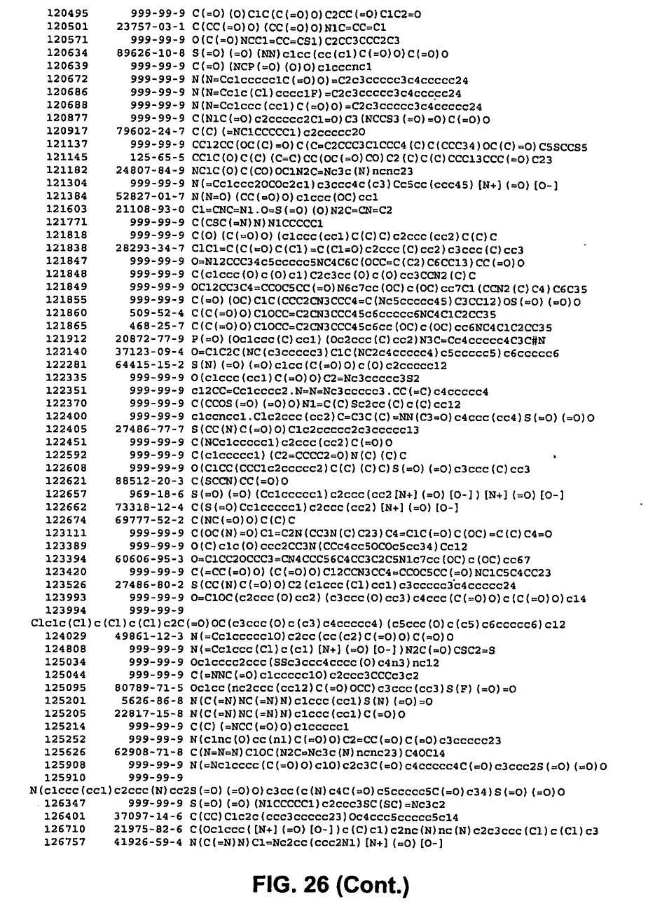

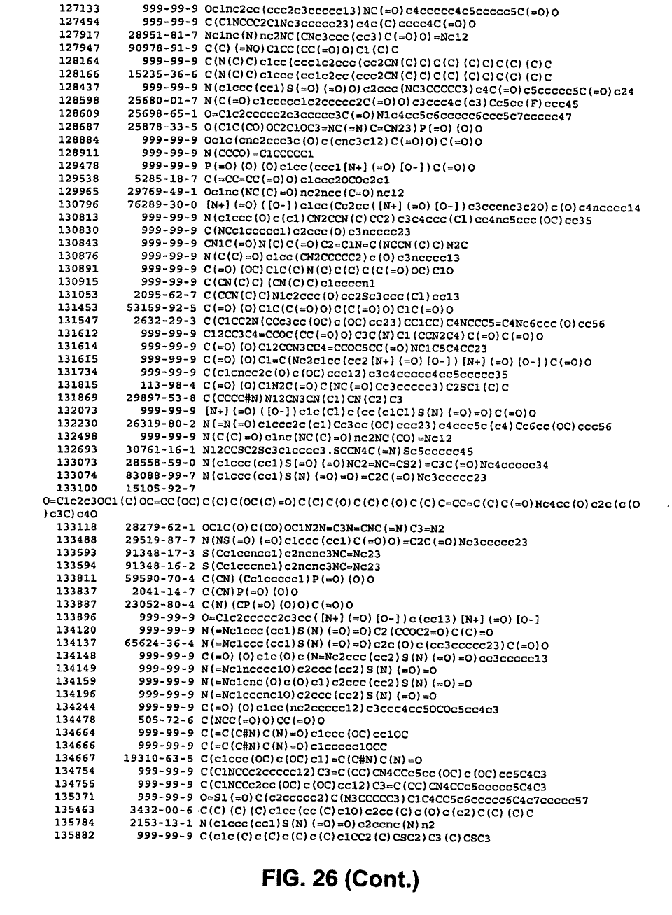

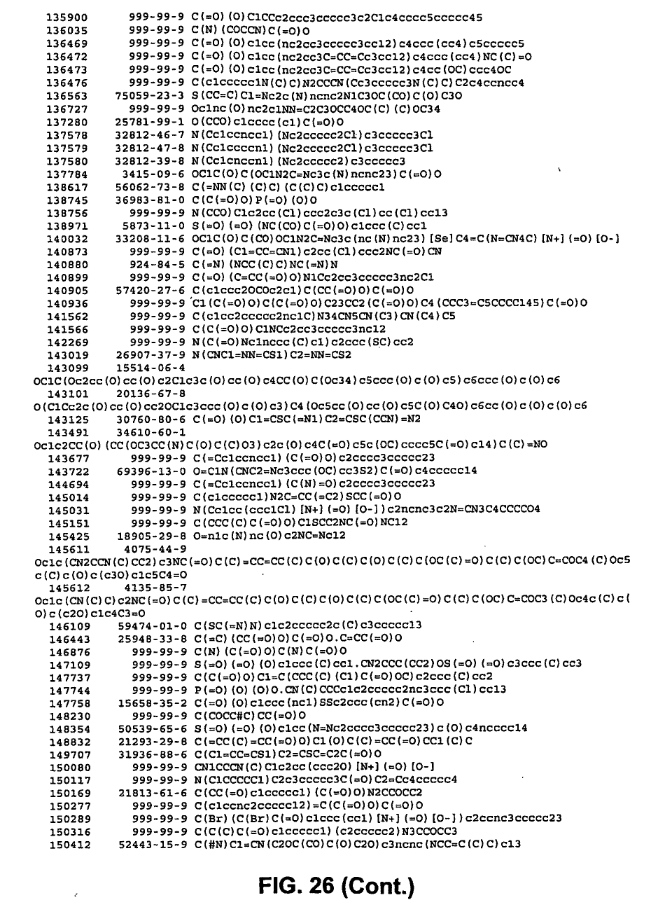

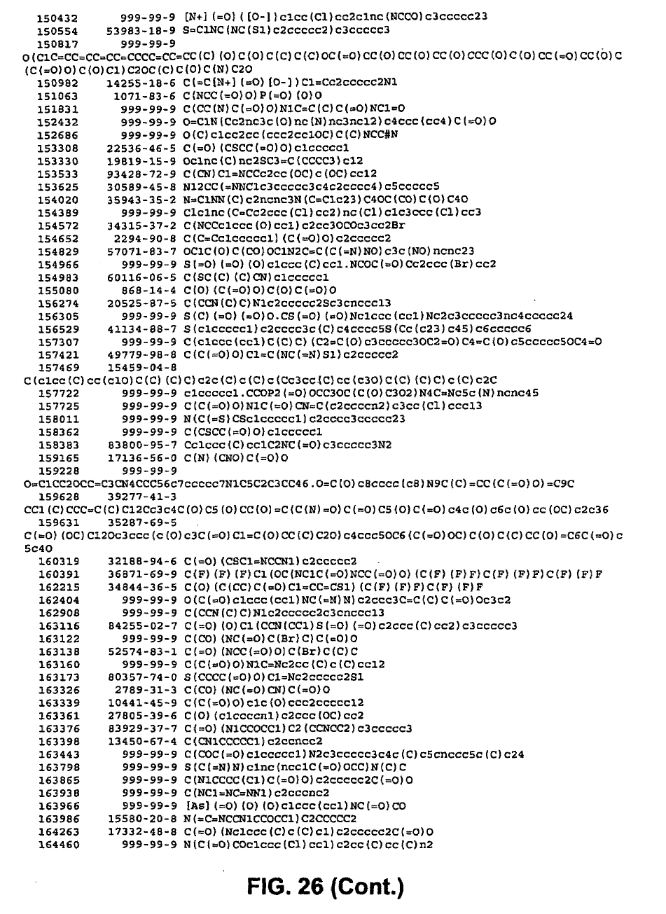

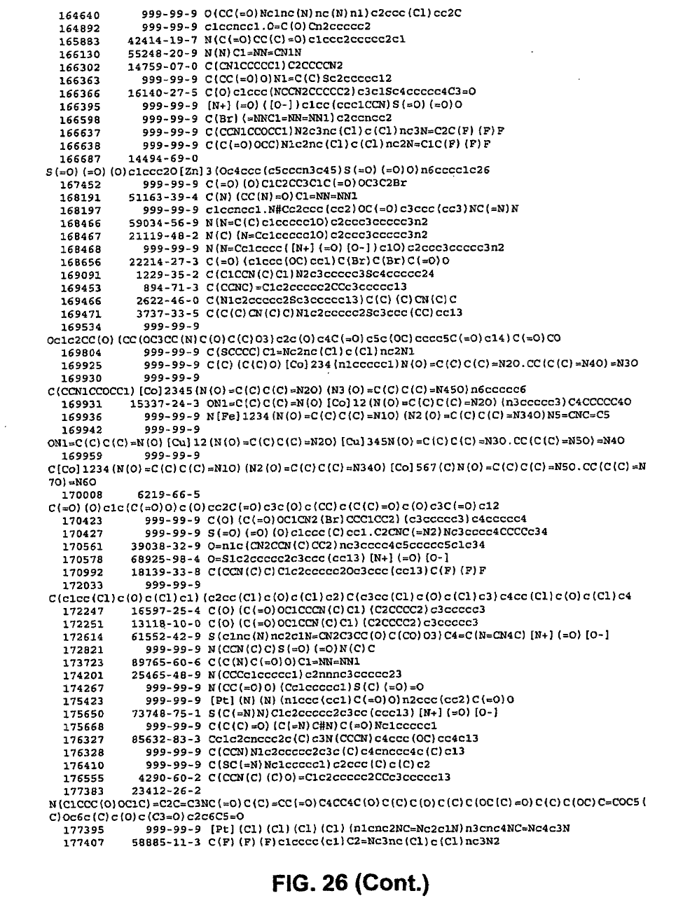

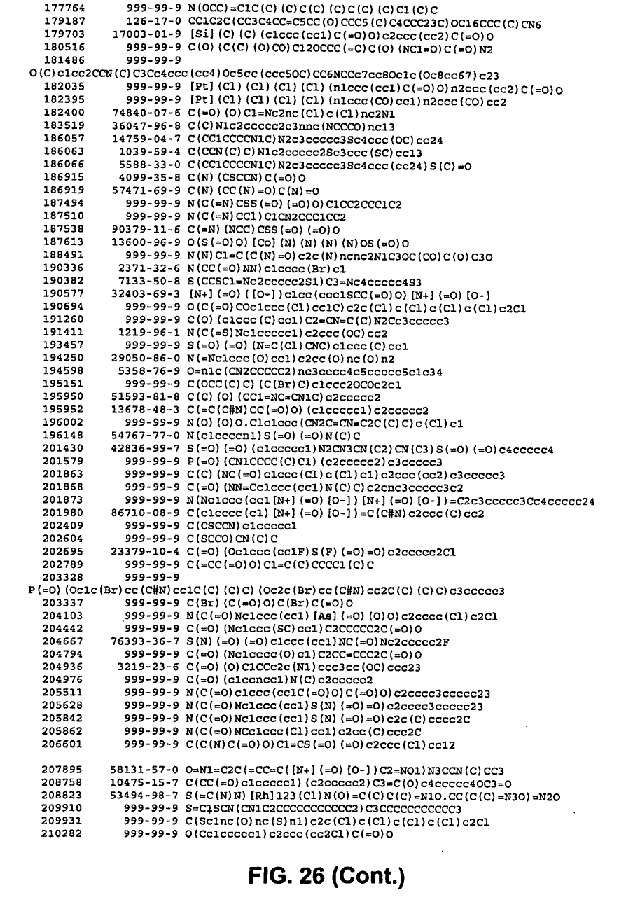

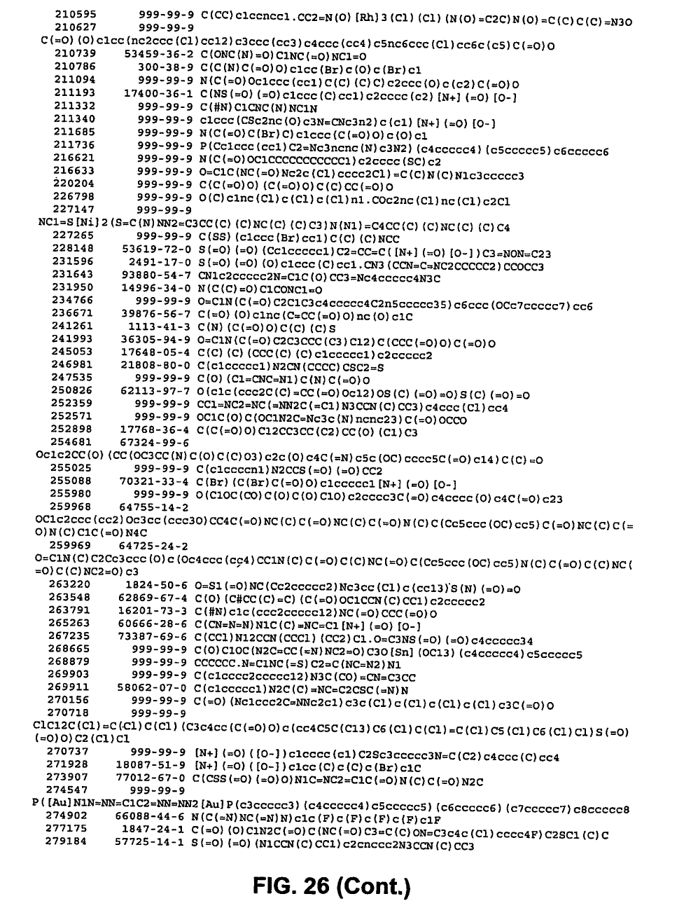

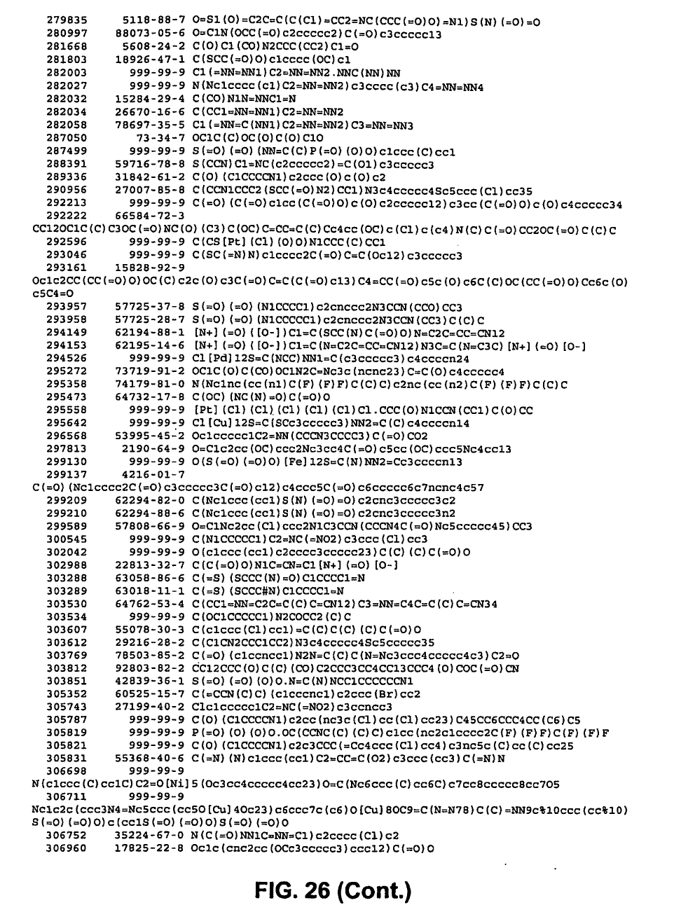

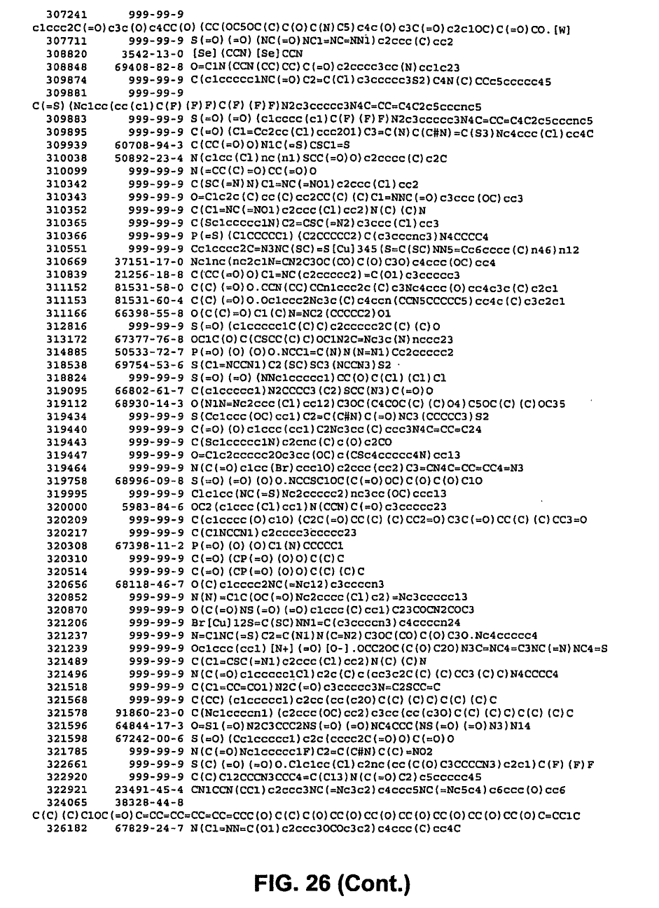

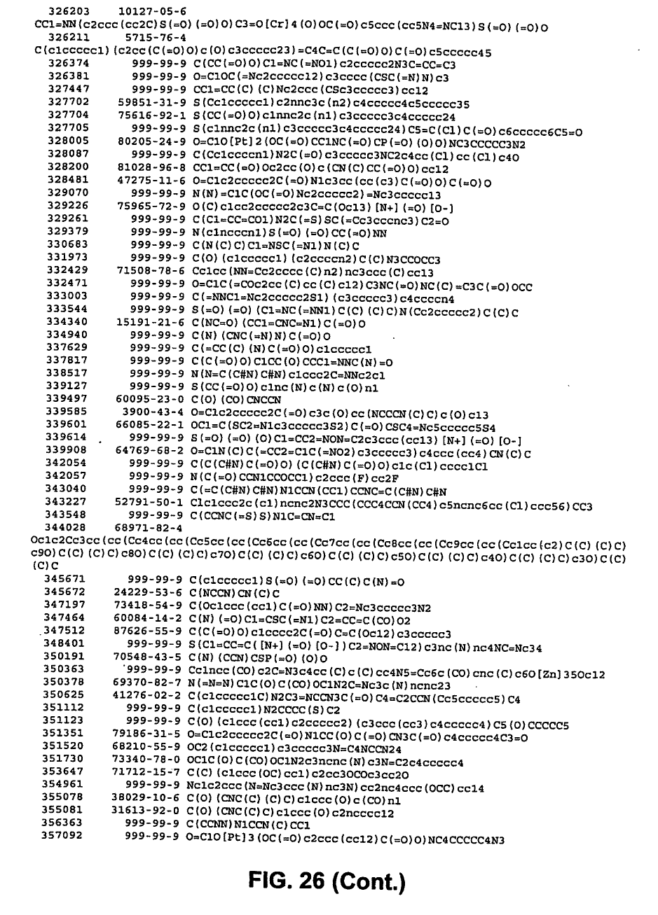

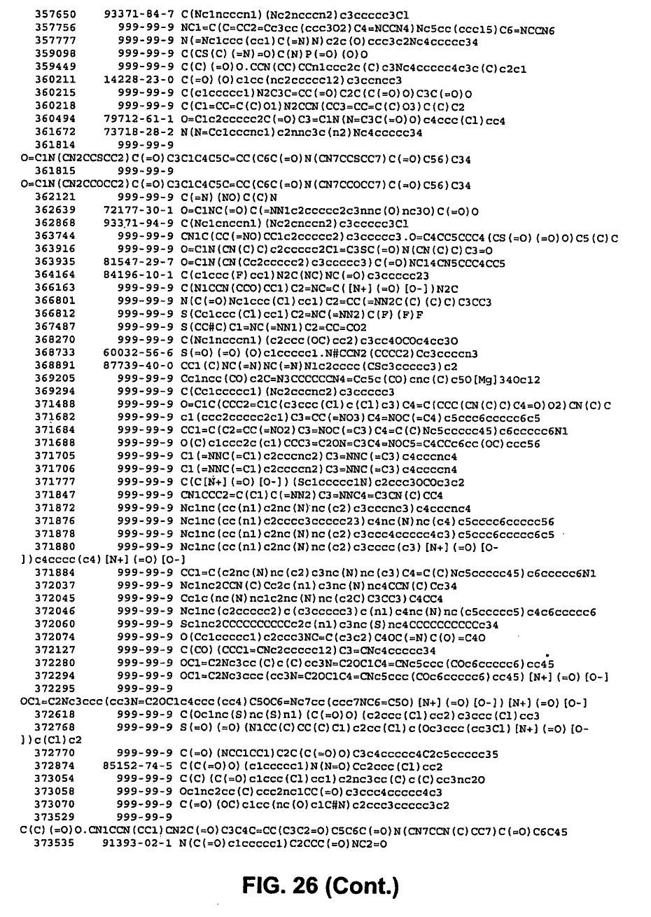

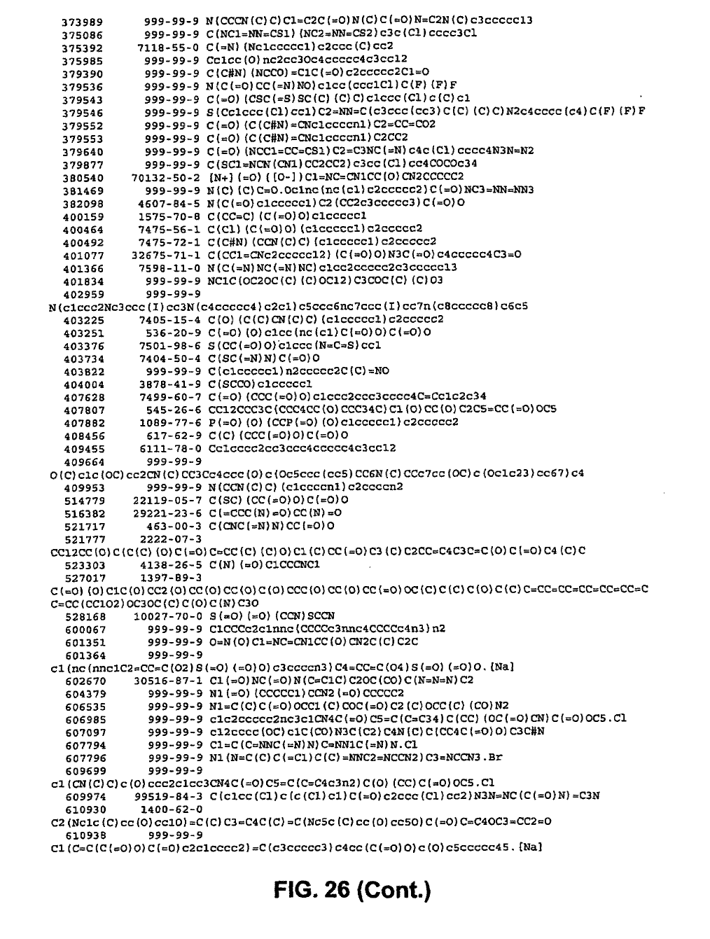

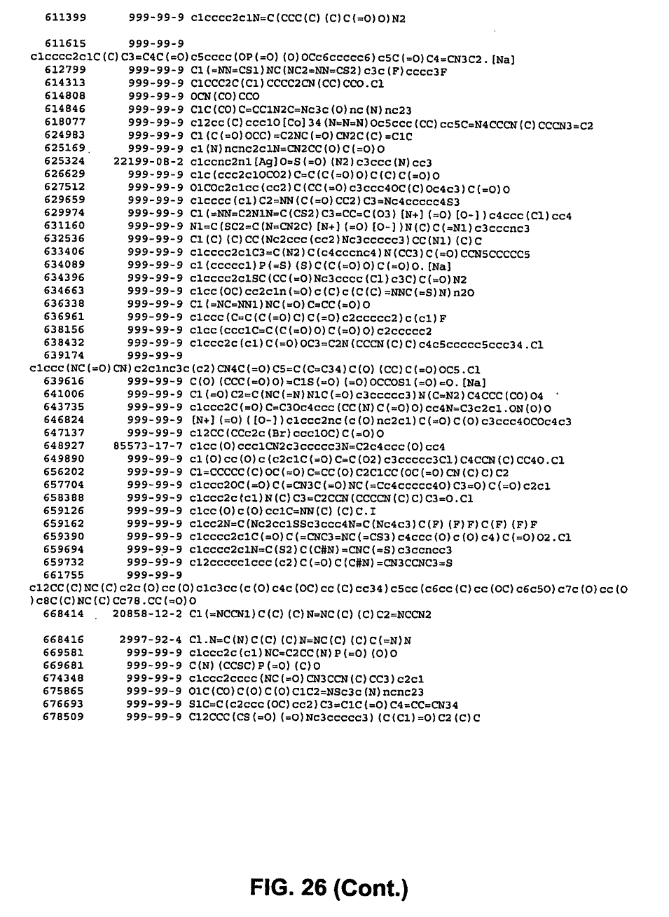

The target for these screening studies is a Dam-AdoHcy complex. Initially we use the crystal structure of T4Dam-AdoHcy complex as a target. An in silico screen (“ISS”) evaluates a library of compounds for their ability to bind the target via computational calculations based on the structure of the compound and target. In silico screens are advantageous over high throughput screening in that any number of compounds can be readily screened without the need for bench-top time and effort associated with high throughput screens. For example, we have used a relatively “small” library, specifically the National Cancer Institute (NCI) “Diversity Set” library. The NCI Diversity set is a subset of approximately 2000 compounds (see FIG. 26) selected from a larger library of about 140,000 compounds. The subset is intended to maximally represent three-dimensional chemical diversity in the 140,000 compound larger library. The NCI Diversity Set is publicly available and has been successfully used in identifying inhibitors of various target molecules, including several potent inhibitors of HIV-1 nucleocapsid (Stephen et al., 2002); the diversity set is publicly available from the NCI Developmental Therapeutics Program (http://dtp.nci.nih.gov). FIG. 22 provides a flowchart summary of the ISS methodology of the present invention

ISS is useful for identifying target compounds and has been addressed by, for example, Pan et al. (2003); Huang et al. (2004). The same inhibitor of the TGF-β receptor kinase has been identified by both ISS and high-throughput screening (Sawyer et al., 2003; Singhe et al., 2003).

When used herein, the term data representation can comprise chemical and/or structural information of a molecule or molecular complex. For example, a data representation can be a set of structure coordinates, a three-dimensional diagram, a two-dimensional diagram, a chemical formula, or other information for a given molecule, molecular complex, or portion thereof.

When used herein, the term structure coordinates will be understood by one of ordinary skill in the art and can refer to mathematical coordinates derived from mathematical equations related to the patterns obtained on diffraction of a monochromatic beam of X-rays by the atoms (scattering centers) of an enzyme or enzyme complex. For example, an enzyme complex can include a methylase, a DNA substrate, and a methyl donor. The diffraction data are used to calculate an electron density map of the repeating unit of the crystal. The electron density maps are used to establish the positions of individual atoms within the unit cell of the crystal. For a set of structure coordinates determined by X-ray crystallography, those of ordinary skill in the art understand that coordinate data is not without standard error. In embodiments of this invention, any set of structure coordinates that have a root mean square deviation of protein backbone atoms (e.g. N,alpha-C, C and O) of less than 0.75 angstroms when superimposed—using backbone atoms—on the referenced structure coordinates shall be considered identical.

In an embodiment, a small molecule and/or small molecule data bases are screened computationally for chemical entities or compounds that can bind in whole, or in part, to an enzyme or enzyme complex as described herein. In a particular embodiment of screening, the quality of fit of such entities or compounds to a binding site of interest may be evaluated either by shape complementarity or by estimated interaction energy. See Meng, E. C. et al., J. Comp. Chem., 13, pp. 505-524 (1992).

The present invention is not limited to the use of any particular method for carrying out the screen. The invention can utilize any docking software algorithm and any scoring algorithm known in the art. U.S. Pat. App. No. 2005/0170379 (Kita et al.) summarizes different techniques suitable to perform docking simulations, including rigid-body pattern-matching algorithms (based on any of surface correlations, geometric hashing, pose clustering, graph pattern-matching), fragmental-based methods (including incremental construction or “place and join” operators), stochastic optimization methods (including Monte Carlo, simulated annealing, genetic (or memetic) algorithms, molecular dynamics simulations, and/or hybrids of any one or more of these techniques. Numerous docking programs are available and continue to be developed in terms of algorithms and efficiency. The program DOCK (Ewing et al., 2001) is used in our inhibitor screening studies because of its free distribution. The program performs the following computational tasks: first, an orientation search of a small molecule in a chosen site or pocket, which is a fundamental process of docking; second, a conformational search of a molecule, leading to identification of the best conformation to fit in the target site. More importantly, it can utilize a database of compounds for docking tests, meeting the basic need for virtual screening.

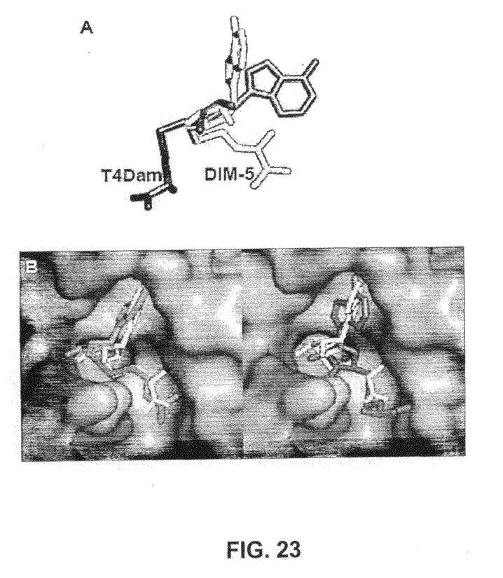

In DOCK based screening, sphere centers are generated based on the Connolly surface of the binding site of interest and the compounds from the database are then docked into the binding site by matching sphere centers with compound atoms. D Selection of the site for docking is typically based on biological data, including homology information, as well as based on the quality of a site for binding inhibitors. Such binding capacity may ideally be validated based on the ability of the docking algorithm to reproduce the bound conformation of a known ligand, such as the ability to reproduce the experimentally determined binding mode of AdoHcy (see FIG. 23). Strategies are available for selecting putative inhibitor binding pockets to block both protein-protein and protein-DNA interactions (Chen, et al., 2000; Hancock et al., 2005; Huang et al., 2003; Markowitz et al., 2004), which will facilitate site selection in the present study.

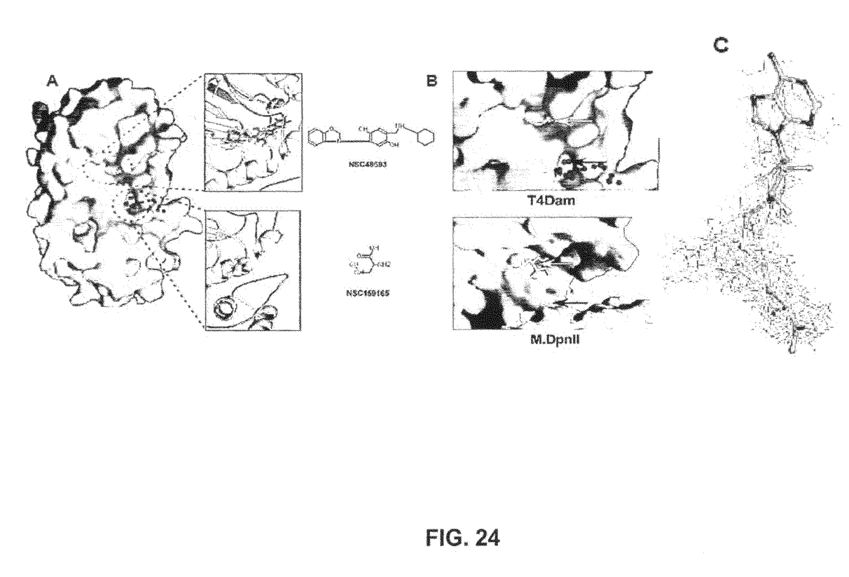

Preliminary molecular docking was conducted targeting the AdoHcy binding site (dark), and a cavity between the two domains (dark and gray, FIG. 24A). The location of this cavity is large enough to bind a glycerol molecule (as visualized in one of the T4Dam structures, not shown), suggesting that a small molecule may block the hinge movement between the two domains. The apparent hinge mobility of the two domains may reflect a functional importance during the reaction cycle. Interestingly, in the structure of DpnII—a related GATC restriction modification enzyme—the corresponding position shows a “dent” on the surface (indicated by a black arrow in FIG. 24B) but not as deep a cavity where a small molecule can bind. This observation raises an interesting possibility that the cavity is a unique property for T4Dam that could be targeted for selectivity of inhibition.

In an embodiment, docking sites are specified based on the experimentally bound AdoHcy and active site. The region within 8A around the ligand is considered. A 0.3 Å grid is used in all the docking studies to compute interaction energy, a grid. Energy scoring grids are obtained by using a united-atom model, a distance-dependent dielectric function (∈=4r), and 6-12 Lennard-Jones van der Waals potentials. The flexible ligand docking is performed using an Anchor-First mechanism with a minimum anchor fragment size of 7 atoms and a sampling of 25 conformations. The maximum orientations are set to 5000 during docking an anchor fragment.

Energy minimization is performed using the grid-based rigid body simplex algorithm. One cycle of 100 simplex minimization steps are applied to adjust the compound's orientation and conformation, and to locate the nearest local energy minimum to a convergence of 0.5 kcal/mol. The minimization is calculated on-the-fly in the program DOCK and only the final energy scores are documented.

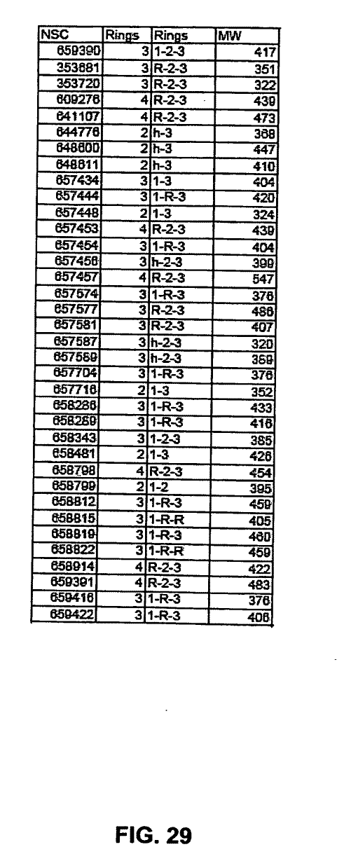

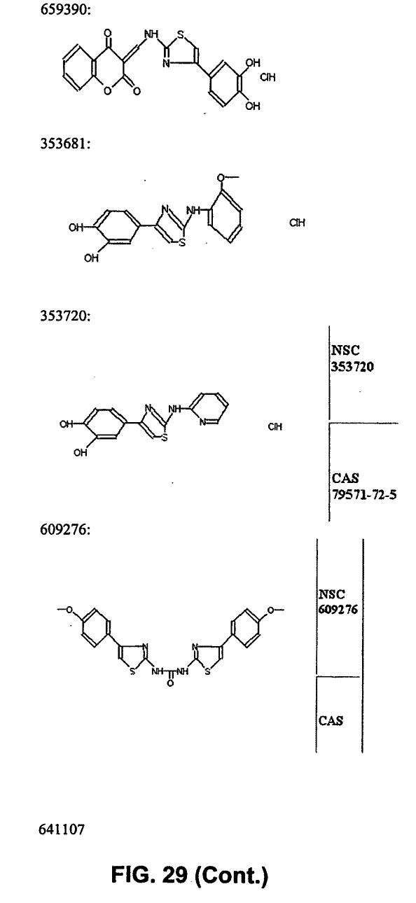

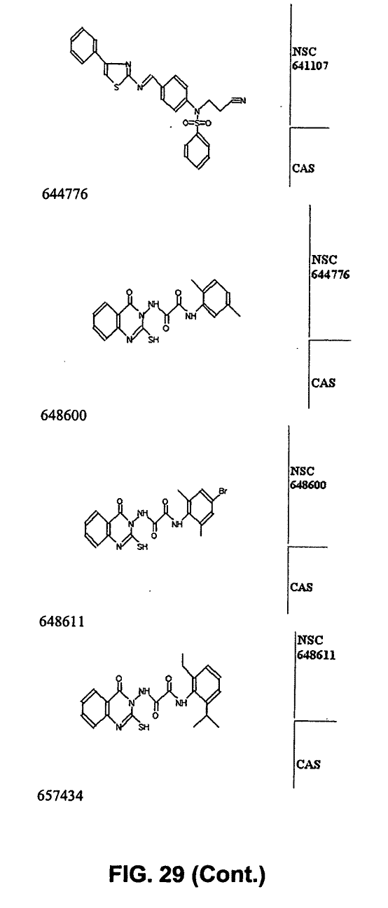

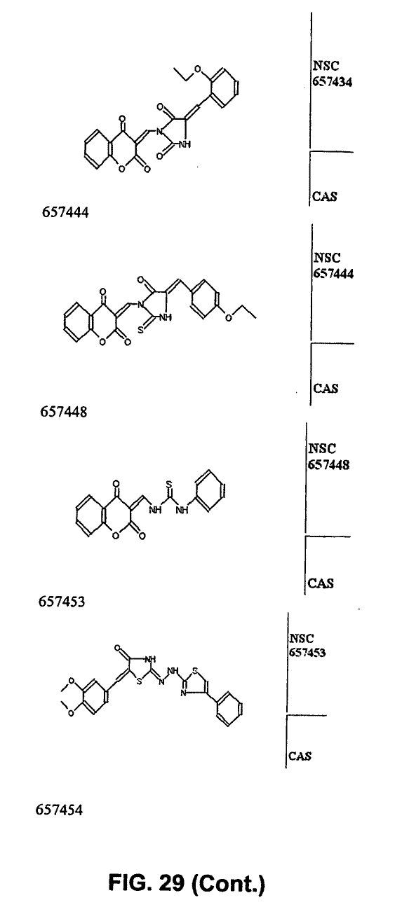

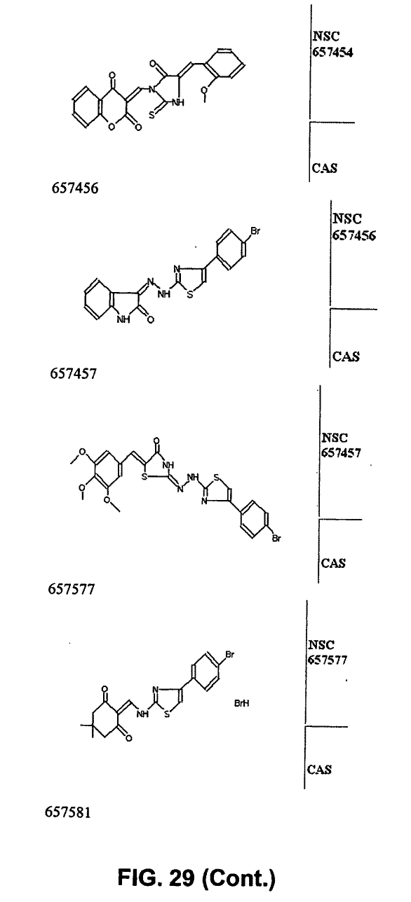

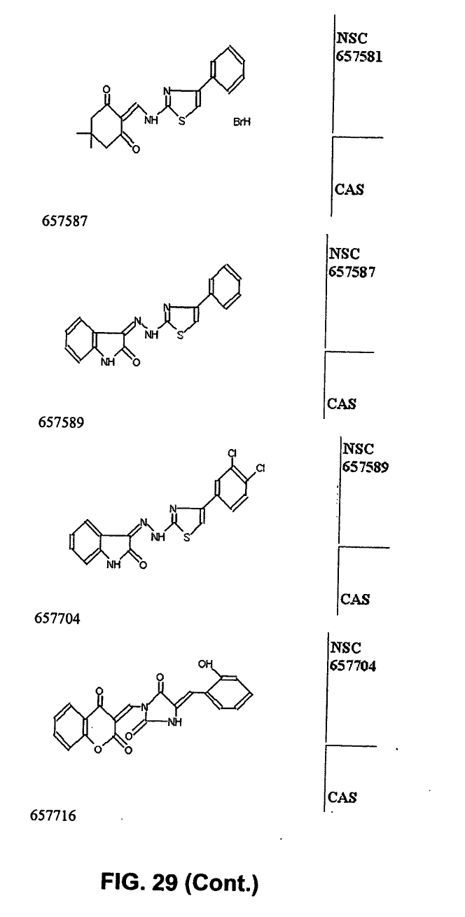

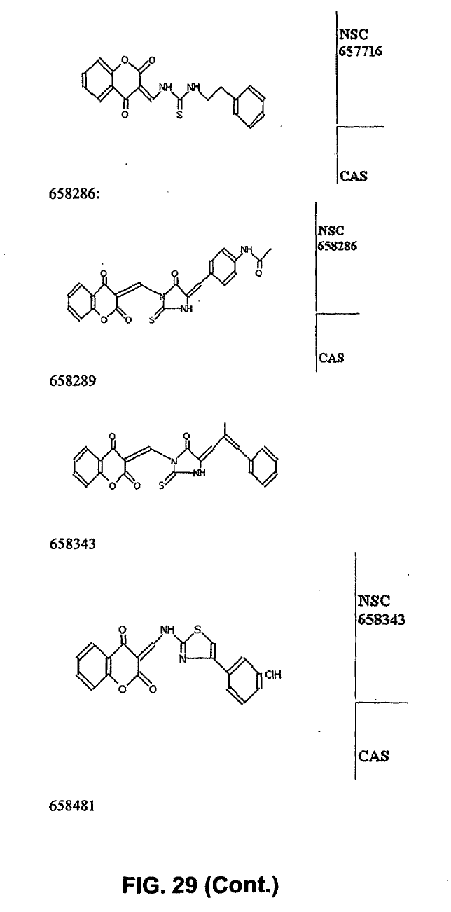

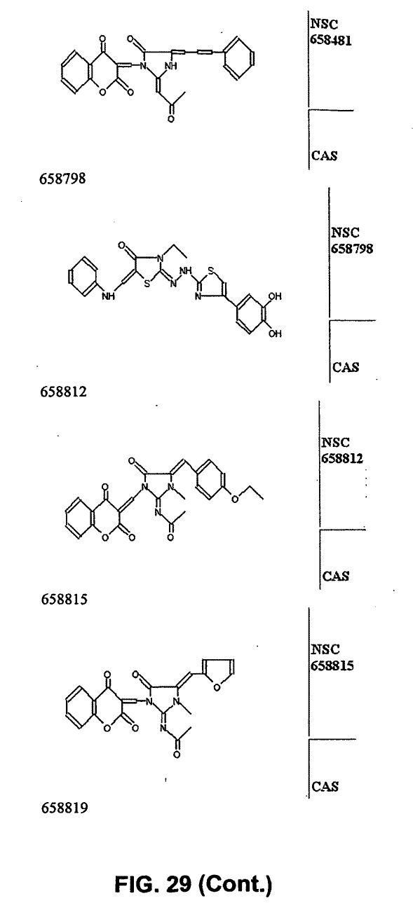

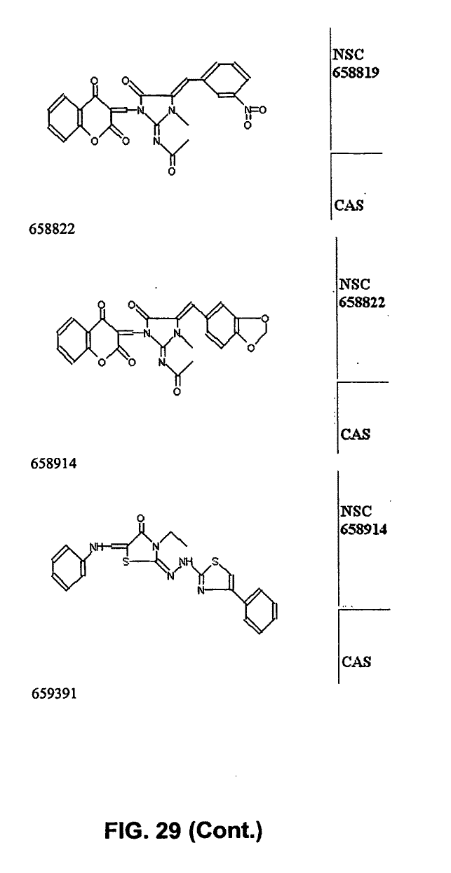

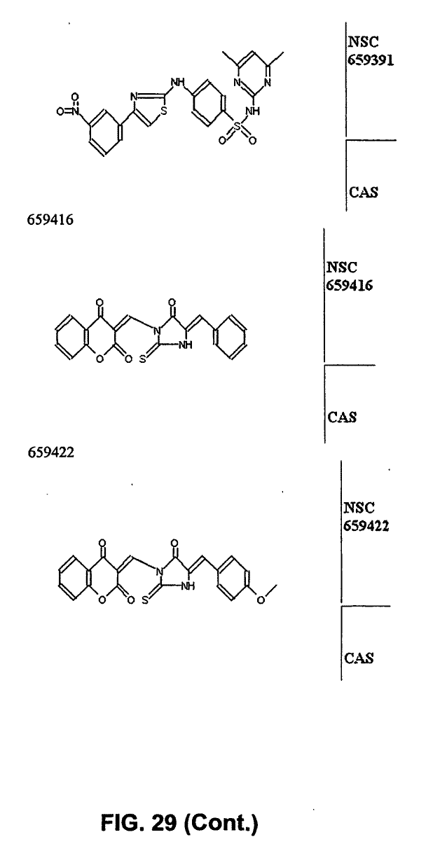

Further investigation is conducted using related compounds having some chemical similarity to the lead compound (e.g. compound #78 (NCI 659390). Related compounds are identified using SMILES string-based pattern recognition (see FIG. 26) for compounds with the same or similar core fragment structure. See FIG. 29 for compounds related to compound #78 (NCI 659390) based on this string-based pattern recognition.

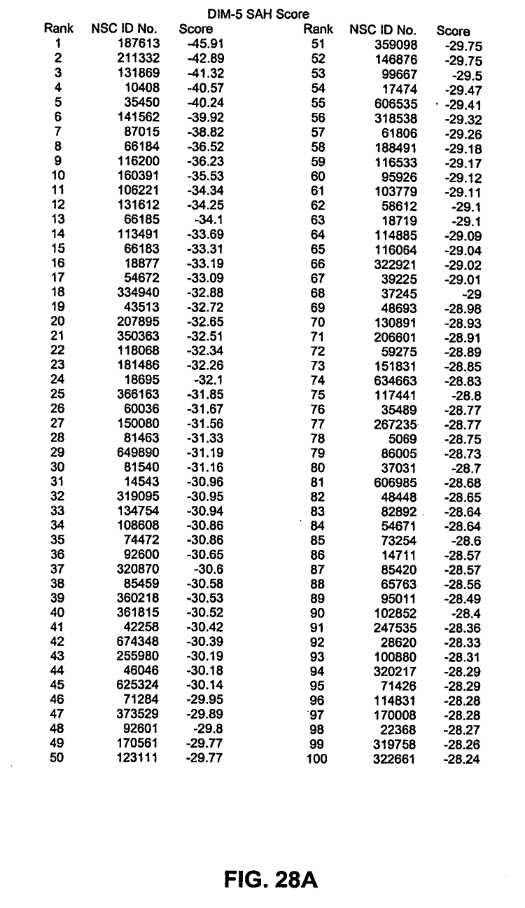

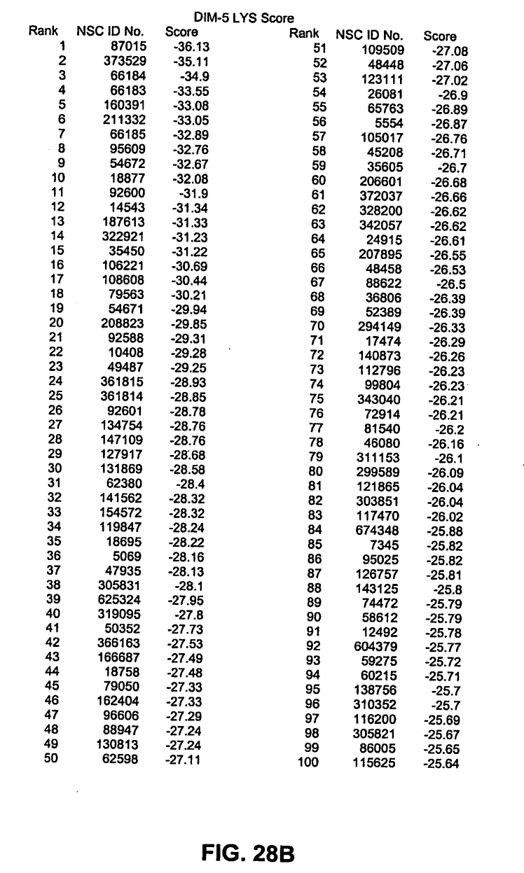

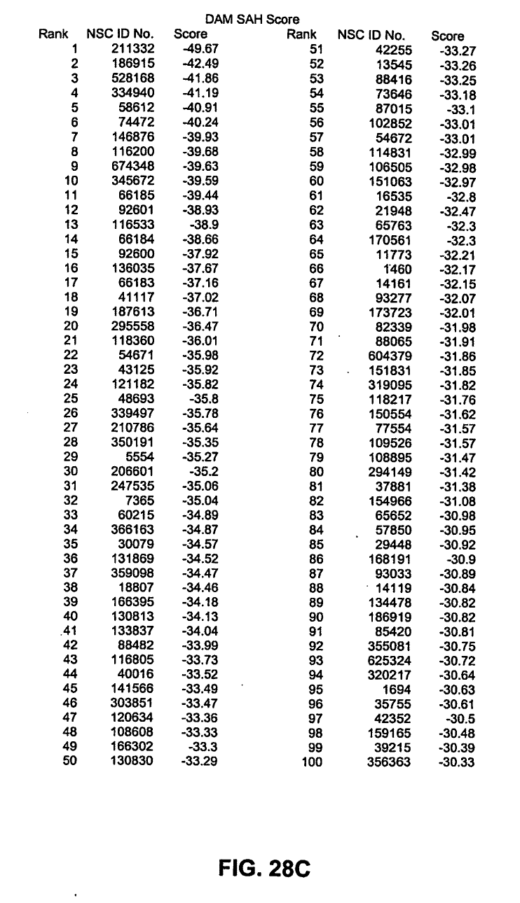

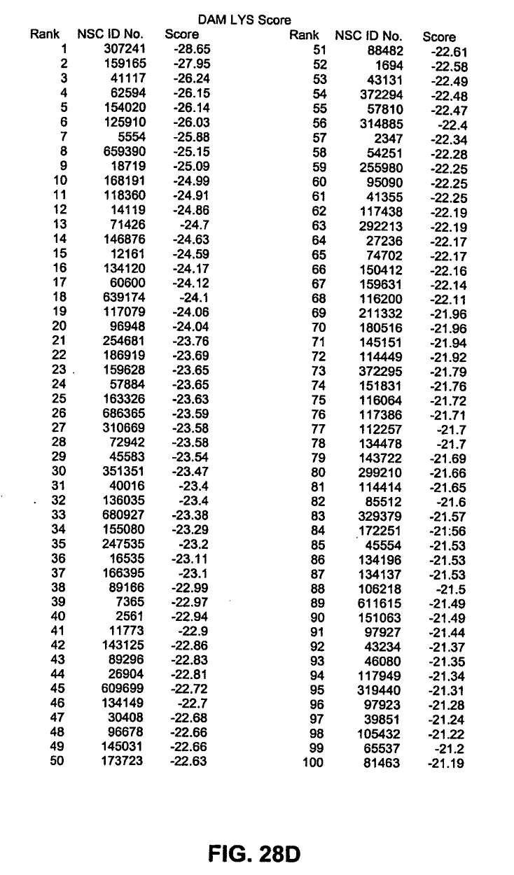

Scoring, After docking all 2000 compounds in the database into the cofactor-binding site or the putative binding cavity in the hinge region, the program DOCK was applied to calculate the ligand-protein interaction energy to rank the docked ligands (see FIG. 28). The energy score is based on van der Waals and electrostatic interaction energy components. As a final step in the docking process, the orientation of the ligands was subjected to additional energy minimization prior to obtaining the final energy score. The top solutions corresponding to the best DOCK energy scores were then sorted and stored. Currently, we select 18 hits for the cofactor binding site and 22 hits for the cavity; examples are shown in FIG. 24. Interestingly, among the top 30 hits for the cofactor-binding site, 10 compounds have amino acid-like chemical structures (superimposed with the methionine moiety of the cofactor in FIG. 24C). This is enriched approximately 20-fold from 40 such compounds in the library of 2000 compounds.

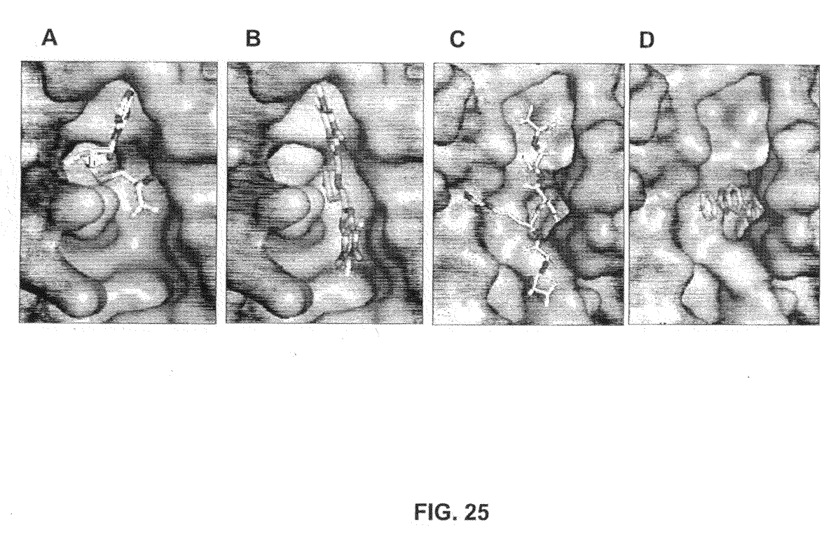

Specificity against other structurally characterized non-Dam MTases: Following the same approach the 2000 compounds were docked against the cofactor site and a peptide binding site (FIG. 25) on the ternary structure of Neurospora crassa DIM-5—a histone H3 Lys9 MTase (Zhang et al 2002) (Zhang et al., 2003). Dim-5 and T4Dam belong to two different classes of AdoMet-dependent MTases (Schubert et al., 2003), with different structural folds and different cofactor conformation (see FIG. 23). We also used an irrelevant, hypothetical site in the Dim-5 surface as an internal control to eliminate “frequent hitters”. We used a three-layered neural network-based method that was developed for rapid and automatic identification of potential frequent hitters (Roche et al., 2002). From these studies, we selected 20 hits for the cofactor binding site, 11 hits for the substrate Lys-binding site, and 9 hits for the zinc-binding site (not shown); examples are shown in FIG. 25.

It should be noted that the majority of the compounds identified from the cofactor sites of Dim-5 and T4Dam are different, indicating that specificity can be achieved via docking. We also note that Roche et al. (2002) predict a high percentage of frequent hitters in databases are comprised of real drugs; thus removing frequent hitters could miss real marketable drugs. Because the goal of docking is to identify compounds complementary to the binding pocket, we evaluate some of the top, but frequent, hits in the biochemical assays.

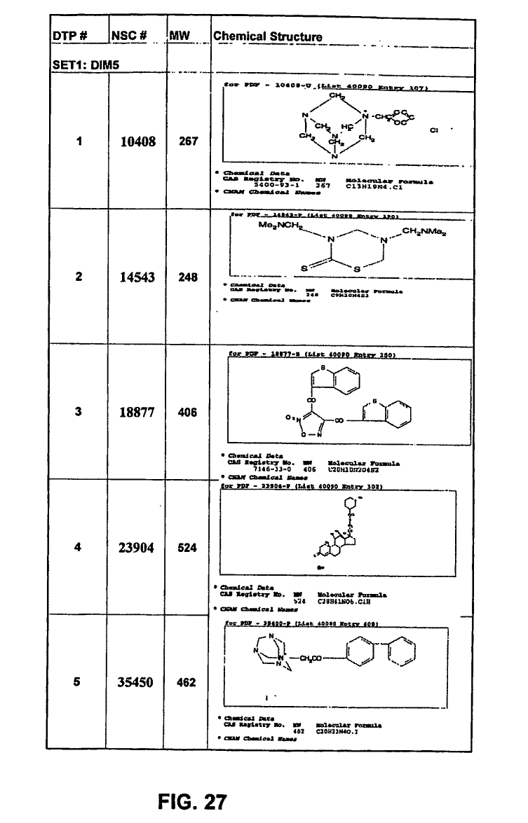

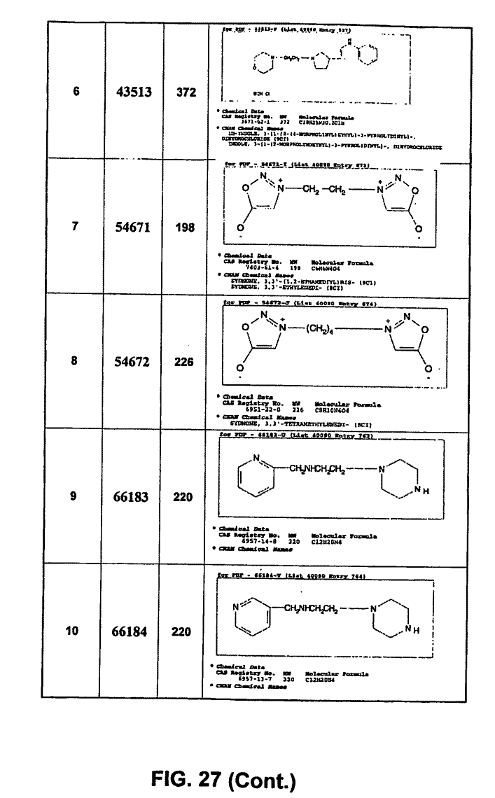

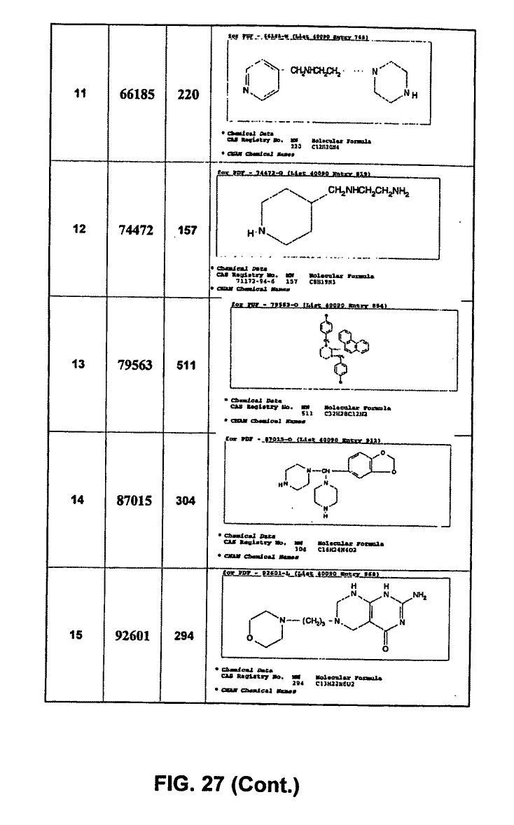

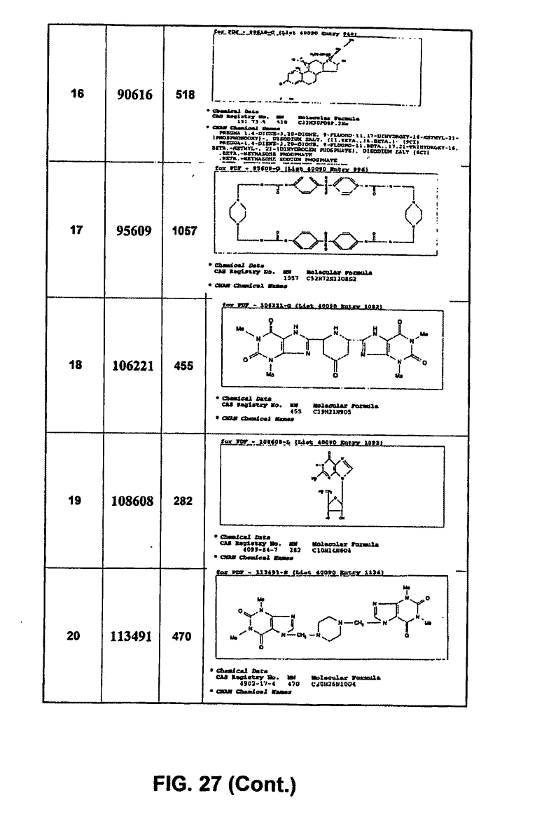

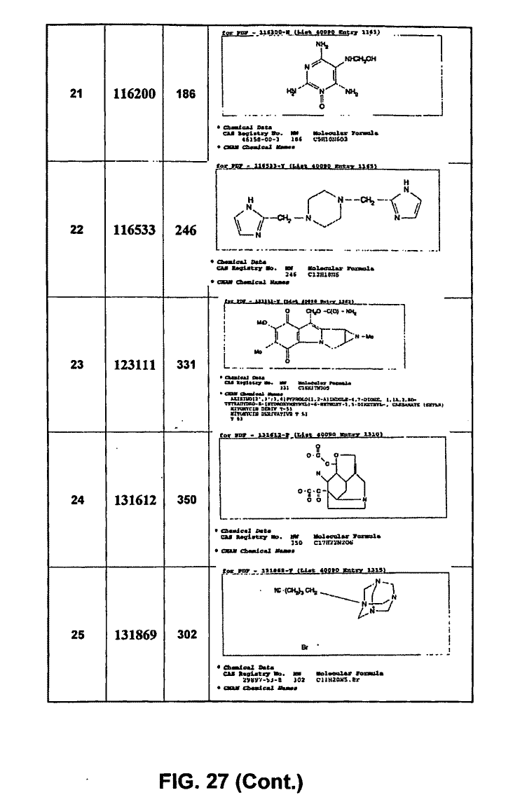

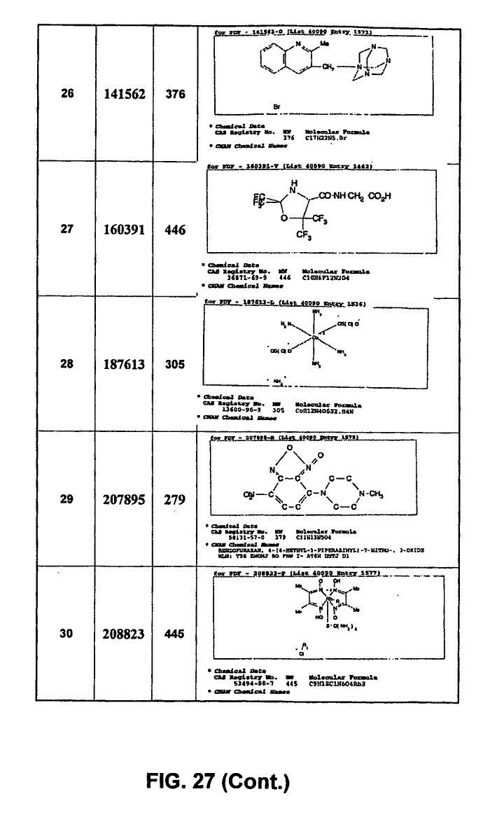

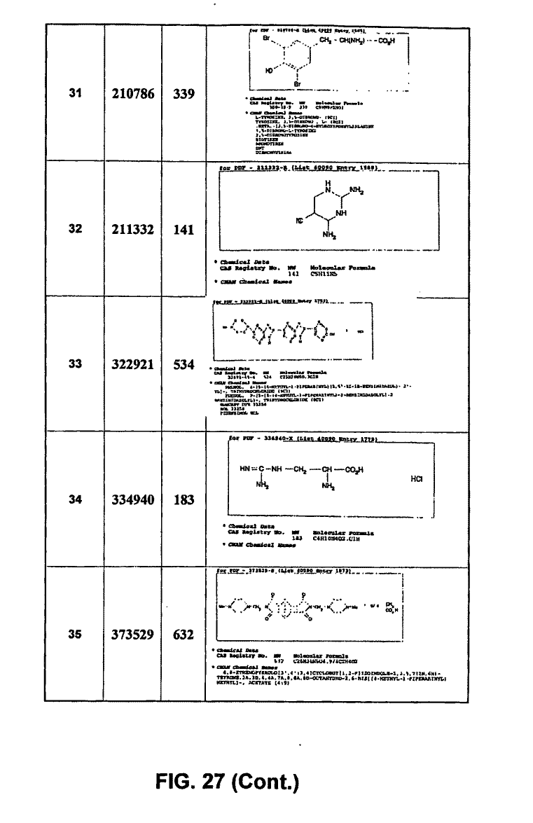

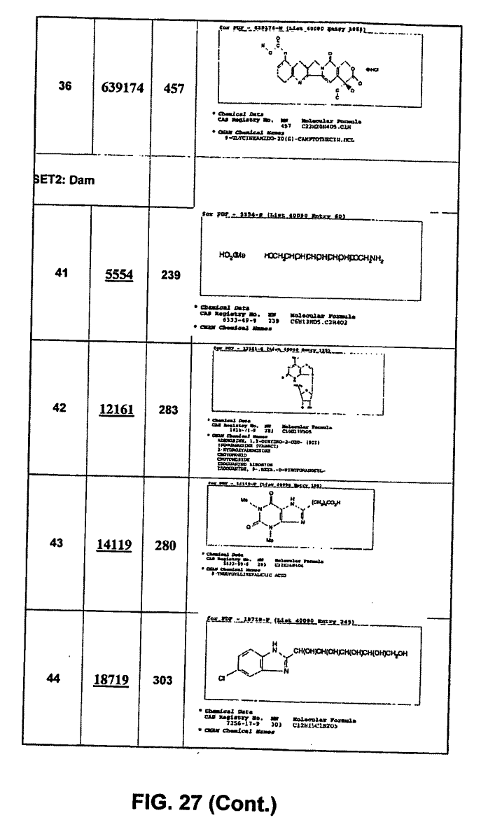

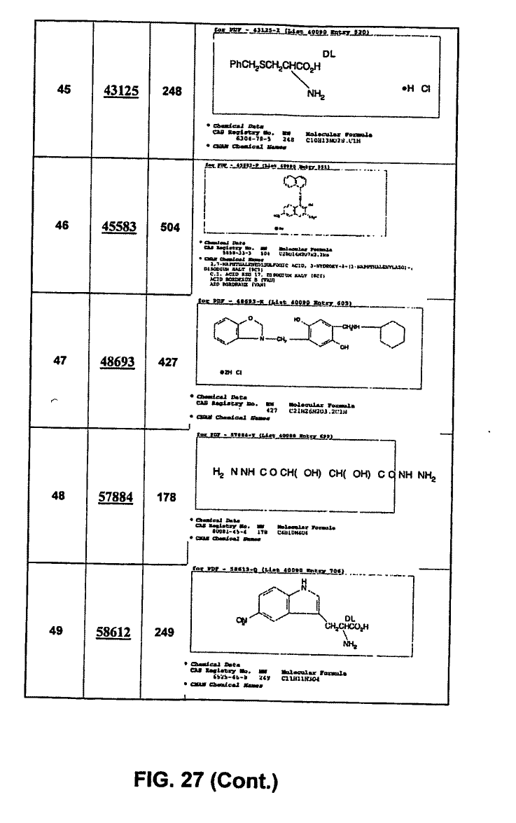

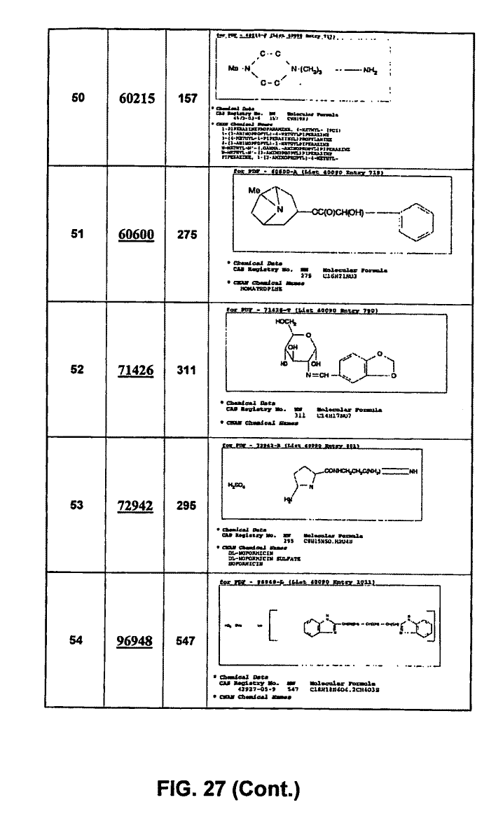

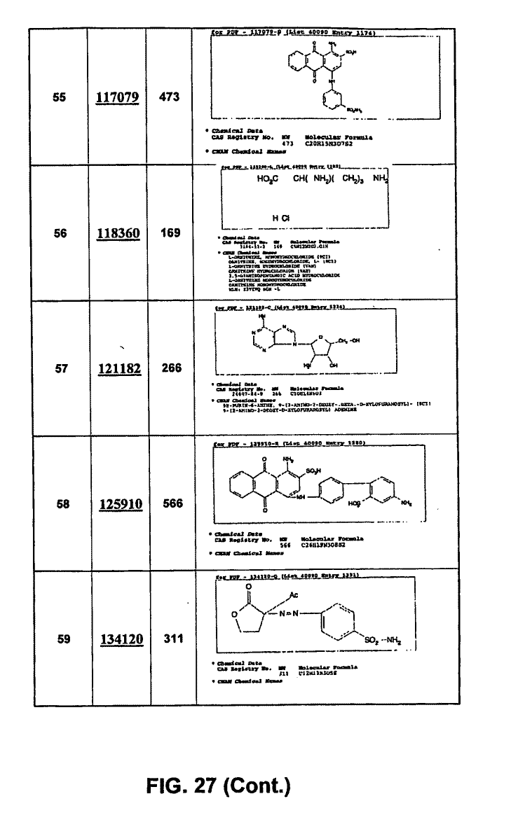

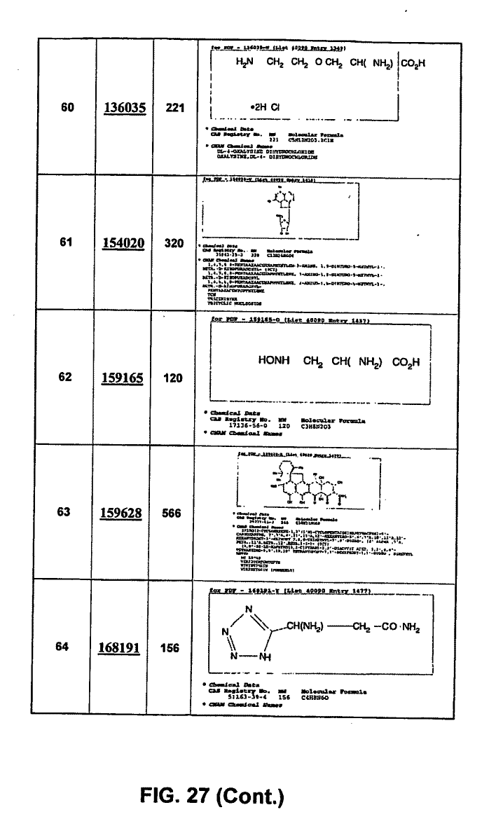

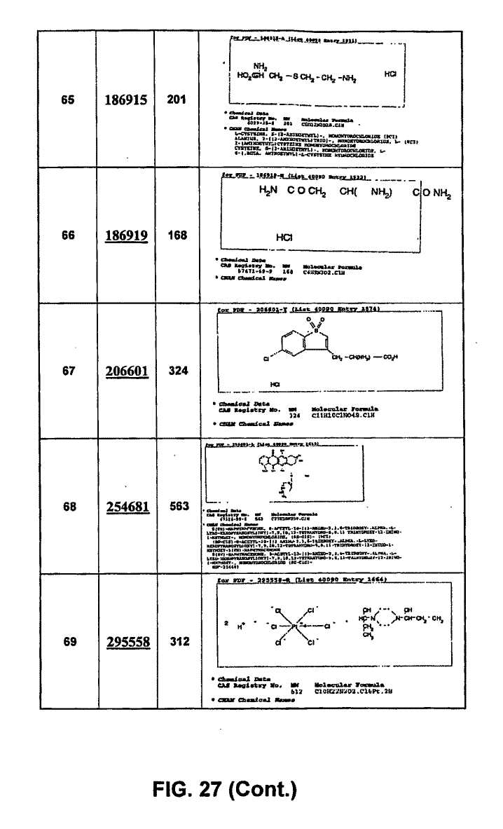

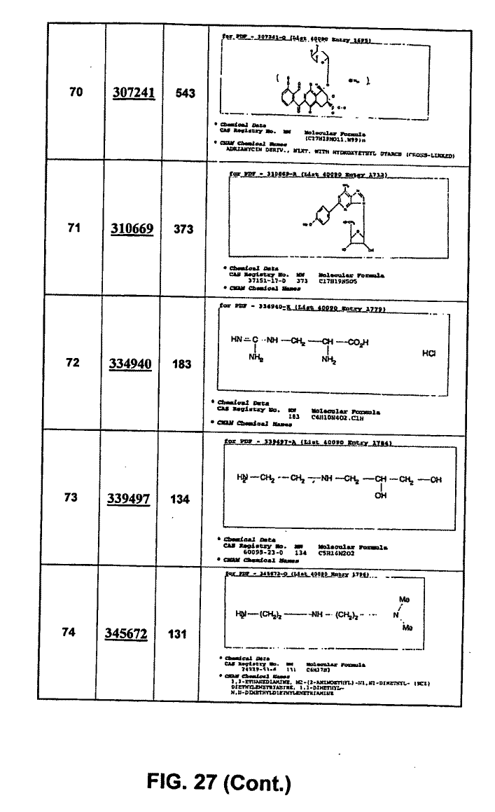

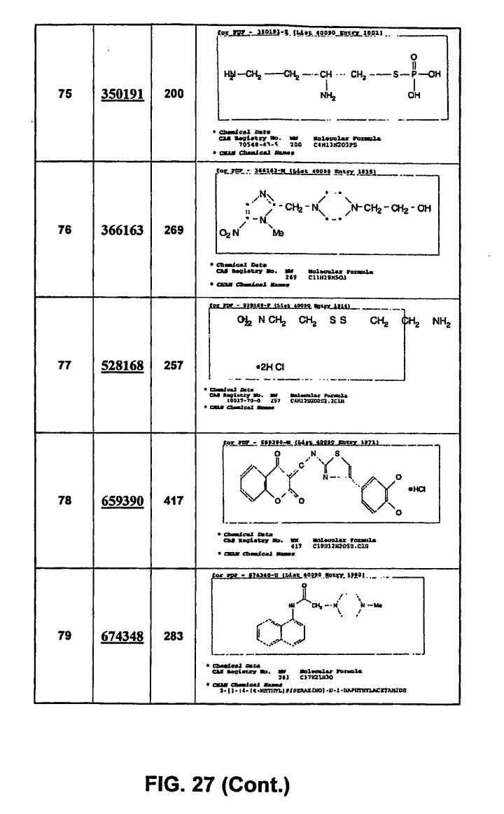

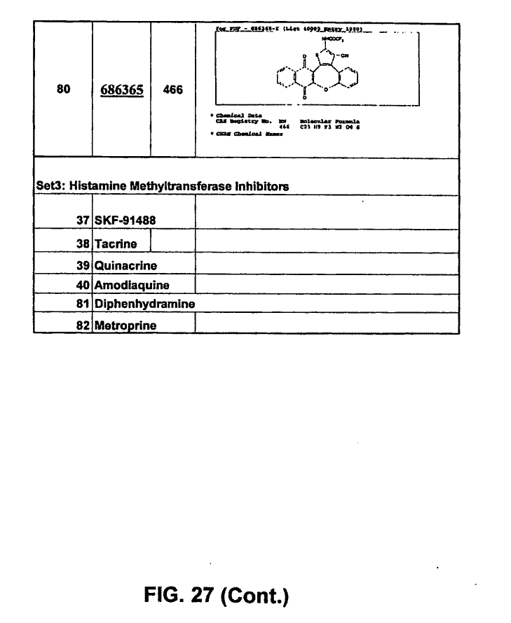

Summary of DOCK results: Currently we have identified 82 compounds (36 identified for DIM-5, 40 identified for T4Dam, and 6 known inhibitors for histamine MTase, a small molecule MTase (Horton et al., 2001)) (FIG. 27). The results of the DOOK analysis are summarized in FIG. 28 that shows the top 100 compounds ranked by energy score. Among these, nine compounds showed modest inhibition in in vitro assays, and one of them (NCI 659390) was found to inhibit actin pedestal formation in a cell-based virulence inhibition (see Table 7). Based on a lead compound structure, other structures related to the lead compound structure can be screened. For example, the structure of compound 78 from Table 7 (NSC 659390), was used to identify other related compounds from the 140,000 compound library (FIG. 29).

It is interesting to note that an alternative functional core structure is defined by two of the nine compounds identified in Table 7 (compound numbers 55 and 58). Similar analysis can identify other functional core structures. In addition to compound Dam-iZ1, compound Dam-iZ2 can function as a Dam inhibitor:

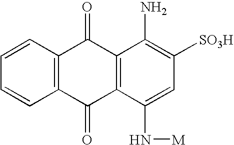

M can be an aryl or heteroaryl, wherein aryl is one or more rings, preferably one or two aromatic rings wherein each ring is optionally and independently substituted. A heteroaryl has an aromatic ring containing one or two heteroatoms. In an embodiment, M is:

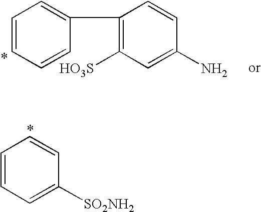

In an embodiment, the “*” indicates the attachment location of M to the nitrogen atom.

Computer aided drug design (CADD) lead identification via database screening: Identification of novel lead compounds with the potential to bind to Dam is performed via database searching of the virtual NCI Diversity Set and a 3D chemical database of over 3 million commercially available compounds. The 3-million-compound database has been compiled and converted from 2D structures to 3D structures (Huang et al., 2004; Pan et al., 2003) in the University of Maryland CADD Center headed by Dr. MacKerell. The majority of the compounds in the database have recently been shown to have drug-like properties (Sirois et al., 2005). The target for the initial database search is the catalytic site of EcoDam; later searches target novel binding sites as determined via the proposed crystallographic studies. Database searching is performed using the program Dock (Kuntz et al., 1982) using the anchor based search approach to account for ligand flexibility (DesJarlais et al., 1986; Ewing and Kuntz, 1997). Prescreening is performed against select compounds that contain 10 or less rotatable bonds and between 10 and 40 non-hydrogen atoms and energy scoring is based on the GRID (Goodford, 1984) method implemented in Dock. From the initial docking, the top 50,000 compounds are selected based on normalized van der Waals (vdW) attractive interactive energies. Use of the vdW attractive energy, versus total energy or electrostatic energy, forces the procedure to select compounds with structures that sterically complement the binding site (Huang et al., 2004). The normalization procedure is designed to control the molecular weight (MW) of the selected compounds (Pan et al., 2003); use of N^{1/2} normalization where N is the number of non-hydrogen atoms in the compounds, selects compounds with an average MW of 320 daltons. Such compounds are smaller than the average MW of pharmaceutically active compounds based on the World Drug index. Smaller MW compounds are desirable at this stage of a drug design project as they are more amenable to modification at later stages of the project (Oprea et al., 2001).

Secondary virtual searching of the top 50,000 compounds selected from the initial screen includes simultaneous energy minimization of the anchor during the iterative build-up procedure (Chen et al., 2000; Huang et al., 2004). The secondary screening is performed against the non-Dam MTases, DpnII (Tran et al., 1998), PRMT1 (Zhang and Cheng, 2003), and DIM-5 (Zhang et al., 2002; Zhang et al., 2003) as well as EcoDam in order to include specificity in the compound selection. The final score for each compound is obtained by summing the total interaction energies for each compound with EcoDam and the weighted sum of the difference between EcoDam and non-Dam MTase interaction energies, as follows:

I.E._{Final}=I.E._{Dam}+Σw(I.E._{DAM}−I.E.ᵢ)

where I.E. is the total interaction energy, i represents each of the non-Dam MTases being used for the selectivity screen, and w is a weighting factor equivalent to 1/n, where n is the number of non-Dam MTases. In this scheme, the absolute binding to EcoDam is combined with the relative binding to EcoDam with respect to the non-Dam MTases. Thus, if a compound binds very favorably to EcoDam as well as to the non-Dam MTases its overall score will be relatively low, while a compound that binds less favorably to non-Dam MTases but binds even less favorably to the on-Darn MTases will score higher. If deemed necessary the weighting of the EcoDam interaction energy versus that of the non-Dam MTases can be adjusted. For example, if specificity problems are particularly problematic with respect to one of the non-Dam MTases, its weighting can be increased relative to the others, causing selectivity with respect to it to have a larger impact on the final score. The use of the total interaction energy, versus the vdW interaction energy used in the initial, method 1 screen, allows both electrostatic and vdW contributions to be taken into account during the second stage of the screening process. This is appropriate as compounds whose binding is dominated by non-specific electrostatics are eliminated in the initial screen. From the method 2 selectivity screen the top 1000 compounds are chosen for the chemical similarity analysis (Butina, 1999), a step that maximizes the chemical diversity of the final compounds selected for biological assay that has been shown to improve screening hit rates (Huang et al., 2004). In this process, chemical similarity is quantified based on chemical fingerprints in combination with the Tanimoto index yielding approximately 100 clusters of chemically similar compounds. One or two compounds are selected from each cluster for biological assay. This final selection process considers stability, potential toxicity, and solubility [i.e. Lipinski's rule of 5 (Lipinski, 2000)], where solubility is estimated via calculated log P values using the Molecular Operating Environment (MOE, Chemical Computing Group). Selected compounds are purchased from the appropriate vendors.

Lead identification potential pitfalls and alternatives. ISS via database searching makes a number of simplifications in order to minimize computer requirements, allowing for the database of 3 million compounds to be searched. Of these simplifications the two most important are (1) the lack of conformational flexibility in the protein (Carlson, 2002) and (2) the simplified scoring function. If either of these assumptions is indicated to be problematic due to a low number of active compounds identified in method 2, the following steps are taken.

To account for protein flexibility multiple structures are used for the method 2 docking. Additional conformations (typically 5) for EcoDam are obtained from a molecular dynamics (MD) simulation and included in the method 2 search, such that each of the 50,000 compounds are screened against each conformation, with the most favorable score for each compound used for the final ranking. The additional conformations are generated via molecular dynamics (MD) simulations of the region of the protein being targeted, using the molecular modeling program CHARMM (Brooks et al., 1983; MacKerell et al., 1998b). These simulations are performed as previously described (Huang et al., 2003) for 5 ns in explicit solvent using the CHARMM22 protein force field (MacKerell et al., 1998a) that includes the recent revision to the treatment of the protein backbone (MacKerell et al., 2004).

Alternate scoring methods are attempted if the hit rate (i.e. number of active compounds selected) is deemed inadequate. One alternate approach is consensus scoring (Charifson et al., 1999), a method that applies multiple scoring function to rank compounds. This approach includes knowledge-based scoring methods that have been shown to yield improvements in the selection of correct orientations of ligands and have the advantage that they implicitly include certain aspects of salvation effects. Additional alternate approaches include generalized linear response methods (Åqvist et al., 1994; Lamb et al., 1999) and free energy of salvation based on the Generalized Born (GB) model (Feig and Brooks, 2004), including a GB version recently implemented in the program DOCK (Kang et al., 2004; Zou et al., 1999).

FIG. 22 (flow chart) summarizes our overall strategy to identify and characterize suitable lead candidates for the development of novel Dam inhibitors. The availability of high-resolution structure of EcoDam (and its many complexes with DNA along the recognition pathway—specific aim 1) paves the way for structure-based virtual screening. ISS is conducted against a small chemical “diversity” library representing the major chemical types in the NCI database and a large database of 3,000,000 commercially available compounds. Compounds identified using the virtual screening are grouped into chemical classes, verified by three assays in parallel: an in vitro methylation assay to determine IC₅₀ values, a bacterial-based in vivo methylation inhibition assay, and cell-based virulence inhibition assay. Best candidates are analyzed to determine their mechanism of inhibition and their selectivity against non-DAM MTases (such as mammalian DNA cytosine MTases, histone lysine MTases, and protein arginine MTases).

Each lead compound identified by ISS is evaluated for its potential to be chemically optimized (guided by the Lipinski parameters for the most desirable properties of lead-like molecules). The toxicity is determined first in cells, then worm (Anyanful et al., 2005), then mice. In vitro and in vivo efficacy is evaluated by the ability to prevent and treat disease caused by particular infections pathogens, like pathogenic E. coli and Salmonella, using mouse models. The primary criteria for optimization are potency against the target enzyme (Dam), negative selectivity against the mammalian MTases, no or low host toxicity. Lastly, co-crystal structures of Dam with lead inhibitors are determined and an iterative approach used to design derivative analogs around the core structure with more desirable properties.

### Example 4

### Activity Testing of Inhibitors

Activity testing encompasses biochemical, in vitro and in vivo assays. These assays are available to test Dam inhibitors identified by ISS and/or computer-aided drug design. For example, the assay can assess DNA methylation in a biochemical system, pedestal formation in whole cells in vitro, or the mouse pathogen Citrobacter rodentium as a model of pathogenic E. coli disease in vivo. See, for example, Swimm et al. (2004); Wei et al. (2005).

High throughput assays (HTA) that allow screening of several hundred to thousand compounds are used. In particular, two HTA assays are used: (1) in vitro HTA in microplate format; and (2) cell-based high throughput virulence inhibition assay.

In vitro high throughput assay in microplate format: For the analysis of DNA methylation, we use a microplate assay that utilizes a biotinylated oligonucleotide substrates for analysis of DNA methylation (Roth 2000) The assay uses labeled [methyl-³H]-AdoMet. After the methylation reaction the oligonucleotides are immobilized on an avidin-coated microplate. The incorporation of [3H] into the DNA is quenched by addition of unlabeled AdoMet to the binding buffer. Unreacted AdoMet and enzyme are removed by washing. To release the radioactivity incorporated into the DNA, the wells are incubated with a non-specific endonuclease and the radioactivity determined by liquid scintillation counting. As an example, we studied methylation of DNA by the EcoDam shown in FIGS. 8B and 9. The biotin-avidin assay is inexpensive, convenient, quantitative, fast and well suited to process 96 samples in parallel. The accuracy of the assay is high, with results reproducible to within +/−10%. Single point methylation assays are employed for initial screening for compounds that sow an inhibition potential of the MTase reaction. Steady-state kinetics are then conducted to determine the IC50 of each compound that scores positively in the initial screening.

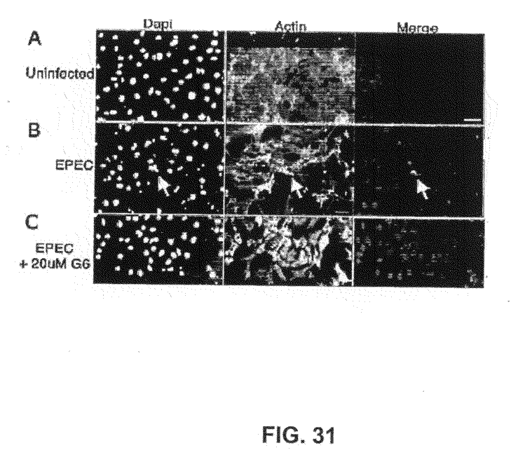

82 compounds were screened, at a compound concentration of 200 μM, in a microplate assay to assess their ability to inhibit Dam. The test was repeated using only the positives (with > 2 fold inhibition). All 9 positive compounds (Table 8) were “identified” by DOCK as potential inhibitors of Dam. This is a good validation of the ISS. The microplate assay uses labeled [methyl-3H]-AdoMet. After the methylation reaction the oligonucleotides are immobilized on an avidin-coated microplate. The incorporation of [3H] into the DNA is quenched by addition of unlabeled AdoMet to the binding buffer. Unreacted AdoMet and enzyme are removed by washing. To release the radioactivity incorporated into the DNA, the wells are incubated with a non-specific endonuclease and the radioactivity determined by liquid scintillation counting. Compound #78 (e.g. NCI 659390) was found also to inhibit actin pedestal formation in a cell-based virulence inhibition assay (see FIG. 31).

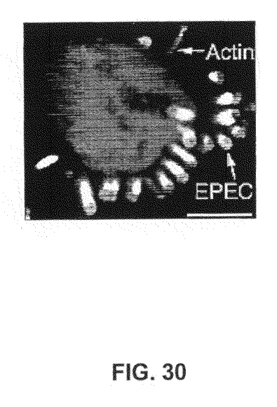

Cell and animal models for pathogenic E. coli infection: Enteropathogenic E. coli (EPEC), which is closely related to enterohemorrhagic E. coli O157:H7 (EHEC), and the closely related mouse pathogen Citrobacter rodentium all cause attaching and effacing (A/E) lesions, characterized by flattening of intestinal microvilli, adherence of the bacteria to epithelial cells, and reorganization of the host actin cytoskeleton, which result in the formation of an actin-filled membrane protrusion or “pedestal” beneath each bacterium (Goosney et al., 2000; Knutton et al., 1989). Pedestal formation is readily detected on cultured fibroblasts (see FIG. 30) exposed to EPEC, and then stained with antibodies that recognized outer membrane proteins in the bacterium (green in FIG. 30) or DAPI, which recognizes bacterial and cellular DNA (blue in FIG. 30), together with phalloidin to recognize actin (red in FIG. 10). Pedestals are seen as intense actin staining directly opposed to the bacterium.

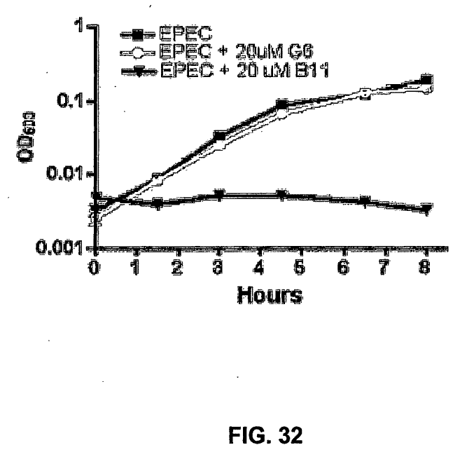

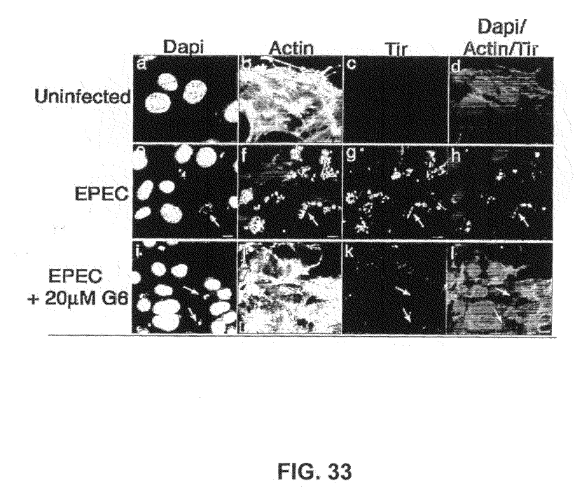

To determine whether formation of actin pedestals by EPEC could be used to screen for drugs which inhibit virulence, we tested each of the 82 compounds. 3T3 cells were plated in 96 well optical dishes. In this “proof of principle” experiment, each of the 82 compounds was added to a well at 20 μM, and cells were infected with EPEC for 6 hrs. The optical density (OD₆₀₀) of the supernatant was assessed to estimate effects on bacterial growth, and the cells were fixed and stained with DAPI to recognize bacteria (and cell nuclei), and FITC-phalloidin to recognize filamentous actin. Inhibition of actin pedestals is readily identifiable as the loss of intense actin staining (Kalman et al., 1999; Swimm et al., 2004a). The plate was then scanned visually on an inverted Zeiss 200M fluorescence microscope with a 20× objective. All the wells on the dish were examined with 5 minutes (a high throughput format). At low power, actin pedestals are seen as intense fluorescence apposed to groups of bacteria (see FIG. 31B, arrow). No such staining was evident in uninfected cells. We identified one compound, B11 (well position, compound #23) that blocked bacterial cell growth measured by OD₆₀₀, and pedestal formation (not shown). B11 turned out to be a derivative of antibiotic mitomycin. We identified a second compound, G6 (compound #78), which appeared to inhibit actin pedestals but did not affect bacterial growth (FIG. 31C). This compound was identified by DOCK against T4Dam and selected for more detailed analysis. OD₆₀₀ measurements at several time points indicated that G6 had no effect on bacterial growth (red line, FIG. 32). Moreover, no pedestals were evident next to attached bacteria even at high magnification (63x; FIG. 33J), and G6 had no gross cytopathological effects on 3T3 cells.

Formation of pedestals is highly correlated with the development of diarrhea, but its relationship to the onset of disease is poorly understood. Of importance here, pedestal formation is an indicator of pathogenic E. coli virulence and is readily amenable to high throughput drug screening protocols. To make pedestals, EPEC initially attaches loosely to epithelial cells and then inserts its Type III secretion system into the host cell plasma membrane, and secretes several virulence factors into the host cytoplasm and membrane (Goosney et al., 2000), including the translocated intimin receptor (Tir) (Kenney et al., 1999). We also assessed effect of compound G6 on expression of the bacterial virulence factor Tir. As seen in FIG. 33g, Tir staining was evident in the actin pedestals of infected cells. However, in cells treated with compound G6, no Tir staining was evident next to attached bacteria (FIG. 33k). These data indicate that the compound G6 directly or indirectly inhibited expression or secretion of Tir, and provide “proof of principle” that actin pedestals can be used to screen for compounds that inhibit bacterial virulence without affecting growth. Thus pedestal formation in cultured fibroblasts is an indicator of virulence factor expression in the bacteria, and is highly correlated with development of disease in animal models. Accordingly, pedestal formation is an appropriate assay to assess Dam MTase inhibitor function.

As demonstrated FIGS. 31-33, the formation of actin pedestals by (EPEC) can be used to screen for drugs that inhibit virulence. As a control, we assess pedestal formation with an EPEC strain having a nonpolar deletion of the gene encoding the Dam MTase. To do this, we employ the methods of Datsenko and Wanner, a highly efficient means of inactivating E. colligenes. This method has been used to generate a nonpolar deletion in tnaA. Briefly, the method utilizes PCR and the bacteriophage a Red recombination system, and it avoids the labor-intensive process of creating the gene disruption on a plasmid vector. The Red system encodes three gene products, Gam, Bet, and Exo. Gam inhibits the host RecBCD exonucleaseV so that Bet and Exo can gain access to linear DNA ends to promote recombination. We use PCR to generate linear DNA containing Dam adjacent sequences flanking two FRT (FLP recognition target) sites that surround the chloramphenicol (Cm) resistance gene. The product is treated with DpnI (to eliminate methylated (unamplified) template DNA), re-purified, and then electroporated into an EPEC strain, which contains the Red helper plasmid pKD46. Mutants are selected on LB agar containing chloramphenicol and ampicillin plus 1 mM L-arabinose to induce the Red genes. Mutants are passaged on nonselective medium to allow segregational loss of the Red helper plasmid (rendering them Amp^{s}). Recombinational insertion of the deleted gene is verified by PCR analysis utilizing appropriate primers. Elimination of car^{R} is performed using a helper plasmid encoding the FLP recombinase, which is curable by growth at 43° C. PCR products generated from this region are sequenced for verification of the deletion. Dam⁻ phenotype is demonstrated by resistance to DpnI digestion and sensitivity to DpnII digestion. Complementation assays of the deletion strain are performed by expression of the Dam protein from an appropriate plasmid. The Dam-deletion strain is used as a control in the actin pedestal assay and in the mouse virulence assay.

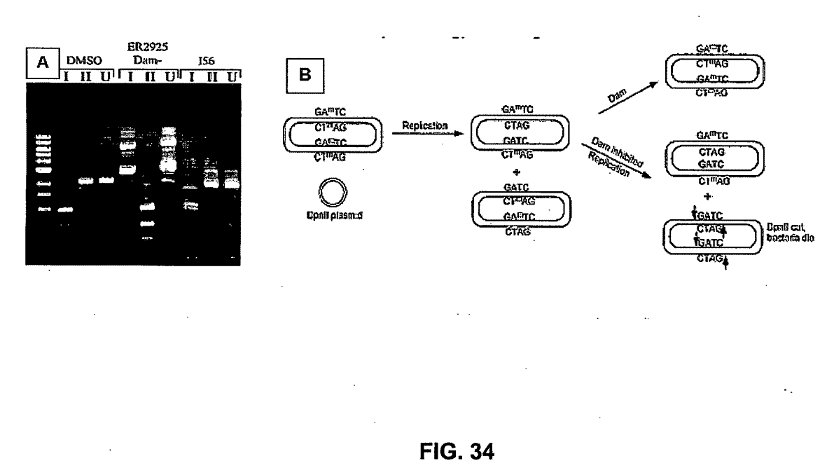

Compounds identified as inhibitory in the in vitro assay, but having no affect on actin pedestal formation in cell-based assay, the likely reason is inability of the compound to enter the bacteria. If this occurs, the compounds can be optimized, as known in the art, to permit entry into the bacteria. To test whether these compounds affect bacterial growth and methylation, E. coli cultures carrying pUC19 plasmid are grown in the presence of various concentrations of the inhibitors. The plasmid is isolated and digested with restriction enzyme DpnI (cuts only methylated DNA) and KpnII (cuts only unmethylated DNA) to analyze its methylation status. Any loss of methylation in this assay indicates that the inhibitor can enter the bacterial cell and inhibit Dam activity. We assay 40 NCI compounds (identified by DOCK for T4Dam) at 50 μM, and some compounds displayed slight incomplete DpnI digestion (I56 in FIG. 34A).

If more sensitive assays are required arising from the requirement of inhibition of the majority of methylation activity in bacteria, other bacterial-based assays can be employed. One such assay is outlined in FIG. 34B. Plasmid expressing DpnII restriction enzyme are introduced into DH5a. Since the genome of DH5a is normally methylated, the gene for DpnII that only cuts unmethylated DNA should be well tolerated (as shown by the availability of DpnII overexpression plasmid). If the bacterial Dam activity is blocked by inhibitors, unmethylated DNA can be generated and cut by DpnII, resulting in cell death. DH5α cells with and without DpnII plasmid are grown in the presence of potential inhibitors in microtiter plates, and the growth rate monitored. Any inhibitors that affect only the growth of DpnII containing culture are likely inhibitors of Dam MTase.

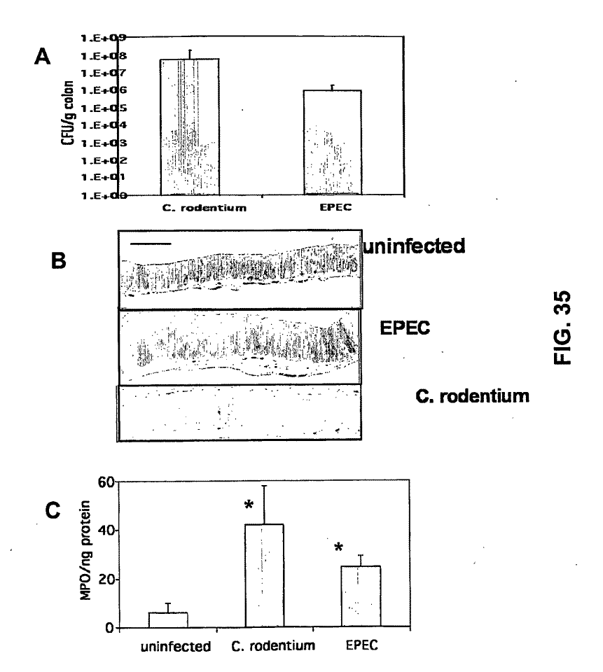

Screening of lead Dam MTase inhibitory compounds in mice. The mouse pathogen Citrobacter nodentium is a model of pathogenic E. coli disease. C. rodentium colonizes the colon, and causes A/E lesions, colonic hyperplasia, and inflammation reminiscent of EPEC effects in humans. C57BL/6 mice have been infected with EPEC using an approach developed for Salmonella infection of mice. Barthel et al. 2003. This approach utilizes short-term streptomycin treatment to reduce, though not eliminate, intestinal flora. Upon infection of streptomycin-treated mice with EPEC (Str^{r}), colons became colonized, developed epithelial hyperplasia at 7 days post infection (=pi), and had elevated neutrophil recruitment as measured by myeloperoxidase (MPO) levels (FIG. 35). This in vivo model of EPEC pathogenesis is used to establish the efficacy of the leading Dam MTase inhibitors as antimicrobials.

Attaching and effacing (A/E) lesions are essential for EPEC and C. rodentium to colonize the colon, so we expect that Dam MTase inhibitors that block A/E lesions in vitro (see preliminary results) to decrease bacterial load in vivo. Oral ingestion of EPEC or C. rodentium results in 10⁹-10¹⁰ colony forming units (CFU) per gram of colon tissue by 10 days pi. Typically, the pathogen is cleared by 6 weeks pi in normal adult mice. Initial experiments determine safety of compounds in non-infected mice and bioavailability. We deliver the compound by Alzet osmotic pumps placed subcutaneously (Reeves et al. 2005). These pumps have the capacity to deliver drug continuously, thus minimizing the effects of drug half life on bioavailability. Serum drug levels are measured by Liquid Chromatography Mass Spectroscopy. We conduct a thorough examination of blood enzyme levels and look for other evidence of pathology by autopsy.

Next we determine the effect of the leading compounds on bacterial levels in EPEC- or C. rodentium-infected mice. C57BL/6 mice are orally infected with 2.5×10⁸ CFU in 200 μL phosphate buffered saline (PBS). The mice are treated with drug or carrier for ten days. At day 10 pi, mice are sacrificed and colons harvested, homogenized mechanically, and serially diluted. The number of viable bacteria is determined by plating on MacConkey agar, which is selective for gram negative organisms. C. rodentium colonies are easily distinguished by their pink centers rimmed with white (Wei et al. 2005). We then determine whether the drug reduces bacterial-associated pathology in infected mice. C. rodentium or EPEC infection in mice causes weight loss, reduced activity, diarrhea, ruffled fur, and a hunched posture (data not shown). Immunocompetent mice are able to resolve the infection by six weeks pi and recover normal appearance and activity. In mice sacrificed prior to recovery, histological analysis of the colon reveals an obvious increase in mass (hyperplasia), crypt heights, and infiltration of lymphocytes and granulocytes (Wei et al. 2005).

Drugs that prevent A/E lesion formation reduce EPEC- or C. rodentium-associated disease parameters in infected mice. Mice are orally infected and treated with drug or carrier. To approximate a more realistic clinical scenario, a second group of mice are treated with drug or carrier upon display of disease symptoms (typically by day 10 pi). Mice are weighed every day and visually observed for signs of physical distress (listlessness, hunched posture, perianal fecal staining). Mice are sacrificed on days 14 and 24 pi and their colons examined histologically for signs of disease (3 μm sections cut and stained with hematoxylin and eosin). Crypt heights are measured by micrometry and appearance of mice are graded by an observer blind to the treatment group: One point is assigned to each condition: listlessness, ruffled coat, prolapsed rectum, perianal fecal staining (maximum score=4; minimum score (robust health)=0). Crypt height and body weight results are expressed as average values+/−one standard error. Treatment groups include at least ten mice. Statistical analysis is calculated by the Mann-Whitney t test, with p<0.01 considered significant. If a drug treated group reduces pathology scores, we can conclude that drug therapy positively affects C. rodentium disease outcome.

Methodologies for drug delivery in mice. Infection models together with drug delivery and detection systems are available. For example, the role of Abl-family tyrosine kinases in poxvirus virulence (Reeves et al., 2005) has been established by examining the effects of Abl-kinase inhibitors (e.g. Gleevec and PD-166326) on viral spread and survival in mice. Methodologies were developed to detect these compounds in mouse serum using Liquid Chromatography Mass Spectroscopy and to measure half-life of these compounds in mice (Wolff et al. 2005). These technologies are readily applicable to the detection of DAM MTase inhibitory compounds in serum samples. Some compounds can be delivered by oral lavage, but for others, the measured half-life in mice is short (about 4 hrs), and require delivery via continuous release Azlet osmotic pumps placed subcutaneously prior to or after infection. Other methodologies that solubilize compounds or otherwise improve their bioavailability can be utilized. Together, these methodologies have allowed successful treatment of infections caused by pathogenic microbes in mice.

Investigation of Inhibition mechanisms. The mechanism(s) of action of the three leading compounds are analyzed kinetically to address the mechanism of inhibition: competitive, uncompetitive or non-competitive with AdoMet or DNA. This information on whether the inhibitor interferes with coenzyme binding, specific DNA binding or conformational changes that can be compared with the predictions based on the docking studies. Finally, co-crystallization of Dam-inhibitor complexes are conducted. The information derived from Dam-inhibitor complex structure is used to identify site(s) of structural variability to generate derivatives around the same functional core structure, via synthesis of a compound library, with more desirable pharmacological properties.

Kinetic studies can be complemented by various additional established assays to further investigate coenzyme and DNA binding:

DNA binding studies. DNA binding by EcoDam and other MTases can be studied using nitrocellulose filter binding and surface plasmon resonance (BiaCore). These assays allow fast and reliable determination of equilibrium binding constants and the effects of inhibitors on the binding equilibrium. SPR BiaCore also permits determination of rate constants of DNA binding and release.

AdoMet binding studies. The kinetics of AdoMet binding to EcoDam can be monitored directly by a change of the intrinsic fluorescence to Trp10 (Liebert and Jeltsch, unpublished). Fluorescence effects are detectable in binary as well as ternary complexes. Therefore, this assay permits measurement of any effect of the inhibitors on AdoMet binding directly and with high sensitivity.

Target base flipping (2AP-based assay). An objective of the present invention is the development of inhibitors that specifically interfere with binding of the Dam enzyme to specific GATC sites and conformational changes. One of the most impressive conformational changes of the enzyme-DNA complex that precedes methylation is the flipping of the target base out of the DNA helix. The mechanism of base flipping has been studied by stopped-flow kinetics using a substrate that contains 2-aminopurine. This base analog provides strong fluorescence signal after DNA bending and base flipping, correlating the 2-aminopurine signal in EcoDam to base flipping (Liebert et al. 2004). Results of this assay indicate that base flipping and DNA recognition are tightly coupled and interwoven processes. Base flipping takes place in a biphasic manner, first the target base is rotated out of the DNA in a very fast reaction and later the target base is tightly contacted by the enzyme and positioned in the active site pocket (Liebert et al. 2004). An inhibitor that binds into the binding pocket of the target base may specifically prevent the base flipping. By following 2-aminopurine fluorescence in equilibrium and using rapid kinetics approaches, the possible influence of MTase inhibitors on this conformational change can be examined.

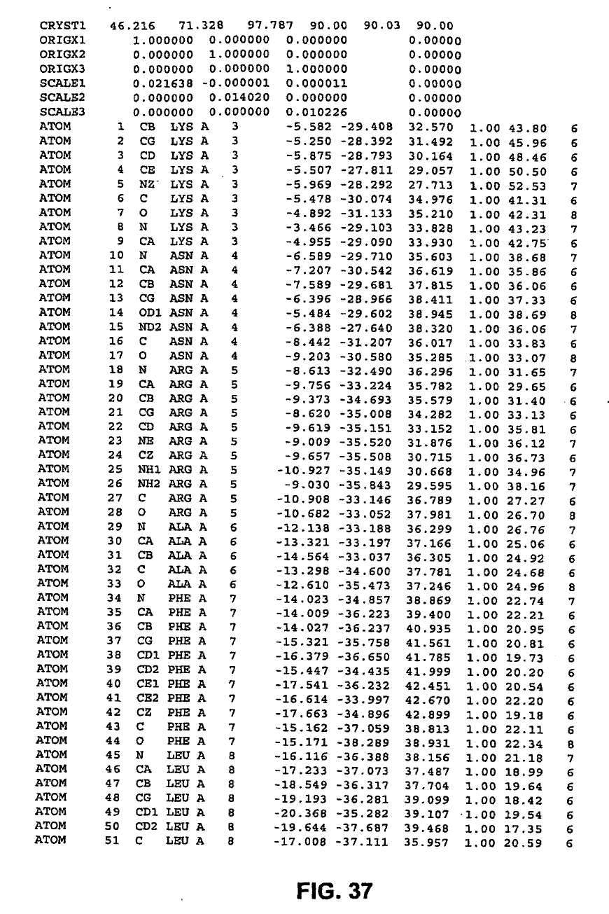

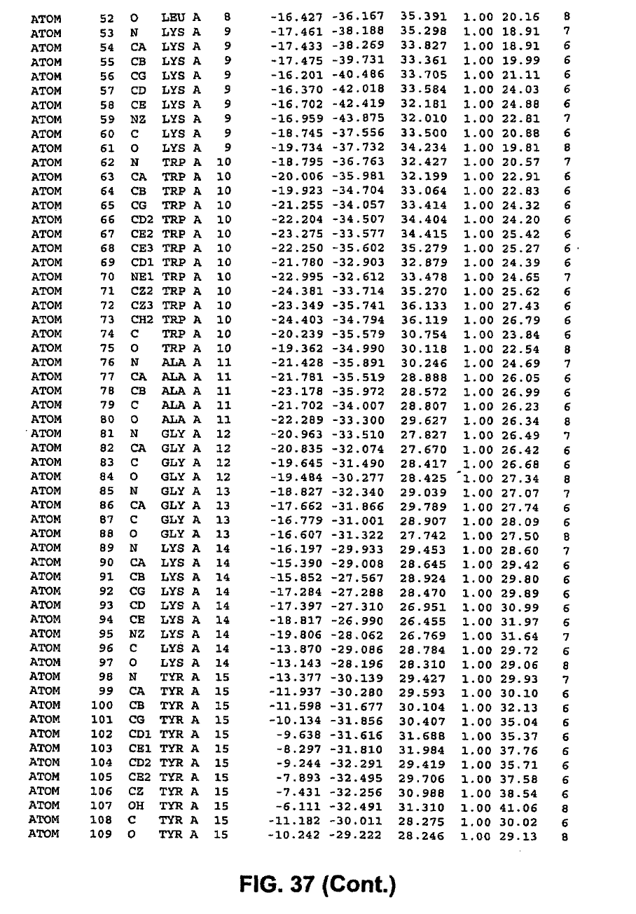

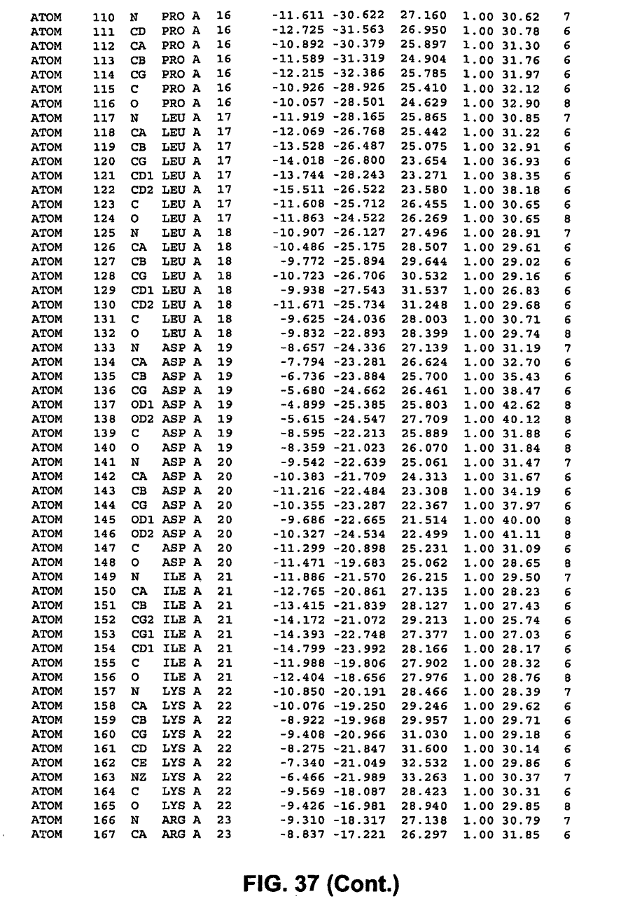

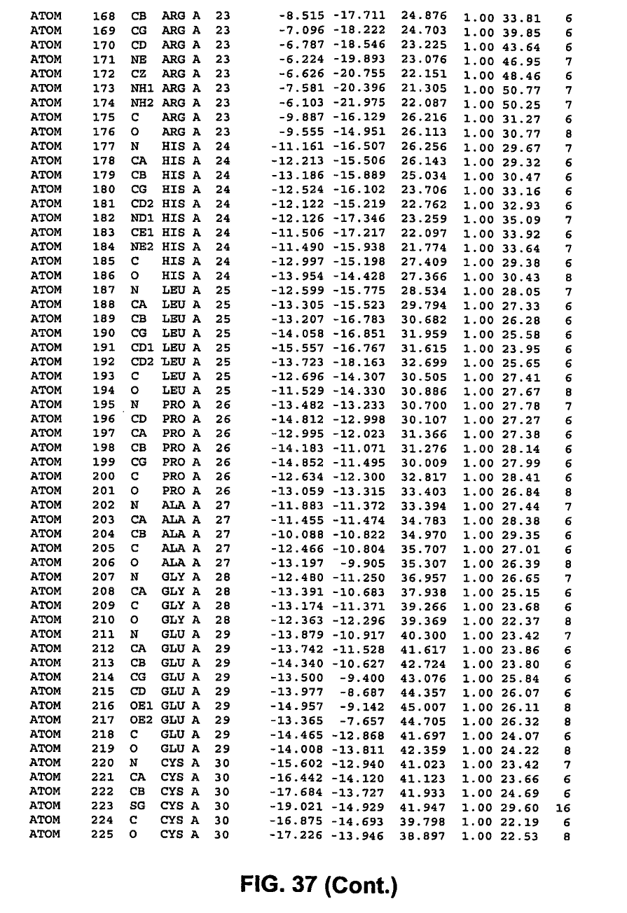

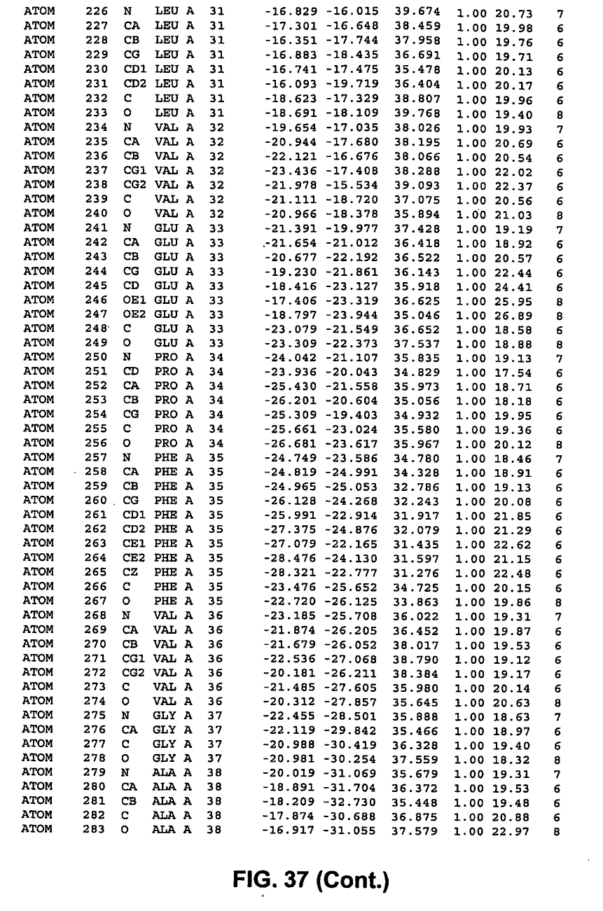

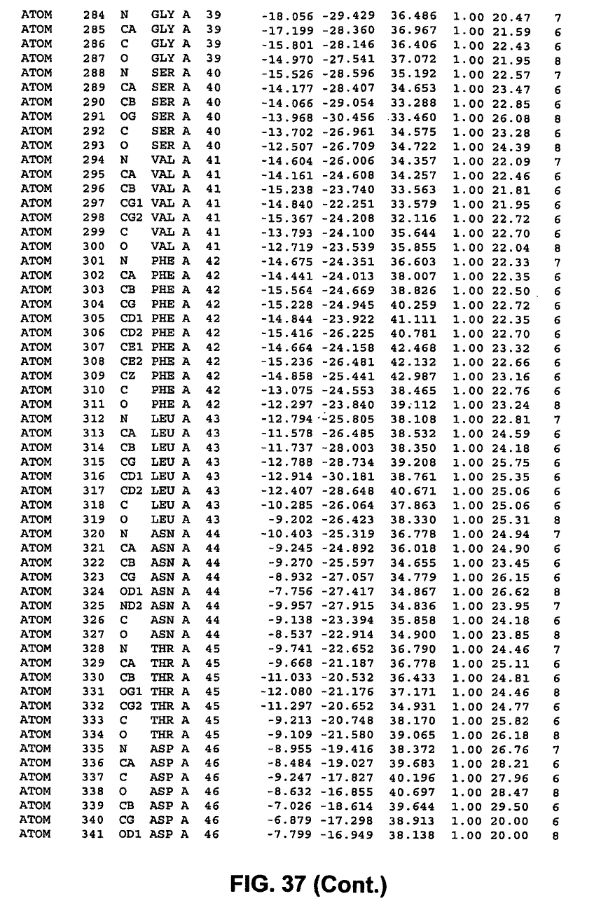

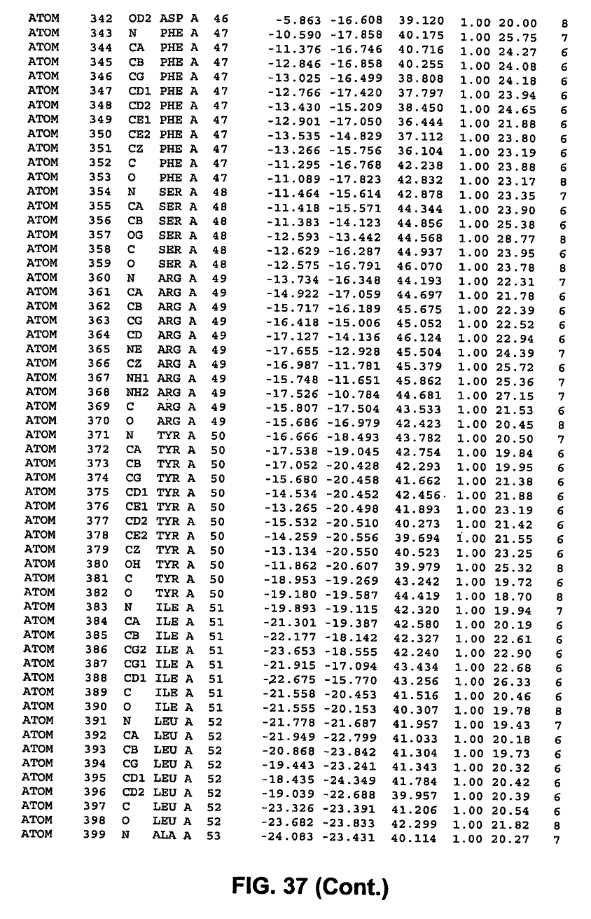

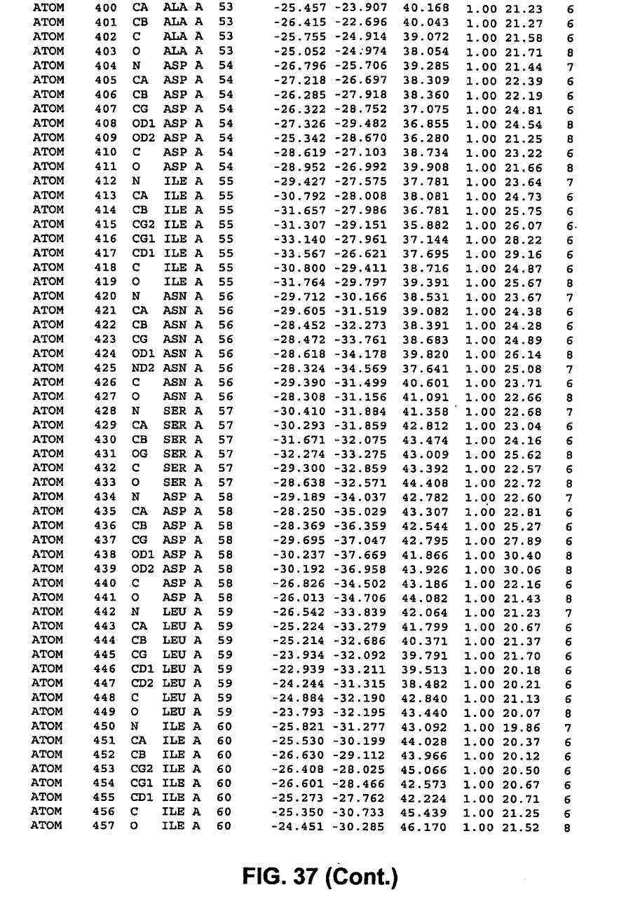

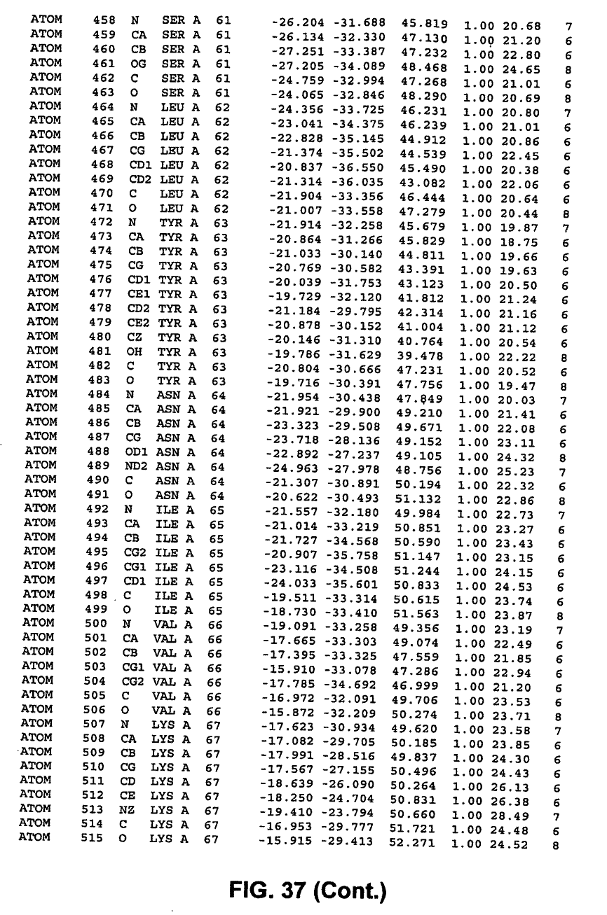

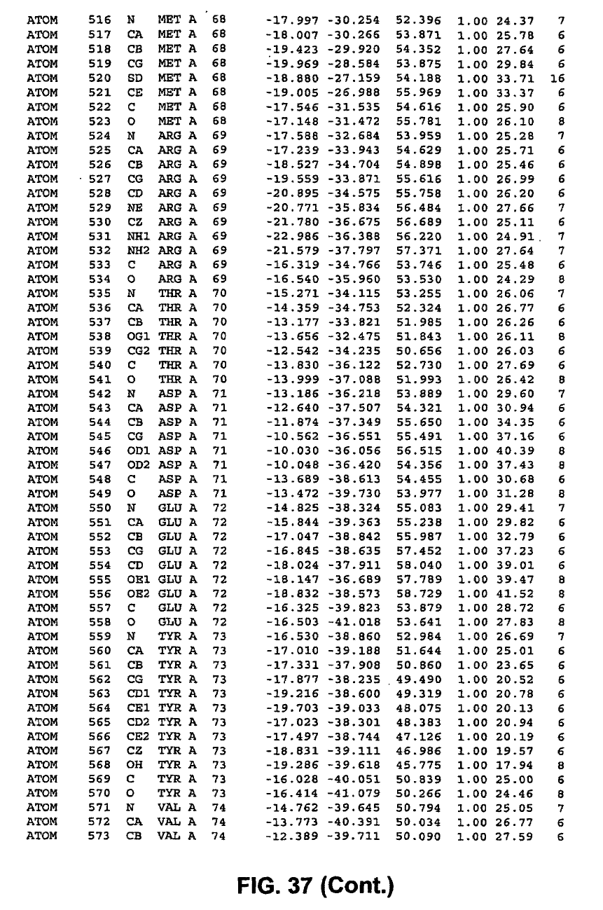

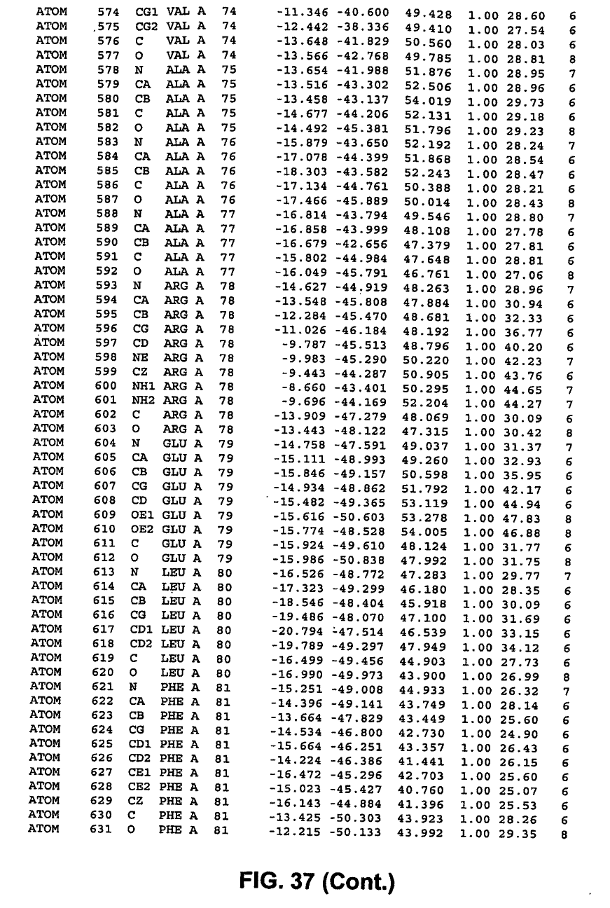

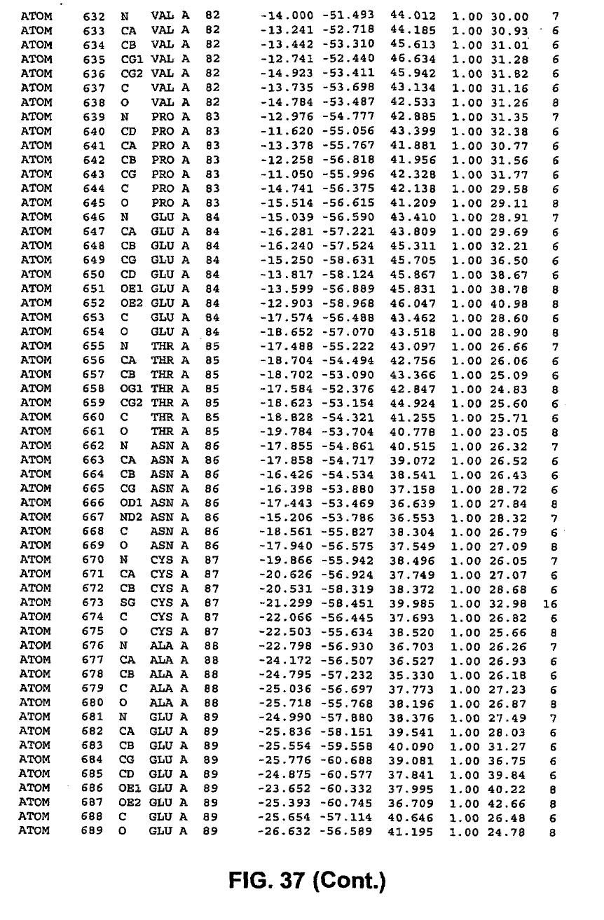

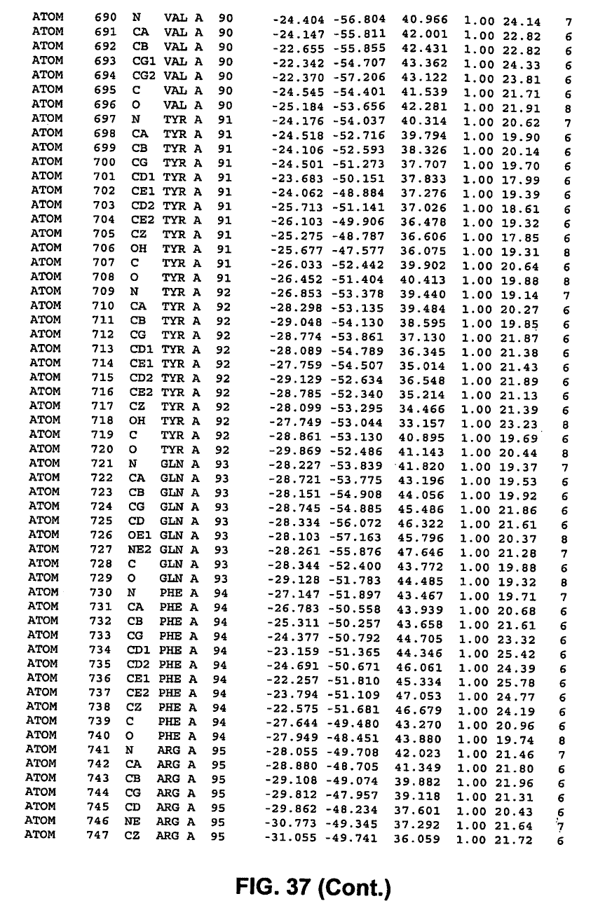

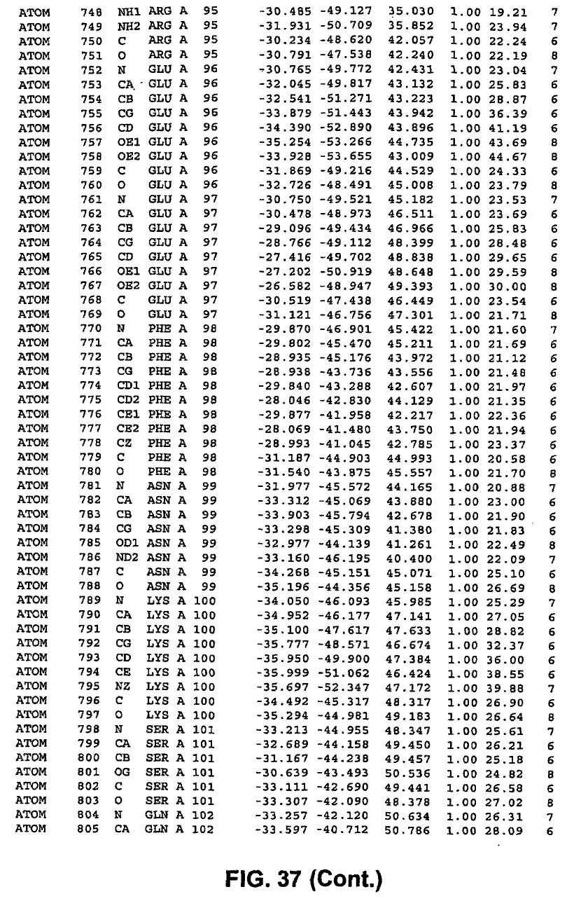

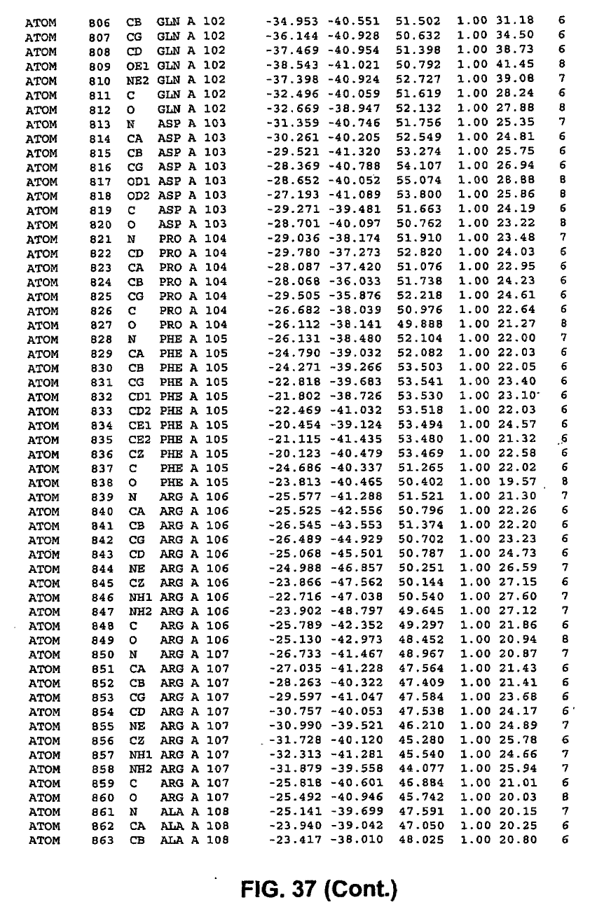

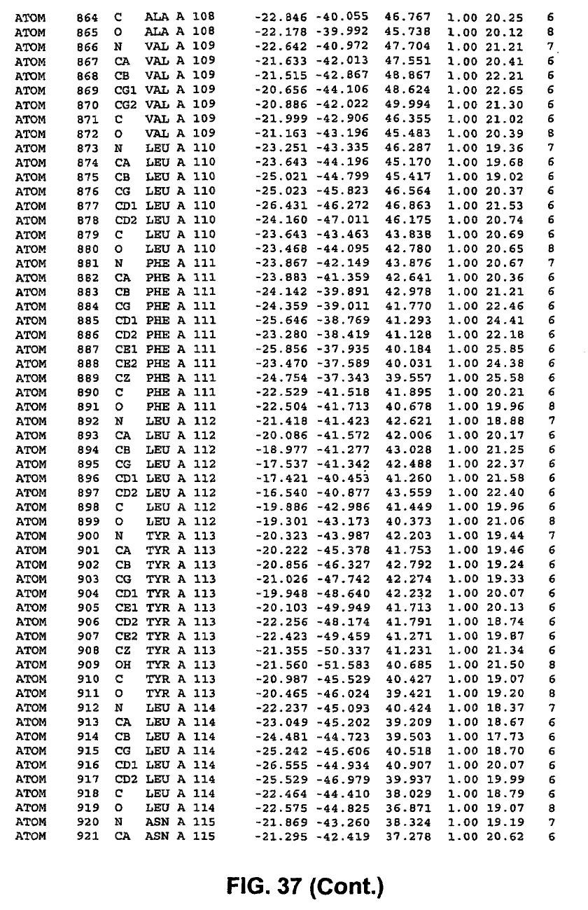

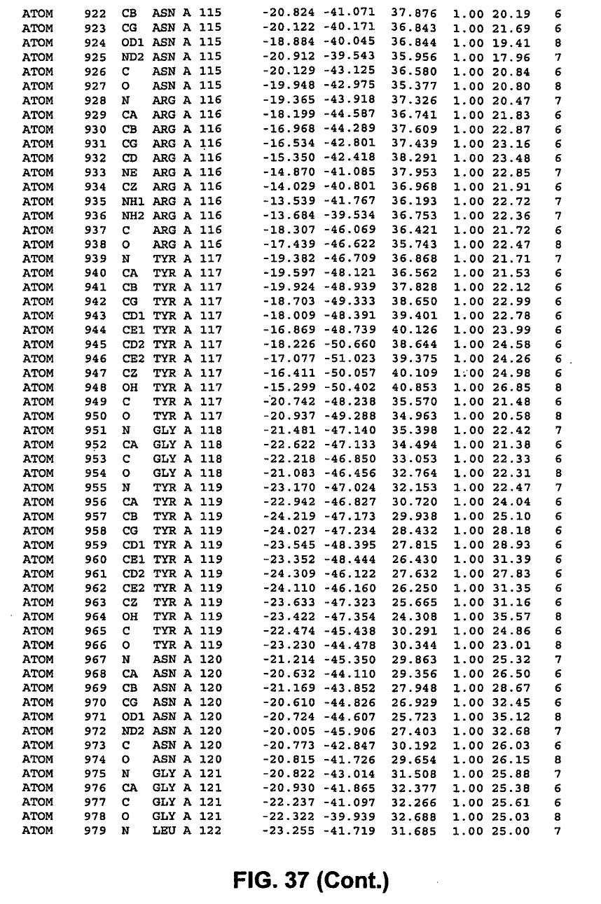

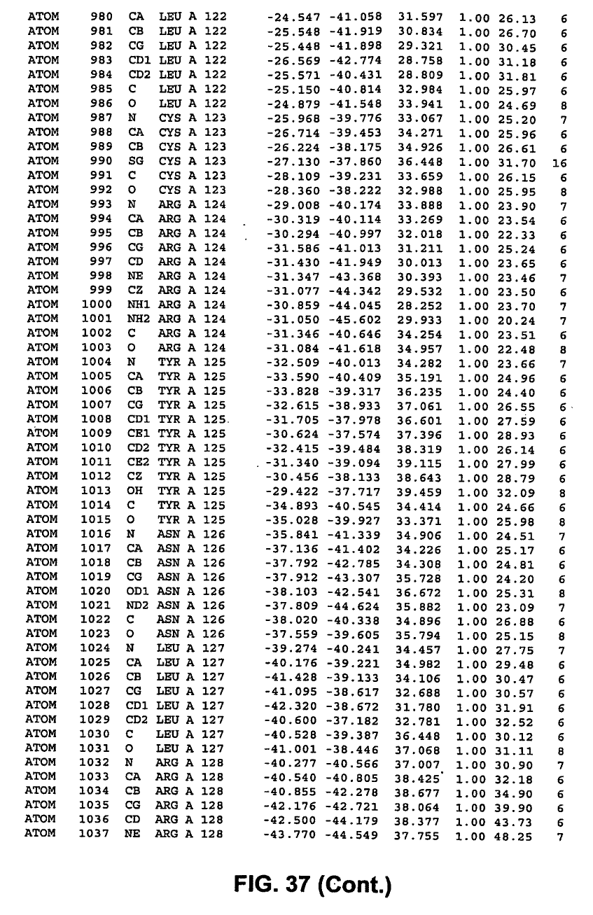

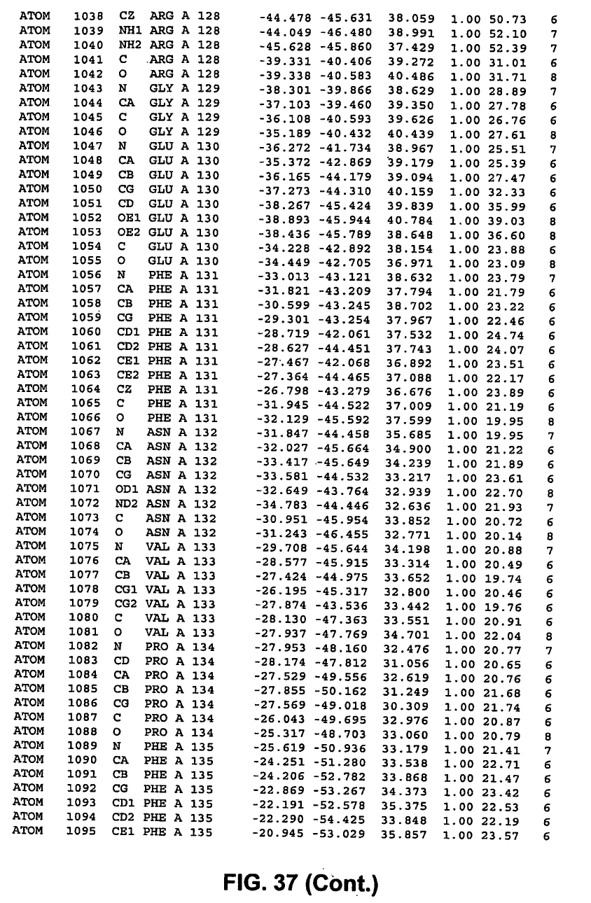

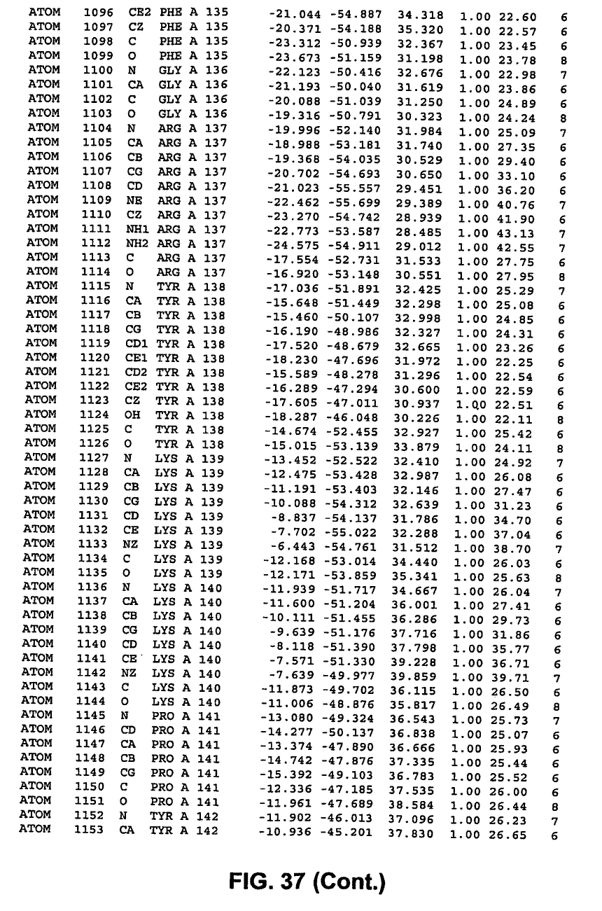

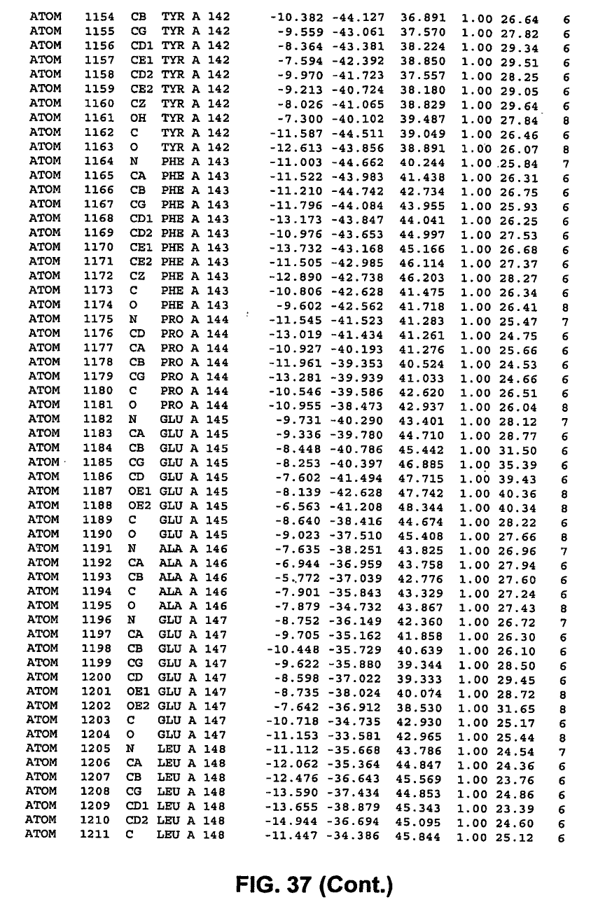

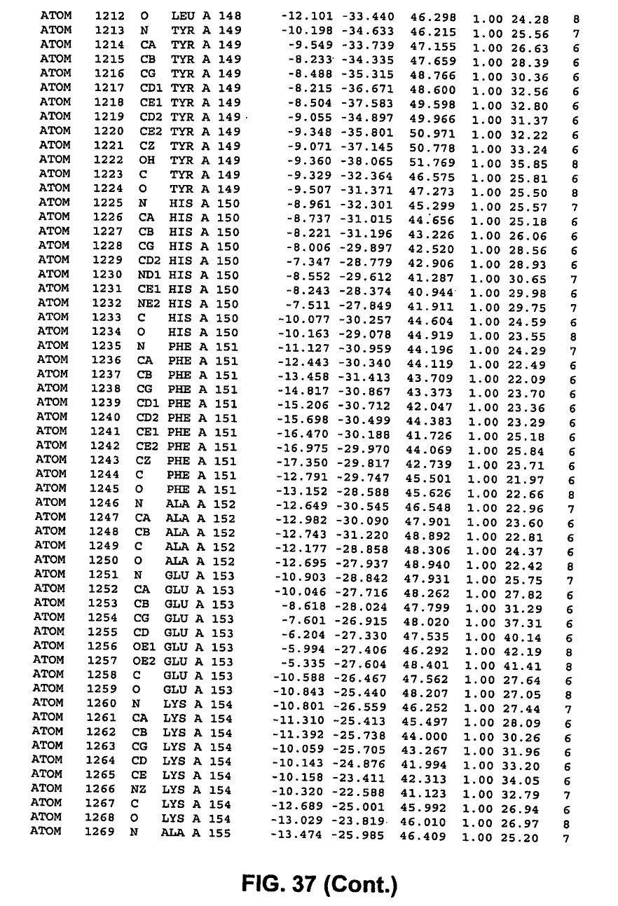

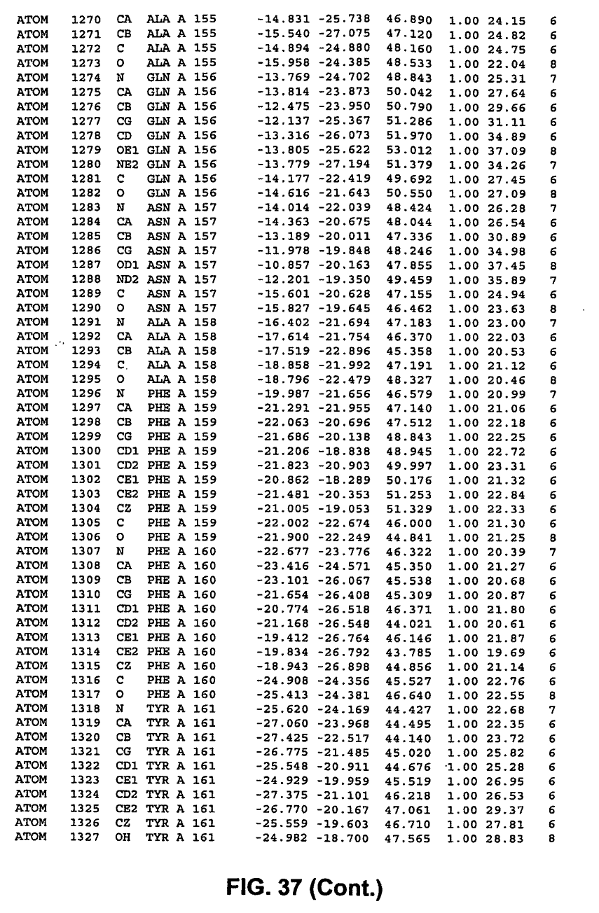

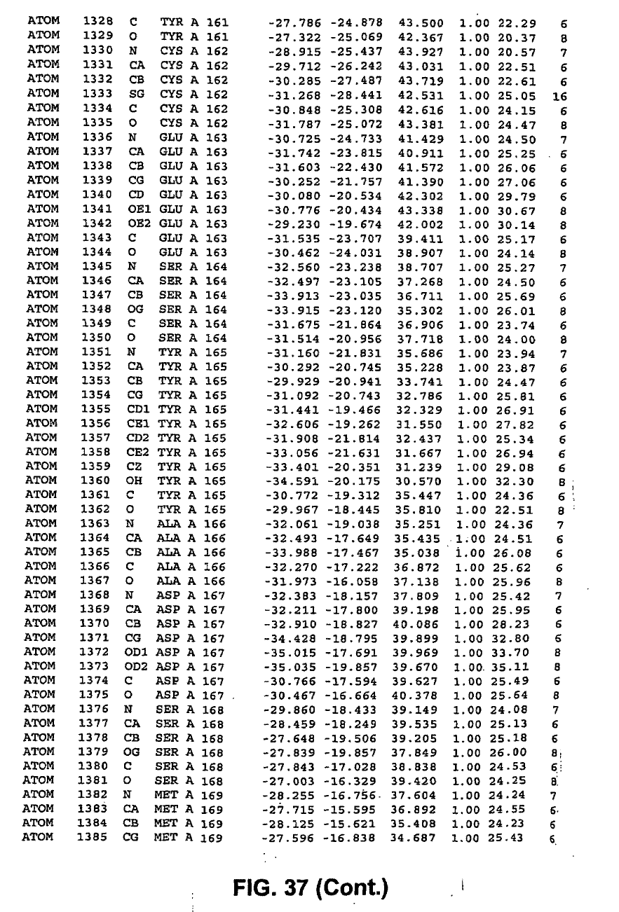

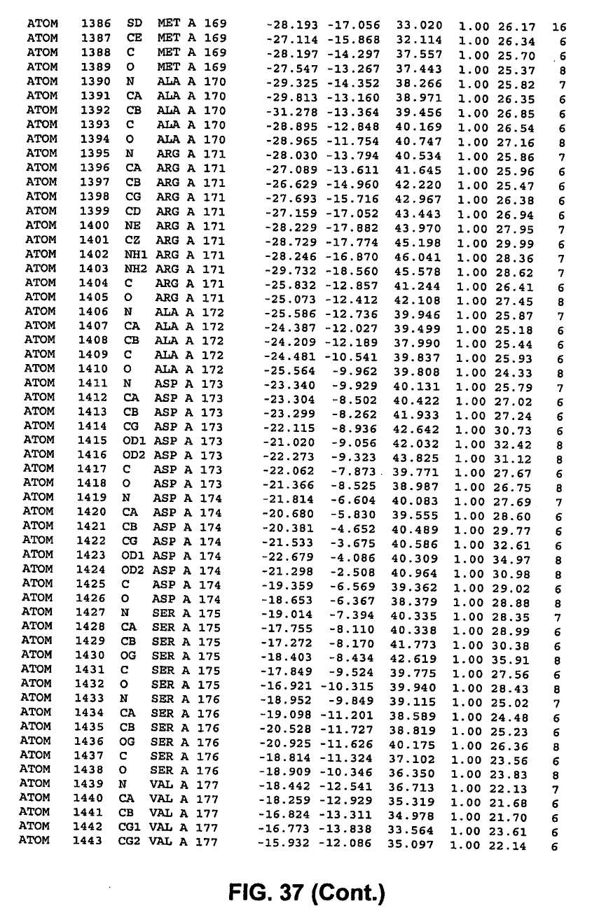

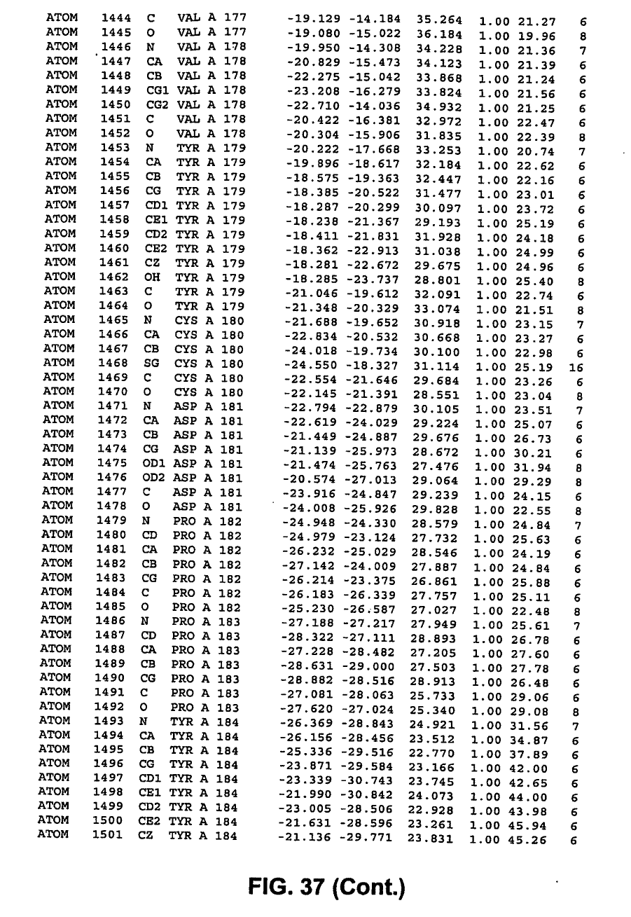

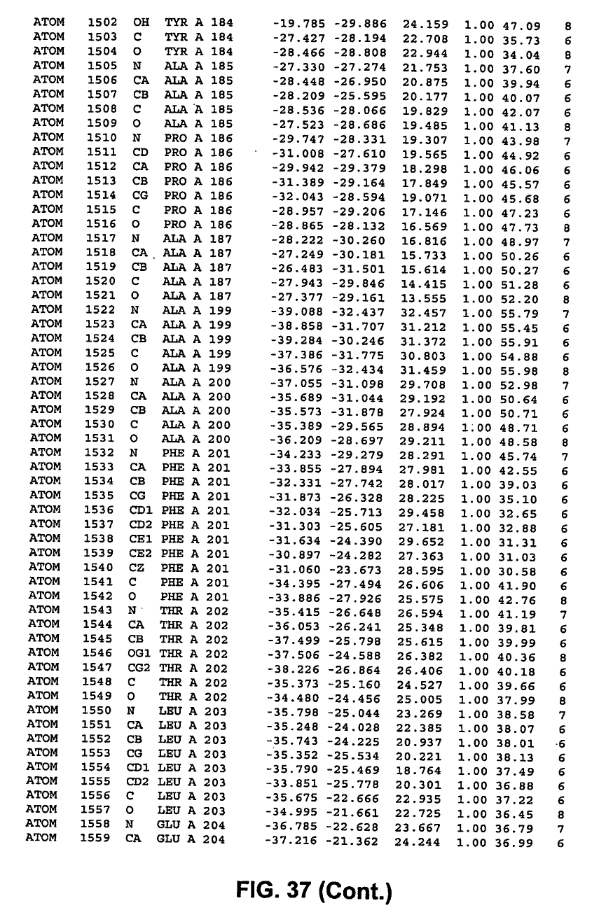

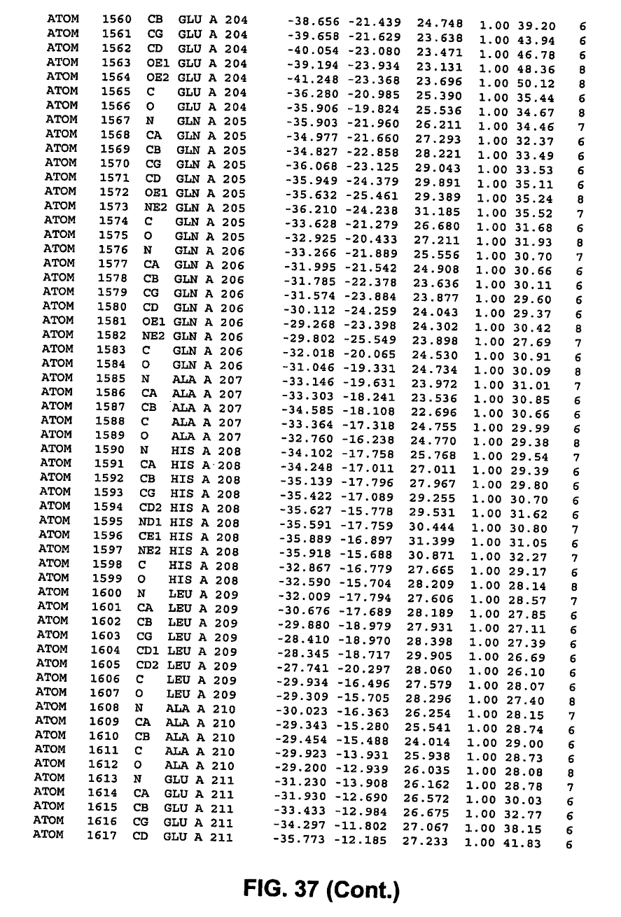

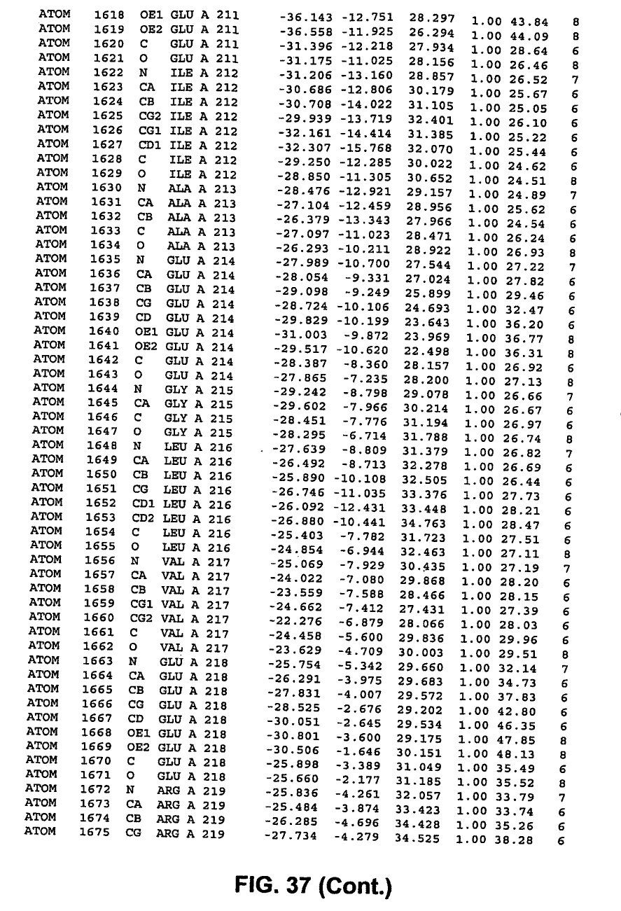

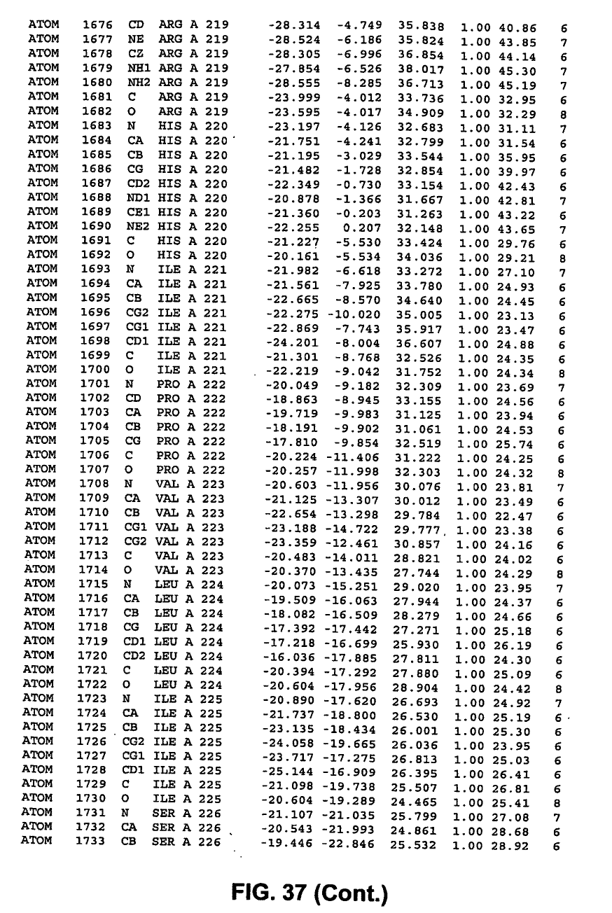

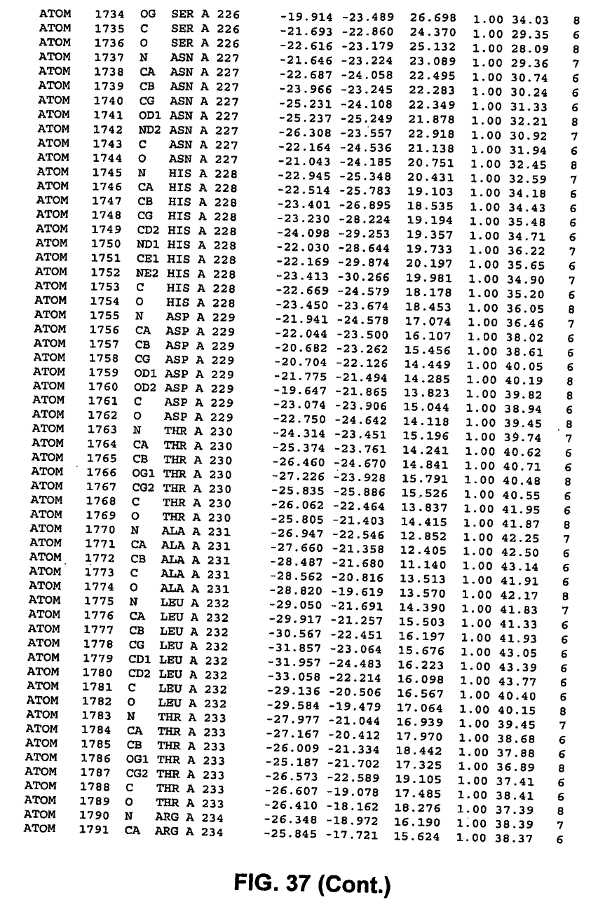

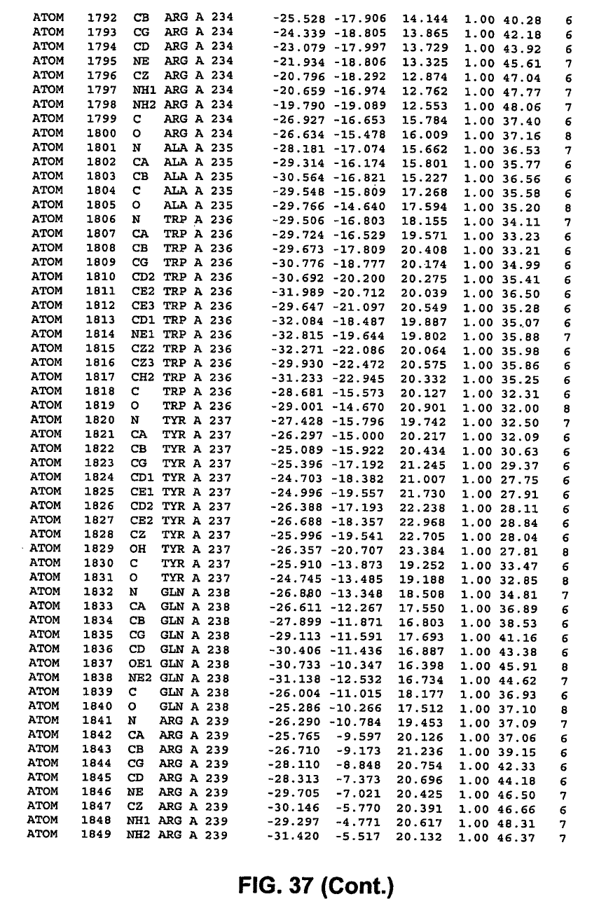

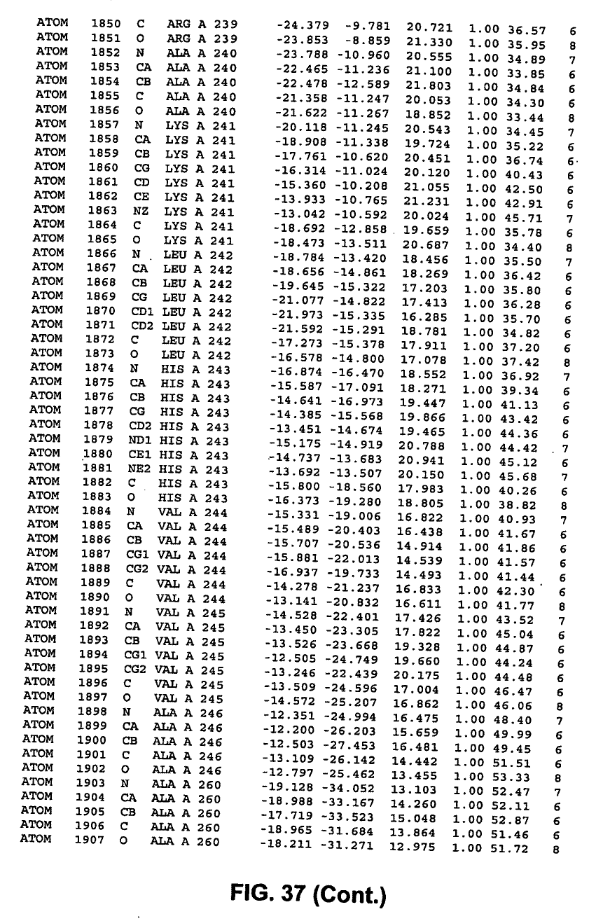

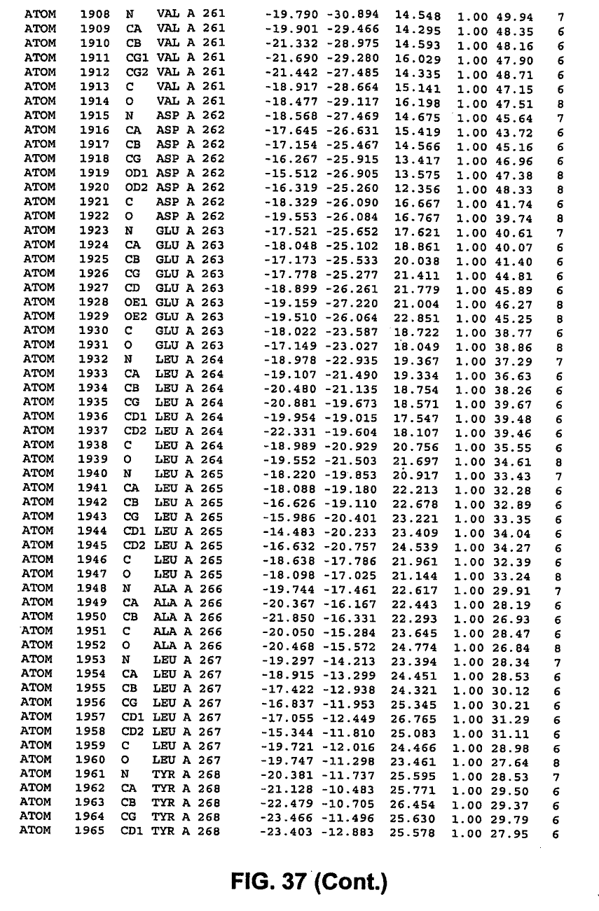

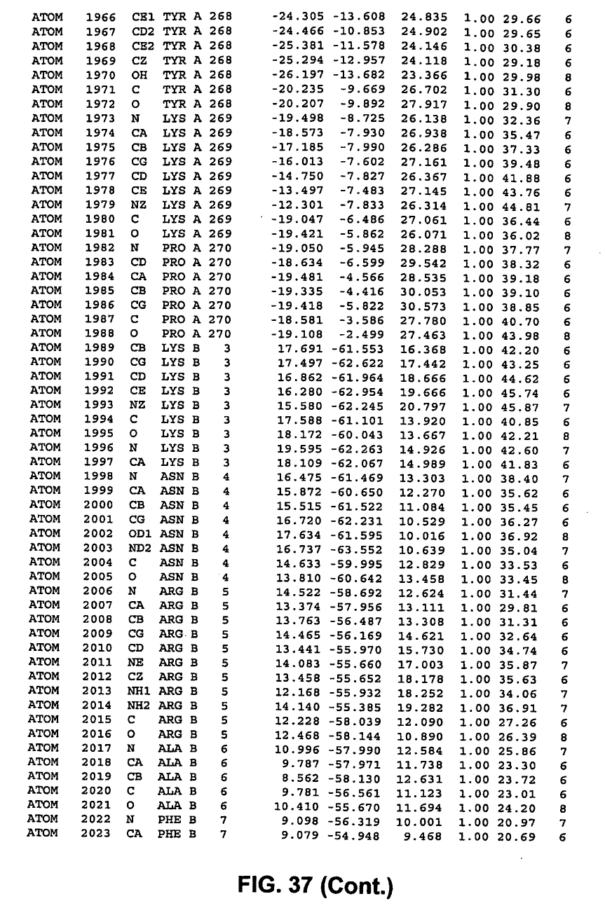

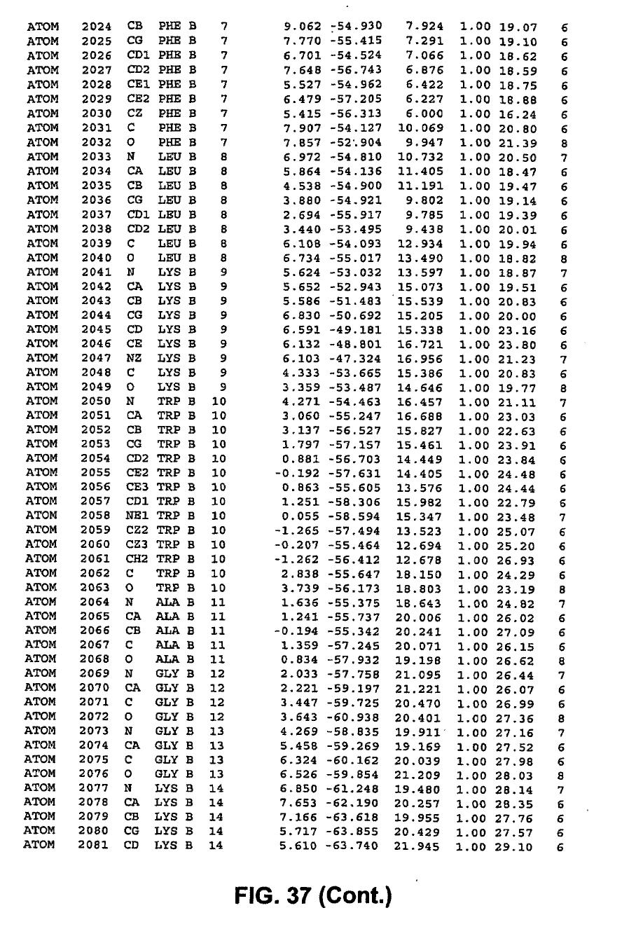

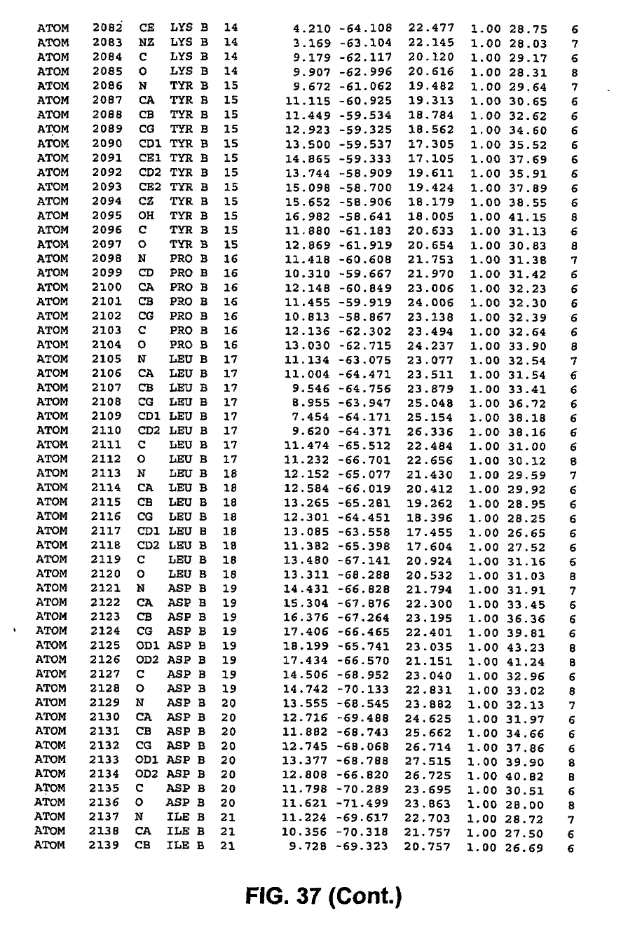

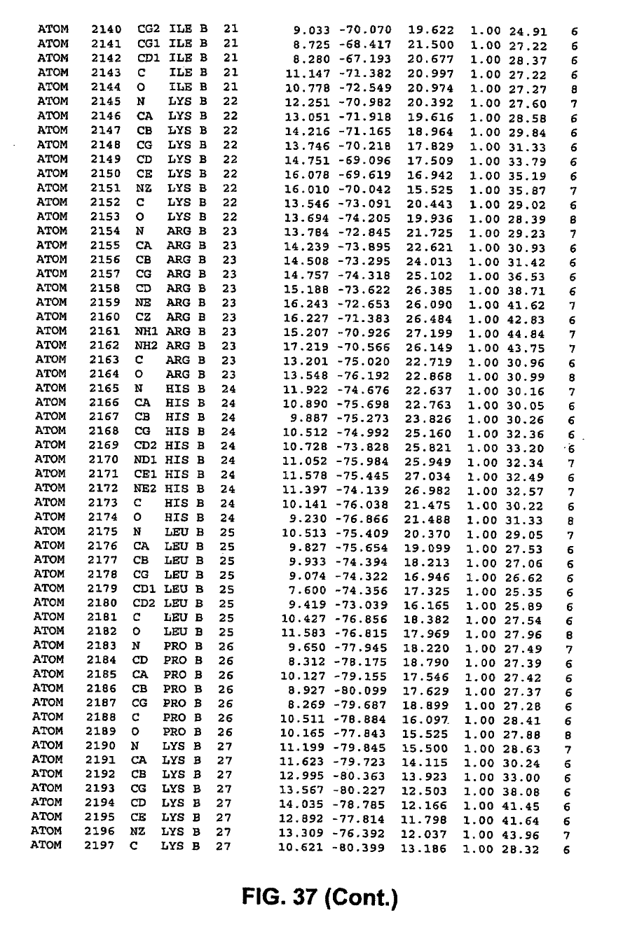

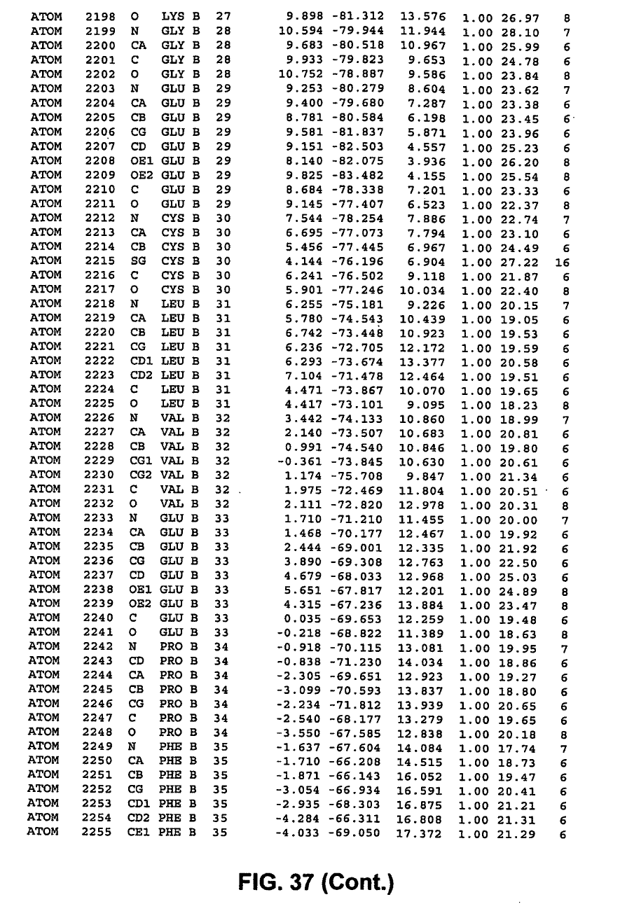

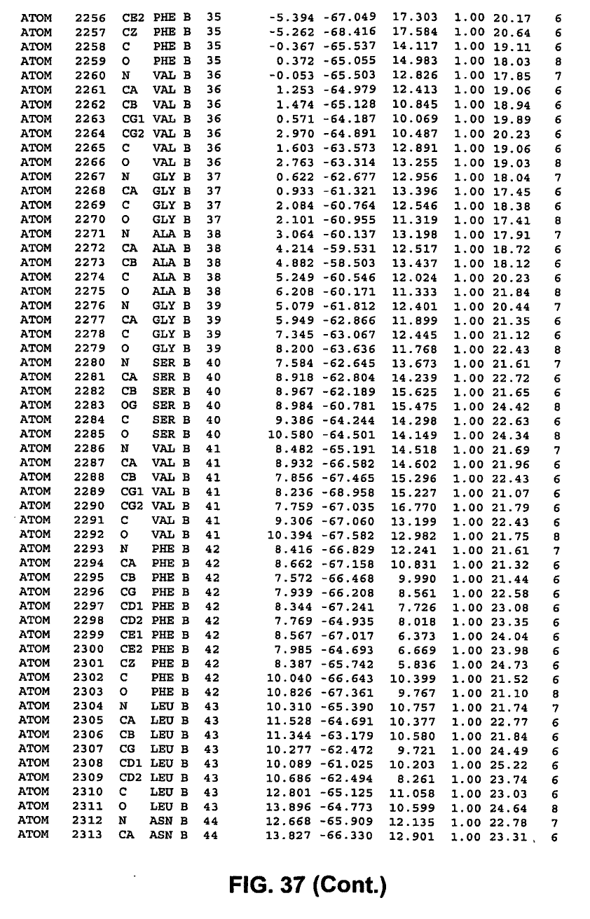

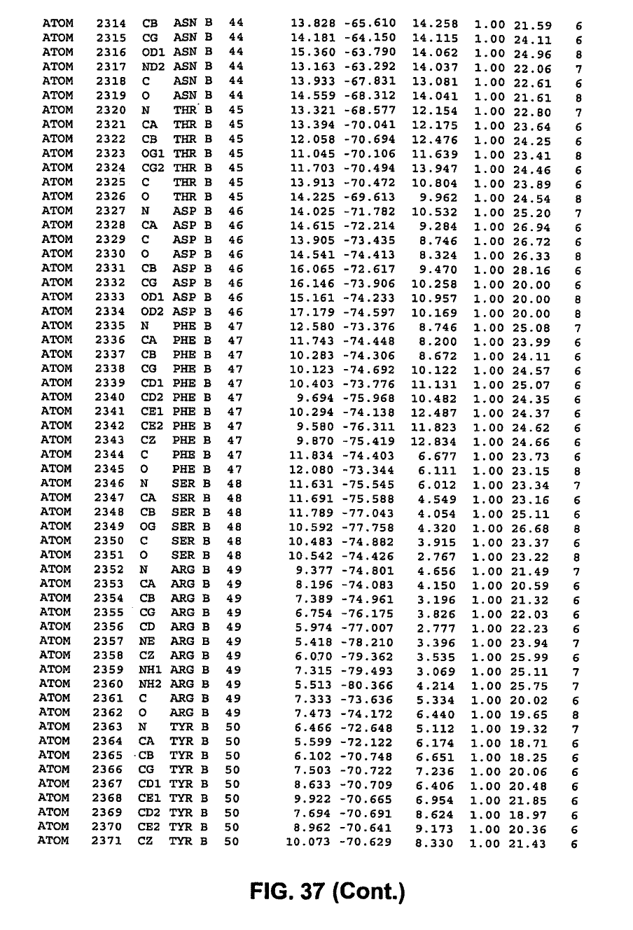

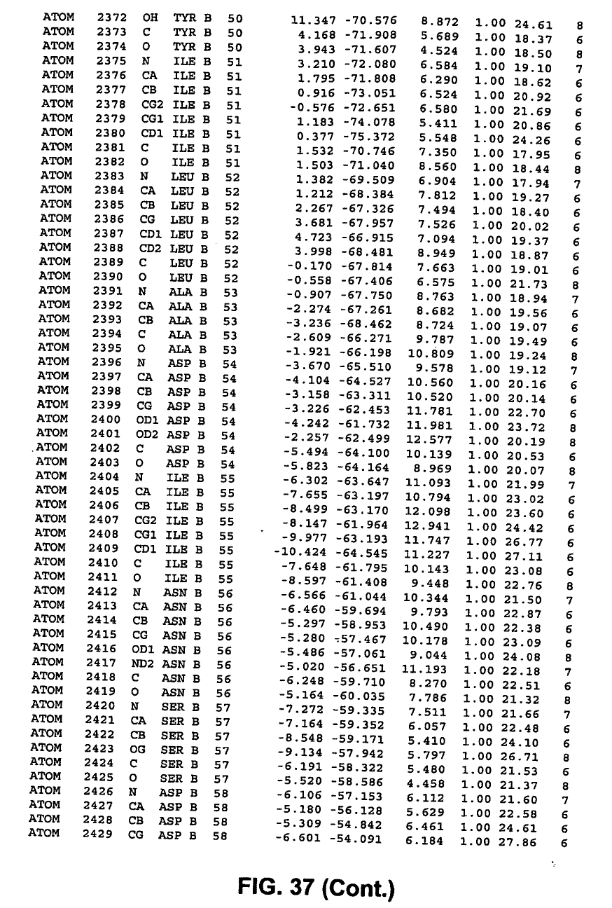

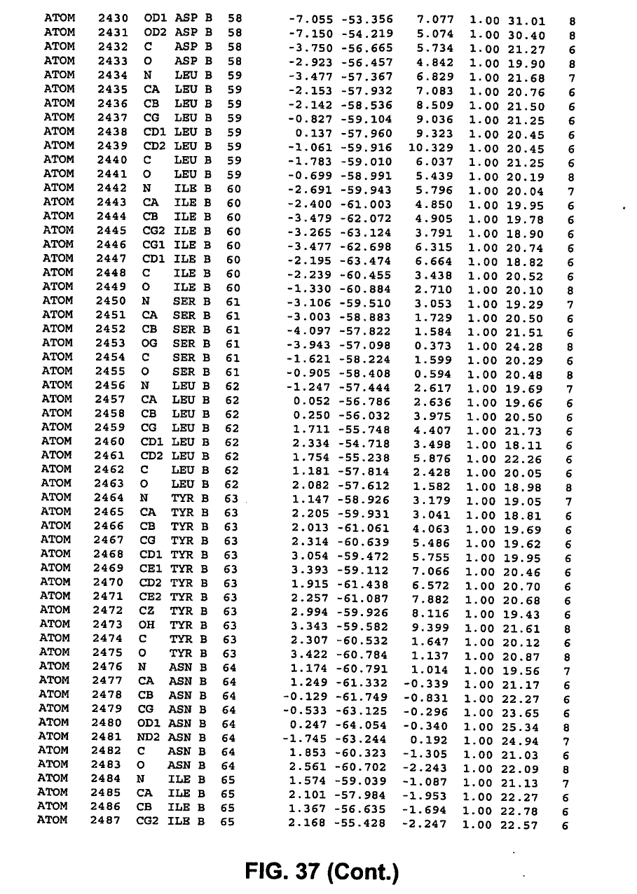

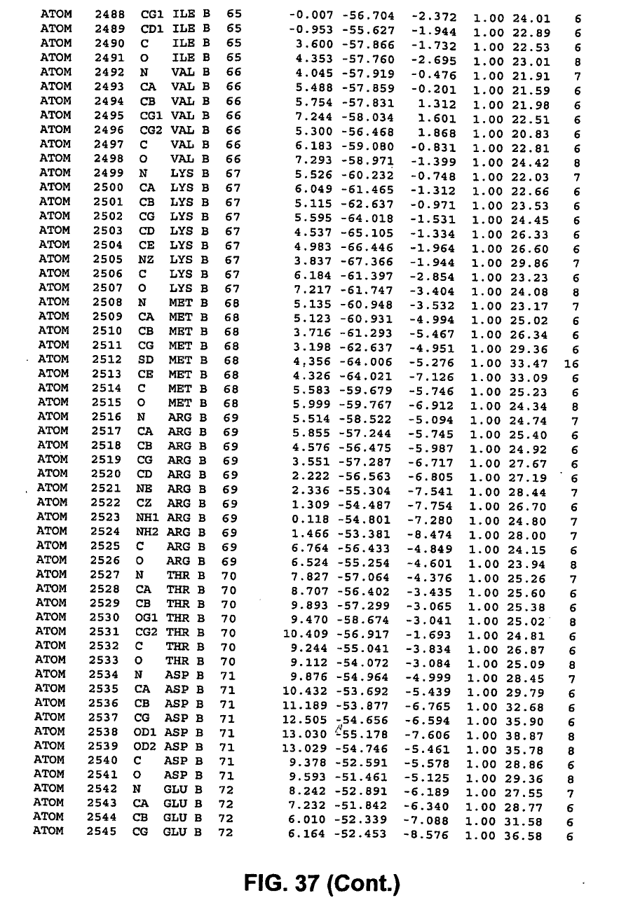

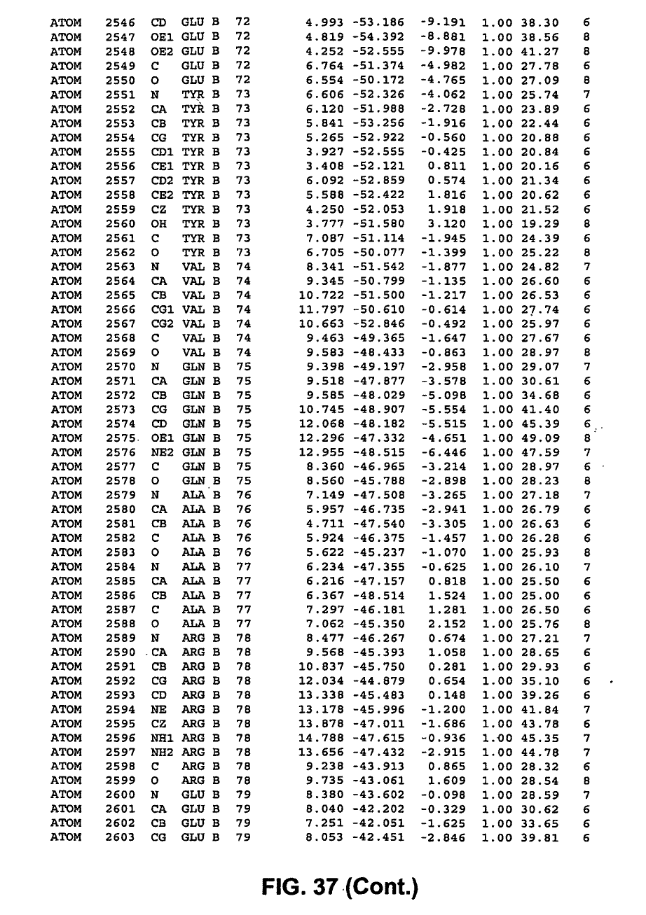

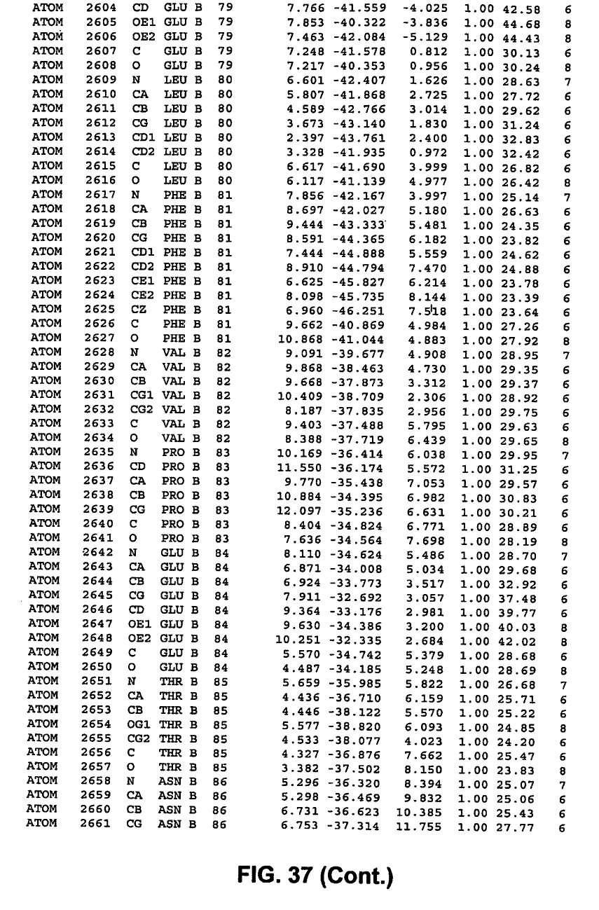

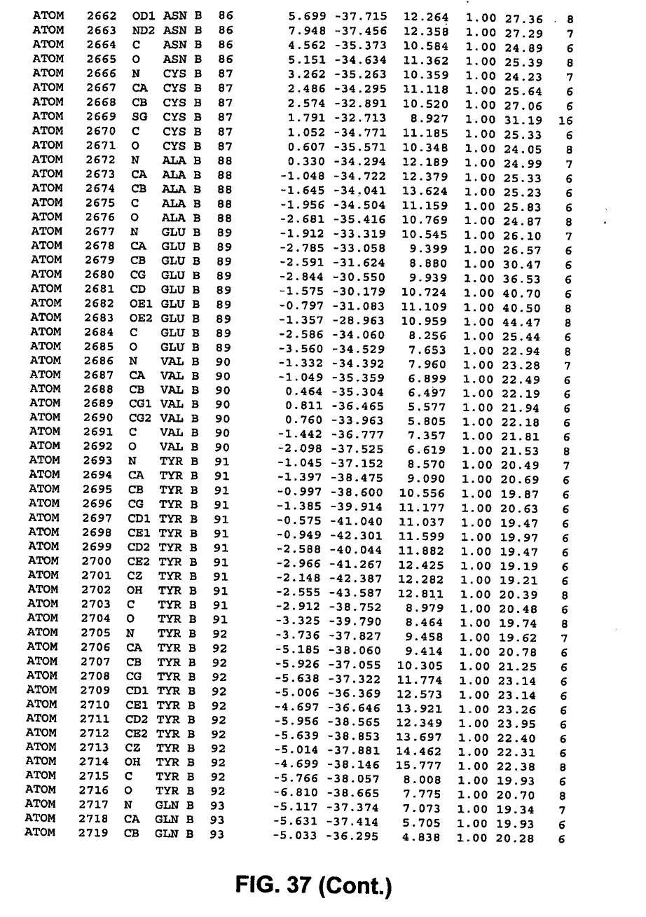

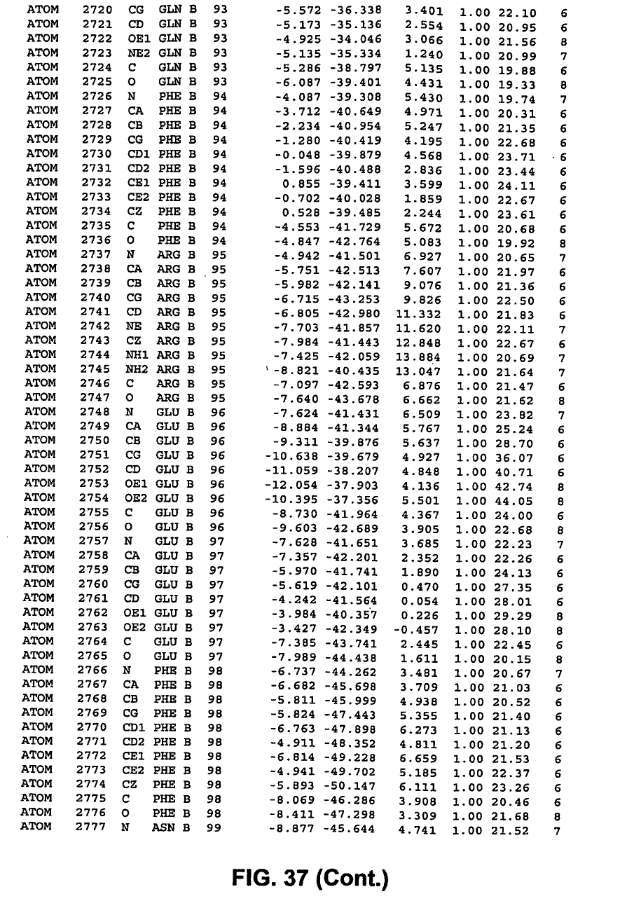

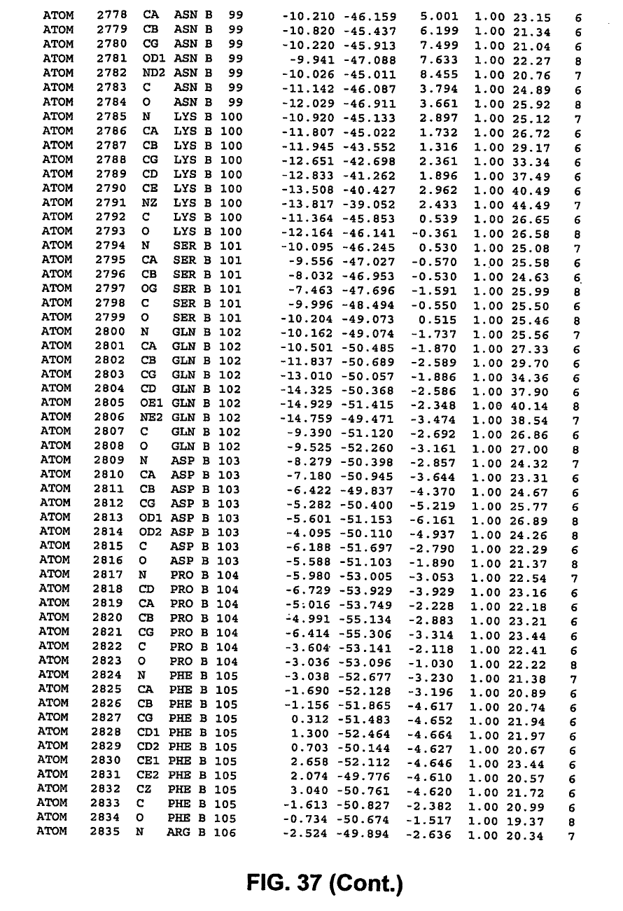

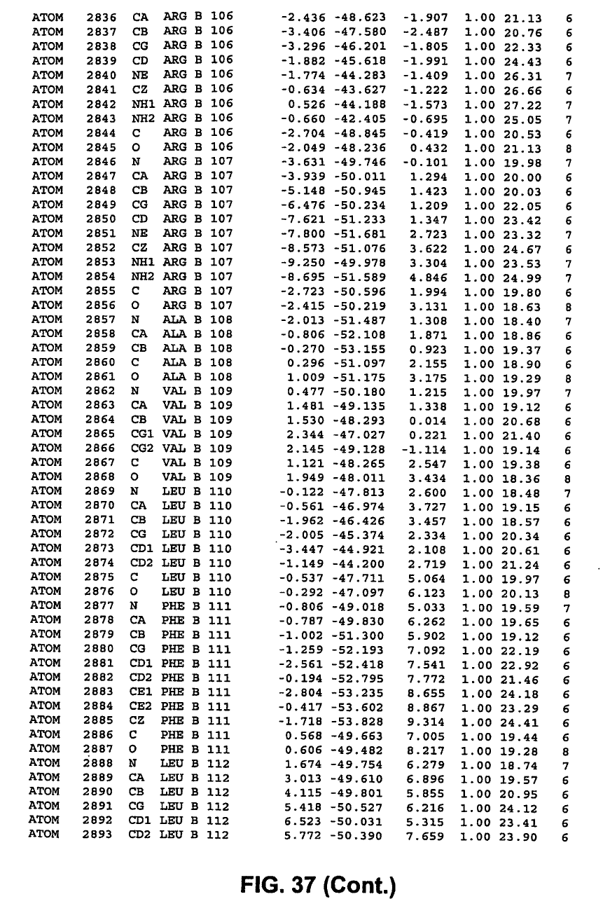

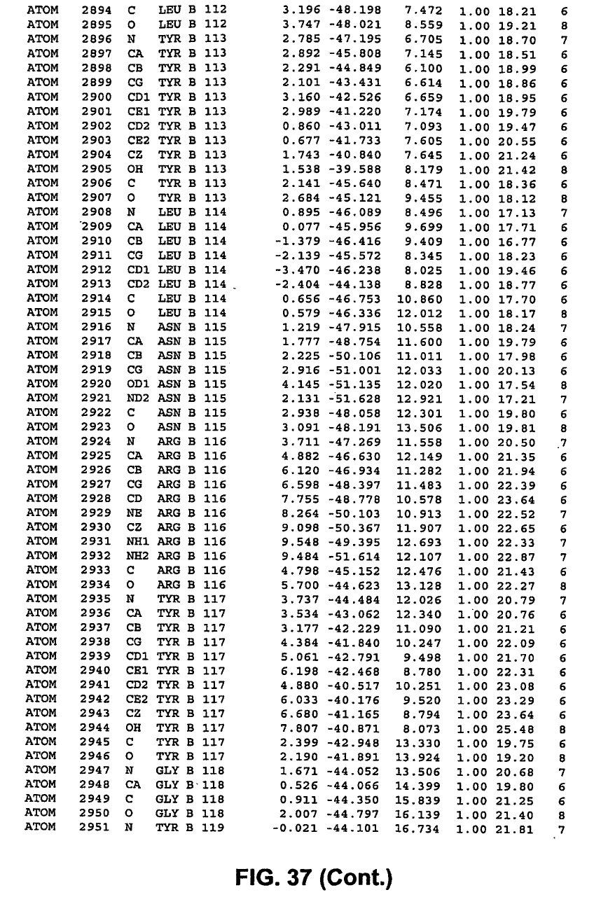

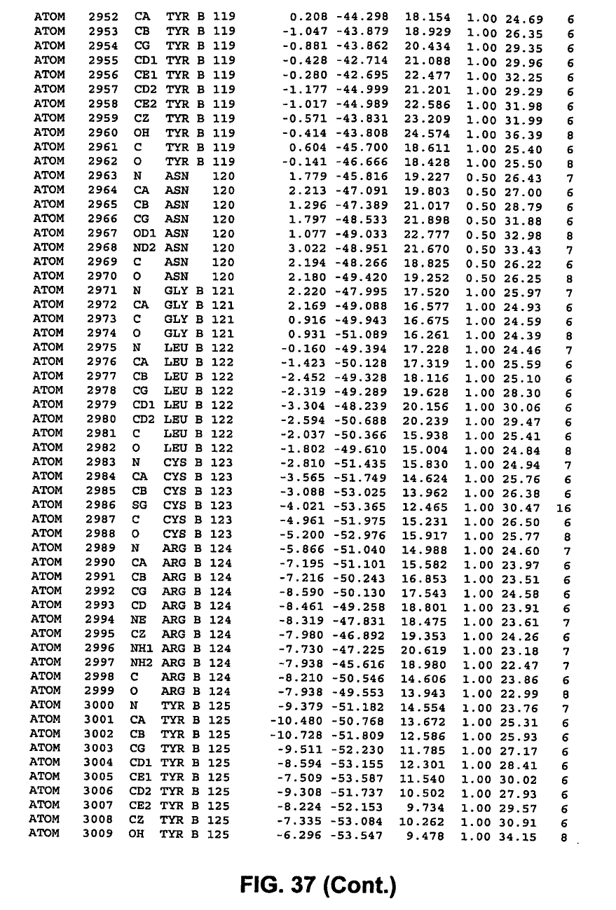

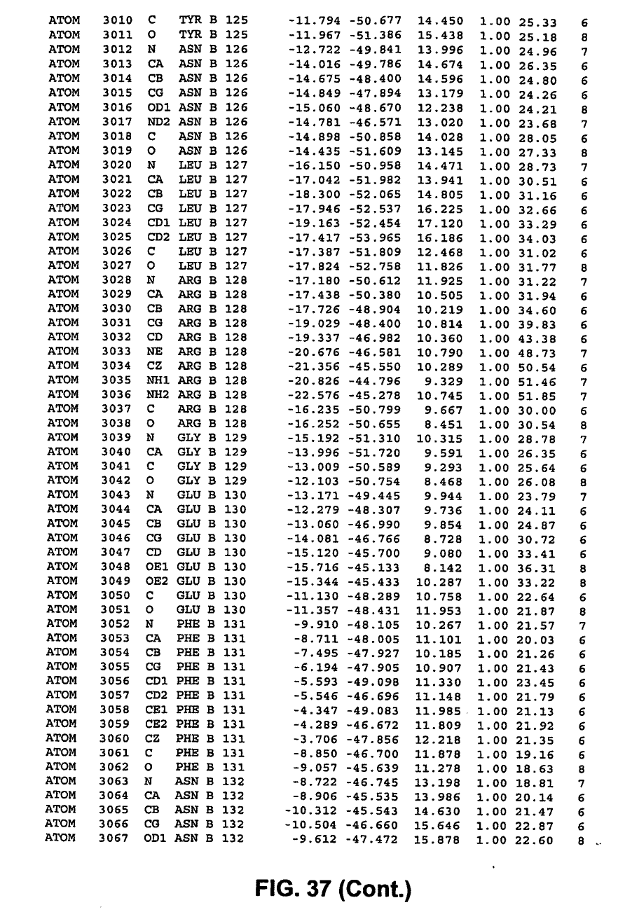

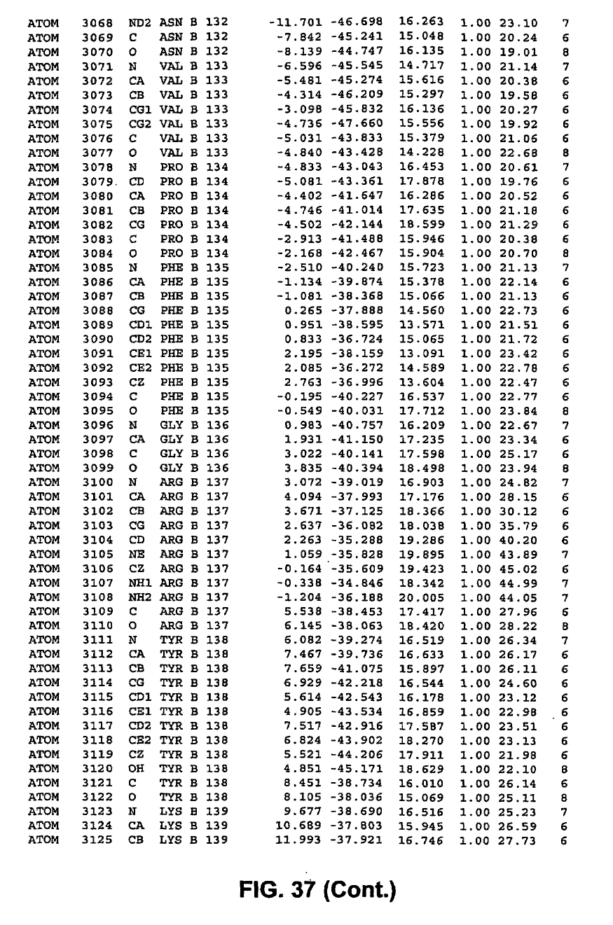

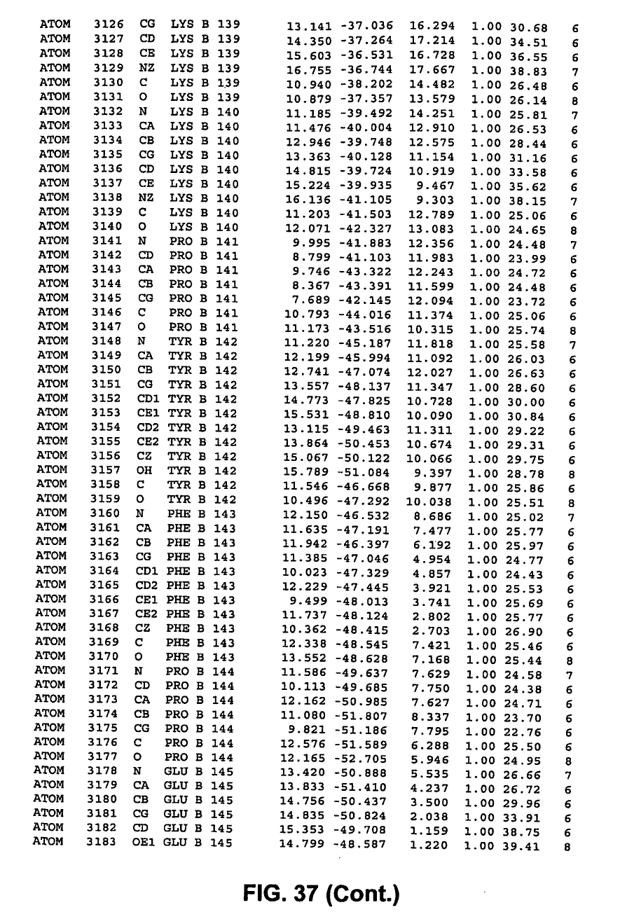

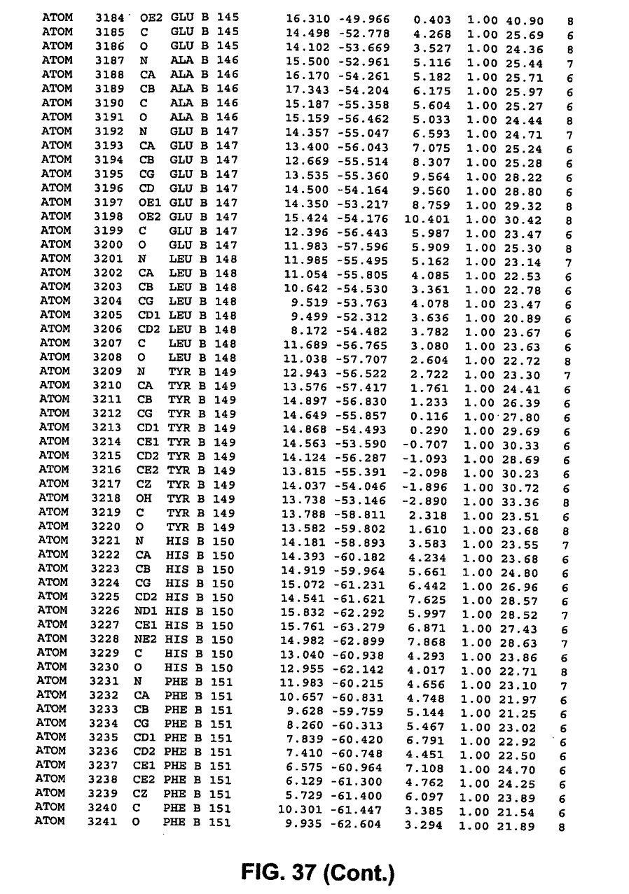

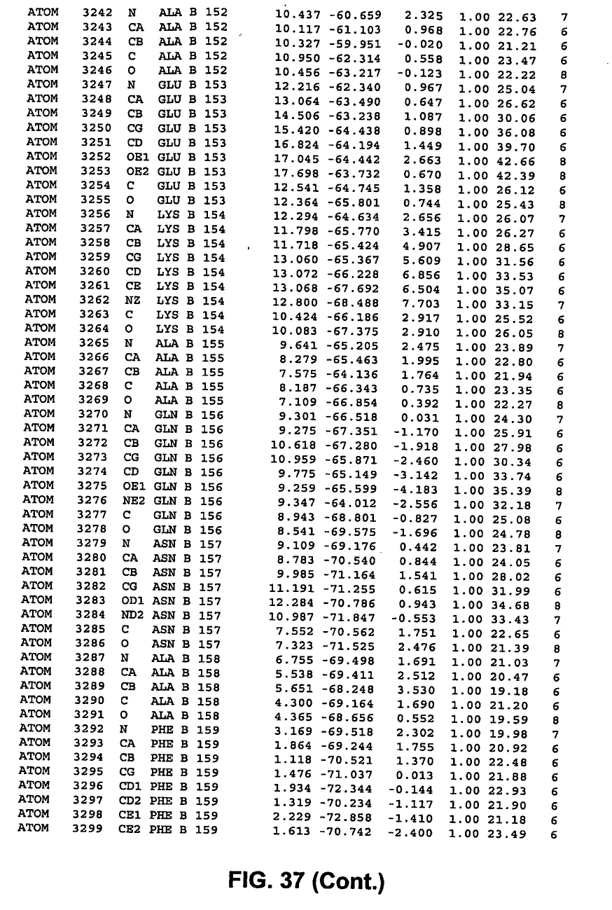

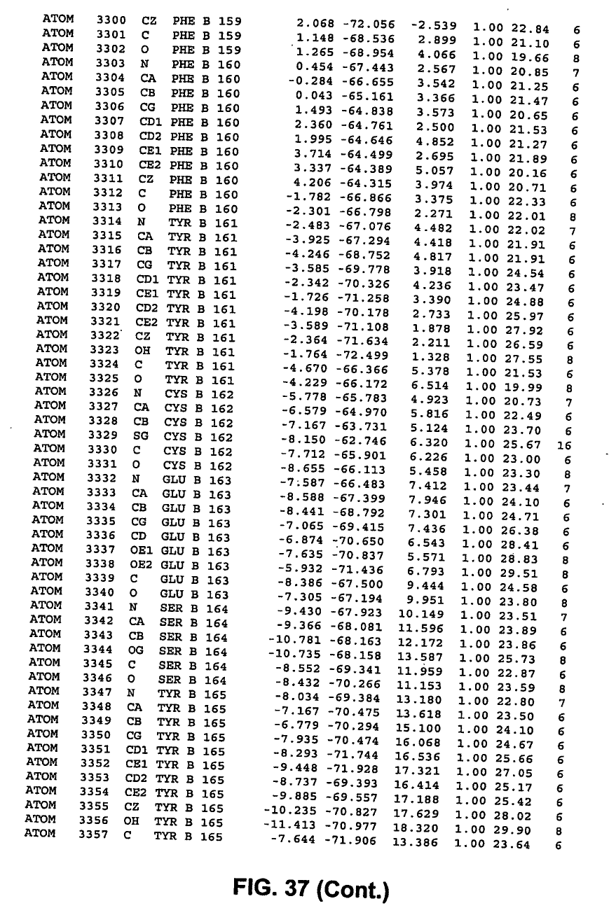

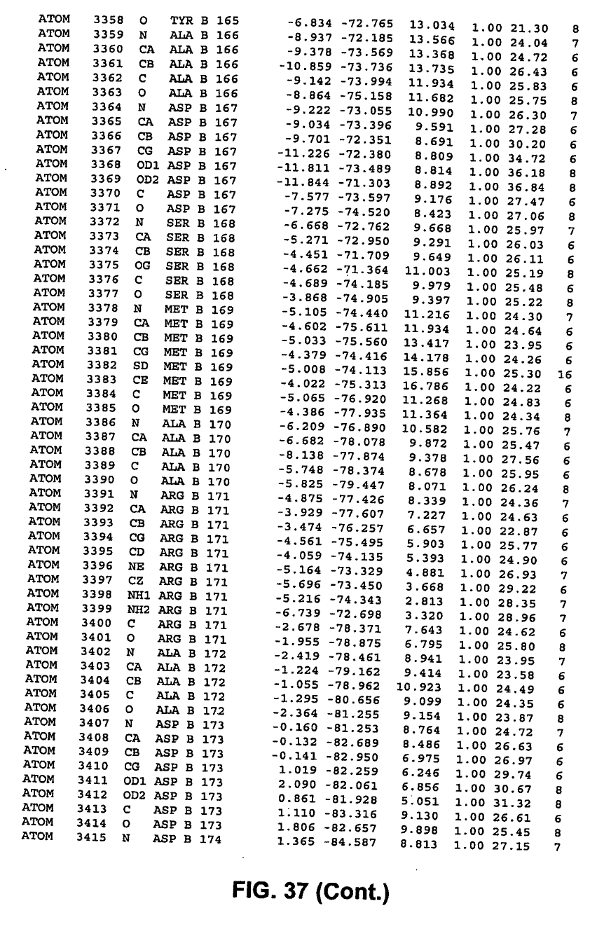

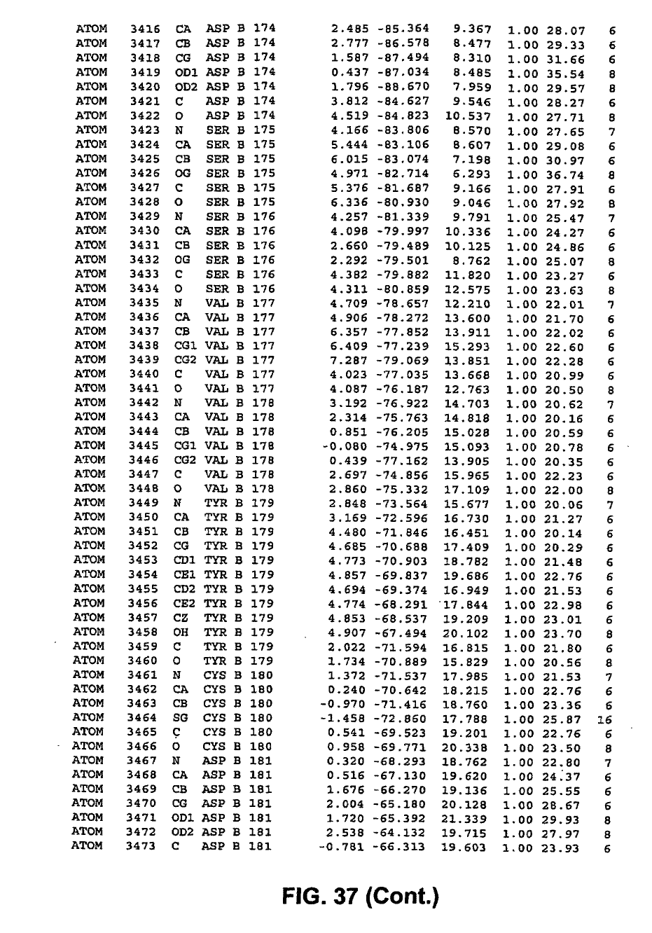

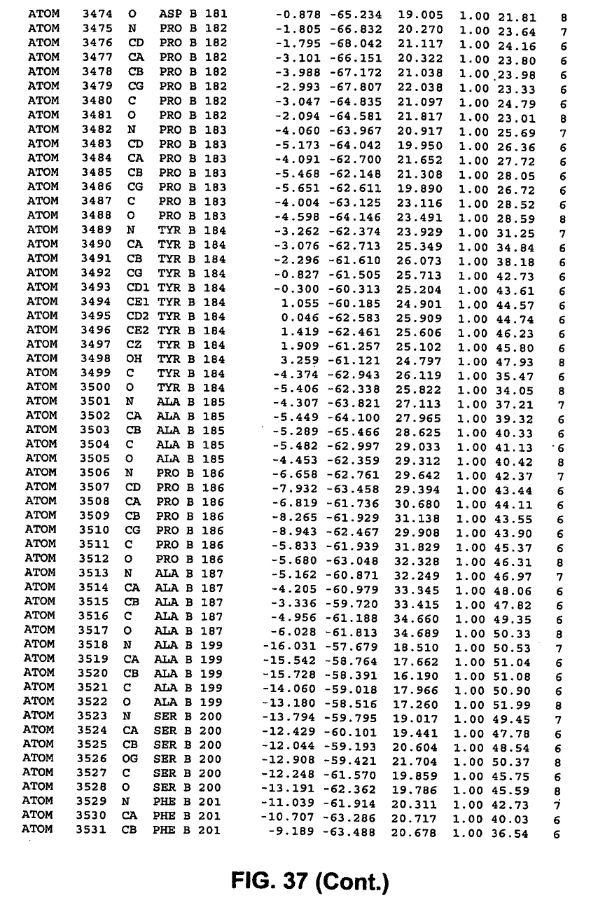

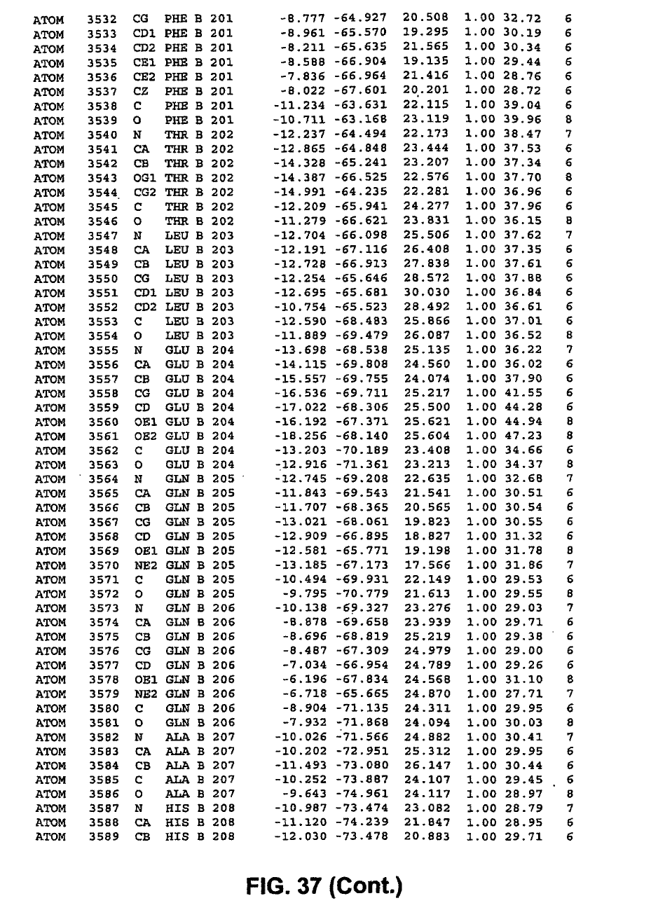

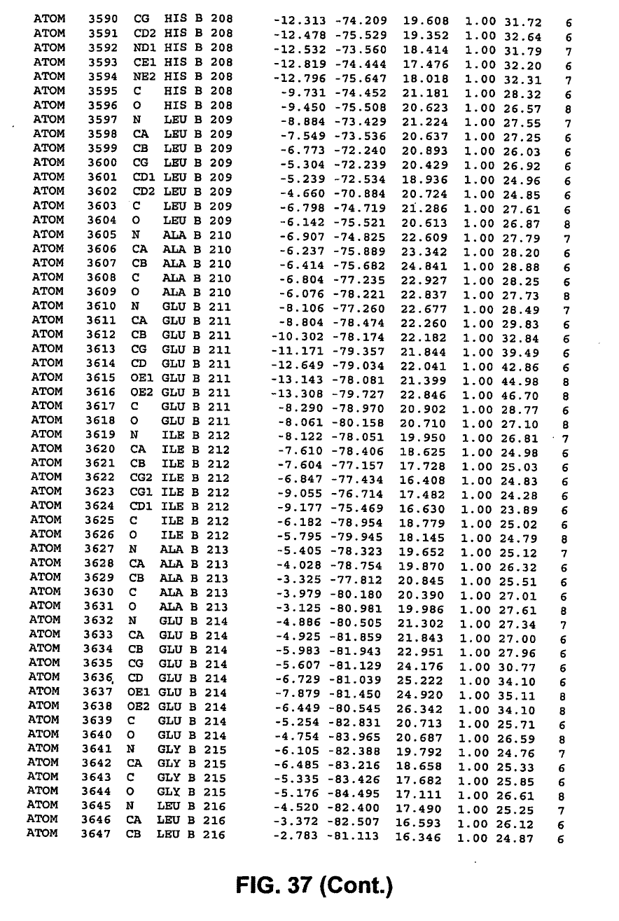

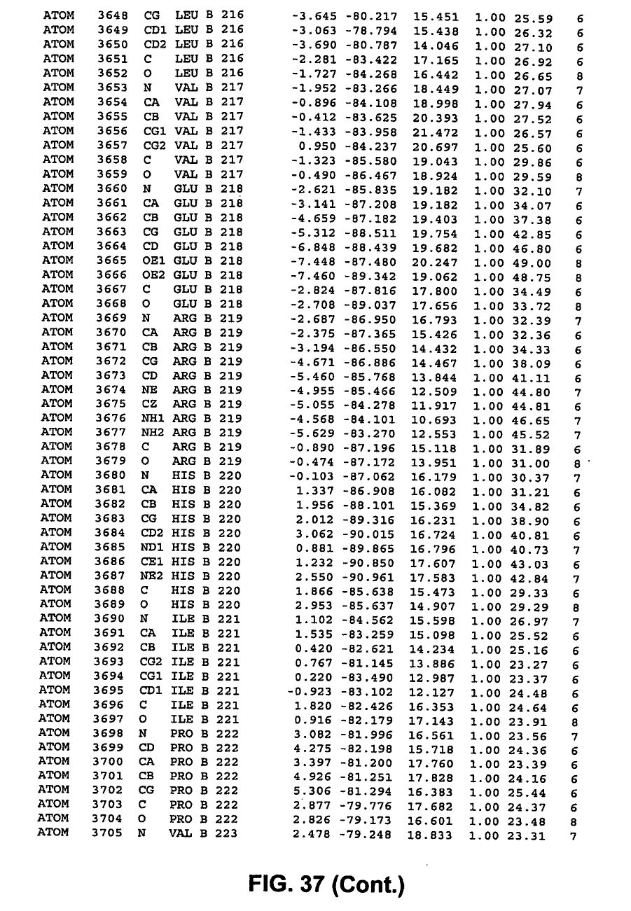

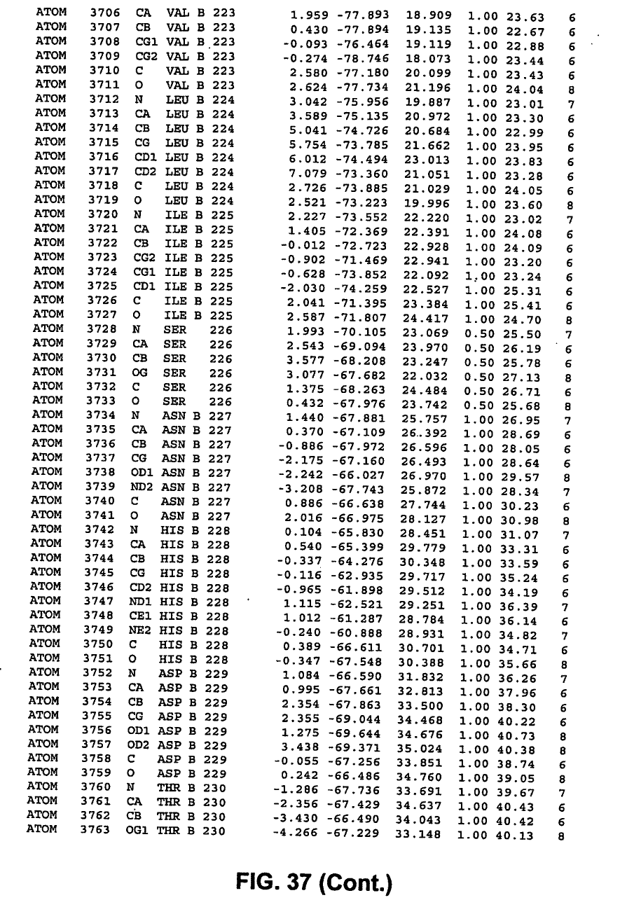

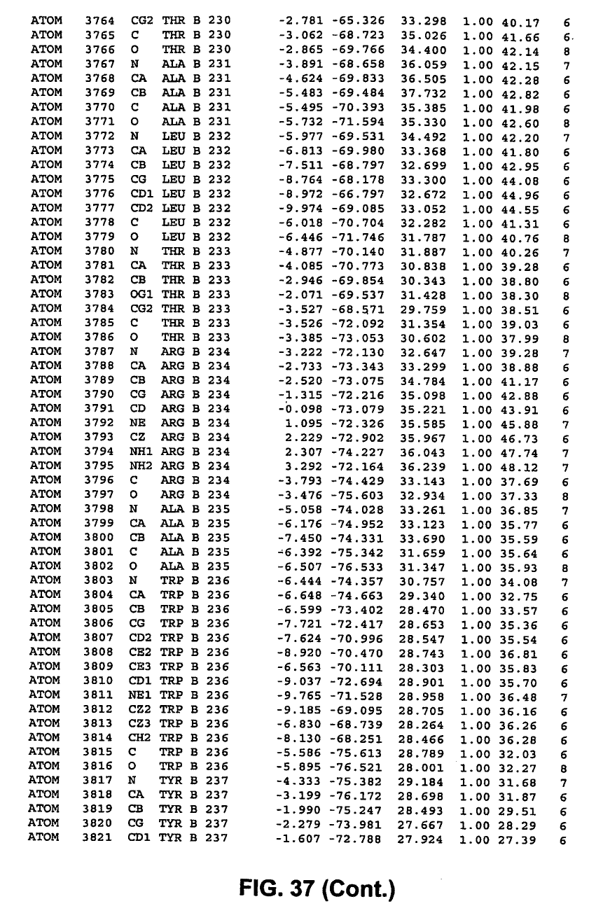

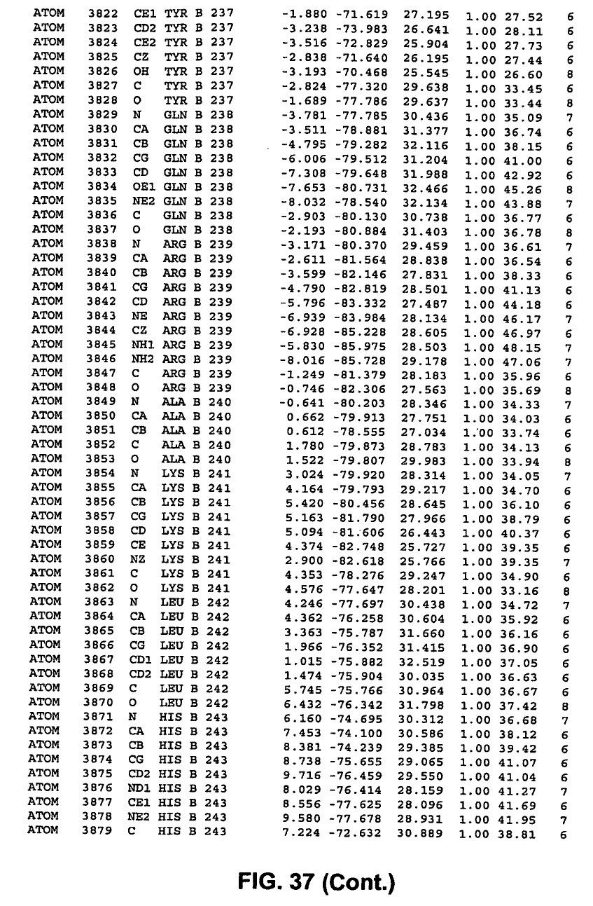

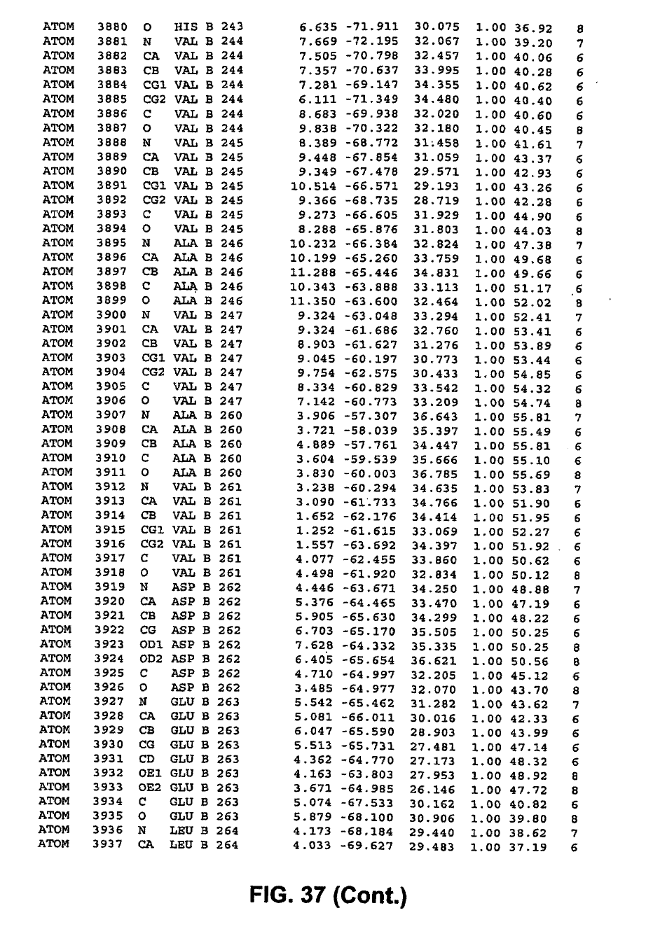

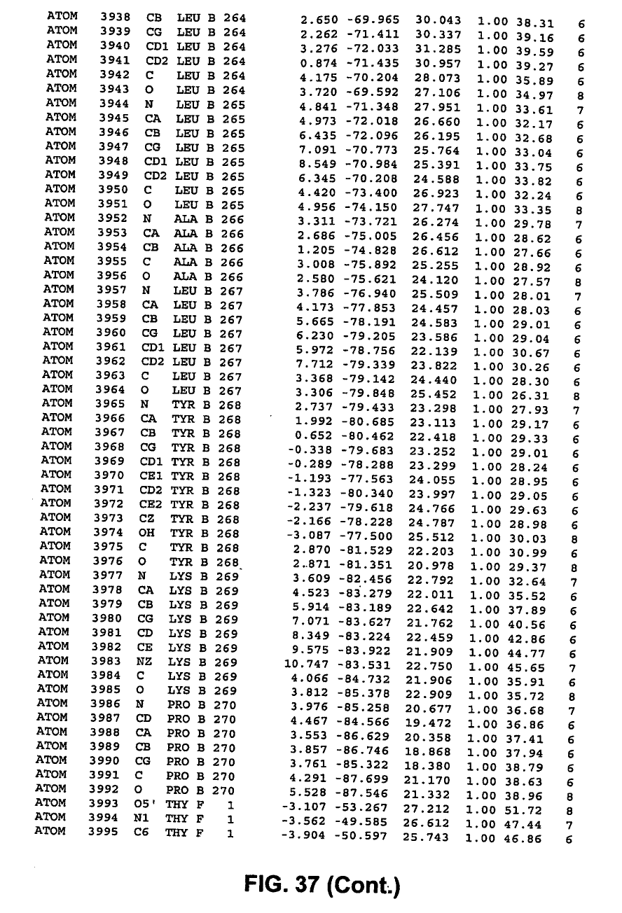

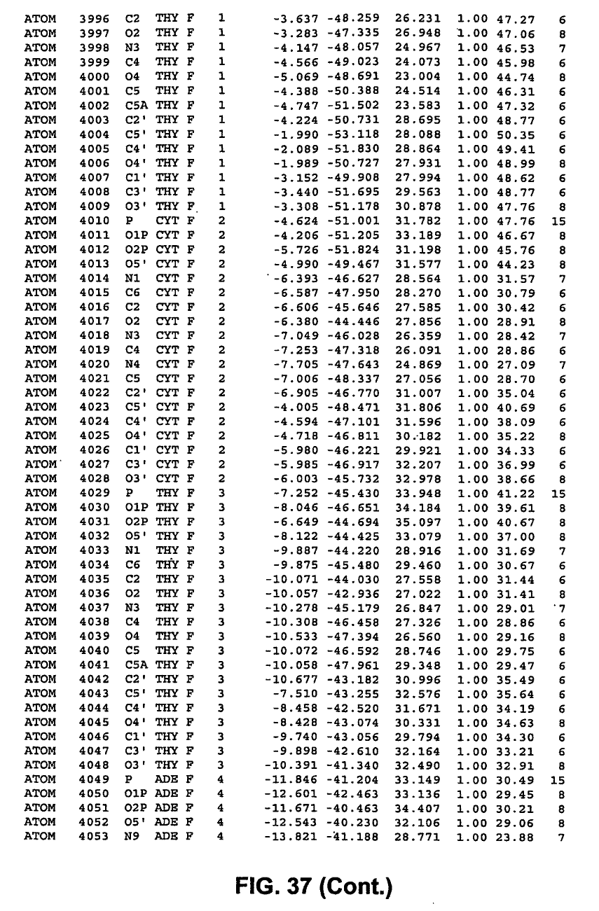

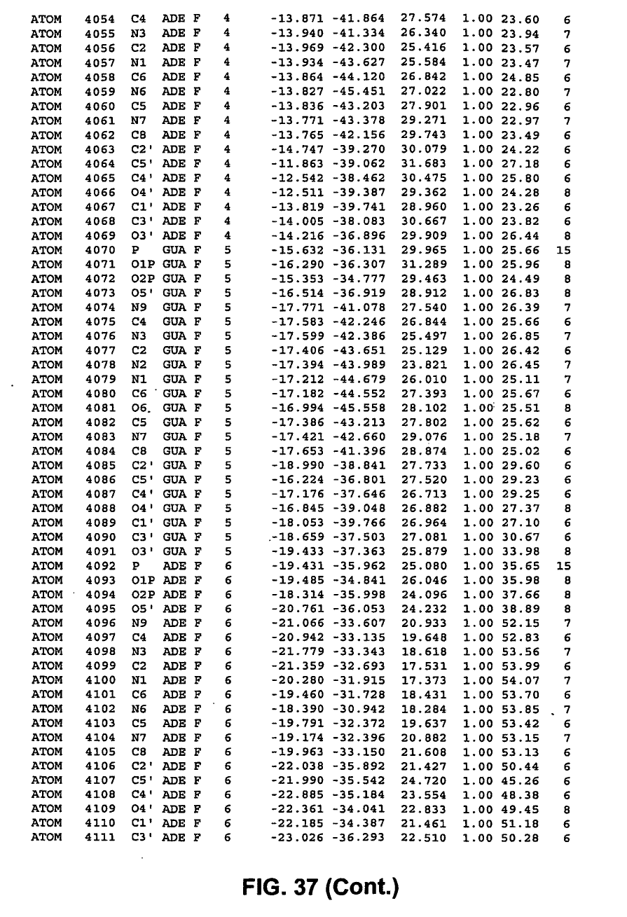

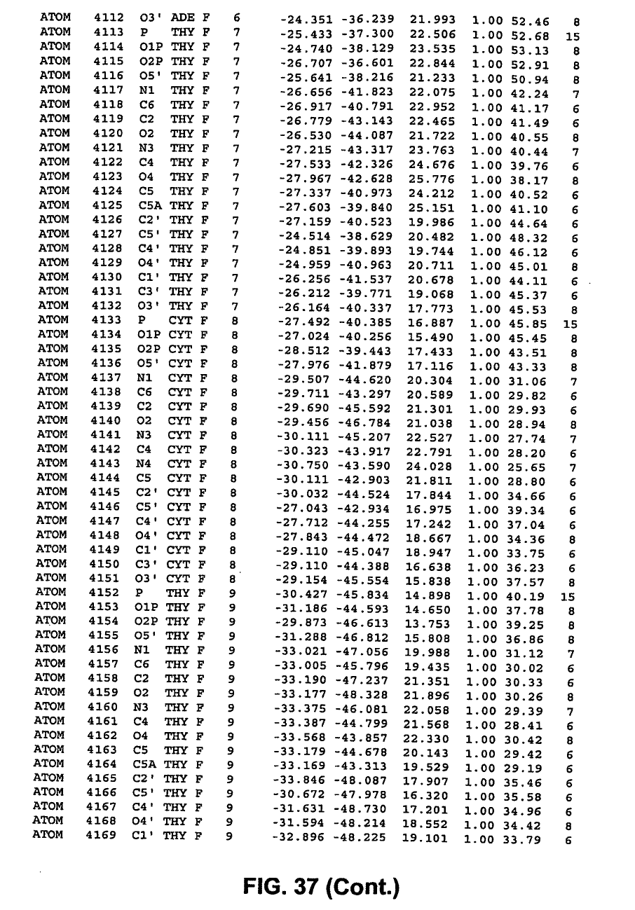

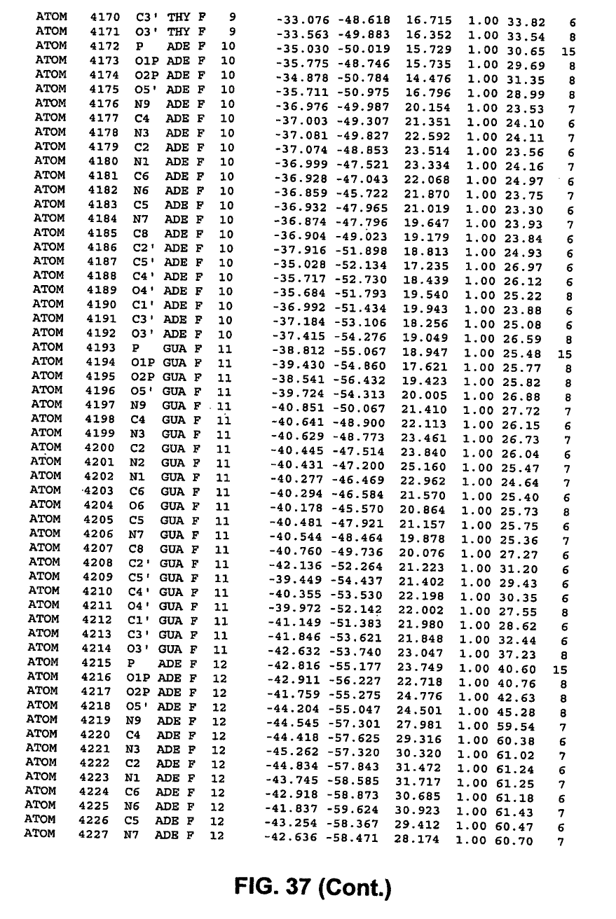

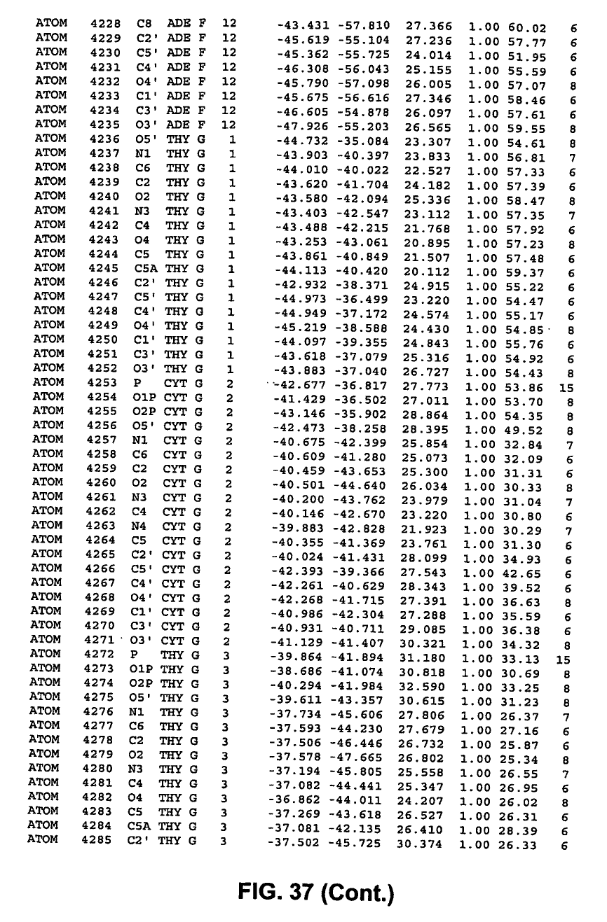

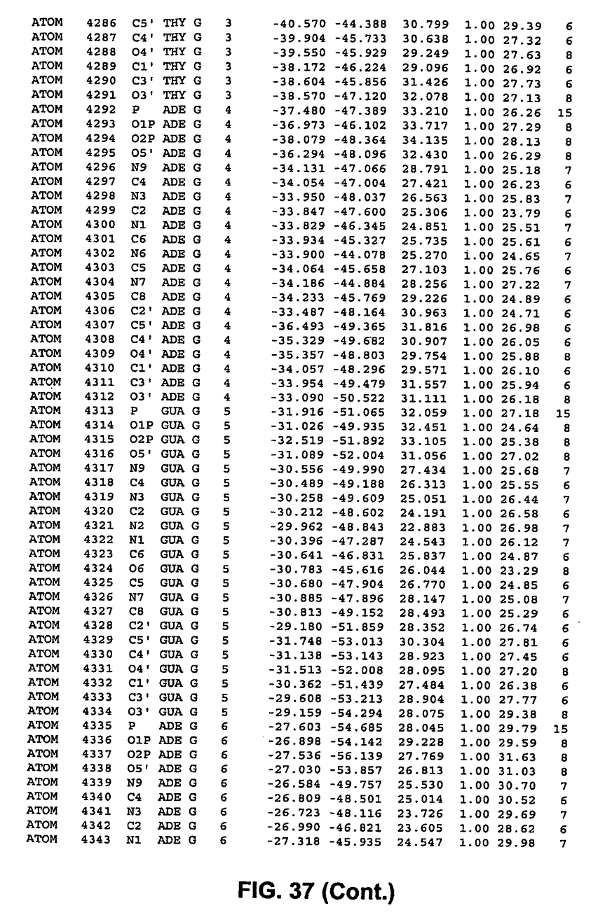

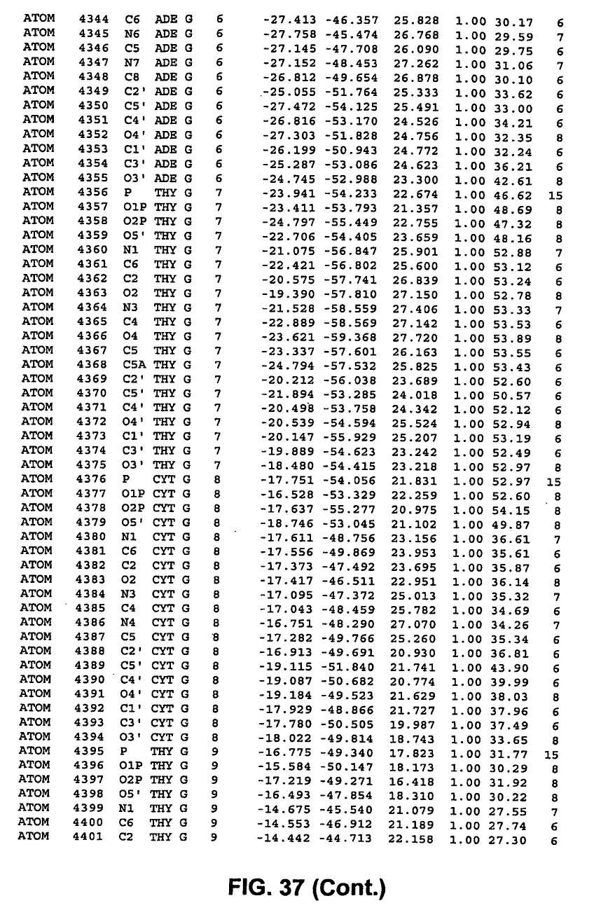

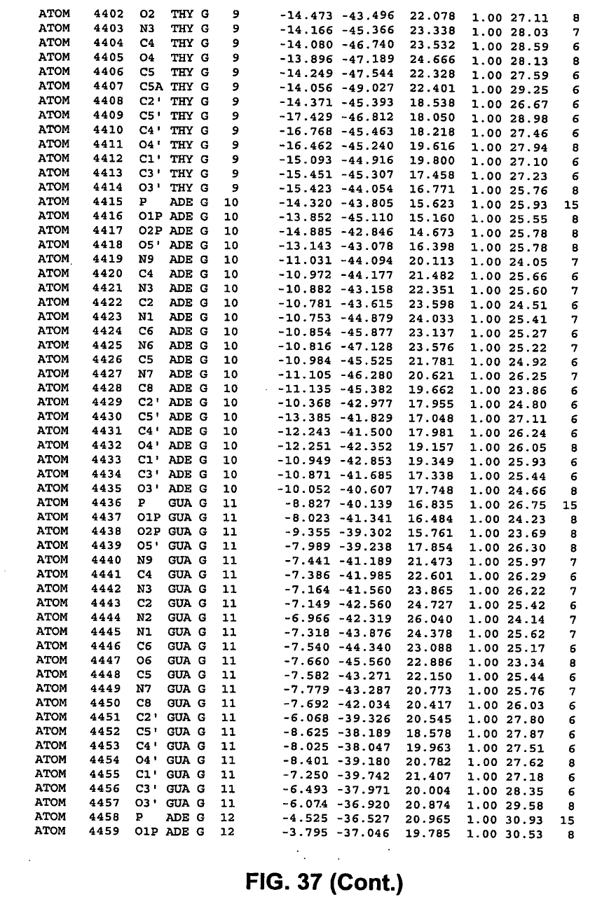

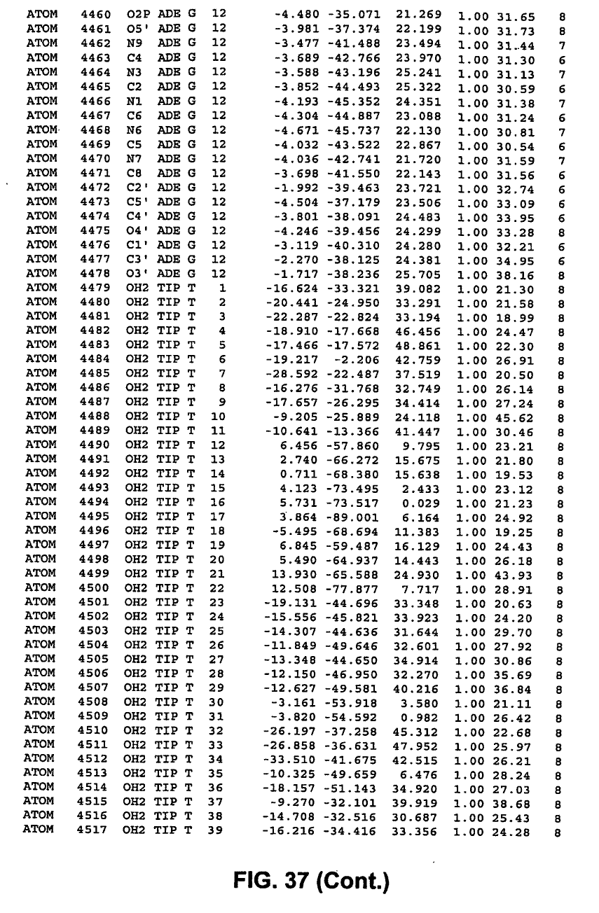

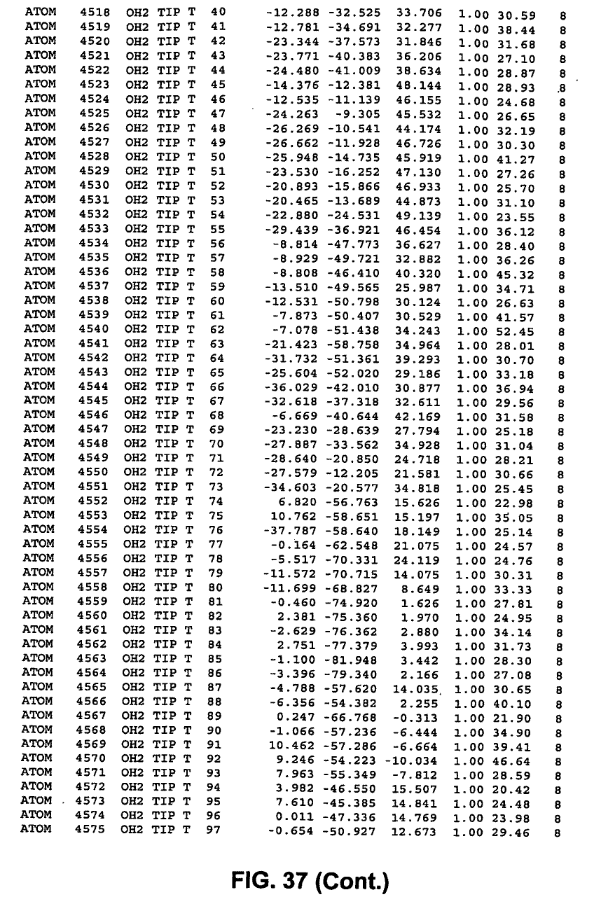

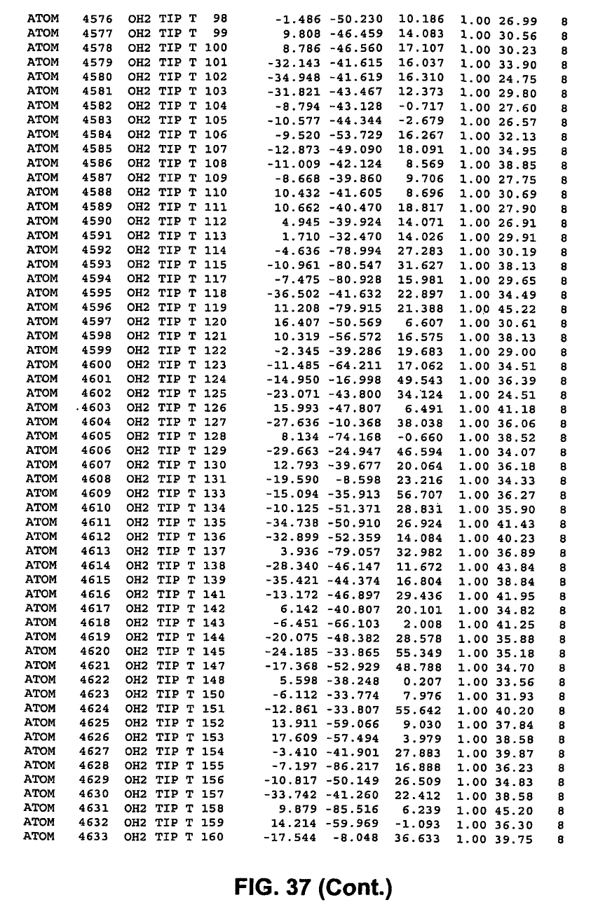

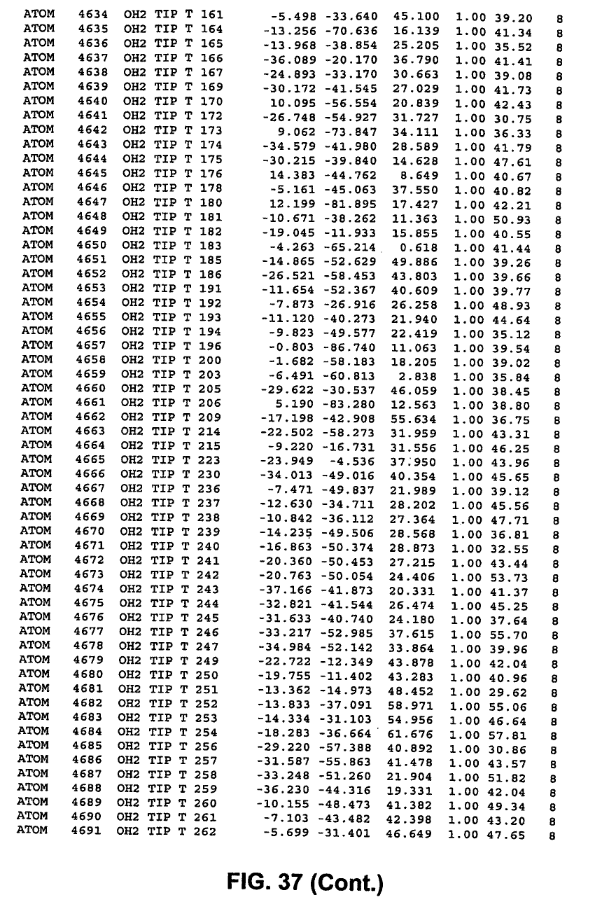

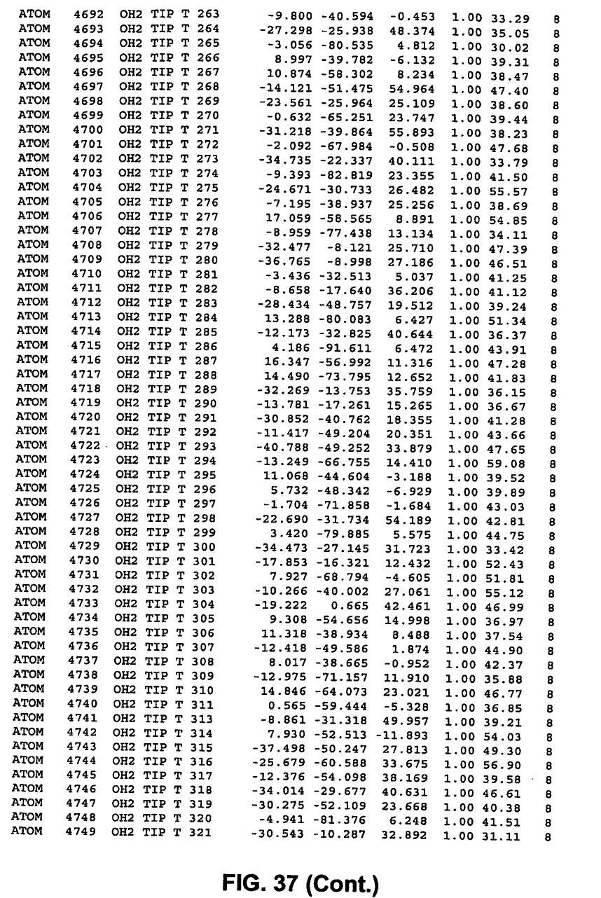

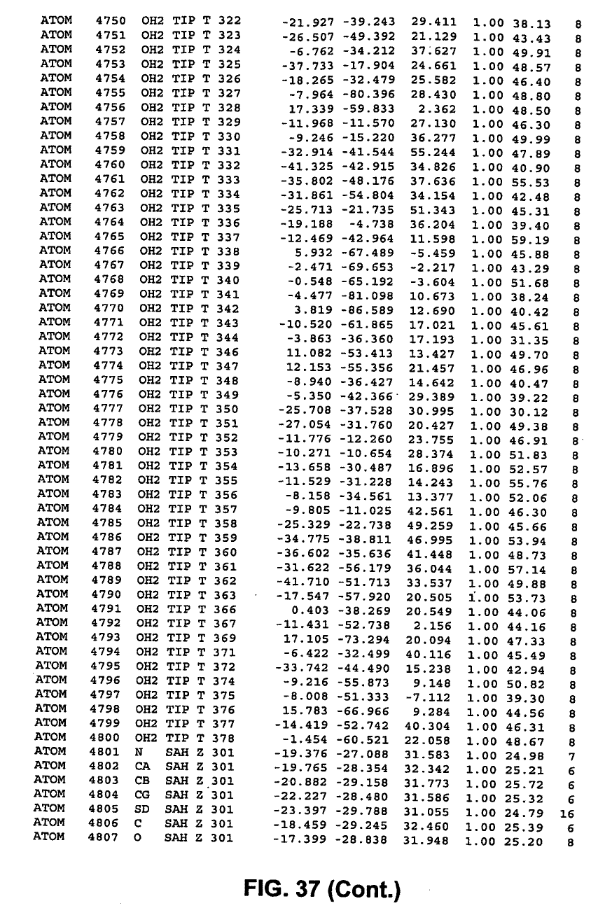

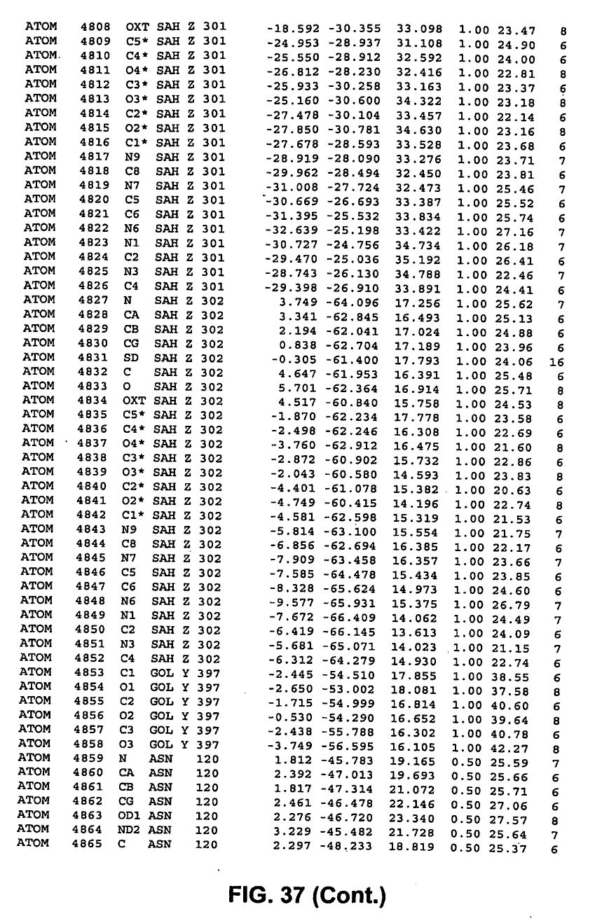

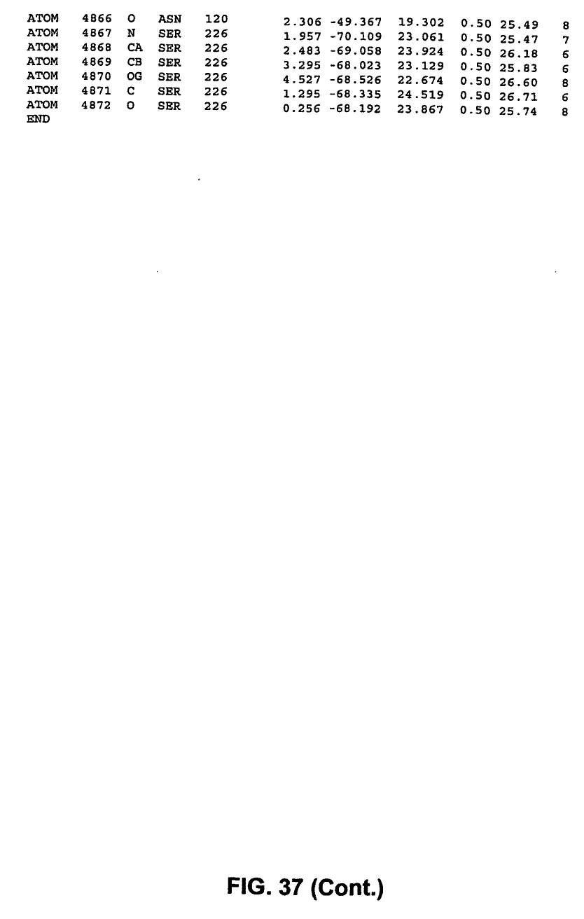

FIG. 36 (from Yang et al. Nature Structural Biology, 10: 849-855) summarizes the sequence data for selected Dam MTase orthologs. The SWISSPROT database accession numbers are: bacteriophage T4 (T4dam -P04392); Escherichia coli (EcoDam-P00475); restriction-modification MTases (EcoRV-P04393) and DpnIIA (P04043). The secondary structure for T4Dam is indicated above the sequence (cylinders for helices, arrows for strands). FIG. 37 is the three-dimensional structure of EcoDam-AdoMet-DNA ternary complex obtained by X-ray diffraction.

When a group of substituents is disclosed herein, it is understood that all individual members of those groups and all subgroups, including any isomers and enantiomers of the group members, and classes of compounds that can be formed using the substituents are disclosed separately. When a compound is claimed, it should be understood that compounds known in the art including the compounds disclosed in the references disclosed herein are not intended to be included. When a Markush group or other grouping is used herein, all individual members of the group and all combinations and subcombinations possible of the group are intended to be individually included in the disclosure.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination. One of ordinary skill in the art will appreciate that methods, device elements, starting materials, synthetic methods, structures, libraries and assays other than those specifically exemplified can be employed in the practice of the invention without resort to undue experimentation. All art-known functional equivalents, of any such methods, device elements, starting materials, synthetic methods, and structures, libraries and assays are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The exact formulation, route of administration and dosage can be chosen by the individual physician in view of the patient's condition (see e.g. Fingl et. al., in The Pharmacological Basis of Therapeutics, 1975, Ch. 1 p. 1).

It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above also may be used in veterinary medicine.

Depending on the specific conditions being treated and the targeting method selected, such agents may be formulated and administered systemically or locally. Techniques for formulation and administration may be found in Alfonso and Gennaro (1995). Suitable routes may include, for example, oral, rectal, transdermal, vaginal, transmucosal, or intestinal administration; parenteral delivery, including intramuscular, subcutaneous, or intramedullary injections, as well as intrathecal, intravenous, or intraperitoneal injections.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular those formulated as solutions, may be administered parenterally, such as by intravenous injection. Appropriate compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve the intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions, including those formulated for delayed release or only to be released when the pharmaceutical reaches the small or large intestine.

The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers: In soft capsules, the active compounds may be dissolved or suspended in suitable liquids, such as fatty oils, liquid paraffin, or liquid polyethylene glycols. In addition, stabilizers may be added.

All references throughout this application, for example patent documents including issued or granted patents or equivalents; patent application publications; and non-patent literature documents or other source material, including Table 9 listing of references; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

TABLE 1
Role of Dam molecule in bacterial virulence (modified from Low et al. 2001)

| | Role for MTase In: | | Identity to | |
|---|---|---|---|---|
| Bacterial Pathogen | Growth | Virulence | E. coli Dam (%) | References |
| Escherichla coli | No | Yes | 100 | (Krabbe et al., 2000) |
| Erwinia chrysanthemi | No | Yes | 99 | C.-H. Yang and N. Keen, |
| | | | | unpublished data |
| Salmonella enterica | No | Yes | 92 | (Garcia-Del Portillo et al., 1999) |
| serovar Typhimurium | | | | (Heithoff et al., 2001; Heithoff et |
| | | | | al., 1999) |
| Klebsiella pneumoniae | N/D | N/D | 82 | |
| Yersinia | Yes | Yes | 71 | (Julio et al., 2001) |
| pseudotuberculosis | | | | |
| Vibrio cholerae | Yes | Yes | 64 | (Julio et al., 2001) |
| Pasteurella multocida | N/D | Yes | 57 | (Chen et al., 2003) |
| Haemophilis influenzae | No | Yes | 55 | (Watson et al., 2004) |
| Neisseria meningitidis | Yes | Yes | 47 | (Bucci et al., 1999) |

TABLE 2
Summary of properties of various T4DAM-DNA-AdoHcy crystals

| | | | | |
|---|---|---|---|---|
| Reso- | | | | |
| lution | Space | Cell dimensions | Crystallization | Binding |

| | | | | | | | | |
|---|---|---|---|---|---|---|---|---|
| DNA sequence | Å | group | a(Å) | b(Å) | c(Å) | β(°) | Conditions | mode |

| | | | | | | | | |
|---|---|---|---|---|---|---|---|---|
| Duplex I (12-mer) | 3.1 | P2₁ | 39.7 | 109.7 | 73.6 | 104.5 | 20-25% PEG | Non- |
| ACAGGATCCTGT | | | | | | | 8000 | specific |
| TGTCCTAGGACA | | | | | | | 100 mM | |
| | | | | | | | HEPES pH 7.5, | |
| | | | | | | | 10 mM | |
| | | | | | | | ammonium | |
| | | | | | | | sulfate | |
| Duplex II (13-mer) | 2.3 | P2₁ | 38.9 | 125.8 | 73.2 | 104.7 | 20% PEG MME | ¼ site |
| TGTCAGATCATGG | | | | | | | 5000, 100 mM | |
| CAGTCTAGTACCA | | | | | | | citrate pH 6.4, | |
| | | | | | | | 30 mM | |
| | | | | | | | ammonium | |
| | | | | | | | sulfate | |
| Duplex III-1 (15-mer): | 2.7 | P2₁2₁2₁ | 111.4 | 133.2 | 191.2 | | 11.5-19% PEG | ½ and |
| TCACAGGATCCTGTG | | | | | | | 6K, 100 mM | ¾ sites |
| GTGTCCTAGGACACT | | | | | | | MES pH 5.4- | |
| | | | | | | | 6.4, | |
| | | | | | | | 180-200 mM | |
| | | | | | | | ammonium | |
| | | | | | | | acetate, | |
| | | | | | | | 10 mM CaCl₂, | |
| | | | | | | | 5-10% ethylene | |
| | | | | | | | glycol | |
| Duplex III-2 | 3.2 | P2₁ | 87.1 | 118.6 | 87.2 | 90.3 | 7% PEG 6K, | ¾ and |
| TCACAGGATCCTGTG | | | | | | | 100 mM MES | full |
| GTGTCCTAGGACACT | | | | | | | pH 6.0, | site |
| | | | | | | | 200 mM | |
| | | | | | | | ammonium | |
| | | | | | | | acetate, | |
| | | | | | | | 10 mM CaCl₂ | |
| Duplex III-3 | 4.4 | P3₁21 | 118.8 | 118.8 | 201.6 | | 7% PEG 6000, | unknown |
| TCACAGGATCCTGTG | | or | | | | | 100 mM MES | |
| GTGTCCTAGGACACT | | P3₂21 | | | | | pH 5.6, | |
| | | | | | | | 200 mM | |
| | | | | | | | ammonium | |
| | | | | | | | acetate, | |
| | | | | | | | 10 mM CaCl2, | |
| | | | | | | | 20% glycerol | |

TABLE 3
Crystallographic Data and Refinement Statistics of T4Dam Crystals

| | Crystal | | |
|---|---|---|---|
| | T4Dam-AdoHcy- | T4Dam-AdoHcy- | T4Dam-sinefungin- |
| | 13mer DNA | 15mer DNA | 16mer DNA^{a} |
| PDB code | 1YF3 | 1YFJ | 1YFL |
| Space group | P2₁ | P2₁2₁2₁ | P2₁ |
| Unit cell dimensions | | | |
| a (Å) | 38.9 | 111.4 | 87.9 |
| b (Å) | 125.8 | 133.2 | 117.7 |
| c (Å) | 73.2 | 191.2 | 87.9 |
| β (°) | 104.7 | — | 90.0 |
| Beamline | IMCA-CAT (APS) | SER-CAT (APS) | SER-CAT, APS |
| Resolution range (Å) | 28-2.29 (2.37-2.29) | 35-2.69 (2.79-2.69) | 30-3.09 (3.29-3.09) |
| (Highest resolution) | | | |
| Measured reflections | 109,113 | 647,805 | 179,628 |
| Unique reflections | 29,474 | 78,518 | 33,149 |
| <I/σ> | 22.3 | 12.9 | 10.9 |
| Completeness (%) | 95.7 (97.3) | 99.9 (100) | 99.9 (100) |
| R-linear = Σ\|I − | 0.047 (0.187) | 0.070 (0.323) | 0.081 (0.314) |
| <I>\|/Σ<I> | | | |
| R-factor = Σ\|Fₒ − | 0.214 (0.273) | 0.202 (0.281) | 0.214 (0.362) |
| F_{c}\|/Σ\|F_{c}\| | | | |
| R-free (5% of data) | 0.270 (0.349) | 0.250 (0.341) | 0.265 (0.392) |
| Molecules/asymmetric | 2 protein and 1 | 6 protein and 5 DNA | 4 protein and 2 DNA |
| unit | DNA | | |
| Non-hydrogen atoms | | | |
| Protein | 4032 | 9520 | 3940 |
| Nucleic acid | 527 | 2155 | 2009 |
| Hetrogen | 64 (2 AdoHcy) | 178 (6 AdoHcy) | 108 (4 sinefungin) |
| Water | 204 | 327 | 2 |
| Root-mean-square | | | |
| deviation from ideal | | | |
| Bond lengths (Å) | 0.02 | 0.01 | 0.01 |
| Bond angles (°) | 1.7 | 2.0 | 1.5 |
| Dihedral (°) | 21.4 | 22.2 | 22.6 |
| Improper (°) | 1.53 | 1.08 | 1.09 |
| Estimated coordinate | | | |
| error | | | |
| From Sigmaa (Å) | 0.22 | 0.21 | 0.36 |

TABLE 4
Crystallographic Data and Refinement Statistics

| | Crystal^{(a)} | | | |
|---|---|---|---|---|
| | EcoDam-DNA (cognate)-AdoHcy | | EcoDam-DNA (noncognate)-sinefungin | |
| PDB code | — | | — | |
| Beamline (wavelength Å) | APS 22-ID (0.97179) | | APS 22-ID (1.0) | |
| Space group | P2₁^{(b)} | | P2₁2₁2₁ | |
| Unit cell dimensions (Å) | 46.2, 71.3, 97.8 | | 36.3, 63.1, 145.0 | |
| and angles (°) | 90, 90.03, 90 | | 90, 90, 90 | |
| Resolution Range (Å) | 33.6-1.89 | (1.92-1.89) | 32.51-1.99 | (2.06-1.99) |
| (highest resolution shell) | | | | |
| Measured reflections | 377,638 | | 181,130 | |
| Unique reflections | 51,126 | | 23,469 | |
| <I/σ> | 9.1 | | 20.9 | |
| Completeness (%) | 99.9 | (98.7) | 98.2 | (97.8) |
| Rₗᵢₙₑₐᵣ = Σ\|I − <I>\|/Σ<I> | 0.102 | (0.575) | 0.051 | (0.325) |
| R-factor = Σ\|Fₒ − F_{c}\|/Σ\|F_{c}\| | 0.188 | (0.278) | 0.2332 | (0.274) |
| R-free (5% data) | 0.216 | (0.287) | 0.266 | (0.280) |
| Non-hydrogen atoms | | | | |
| Protein | 3996 | (2 EcoDam) | 2008 | (1 EcoDam) |
| DNA | 486 | (1 DNA duplex) | 486 | (1 DNA duplex) |

| | | | |
|---|---|---|---|
| Hetrogen | 52 (2 AdoHcy) + 6 (1 glycerol) | 27 | (1 sinefungin) |

| | | |
|---|---|---|
| Water | 321 | 100 |

Room-mean-square deviation from ideality

| | | |
|---|---|---|
| Bond lengths (Å) | 0.010 | 0.011 |
| Bond angles (°) | 1.4 | 1.4 |
| Dihedral (°) | 22.0 | 22.0 |
| Improper (°) | 1.0 | 1.2 |

Estimated coordinate error

| | | |
|---|---|---|
| From Luzzati plot (Å) | 0.20 | 0.27 |
| From Sigmaa (Å) | 0.19 | 0.18 |

TABLE 5
E. coli Crystallization Summary

| | Reflections | Average | | Linear R- |
|---|---|---|---|---|
| Resolution (Å) | (numbers) | Redundancy | Complete | Factor |
| 35.00-1.89 | 26570 | 6.0 | 99.8% | 0.089 |

TABLE 6
Examples of TANAC elements in the EcoDam-regulated promoters

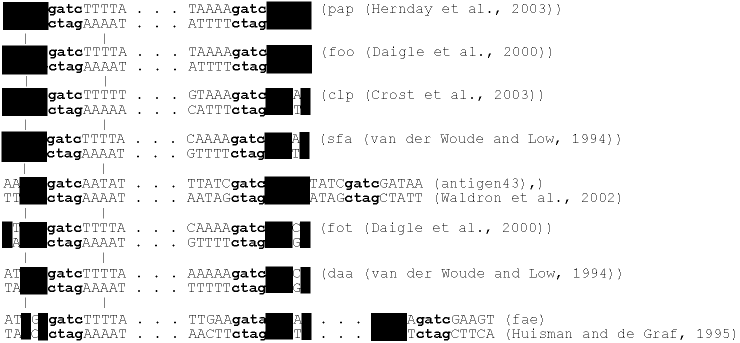

TABLE 7
Preliminary results derived from the microplate assay

| Compound # | 45 | 49 | 53 | 55 | 56 | 58 | 69 | 78 | 80 |
|---|---|---|---|---|---|---|---|---|---|
| Factor of inhibition | 3.5 | 4 | 2.5 | 3 | 4-5 | >5 | >2 | 5 | >5 |

Note:
Corresponding structures are provided in FIG. 27.

TABLE 8
Summary of PDB ID Codes

| | |
|---|---|
| T4Dam-AdoHcy-13-mer DNA | 1YF3 |
| T4Dam-AdoHcy-15-mer DNA | 1YFJ |
| T4Dam-sinefungen-16-mer DNA | 1YFL |
| T4Dam-AdoHcy | 1QOT |
| T4Dam-AdoHcy-12-mer DNA | 1QOS |

TABLE 9
References

Allan, B. W., Reich, . N. O. and Beechem, J. M., Measurement of the absolute temporal coupling between
DNA binding and base flipping, Biochemistry 38 (1999), pp. 5308-5314.
Allan, B. W., Beechem, J. M., Lindstrom, W. M., and Reich, N. O. (1998). Direct real time observation of
base flipping by the EcoRI DNA methyltransferase. J Biol Chem 273, 2368-2373.
Allan, B. W., and Reich, N. O. (1996). Targeted base stacking disruption by the EcoRI DNA
methyltransferase. Biochemistry 35, 14757-14762.
Anyanful, A., Dolan-Livengood, J., Lewis, T., Sheth, S., DeZalla, M. N., Sherman, M., Kalman, L. V.,
Benian, G. M., and Kalman, D. (2005). Paralysis and killing of C. elegans by enteropathogenic E. coli
requires the bacterial tryptophanase gene. Molecular Microbiology (in press).
Åqvist, J., Medina, C., and Samuelsson, J. E. (1994). A new method for predicting binding affinity in
computer-aided drug design. Protein Eng 7, 385-391.
Banerjee, A., Yang, W., Karplus, M., and Verdine, G. L. (2005). Structure of a repair enzyme interrogating
undamaged DNA elucidates recognition of damaged DNA. Nature 434, 612-618.
Barthel, M., Hapfelmeier, S., Quintanilla-Martinez, L., Kremer, M., Rohde, M., Hogardt, M., Pfeffer, K.,
Russmann, H., and Hardt, W. D. (2003). Pretreatment of mice with streptomycin provides a Salmonella
enterica serovar Typhimurium colitis model that allows analysis of both pathogen and host. Infect Immun
71, 2839-2858.
Bayliss, C. D., van de Ven, T., and Moxon, E. R. (2002). Mutations in poll but no mutSLH destabilize
Haemophilus influenzae tetranucleotide repeats. Embo J 21, 1465-1476.
Beck, C. and Jeltsch, A., Probing the DNA interface of the EcoRV DNA-(adenine-N6)-methyltransferase
by site-directed mutagenesis, fluorescence spectroscopy, and UV cross-linking, Biochemistry 41 (2002),
pp. 14103-14110.
Berdis, A. J., Lee, I., Coward, J. K., Stephens, C., Wright, R., Shapiro, L., and Benkovic, S. J. (1998). A
cell cycle-regulated adenine DNA methyltransferase from Caulobacter crescentus processively
methylates GANTC sites on hemimethylated DNA. Proc Natl Acad Sci USA 95, 2874-2879.
Bergerat, A., Kriebardis, A., and Guschlbauer, W. (1989). Preferential site-specific hemimethylation of
GATC sites in pBR322 DNA by Dam methyltransferase from Escherichia coli. J Biol Chem 264, 4064-
4070.
Bernards, A. S., Miller, J. K., Bao, K. K., and Wong, I. (2002). Flipping duplex DNA inside out: a double
base-flipping reaction mechanism by Escherichia coli MutY adenine glycosylase. J Biol Chem 277, 20960-
20964.
Brooks, B. R., Bruccoleri, R. E., Olafson, B. D., States, D. J., Swaminathan, S., and Karplus, M. (1983).
CHARMM: A Program for Macromolecular Energy, Minimization, and Dynamics Calculations. J Comput
Chem 4, 187-217.
Brooks, J. E. and Hattman, S., In vitro methylation of bacteriophage lambda DNA by wild type (dam+) and
mutant (damh) forms of the phage T2 DNA adenine methylase, J. Mol. Biol. 126 (1978), pp. 381-394.
Bruner, S. D., Norman, D. P. and Verdine, G. L., Structural basis for recognition and repair of the
endogenous mutagen 8-oxoguanine in DNA, Nature 403 (2000), pp. 859-866.
Brunger, A. T., Adams, P. D., Clore, G. M., DeLano, W. L., Gros, P., Grosse-Kunstleve, R. W., Jiang, J.
S., Kuszewski, J., Nilges, M., Pannu, N. S., et al. (1998). Crystallography & NMR system: A new software
suite for macromolecular structure determination. Acta Crystallogr D Biol Crystallogr 54 (Pt 5), 905-921.
Bucci, C., Lavitola, A., Salvatore, P., Del Giudice, L., Massardo, D. R., Bruni, C. B., and Alifano, P. (1999).
Hypermutation in pathogenic bacteria: frequent phase variation in meningococci is a phenotypic trait of a
specialized mutator biotype. Mol Cell 3, 435-445.
Butina, D. (1999). Unsupervised Data Base Clustering on Daylight's Fingerprint and Tanimoto Similarity:
A Fast and Automated Way to Cluster Small and Large Data Sets. J Chem Inf Comput Sci 39, 747-750.
Carlson, H. A. (2002). Protein flexibility and drug design: how to hit a moving target. Curr Opin Chem
Biol 6, 447-452.
Charifson, P. S., Corkery, J. J., and Murcko, M. A. (1999). Consensus scoring: A method for obtaining
improved hit rates from docking databases of three-dimensional structures into proteins. J Med Chem 42,
5100-5109.
Chen, I. J., Neamati, N., Nicklaus, M. C., Orr, A., Anderson, L., Barchi, J. J., Jr., Kelley, J. A., Pommier,
Y., and MacKerell, A. D., Jr. (2000). Identification of HIV-1 Integrase Inhibitors via Three-Dimensional
Database Searching using ASV and HIV-1 Integrases as Targets. Biorg & Med Chem 8, 2385-2398.
Chen, L., Paulsen, D. B., Scruggs, D. W., Banes, M. M., Reeks, B. Y., and Lawrence, M. L. (2003)
Alteration of DNA adenine methylase (Dam) activity in Pasteurella multocida causes increased
spontaneous mutation frequency and attenuation in mice. Microbiology 149, 2283-2290.
Cheng, X., and Roberts, R. J. (2001). AdoMet-dependent methylation, DNA methyltransferases and base
flipping. Nucleic Acids Res 29, 3784-3795.
Crost, C., Garrivier, A., Harel, J., and Martin, C. (2003). Leucine-responsive regulatory protein-mediated
repression of clp (encoding CS31A) expression by L-leucine and L-alanine in Escherichia coli. J Bacteriol
185, 1886-1894.
Daigle, F., Forget, C., Martin, C., Drolet, M., Tessier, M. C., Dezfulian, H., and Harel, J. (2000). Effects of
global regulatory proteins and environmental conditions on fimbrial gene expression of F165(1) and
F165(2) produced by Escherichia coli causing septicaemia in pigs. Res Microbiol 151, 563-574.
Daniels, D. S., Woo, T. T., Luu, K. X., Noll, D. M., Clarke, N. D., Pegg, A. E. and Tainer, J. A., DNA binding
and nucleotide flipping by the human DNA repair protein AGT, Nat. Struct. Mol. Biol. 11 (2004), pp. 714-720.
Datsenko, K. A., and Wanner, B. L (2000). One-step inactivation of chromosomal genes in Escherichia
coli K-12 using PCR products. Proc Natl Acad Sci USA 97, 6640-6645.
Deng, W., Puente, J. L., Gruenheid, S., Li, Y., Vallance, B. A., Vazquez, A., Barba, J., Ibarra, J. A.,
O'Donnell, P., Metalnikov, P., et al. (2004). Dissecting virulence: systematic and functional analyses of a
pathogenicity island. Proc Natl Acad Sci USA 101, 3597-3602.
DesJarlais, R. L., Sheridan, R. P., Dixon, J. S., Kuntz, I. D., and Venkataraghaven, R. (1986). Docking
flexible ligands to macromolecular receptors by molecular shape. J Med Chem 29, 2149-2153.
Dueger, E. L., House, J. K., Heithoff, D. M., and Mahan, M. J. (2001). Salmonella DNA adenine methylase
mutants elicit protective immune responses to homologous and heterologous serovars in chickens. Infect
Immun 69, 7950-7954.
Dueger, E. L., House, J. K., Heithoff, D. M., and Mahan, M. J. (2003). Salmonella DNA adenine methylase
mutants prevent colonization of newly hatched chickens by homologous and heterologous serovars. Int J
Food Microbiol 80, 153-159.
Ewing, T. J., Makino, S., Skillman, A. G., and Kuntz, I. D. (2001). DOCK 4.0: search strategies for
automated molecular docking of flexible molecule databases. J Comput Aided Mol Des 15, 411-428.
Ewing, T. J. A., and Kuntz, I. D. (1997). Critical evaluation of search algorithms used in automated
molecular docking. J Comput Chem 18, 1175-1189.
Falker, S., Schmidt, M. A., Heusipp, G., (2005). DNA methylation in Yersinia enterocolitica: role of the
DNA adenine methyltransferase in mismatch repair and regulation of virulence factors. Microbiology.
151: 2291-9.
Fatemi, M., Hermann, A., Pradhan, S., and Jeltsch, A. (2001). The activity of the murine DNA
methyltransferase Dnmt1 is controlled by interaction of the catalytic domain with the N-terminal part of the
enzyme leading to an allosteric activation of the enzyme after binding to methylated DNA. J Mol Biol 309,
1189-1199.
Feig, J M., and Brooks, C. L., III (2004). Recent advances in the development and application of implicit
solvent models in biomolecular simulations. Cur Opin Struct Biol 14, 217-224.
Fersht, A. R., Matouschek, A., and Serrano, L. (1992). The folding of an enzyme. I. Theory of protein
engineering analysis of stability and pathway of protein folding. J Mol Biol 224, 771-782.
Floersheim et al., U.S. Patent Application, Publication No. US2005/020205 A1.
Fromme, J. C., Banerjee, A., Huang, S. J., and Verdine, G. L. (2004). Structural basis for removal of
adenine mispaired with 8-oxoguanine by MutY adenine DNA glycosylase. Nature 427, 652-656.
Garcia-Del Portillo, F., Pucciarelli, M. G., and Casadesus, J. (1999). DNA adenine methylase mutants of
Salmonella typhimurium show defects in protein secretion, cell invasion, and M cell cytotoxicity. Proc Natl
Acad Sci USA 96, 11578-11583.
Gill, C. J., and Hamar, D. H. (2001). Foodborne Illnesses. 4, 23-38.
Goedecke, K., Pignot, M., Goody, R. S., Scheidig, A. J., and Weinhold, E. (2001). Structure of the N6-
adenine DNA methyltransferase M.TaqI in complex with DNA and a cofactor analog. Nat Struct Biol 8,
121-125.
Goodford, P. J. (1984). A computational procedure for determining energetically favorable binding sites on
biologically important macromolecules. J Med Chem 28, 849-857.
Goosney, D. L., Gruenheid, S., and Finlay, B. B. (2000). Gut feelings: enteropathogenic E. coli (EPEC)
interactions with the host. Annu Rev Cell Dev Biol 16, 173-189.
Gowher, H., Zhang, X., Cheng, X., and Jeltsch, A. (2005). Avidin plate assay system for enzymatic
characterization of a histone lysine methyltransferase. Anal Biochem. May 31 [epub ahead of print].
Grace Goll, M., and Bestor, T. H. (2005). Eukaryotic cytosine methyltransferases. Annu Rev Biochem 74,
481-514.
Guarne, A., Zhao, Q., Ghirlando, R., and Yang, W. (2002). Insights into negative modulation of E. coli
replication initiation from the structure of SeqA-hemimethylated DNA complex. Nat Struct Biol 9, 839-843.
Halford, S. E. and Marko, J. F., How do site-specific DNA-binding proteins find their targets?, Nucleic Acids
Res. 32 (2004), pp. 3040-3052.
Hancock, C. N., Macias, A., Lee, E. K., Yu, S. Y., MacKerell, J. A. D., and Shapiro, P. (2005).
Identification of novel extracellular signal-regulated kinase (ERK) docking domain inhibitors. J Med Chem
In press.
Hattman, S., Brooks, J. E., and Masurekar, M. (1978). Sequence specificity of the P1 modification
methylase (M.Eco P1) and the DNA methylase (M.Eco dam) controlled by the Escherichia coli dam gene.
J Mol Biol 126, 367-380.
Hattman. S., and Malygin, E. G. (2004). Bacteriophage T2Dam and T4Dam DNA-[N6-adenine]-
methyltransferases. Progress in Nucleic Acid Research and Molecular Biology 77, 67-126.
Hattman, S., DNA methylation of T-even bacteriophages and of their nonglucosylated mutants: its role in
P1-directed restriction, Virology 42 (1970), pp. 359-367.
Hattman, S., Unusual transcriptional and translational regulation of the bacteriophage Mu mom operon,
Pharmacol. Ther. 84 (1999), pp. 367-388.
Hattman, S., Wilkinson, J., Swinton, D., Schlagman, S., Macdonald, P. M. and Mosig, G., Common
evolutionary origin of the phage T4 dam and host Escherichia coli dam DNA-adenine methyltransferase
genes, J. Bacteriol. 164 (1985), pp. 932-937.
Heithoff, D. M., Enioutina, E. Y., Daynes, R. A., Sinsheimer, R. L., Low, D. A., and Mahan, M. J. (2001).
Salmonella DNA adenine methylase mutants confer cross-protective immunity. Infect Immun 69, 6725-6730.
Heithoff, D. M., Sinsheimer, R. L., Low, D. A., and Mahan, M. J. (1999). An essential role for DNA adenine
methylation in bacterial virulence. Science 284, 967-970.
Hermann, A., Goyal, R., and Jeltsch, A. (2004). The Dnmt1 DNA-(cytosine-C5)-methyltransferase
methylates DNA processively with high preference for hemimethylated target sites. J Biol Chem 279,
48350-48359.
Hernday, A. D., Braaten, B. A., and Low, D. A. (2003). The mechanism by which DNA adenine methylase
and PapI activate the pap epigenetic switch. Mol Cell 12, 947-957.
Holz, B., Klimasauskas, S., Serva, S., and Weinhold, E. (1998). 2-Aminopurine as a fluorescent probe for
DNA base flipping by methyltransferases. Nucleic Acids Res 26, 1076-1083.
Honma, Y., Fernandez, R. E., and Maurelli, A. T. (2004). A DNA adenine methylase mutant of Shigella
flexneri shows no significant attenuation of virulence. Microbiology 150, 1073-1078.
Horton, J. R., Liebert, K., Hattman, S., Jeltsch, A., and Cheng, X. (2005). Transition from nonspecific to
specific DNA interactions along the substrate-recognition pathway of dam methyltransferase. Cell 121,
349-361.
Horton, N. C., Dorner, L. F. and Perona, J. J., Sequence selectivity and degeneracy of a restriction
endonuclease mediated by DNA intercalation, Nat. Struct. Biol. 9 (2002), pp. 42-47.
Horton, J. R., Sawada, K., Nishibori, M., Zhang, X., and Cheng, X. (2001). Two polymorphic forms of
human histamine methyltransferase: structural, thermal, and kinetic comparisons. Structure 9, 837-849.
Horton, J. R.. Ratner, G., Banavali, N. K., Huang, N., Choi, Y., Maier, M. A., Marquez, V. E., MacKerell, A.
D., Jr., and Cheng, X. (2004). Caught in the act: visualization of an intermediate in the DNA base-flipping
pathway induced by HhaI methyltransferase. Nucleic Acids Res 32, 3877-3886.
Hosfield, D. J., Guan, Y., Haas, B. J., Cunningham, R. P., and Tainer, J. A. (1999). Structure of the DNA
repair enzyme endonuclease IV and its DNA complex: double-nucleotide flipping at abasic sites and
three-metal-ion catalysis. Cell 98, 397-408.
Huang, N., Banavali, N. K., and MacKerell, A. D., Jr. (2003). Protein-facilitated base flipping in DNA by
cytosine-5-methyltransferase. Proc Natl Acad Sci USA 100, 68-73.
Huang, N., Nagarsekar, A., Xia, G., Hayashi, J., and MacKerell, Jr., A. D. (2004). Identification of
Inhibitors Targeting the pY+3 Binding Site of the Tyrosine Kinase p53lck SH2 domain. J Med Chem 47,
3502-3511.
Huisman, T. T., and de Graaf, F. K. (1995). Negative control of fae (K88) expression by the ‘global’
regulator Lrp is modulated by the ‘local’ regulator FaeA and affected by DNA methylation. Mol Microbiol
16, 943-953.
Humeny, A., Beck, C., Becker, C. M., and Jeltsch, A., (2003) Detection and analysis of enzymatic DNA
methylation of oligonucleotide substrates by matrix-assisted laser desorption ionization time-of-flight mass
spectrometry. Anal Biochem. 313, 160-6.
Jeltsch, A. (2002). Beyond Watson and Crick: DNA methylation and molecular enzymology of DNA
methyltransferases. Chembiochem 3, 274-293.
Jeltsch, A., and Lanio, T. (2002). Site-directed mutagenesis by polymerase chain reaction. Methods Mol
Biol 182, 85-94.
Jones, P. A., and Takai, D. (2001). The role of DNA methylation in mammalian epigenetics. Science 293,
1068-1070.
Jones, T. A., Zou, J. Y., Cowan, S. W., and Kjeldgaard (1991). Improved methods for building protein
models in electron density maps and the location of errors in these models. Acta Crystallogr A 47 (Pt 2)
110-119.
Julio, S. M., Heithoff, D. M., Provenzano, D., Klose, K. E., Sinsheimer, R. L, Low, D. A., and Mahan, M. J.
(2001). DNA adenine methylase is essential for viability and plays a role in the pathogenesis of Yersinia
pseudotuberculosis and Vibrio cholerae. Infect Immun 69, 7610-7615.
Julio, S. M., Heithoff, D. M., Sinsheimer, R. L., Low, D. A., and Mahan, M. J. (2002). DNA adenine
methylase overproduction in Yersinia pseudotuberculosis alters YopE expression and secretion and host
immune responses to infection. Infect Immun 70, 1006-1009.
Kalman, D., Weiner, O. D., Goosney, D. L., Sedat, J. W., Finlay, B. B., Abo, A., and Bishop, J. M. (1999).
Enteropathogenic E. coli acts through WASP and Arp2/3 complex to form actin pedestals. Nat Cell Biol 1,
389-391.
Kalodimos, C. G., Biris, N., Bonvin, A. M., Levandoski, M. M., Guennuegues, M., Boelens, R., and
Kaptein, R. (2004). Structure and flexibility adaptation in nonspecific and specific protein-DNA
complexes. Science 305, 386-389.
Kang, S., Lee, H., Han, J. S., and Hwang, D. S. (1999). Interaction of SeqA and Dam methylase on the
hemimethylated origin of Escherichia coli chromosomal DNA replication. J Biol Chem 274, 11463-11468.
Kang, X., Shafer, R. H., and Kuntz, I. D. (2004). Calculation of ligand-nucleic acid binding free energies
with the generalized-born model in DOCK. Biopolymers 73, 192-204.
Kenny, B., Devinney, R., Stein, M., Reinscheid, D. J., Frey, E. A., and Finlay, B. B. (1999).
Enteropathogenic E. coli (EPEC) transfers its receptor for intimate adherence into mammalian cells. Cell
91, 511-520.
Kita et al., U.S. Patent Application, Publication No. 2005/0170379 A1.
Klimasauskas, S., Kumar, S., Roberts, R. J., and Cheng, X. (1994). HhaI methyltransferase flips its target
base out of the DNA helix. Cell 76, 357-369.
Klimasauskas, S., and Roberts, R. J. (1995). M.HhaI binds tightly to substrates containing mismatches at
the target base. Nucleic Acids Res 23, 1388-1395.
Knutton, S., Shaw, R., McNeish, A. S., Phliips, A., Price, E., and Watson, P. (1989). Diagnosis of
enteropathogenic Escherichia coli. Lancet 2, 218.
Kossykh, V. G., Schlagman, S. L. and Hattman, S., Phage T4 DNA [N6-adenine]methyltransferase.
Overexpression, purification, and characterization, J. Biol. Chem. 270 (1995), pp. 14389-14393.
Krabbe, M., Weyland, N., and Low, D. (2000). Environmental control of pilus gene expression. In
Bacterial stress responses (Washington, D.C., ASM Press; G. Storz, and R. Hengge-Aronis, eds), 305-321.
Kuntz, I. D., Blaney, J. M., Oatley, S. J., Langridge, R., and Ferrin, T. E. (1982). A Geometric Approach to
Macromolecule-Ligand Interactions. Journal of Molecular Biology 161, 269-288.
Lacks, S., and Greenberg, B. (1977). Complementary specificity of restriction endonucleases of
Diplococcus pneumoniae with respect to DNA methylation. J Mol Biol 114, 153-168.
Lamb, M. L, Tirado-Rives, J., and Jorgensen, W. L. (1999). Estimation of the binding affinities of FKBP12
inhibitors using a linear response method. Bioorg Med Chem 7, 851-860.
Lau, A. Y., Scharer, O. D., Samson, L., Verdine, G. L. and Ellenberger, T., Crystal structure of a human
alkylbase-DNA repair enzyme complexed to DNA: mechanisms for nucleotide flipping and base excision,
Cell 95 (1998), pp. 249-258.
Lauster, R., Kriebardis, A. and Guschlbauer, W., The GATATC-modification enzyme EcoRV is closely
related to the GATC-recognizing methyltransferases DpnII and dam from E. coli and phage T4, FEBS
Lett. 220 (1987), pp. 167-176.
Levine, M. M., and Edelman, R. (1984). Enteropathogenic Escherichia coli of classic serotypes
associated with infant diarrhea: epidemiology and pathogenesis. Epidemiologic Reviews 6, 31-51.
Liebert, K., Hermann, A., Schlickenrieder, M., and Jeltsch, A. (2004). Stopped-flow and mutational
analysis of base flipping by the Escherichia coli Dam DNA-(adenine-N6)-methyltransferase. J Mol Biol
341, 443-454.
Lipinski, C. A. (2000). Drug-like properties and the causes of poor solubility and poor permeability. J
Pharmacol Toxicol Methods 44, 235-249.
Liu, R., Hsieh, C. Y., and Lam, K. S. (2004). New approaches in identifying drugs to inactivate oncogene
products. Semin Cancer Biol 14, 13-21.
Lobner-Olesen, A., Marinus, M. G., and Hansen, F. G. (2003). Role of SeqA and Dam in Escherichia coli
gene expression: a global/microarray analysis. Proc Natl Acad Sci USA 100, 4672-4677.
Lobner-Olesen, A., Skovgaard, O., and Marinus, M. G. (2005). Dam methylation: coordinating cellular
processes. Curr Opin Microbiol 8, 154-160.
Low, D. A., Weyand, N. J., and Mahan, M. J. (2001). Roles of DNA adenine methylation in regulating
bacterial gene expression and virulence. Infect Immun 69, 7197-7204.
Lu, M., Campbell, J. L., Boye, E., and Kleckner, N. (1994). SeqA: a negative modulator of replication
initiation in E. coli. Cell 77, 413-426.
Luperchio, S. A., and Schauer, D. B. (2001). Molecular pathogenesis of Citrobacter rodentium and
transmissible murine colonic hyperplasia. Microbes Infect 3, 333-340.
MacKerell, A. D., Jr., Bashford, D., Bellott, M., Dunbrack Jr., R. L., Evanseck, J., Field, M. J., Fischer, S.,
Gao, J., Guo, H., Ha, S., et al. (1998a). All-atom empirical potential for molecular modeling and dynamics
studies of proteins. J Phys Chem B 102, 3586-3616.
MacKerell, A. D., Jr., Brooks, B., Brooks, C. L., III, Nilsson, L., Roux, B., Won, Y., and Karplus, M.
(1998b). CHARMM: The Energy Function and Its Paramerization with an Overview of the Program. In
Encyclopedia of Computational Chemistry, P. R. Schreiner, ed. (Chichester, John Wiley & Sons) pp 271-277.
MacKerell, A. D., Jr., Feig, M., and Brooks, C. L., III (2004). Accurate treatment of protein backbone
conformational energetics in empirical force fields. J Am Chem Soc 126, 698-699.
Malone, T., Blumenthal, R. M. and Cheng, X., Structure-guided analysis reveals nine sequence motifs
conserved among DNA amino-methyltransferases, and suggests a catalytic mechanism for these
enzymes, J. Mol. Biol. 253 (1995), pp. 618-632.
Malygin, E. G., Zinoviev, V. V., Petrov, N. A., Evdokimov, A. A., Jen-Jacobson, L., Kossykh, V. G. and
Hattman, S., Effect of base analog substitutions in the specific GATC site on binding and methylation of
oligonucleotide duplexes by the bacteriophage T4 Dam DNA-[N6-adenine] methyltransferase, Nucleic
Acids Res. 27 (1999), pp. 1135-1144.
Markowitz, J., Chen, I., Gitti, R., Baldisseri, D. M., Pan, Y., Udan, R., Carrier, F., MacKerell, A. D., Jr., and
Weber, D. J. (2004). Identification and characterization of small molecule inhibitors of the calcium-
dependent S100B-p53 tumor suppressor interaction. J Med Chem 47, 5085-5093.
Mashhoon, N., Carroll, M., Pruss, C., Eberhard, J., Ishikawa, S., Estabrook, R. A., and Reich, N. (2004).
Functional characterization of Escherichia coli DNA adenine methyltransferase, a novel target for
antibiotics. J Biol Chem 279, 52075-52081.
Mead, P. S., Slutsker, L., Dietz, V., McCaig, L. F., Bresee, J. S., Shapiro, C., Griffin, P. M., and Tauxe, R.
V. (1999). Food-related illness and death in the United States. Emerg Infect Dis 5, 607-625.
Messer, W., and Noyer-Weidner, M. (1988). Timing and targeting: the biological functions of Dam
methylation in E. coli. Cell 54, 735-737.
Miner, Z., Schlagman, S. L. and Hattman, S., Single amino acid changes that alter the DNA sequence
specificity of the DNA-[N6-adenine] methyltransferase (Dam) of bacteriophage T4, Nucleic Acids Res. 17
(1989), pp. 8149-8157.
Modrich, P., and Lahue, R. (1996). Mismatch repair in replication fidelity, genetic recombination, and
cancer biology. Annu Rev Biochem 65, 101-133.
Modrich. P. (1989). Methyl-directed DNA mismatch correction. J Biol Chem 264, 6597-6600.
Murphy, K. C. (1998). Use of bacteriophage lambda recombination functions to promote gene
replacement in Escherichia coli. J Bacteriol 180, 2063-2071.
Oprea, T. I., Davis, A. M., Teague, S. J., and Leeson, P. D. (2001). Is There a Difference between Leads
and Drugs? A Historical Perspective. J Chem Inf Comp Sci 41, 1308-1315.
Oshima, T., Wada, C., Kawagoe, Y., Ara, T., Maeda, M., Masuda, Y., Hiraga, S., and Mori, H. (2002).
Genome-wide analysis of deoxyadenosine methyltransferase-mediated control of gene expression in
Escherichia coli. Mol Microbiol 45, 673-695.
Oza, J. P., Yeh, J. B., Reich, N. O., (2005) DNA methylation modulates Salmonella enterica serovar
Typhimurium virulence in Caenorhabditis elegans. FEMS Microbiol Lett. 245(1): 53-9.
Pabo, C. O., Peisach, E. and Grant, R. A., Design and selection of novel Cys2His2 zinc finger proteins,
Annu. Rev. Biochem. 70 (2001), pp. 313-340.
Paiva, A. M., Vanderwall, D. E., Blanchard. J. S., Kozarich, J. W., Williamson, J. M., and Kelly, T. M.
(2001). Inhibitors of dihydrodipicolinate reductase, a key enzyme of the diaminopimelate pathway of
Mycobacterium tuberculosis. Biochem Biophys Acta 1545, 67-77.
Pan, Y., Huang, N., Cho, S., and MacKerell, A. D., Jr. (2003). Consideration of Molecular Weight During
Compound Selection in Virtual Target-Based Database Screening. J Chem Inf Comp Sci 43, 267-272.
Pandit, United States Patent Application, Publication No. US2005/0202550 A1.
PCT 03/024567 (Int. Pub No. WO 04/012683); U.S. Pat. Ser. No. 10/636,508.
Reeves, P., Veach, D., Bornmann, W., Sherman, M., and Kalman, D. (2005). Vaccinia virus uses Abl and
Scr family kinases for actin motility and cell-to-cell spread. Submitted.
Reinisch, K. M., Chen, L., Verdine, G. L., and Lipscomb, W. N. (1995). The crystal structure of HaeIII
methyltransferase convalently complexed to DNA: an extrahelical cytosine and rearranged base pairing.
Cell 82, 143-153.
Renbaum, P., and Razin, A. (1992). Mode of action of the Spiroplasma CpG methylase M.Sssl. FEBS
Lett 373, 243-247.
Riley, L. W., Remis, R. S., Helgerson, S. D., McGee, H. B., Wells, J. G., Davis, B. R., Hebert, R. J., Olcott,
E. S., Johnson, L. M., Hargrett, N. T., et al. (1983). Hemorrhagic colitis associated with a rare Escherichia
coli serotype. N Engl J Med 308, 681-685.
Roche, O., Schneider, P., Zuegge, J., Guba, W., Kansy, M., Alanine, A., Bleicher, K., Danel, F.,
Gutknecht, E. M., Rogers-Evans, M., et al. (2002). Development of a virtual screening method for
identification of “frequent hitters” in compound libraries. J Med Chem 45, 137-142.
Roth, M. and Jeltsch, A., Changing the target base specificity of the EcoRV DNA methyltransferase by
rational de novo protein-design, Nucleic Acids Res. 29 (2001), pp. 3137-3144.
Roth, M., and Jeltsch, A. (2000). Biotin-avidin microplate assay for the quantitative analysis of enzymatic
methylation of DNA by DNA methyltransferases. Biol Chem 381, 269-272.
Sali, A., and Blundell, T. L. (1993). Comparative protein modeling by satisfaction of spatial restraints. J
Mol Biol 234, 779-815.
Sawyer, J. S., Anderson, B. D., Beight, D. W., Campbell, R. M., Jones, M. L., Herron, D. K., Lampe, J. W.,
McCowan, J. R., McMillen, W. T., Mort, N., et al. (2003). Synthesis and activity of new aryl- and
heteroaryl-substituted pyrazole inhibitors of the transforming growth factor-beta type I receptor kinase
domain. J Med Chem 46, 3953-3956.
Schubert, H. L., Blumenthal, R. M., and Cheng, X. (2003). Many paths to methyltransfer: a chronicle of
convergence. Trends Biochem Sci 28, 329-335.
Seeman, N. C., Rosenberg, J. M. and Rich, A., Sequence-specific recognition of double helical nucleic
acids by proteins, Proc. Natl. Acad. Sci. USA 73 (1976), pp. 804-808.
Serre, L., Pereira de Jesus, K., Boiteux, S., Zelwer, C. and Castaing, B., Crystal structure of the
Lactococcus lactis formamidopyrimidine-DNA glycosylase bound to an abasic site analogue-containing
DNA, EMBO J. 21 (2002), pp. 2854-2865.
Singh, J., Chuaqui, C. E., Boriack-Sjodin, P. A., Lee, W. C., Pontz, T., Corbley, M. J., Cheung, H. K.,
Arduini, R. M., Mead, J. N., Newman, M. N., et al. (2003). Successful shape-based virtual screening: the
discovery of a potent inhibitor of the type I TGFbeta receptor kinase (TbetaRI). Bioorg Med Chem Lett 13,
4355-4359.
Sirois, S., Hatzakis, G., Wei, D., Du, Q., and Chou, K.-C. (2005). Assessment of chemical libraries for
their druggability. Comp Biol Chem 29, 55-67.
Stephen, A. G., Worthy, K. M., Towler, E., Mikovits, J. A., Sei, S., Roberts, P., yang, Q. E., Akee R. K.,
Klausmeyer, P., McCloud, T. G., et al. (2002). Identification of HIV-1 nucleocapsid protein: nucleic acid
antagonists with cellular anti-HIV activity. Biochem Biophys Res Commun 296, 1228-1237.
Stivers, J. T. (1998). 2-Aminopurine fluorescence studies of base stacking interactions at abasic sites in
DNA: metal-ion and base sequence effects. Nucleic Acids Res 26, 3837-3844.
Studier, F. W. (2005). Protein production by auto-induction in high density shaking cultures. Protein Expr
Purif 41, 207-234.
Swimm, A., Bommarius, B., Li, Y., Cheng, D., Reeves, P., Sherman, M., Veach, D., Bornmann, W., and
Kalman, D. (2004a). Enteropathogenic Escherichia coli use redundant tyrosine kinases to form actin
pedestals. Mol Biol Cell 15, 3520-3529.
Swimm, A., Bommarius, B., Reeves, P., Sherman, M., and Kalman, D. (2004b). Complex kinase
requirements for EPEC pedestal formation. Nat Cell Biol 6, 795; author reply 795-796.
Tong, L., and Rossmann, M. G. (1997). Rotation function calculations with GLRF program. Methods
Enzymol 276, 594-611.
Tran, P. H., Korszun, Z. R., Cerritelli, S., Springhorn, S. S., and Lacks, S. A. (1998). Crystal structure of
the DpnM DNA adenine methyltransferase from the DpnII restriction system of streptococcus pneumoniae
bound to S-adenosylmethionine. Structure 6, 1563-1575.
Tsutakawa, S. E., Jingami, H. and Morikawa, K., Recognition of a TG mismatch: the crystal structure of
very short patch repair endonuclease in complex with a DNA duplex, Cell 99 (1999), pp. 615-623.
Urig, S., Gowher, H., Hermann, A., Beck, C., Fatemi, M., Humeny, A., and Jeltsch, A. (2002). The
Escherichia coli dam DNA methyltransferase modifies DNA in a highly processive reaction. J Mol Biol
319, 1085-1096.
USDA (2002). Food Safety and Inspection Service recall release. FSIS-RC-055-2002.
van der Woude, M. W., and Low, D. A. (1994). Leucine-responsive regulatory protein and
deoxyadenosine methylase control the phase variation and expression of the sfa and daa pili operons in
Escherichia coli. Mol Microbiol 11, 605-618.
Waldron, D. E., Owen, P., and Dorman, C. J. (2002). Competitive interaction of the OxyR DNA-binding
protein and the Dam methylase at the antigen 43 gene regulatory region in Escherichia coli. Mol Microbiol
44, 509-520.
Ward, D. C., Reich, E., and Stryer, L. (1969). Fluorescence studies of nucleotides and polynucleotides. I.
Formycin, 2-aminopurine riboside, 2,6-diaminopurine riboside, and their derivatives. J Biol Chem 244,
1228-1237.
Waszkowycz, B. (2002). Structure-based approaches to drug design and virtual screening. Curr Opin
Drug Discov Devel 5, 407-413.
Watson, M. E., Jarisch, J., and Smith, A. L. (2004). Inactivation of deoxyadenosine methyltransferase
(dam) attenuates Haemophilus influenzae virulence. Mol Microbiol 53, 651-664.
Wei, O. L., Hilliard. A., Kalman, D., and Sherman, M. (2005). Mast cells limit systemic bacterial
dissemination but not colitis in response to Citrobacter rodentium. Infect Immun 73, 1978-1985.
Wolff, N. C., Veach, D. R., Tong, W. P., Bornmann, W. G., Clarkson, B., and Ilaria, R. L., Jr. (2005).
PD166326, a novel tyrosine kinase inhibitor, has greater antileukemic activity than imatinib mesylate in a
murine model of chronic myeloid leukemia. Blood 105, 3995-4003.
Yang, W. (2000). Structure and function of mismatch repair proteins. Mutat Res 460, 245-256.
Yang, Z., Norton, J. R., Zhou, L., Zhang, X. J., Dong, A., Zhang, X., Schlagman, S. L., Kossykh, V.,
Hattman, S., and Cheng, X. (2003). Structure of the bacteriophage T4 DNA adenine methyltransferase.
Nat Struct Biol 10, 849-855.
Yang, Z., Shipman, L., Zhang, M., Anton, B. P., Roberts, R. J., and Cheng, X. (2004). Structural
characterization and comparative phytogenetic analysis of Escherichia coli HemK, a protein (N5)-
glutamine methyltransferase. J Mol Biol 340, 695-706.
Zhang, X., and Cheng, X. (2003) Structure of the predominant protein arginine methyltransferase PRMT1
and analysis of its binding to substrate peptides. Structure 11, 509-520.
Zhang, X., Tamaru, H., Khan, S. I., Horton, J. R., Keefe, L. J., Selker, E. U., and Cheng, X. (2002).
Structure of the Neurospora SET domain protein DIM-5, a histone H3 lysine methyltransferase. Cell 111,
117-127.
Zhang, X., Yang, Z., Khan, S. I., Horton, J. R., Tamaru, H., Selker, E. U., and Cheng, X. (2003).
Structural basis for the product specificity of histone lysine methyltransferases. Mol Cell 12, 177-185.
Zinoviev, V. V., Evdokimov, A. A., Malygin, E. G., Schlagman, S. L. and Hattman, S., Bacteriophage T4 Dam
DNA-(N6-adenine)-methyltransferase. Processivity and orientation to the methylation target, J. Biol.
Chem. 278 (2003), pp. 7829-7833.
Zou, X., Sun, Y., and Kuntz, I. D. (1999). Inclusion of Solvation in Ligand Binding Free Energy
Calculations Using the Generalized-Born Model. J Amer Chem Soc 121, 8033-8043.

1. A method for identifying a compound capable of modifying a Dam enzyme activity comprising:
a. providing a three-dimensional structure of a Dam enzyme or a Dam enzyme complex;
b. providing a modifier candidate structure;
c. determining an interaction energy value from a simulated docking interaction involving the modifier candidate structure and the Dam enzyme structure or Dam enzyme complex structure; and
d. assessing the interaction energy value;
thereby identifying the compound capable of modifying the Dam enzyme activity.

2. The method of claim 1 wherein said modifying comprises inhibiting the Dam enzyme activity.

3. The method of claim 1 further comprising:
e. comparing the determined energy value with a reference value.

4. The method of claim 1 wherein the Dam enzyme is from a bacteriophage T4 or a bacterium.

5. The method of claim 4 wherein the Dam enzyme is from E. coli.

6. The method of claim 4 wherein the three-dimensional structure of the Dam enzyme complex is given by the atomic structure coordinates of FIG. 37.

7. The method of claim 1 wherein the simulated docking interaction of step c occurs at a specified docking site.

8. The method of claim 7 wherein the docking site is constrained to a pocket between the catalytic and DNA binding domains and/or the methyl donor binding site.

9. The method of claim 8 wherein the pocket is located between the catalytic and DNA binding domains as defined by the glycerol binding site bounded by residues Trp10-Ala11 and Leu122-Cys123.

10. The method of claim 8 wherein the active site comprises the Asp-Pro-Pro-Tyr motif (SEQ ID NO: 1) of a AdoHcy binding site.

11. A method for identifying a compound capable of inhibiting a Dam enzyme comprising:
a. providing a three-dimensional structure of the Dam enzyme complex, wherein the Dam enzyme has a pocket site and an active site;
b. selecting computationally an inhibitor candidate of the Dam enzyme by calculating an interaction energy value for a simulated docking interaction involving the inhibitor candidate and the pocket site and/or active site of the Dam enzyme complex;
thereby identifying the compound capable of inhibiting a Dam enzyme.

12. The method of claim 11 wherein the Dam enzyme is from E. coli.

13. A method of inhibiting DNA methylation in a bacterium comprising:
a. providing said compound identified by the method of claim 1;
b. contacting the bacterium with said compound provided in step a so as to inhibit DNA methylation;
thereby inhibiting DNA methylation in said bacterium.

14. The method of claim 13 wherein said bacterium contains a Dam methylase.

15. A method of inhibiting DNA methylation by Dam in an organism comprising contacting the organism with a compound selected from the group consisting of NCI-DTP Diversity Set compound number 659390: 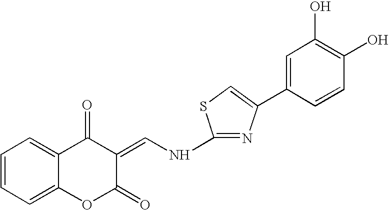
compound number 658343: 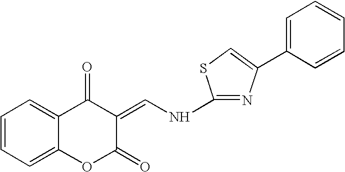
compound number 657589: 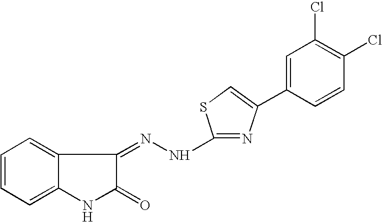
and a compound Dam-iZ1 of structural formula: 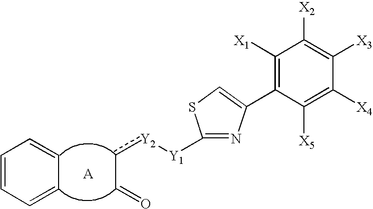
wherein A is a non-aromatic 5 or 6 member ring and wherein one or more of the ring members of A can be C, N, O or S;
wherein each of X₁-X₅ is independently selected from the group consisting of H, halide, OH, OCH₃, alkyl and alkylhalide; Y₁ is NH, CH or CH₂; Y₂ is N, NH, CH or CH₂;

16. The method of claim 15 wherein the organism is a bacterium.

17. The method of claim 16 wherein the bacterium is E. coli.

18. The method of claim 15 wherein the DNA methylation is inhibited by inhibition of a Dam methylase.

19. The method of claim 18 wherein the compound is selected from the group consisting of NCI-DTP Diversity Set compound numbers 659390, 658343 and 657589.

20. A method of treating a host suspected of infection with a pathogenic bacterium comprising administering to the host a compound selected from the group consisting of the compound identified by claim 1, NCI-DTP Diversity Set compound numbers 659390, 658343, 657589, Dam-iZ1, and Dam iZ2; in an amount sufficient to inhibit methylation of DNA within the bacterium.

21. The method of claim 20 wherein said treating reduces a virulence parameter of said bacterium.

22. The method of claim 20 wherein the host is a mammal.

23. The method of claim 20 wherein the host is not a human.

24. The method of claim 20 wherein the host is a human.

25. The method of claim 20 wherein the pathogenic bacterium is selected from the group consisting of Escherichia coli, enteropathogenic Escherichia coli, Salmonella typhimurium, Neisseria meningitidis, Yersinia pseudotuberculosis, Vibrio cholerae, Pasteurella multocida, Haemophilus influenzae and Yersinia enterocolitica.

26. The method of claim 20 wherein said compound further comprises a pharmaceutical formulation.

27. A method of treating a host suspected of infection with a pathogenic bacterium comprising administering to the host a compound capable of inhibiting a Dam methylase.

28. The method of claim 27 wherein the bacterium is a Gram-negative bacterium.

29. A composition comprising compound Dam-iZ1.

30. The composition of claim 29 excepting NCI-DTP Diversity Set compound numbers 659390, 658343, 657589.

31. A method of reducing a virulence parameter of a bacterium comprising contacting said bacterium with a compound capable of inhibiting a Dam methylase

32. The method of claim 1 further comprising measuring an in vitro or in vivo activity of the compound capable of modifying said Dam enzyme activity.

33. The method of claim 32 wherein the measuring of the in vitro activity comprises a biochemical assay or a bacterial assay.

34. A method of treating a host suspected of infection with a pathogenic bacterium comprising administering to the host a compound capable of modification of pathogenesis by inhibiting a methylase.

35. The method of claim 34 wherein the methylase is a Dam methylase.

36. The method of claim 34 wherein the modification of pathogenesis involves a modification of virulence.

37. The method of claim 36 wherein the modification of virulence is without a substantial effect on bacterial cell division.

38. A crystal of Escherichia coli Dam.

39. A crystal of an Escherichia coli Dam complex.

40. The crystal of claim 39 wherein the complex comprises E. coli Dam and cognate DNA.

41. The crystal of claim 39 wherein the complex comprises E. coli Dam and noncognate DNA.

42. The crystal of claim 39 wherein the complex further comprises a cofactor or cofactor analog.

43. The crystal of claim 42 wherein the cofactor or cofactor analog is selected from the group consisting of AdoMet, AdoHcy, and sinefungin.

44. The crystal of claim 43 having a set of atomic structure coordinates of FIG. 37.

45. A data representation of the crystal of claim 39.


SEQUENCE LISTING (45 sequences)

SEQ ID NO: 1 - protein, 4 aa, Escherichia coli
DPPY

SEQ ID NO: 2 - DNA, 12 bp, Artificial Sequence
  Description of Artificial Sequence Synthetic oligonucleotide
acaggatcctgt

SEQ ID NO: 3 - DNA, 13 bp, Artificial Sequence
  Description of Artificial Sequence Synthetic oligonucleotide
accatgatctgac

SEQ ID NO: 4 - DNA, 13 bp, Artificial Sequence
  Description of Artificial Sequence Synthetic oligonucleotide
tgtcagatcatgg

SEQ ID NO: 5 - DNA, 15 bp, Artificial Sequence
  Description of Artificial Sequence Synthetic oligonucleotide
tcacaggatcctgtg

SEQ ID NO: 6 - DNA, 20 bp, Artificial Sequence
  Description of Artificial Sequence Synthetic oligonucleotide
gcgacagtgatcggcctgtc

SEQ ID NO: 7 - DNA, 20 bp, Artificial Sequence
  Description of Artificial Sequence Synthetic oligonucleotide
gacaggccgatcactgtcgc

SEQ ID NO: 8 - DNA, 12 bp, Artificial Sequence
  Description of Artificial Sequence Synthetic oligonucleotide
tctagatctaga

SEQ ID NO: 9 - DNA, 14 bp, Artificial Sequence
  Description of Artificial Sequence Synthetic oligonucleotide
tagacgatctttta

SEQ ID NO: 10 - DNA, 14 bp, Artificial Sequence
  Description of Artificial Sequence Synthetic oligonucleotide
tagacgatcttttt

SEQ ID NO: 11 - DNA, 14 bp, Artificial Sequence
  Description of Artificial Sequence Synthetic oligonucleotide
ttaacgatctttta

SEQ ID NO: 12 - DNA, 14 bp, Artificial Sequence
  Description of Artificial Sequence Synthetic oligonucleotide
aaaacgatcaatat

SEQ ID NO: 13 - DNA, 14 bp, Artificial Sequence
  Description of Artificial Sequence Synthetic oligonucleotide
ataacgatctttta

SEQ ID NO: 14 - DNA, 14 bp, Artificial Sequence
  Description of Artificial Sequence Synthetic oligonucleotide
atagcgatctttta

SEQ ID NO: 15 - DNA, 14 bp, Artificial Sequence
  Description of Artificial Sequence Synthetic oligonucleotide
taaaagatcgttta

SEQ ID NO: 16 - DNA, 14 bp, Artificial Sequence
  Description of Artificial Sequence Synthetic oligonucleotide
gtaaagatcgttaa

SEQ ID NO: 17 - DNA, 14 bp, Artificial Sequence
  Description of Artificial Sequence Synthetic oligonucleotide
caaaagatcgtcaa

SEQ ID NO: 18 - DNA, 27 bp, Artificial Sequence
  Description of Artificial Sequence Synthetic oligonucleotide
ttatcgatcgtttatatcgatcgataa

SEQ ID NO: 19 - DNA, 14 bp, Artificial Sequence
  Description of Artificial Sequence Synthetic oligonucleotide
aaaaagatcgtgca

SEQ ID NO: 20 - DNA, 14 bp, Artificial Sequence
  Description of Artificial Sequence Synthetic oligonucleotide
ttgaagatcgttaa

SEQ ID NO: 21 - DNA, 14 bp, Artificial Sequence
  Description of Artificial Sequence Synthetic oligonucleotide
taaaagatcgaagt

SEQ ID NO: 22 - protein, 22 aa, Enterobacteria phage T4
GFSNMIRINDKGNFTTPFGKRT

SEQ ID NO: 23 - protein, 22 aa, Escherichia coli
GYNGLCRYNLRGEFNVPFGRYK

SEQ ID NO: 24 - protein, 22 aa, Escherichia coli
CFNGMIRFNSKGGFNVPFCKKP

SEQ ID NO: 25 - protein, 22 aa, Streptococcus pneumoniae
NFNGLYRVNSKNQFNVPYGRYN

SEQ ID NO: 26 - protein, 22 aa, Salmonella typhimurium
GYNGLCRYNLRGEFNVPFGRYK

SEQ ID NO: 27 - protein, 22 aa, Serratia marcescens
CYNGLCRYNLRGEFNVPFGRYK

SEQ ID NO: 28 - protein, 22 aa, Yersinia pestis
CYNGLCRYNLSGEFNVPFGRYK

SEQ ID NO: 29 - protein, 22 aa, Vibrio cholerae
GFNGLCRYNKKGGFNVPFGSYK

SEQ ID NO: 30 - DNA, 16 bp, Artificial Sequence
  Description of Artificial Sequence Synthetic oligonucleotide
tcacaggatcctgtga

SEQ ID NO: 31 - protein, 19 aa, Escherichia coli
NRAFLKWAGGKYPLLDDIK

SEQ ID NO: 32 - protein, 19 aa, Enterobacteria phage T4
MLGAIAYTGNKQSLLPELK

SEQ ID NO: 33 - DNA, 20 bp, Artificial Sequence
  Description of Artificial Sequence Synthetic oligonucleotide
atttagacgatcttttatgc

SEQ ID NO: 34 - DNA, 16 bp, Artificial Sequence
  Description of Artificial Sequence Synthetic oligonucleotide
ttaaaagatcgtttaa

SEQ ID NO: 35 - DNA, 18 bp, Artificial Sequence
  Description of Artificial Sequence Synthetic oligonucleotide
aacgatcttttaacccac

SEQ ID NO: 36 - DNA, 18 bp, Artificial Sequence
  Description of Artificial Sequence Synthetic oligonucleotide
gacgatcttttatgctgt

SEQ ID NO: 37 - protein, 259 aa, Enterobacteria phage T4
MLGAIAYTGNKQSLLPELKSHFPKYNRFVDLFCGGLSVSLNVNGPVLANDIQEPIIEMYKRLINVSWDDVLKVIKQYKLSKTSKEEFLKLREDYNKTRDPLLLYVLHFHGFSNMIRINDKGNFTTPFGKRTINKNSEKQYNHFKQNCDKIIFSSLHFKDVKILDGDFVYVDPPYLITVADYNKFWSEDEEKDLLNLLDSLNDRGIKFGQSNVLEHHGKENTLLKEWSKKYNVKHLNKKYVFNIYHSKEKNGTDEVYIFN

SEQ ID NO: 38 - protein, 274 aa, Escherichia coli
RAFLKWAGGKYPLLDDIKRHLPKGECLVEPFVGAGSVFLNTDFSRYILADINSDLISLYNIVKMRTDEYVQAARELFVPETNCAEVYYQFREEFNKSQDPFRRAVLFLYLNRYGYNGLCRYNLRGEFNVPFGRYKKPYFPEAELYHFAEKAQNAFFYCESYADSMARADDASVVYCDPPYAPLSATANFTAYHTNSFTLEQQAHLAEIAEGLVERHIPVLISNHDTMLTREWYQRAKLHVVKVRRSISSNGGTRKKVDELLALYKPGVVSPAKK

SEQ ID NO: 39 - protein, 282 aa, Escherichia coli
FVPPIKSQGIKTKLVPCIKRIVPKNFNGVWVEPFMGTGVVAFNVAPKDALLCDTNPHLISFYNALKNKDITGDLVKDFLYREGEKLLLSNGEYYYEVRERFNNYKEPLDFLFLNRSCFNGMIRFNSKGGFNVPFCKKPNRFAQAYITKISNQVDRISEIISKGNYTFLCQSFEKTIGMVNRDDVVYCDPPYIGRHVDYFNSWGERDERLLFETLSSLNATFITSTWHHNDYRENKYVRDLWSSFRILTKEHFYHVGASEKNRSPMVEALITNIAKDIIDHIE

SEQ ID NO: 40 - protein, 274 aa, Streptococcus pneumoniae
LQPFTKWTGGKRQLLPVIRELIPKTYNRYFEPFVGGGALFFDLAPKDAVINDFNAELINCYQQIKDNPQELIEILKVHQEYNSKEYYLDLRSADRDERIDMMSEVQRAARILYMLRVNFNGLYRVNSKNQFNVPYGRYKNPKIVDEELISAISVYINNNQLEIKVGDFEKAIVDVRTGDFVYFDPPYIPLSETSAFTSYTHEGFSFADQVRLRDAFKRLSDTGAYVMLSNSSSALVEELYKDFNIHYVEATRTNGAKSSSRGKISEIIVTNYEK

SEQ ID NO: 41 - protein, 185 aa, Escherichia coli
KNRAFLKWAGGKYPLLDDIKRHLPAGECLVEPFVGAGSVFLNTDFSRYILADINSDLISLYNIVKMRTDEYVAAARELFVPETNCAEVYYQFREEFNKSQDPFRRAVLFLYLNRYGYNGLCRYNLRGEFNVPFGRYKKPYFPEAELYHFAEKAQNAFFYCESYADSMARADDSSVVYCDPPYAPA

SEQ ID NO: 42 - protein, 48 aa, Escherichia coli
AAFTLEQQAHLAEIAEGLVERHIPVLISNHDTALTRAWYQRAKLHVVA

SEQ ID NO: 43 - protein, 11 aa, Escherichia coli
AVDELLALYKP

SEQ ID NO: 44 - protein, 185 aa, Escherichia coli
KNRAFLKWAGGKYPLLDDIKRHLPKGECLVEPFVGAGSVFLNTDFSRYILADINSDLISLYNIVKMRTDEYVQAARELFVPETNCAEVYYQFREEFNKSQDPFRRAVLFLYLNRYGYNGLCRYNLRGEFNVPFGRYKKPYFPEAELYHFAEKAQNAFFYCESYADSMARADDSSVVYCDPPYAPA

SEQ ID NO: 45 - protein, 49 aa, Escherichia coli
ASFTLEQQAHLAEIAEGLVERHIPVLISNHDTALTRAWYQRAKLHVVAV